(19) United States
(12) Patent Application Publication
(10) Pub. No.: US 2024/0294585 A1
(43) Pub. Date: Sep. 5, 2024

(54) MODULAR BINDING PROTEINS FOR EXTRACELLULAR VESICLES AND USES THEREOF

(71) Applicants: Colin David Gottlieb, San Francisco, CA (US); Mantra Bio, Inc., San Francisco, CA (US)

(72) Inventor: Colin David Gottlieb, San Francisco, CA (US)

(21) Appl. No.: 17/996,066

(22) PCT Filed: Apr. 13, 2021
(86) PCT No.: PCT/US2021/027157

§ 371 Date: Oct. 12, 2022

Related U.S. Application Data
(60) Provisional application No. 63/009,392, filed on Apr. 13, 2020.

(51) Int. Cl. C07K 14/47; A61K 38/00; C07K 14/705

(52) U.S. Cl. CPC C07K 14/47; C07K 14/705; A61K 38/00; C07K 2319/03

(57) ABSTRACT

Disclosed herein are EVs and/or exosomes engineered with targeting moieties. These targeting moieties can be used to target payloads to target cells in a subject. These payloads can be carried in EVs, exosomes, liposomes or other delivery vehicles. The engineered EVs and/or exosomes can also display molecules capable of disrupting EV and/or exosome communication between cells in disease states.

This subject patent application claims the benefit under 35 U.S.C. § 119(e) to U.S. Provisional Application No. 63/009,392, filed Apr. 13, 2020, the contents of which are herein incorporated by reference in their entireties into the present patent application for all purposes.

Throughout this application various publications are referenced. All publications, gene transcript identifiers, patents, and patent applications mentioned in this specification are herein incorporated by reference to the same extent as if each individual publication, gene transcript identifiers, patent, or patent application was specifically and individually indicated to be incorporated by reference.

### BACKGROUND OF THE INVENTION

Intercellular communication is needed for cell development and the maintenance of homeostasis in multicellular organisms. These communications between cells can be localized or distant. Distant intercellular communication is facilitated by molecules like hormones that send signals through circulatory system to other parts of the body. Another case of distant intercellular communication also occurs through extracellular vesicles (EVs), which are membrane-based structures. EVs serve as vehicles to carry different types of cellular cargo—such as lipids, proteins, receptors and effector molecules—to the recipient cells.

EVs include microvesicles or ectosomes, apoptotic bodies, and exosomes. Microvesicles or ectosomes are vesicles assembled at and released from the plasma membrane. They may be formed through the outward budding and fission from plasma membranes. Exosomes originate from the endosome compartment in cells and have a smaller size, ranging from 30 to 200 nm. Exosomes arise as small vesicles within larger membrane structures in the endosome within a cell. The endosome, also called the multivesicular body, can be exocytized, with ensuing release to the extracellular space of their vesicles as exosomes. It has been demonstrated that almost all living cells can secrete exosomes, and exosomes widely exist in various body fluids. EVs carry protein, mRNA, miRNA, tRNA, yRNA, DNA, lipids and other ingredients derived from the secreting cells, and protect them from degradation by the external environment, and are beneficial to their biological function of active ingredients. EVs can be internalized by receptor cells through giant pinocytosis or macropinocytosis, fusion, phagocytosis, raft-mediated endocytosis, lipid rafts, receptor-mediated endocytosis, adhesion, antigen recognition, juxtacrine signaling, and soluble signaling. The internalized EVs can regulate and control multiple biological function of receptor cells and play an important role in intercellular communication.

Both exosomes and microvesicles are known to facilitate intercellular communication processes between cells in close proximity as well as distant cells. Tumor cells have also been shown to exploit EVs to contribute to their progression by inactivating T lymphocytes or natural killer cells as well as promoting differentiation of regulatory T lymphocytes to suppress immune reactions. Moreover, several pathogenic proteins such as prions and β-amyloid peptides have also been reported to exploit exosomes in order to propagate to other cells.

There remains a need for producing EVs having a desired targeting moiety(ies) (such as a peptide or protein that targets a cell, tissue, organ or a specific cell type) on its surface and to be able to change the desired targeting moiety(ies) on the EV quickly and efficiently without affecting the luminal content or membrane composition of the EV. Currently, indication-specific complex biological therapeutic EVs must be generated through engineering individual producer cell lines and characterizing the resulting EVs. Changing the desired targeting moiety(ies) for the EV requires a different producer cell line for every change. This is a time consuming and slow process. In addition, each new producer cell line may produce EVs having different luminal content and/or membrane composition, introducing uncertainty as to the therapeutic suitability or quality of the EV bearing the desired targeting moiety(ies). The discovery herein addresses the problem of producing EVs with different targeting agents while maintaining, e.g., the same luminal content and membrane composition.

### SUMMARY OF THE INVENTION

The invention provides EVs and/or exosomes that are engineered to display desired targeting moiety(ies) and production methods thereof. The desired targeting moiety(ies) can be a polypeptide (such as, a targeting protein or affinity peptide), lipid, carbohydrate, nucleic acid, nucleic acid analog (such as, antisense oligonucleotide (ASO, 2′-O-methyl (OMe), 2′-fluoro (F), and 2′-O-methoxyethyl (MOE) RNA, locked nucleic acid (LNA), constrained ethyl (cEt), phosphorodiamidate morpholinos (PMOs), phosphorothioate, and peptide nucleic acid (PNA)), ligand, aptamer, small molecules, chemical compound or macromolecules. Polypeptides can include, for example, polypeptides that target an EV and/or exosome to a desired location. EVs and/or exosomes may be modified with certain desired molecules. Such molecules may be displayed on the surface of the EVs and/or exosomes. Desired molecules on the EVs and/or exosomes can be used to enrich EVs and/or exosomes, traffic the EV and/or exosomes in the body to a desired site, recognize/bind to target cells, induce a therapeutic effect, and/or fuse the EVs and/or exosomes to a target cell.

Novel and innovative production methods are described. This involves the use of isopeptide domains and isopeptide tags adapted from proteins that spontaneously form isopeptide bonds. The isopeptide domain and complementary isopeptide tag can each be separately fused to proteins of interest so that the two proteins can be joined together through the formation of an isopeptide bond. Isopeptide domains and complementary isopeptide tags can be used to engineer EVs and/or exosomes to display polypeptides of interest. The displayed polypeptides can impart to the EV and/or exosome a desired property, function, and/or characteristic. For example, an isopeptide domain can be displayed by a vesicle localization moiety on an EV or exosome. A targeting moiety (e.g., a targeting polypeptide, affinity peptide, anti-sense oligonucleotide, ligand, aptamer, etc.) can be fused by an isopeptide bond to the vesicle localization moiety through the isopeptide domain by making a desired molecule with an isopeptide tag. The isopeptide domain and the isopeptide tag can associate with one another and form an isopeptide bond linking the two and the vesicle localization moiety and desired molecule (the latter also referred to herein as targeting agent or targeting moiety) together.

Examples of proteins that can be engineered to an EV and/or exosome include, for example, proteins that can traffic and/or target an EV and/or exosome to a desired location in a subject. For example, peptides or scFvs can be engineered onto an EV and/or exosome that specifically bind to a protein target on a cell.

Engineered EVs or exosomes can carry a payload that can be any molecule that can cause a change (e.g., phenotypic or genotypic) in the target cell. Payloads can include, for example, polypeptides (e.g., biologics or membrane associate proteins), small molecules (e.g., drugs), RNA (e.g., siRNA, miRNA, antisense RNA, lncRNA), DNA (e.g., transgenes, expression vectors, DNA constructs), nucleic acid analog (such as, antisense oligonucleotide (ASO, 2′-O-methyl (OMe), 2′-fluoro (F), and 2′-O-methoxyethyl (MOE) RNA, locked nucleic acid (LNA), constrained ethyl (cEt), phosphorodiamidate morpholinos (PMOs), phosphorothioate, and peptide nucleic acid (PNA)), viral vectors (e.g., retroviruses, lentiviruses, adenoviruses, adeno-associated viruses, recombinant viruses and hybrid viruses), oncolytic viruses (e.g., modified herpesvirus (such as, herpes simplex virus-1 (e.g., T-VEC; Imlygic®)), modified adenovirus, modified vaccinia virus, modified reovirus, modified measles virus, modified polio/rhinovirus, modified vesicular stomatitis virus, modified coxsackievirus and modified retrovirus), genome editing systems (e.g., CRISPR/cas9), or a reporter (e.g., GFP or luciferase) or a combination thereof. In some embodiments, engineered EVs and/or exosomes can be used to systemically, intravitreally, or intranasally administer drugs targeting cells in a desired location as these EVs and/or exosomes can traffic to and interact with target cells at the desired location.

Engineered EVs of the invention (e.g., exosomes) may target cells or tissues in a subject and deliver appropriate therapeutic or diagnostic payloads at the target site. The engineered EVs and/or exosomes of the invention can traffic to the desired location in a subject and deliver the therapeutic or diagnostic payload to the target site.

Alternatively, naturally occurring exosomes in diseased subjects have been linked to disease and disease progression. In these situations, engineered EVs of the invention can be used to display markers that are complementary to the diseased EVs and/or exosomes, or the engineered EVs of the invention can display markers complementary to the markers on the target cells that interact with the diseased EVs and/or exosomes. In this way, the interaction and progression of disease through EV and/or exosome trafficking can be inhibited and/or blocked by competition for complementary marker binding. Alternatively, antibodies that bind to a marker (or binding pair protein) and/or EV or exosome markers can be used to inhibit and/or block the interaction between diseased EVs and/or exosomes and target cells.

### BRIEF DESCRIPTION OF THE FIGURES

FIG. 1 is a schematic of a construct map showing the arrangement in an isopeptide domain-vesicle localization moiety (VLM) fusion protein comprising a signal sequence (SS), epitope sequence, Isopeptide(1) domain (Isopeptide-1) and IGSF8 vesicle localization moiety and an expression plasmid for production of the fusion protein. The fusion protein additionally comprises a peptide linker which joins Isopeptide-1 and IGSF. Note as in all fusion proteins with signal sequence expressed in cell culture (such mammalian cells or human cells), the mature fusion protein lacks the signal sequence of the nascent protein, and consequently, an exosome obtained from such a cell culture or cells comprising the fusion protein displays the N-terminally localized isopeptide domain (such as in this case, Isopeptide-1) or isopeptide tag in relation to the vesicle localization moiety (such as IGSF8 in the current case) external to the exosome. Nucleic acid coding sequence and amino acid sequence of the fusion protein are provided in SEQ ID NO: 197 and 198, respectively.

FIG. 2 is a schematic of a construct map showing the arrangement in an isopeptide domain-VLM fusion protein comprising a signal sequence (SS), epitope sequence, Isopeptide(2) domain (Isopeptide-2) and IGSF8 vesicle localization moiety and an expression plasmid for production of the fusion protein. The fusion protein additionally comprises a peptide linker which joins Isopeptide-2 and IGSF. Nucleic acid coding sequence and amino acid sequence of the fusion protein are provided in SEQ ID NO: 199 and 200, respectively.

FIG. 3 is a schematic of a construct map showing the arrangement in an isopeptide domain-VLM fusion protein comprising a signal sequence (SS), epitope sequence, Isopeptide(1) domain (Isopeptide-1), isopeptide(2) domain (Isopeptide-2) and IGSF8 vesicle localization moiety and an expression plasmid for production of the fusion protein. The fusion protein additionally comprises two peptide linkers, one joining Isopeptide-2 and IGSF and another joining Isopeptide-1 and Isopeptide-2. Nucleic acid coding sequence and amino acid sequence of the fusion protein are provided in SEQ ID NO: 201 and 202, respectively.

FIG. 4 is a schematic of a construct map showing the arrangement in an isopeptide domain-VLM fusion protein comprising a signal sequence (SS), epitope sequence, Isopeptide(3) domain (Isopeptide-3), Isopeptide(1) domain (Isopeptide-1), isopeptide(2) domain (Isopeptide-2) and IGSF8 vesicle localization moiety and an expression plasmid for production of the fusion protein. The fusion protein additionally comprises three peptide linkers, one joining Isopeptide-2 and IGSF, another joining Isopeptide-1 and Isopeptide-2, and a third joining Isopeptide-3 and Isopeptide-1. Nucleic acid coding sequence and amino acid sequence of the fusion protein are provided in SEQ ID NO: 203 and 204, respectively.

FIG. 5 is a bar graph showing construct expression levels of isopeptide(1)-IGSF8, isopeptide(2)-IGSF8, isopeptide(1) and (2)-IGSF8 (DiCatcher-IGSF8), isopeptide(3), (1), and (2)-IGSF8 (TriCatcher-IGSF8), and mock transfected (control) on an EV surface. The EVs are stained with a fluorophore-conjugated antibody that recognizes the epitope sequence (Flag) present in the isopeptide domain-IGSF8 fusion proteins. The bar graph indicates the % of EVs that are detectably stained with the antibody (Left vertical axis) and the median intensity of the antibody signal for an exosome positive for the antibody (right vertical axis).

FIG. 6 is a schematic of a construct map showing the arrangement in an isopeptide tag-VLM fusion protein comprising IGSF8 joined to an epitope sequence and isopeptide-1 tag via two linkers. Nucleic acid coding sequence and amino acid sequence of the fusion protein are provided in SEQ ID NO: 215 and 216, respectively.

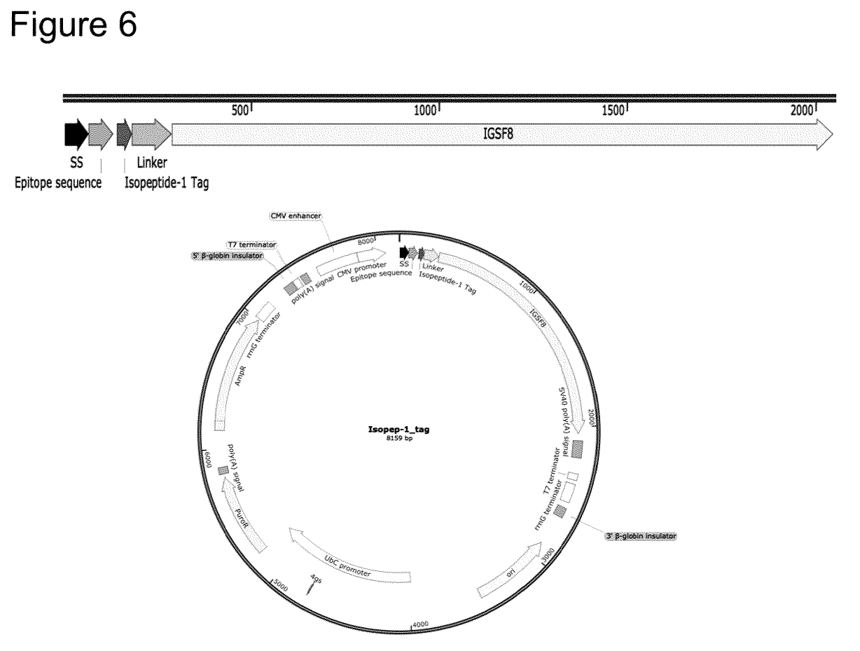

FIG. 7 is a schematic of a construct map showing the arrangement in an isopeptide tag-VLM fusion protein comprising IGSF8 joined to an epitope sequence and isopeptide-2 tag via two linkers. Nucleic acid coding sequence and amino acid sequence of the fusion protein are provided in SEQ ID NO: 217 and 218, respectively.

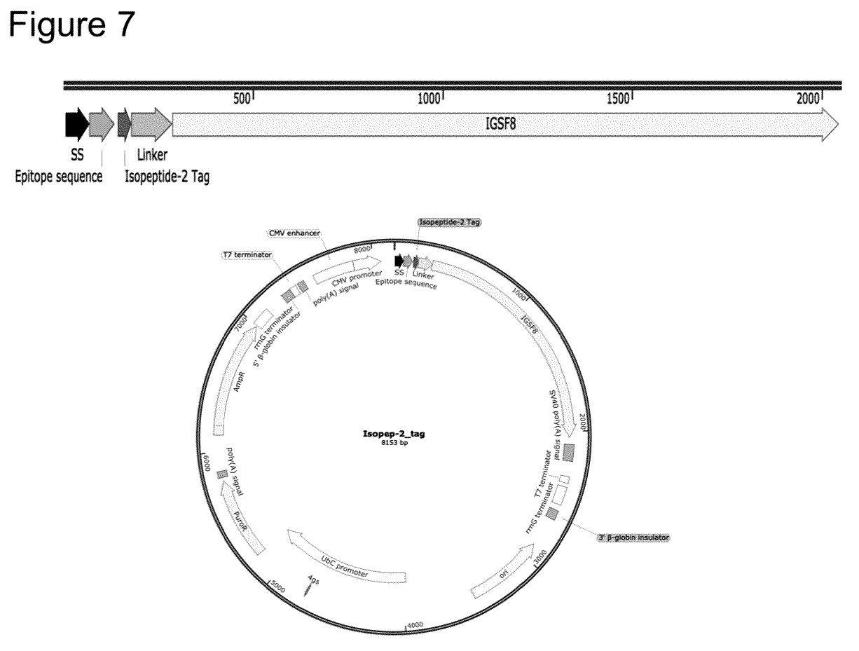

FIG. 8 is a schematic of a construct map showing the arrangement in an isopeptide tag-VLM fusion protein comprising IGSF8 joined to an epitope sequence, isopeptide-1 tag and isopeptide-2 tag via two linkers. Nucleic acid coding sequence and amino acid sequence of the fusion protein are provided in SEQ ID NO: 219 and 220, respectively.

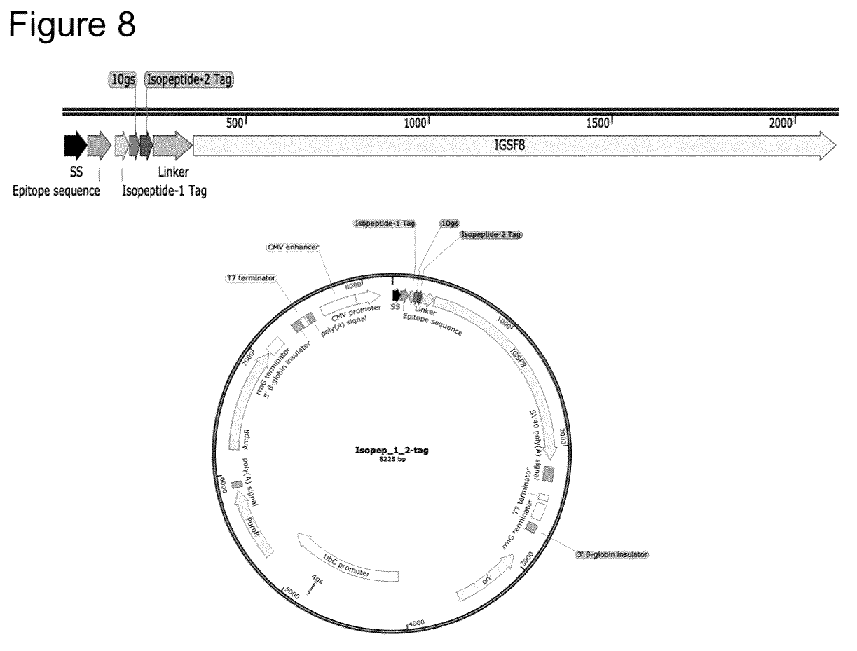

FIG. 9 is a schematic of a construct map showing the arrangement in an isopeptide tag-VLM fusion protein comprising IGSF8 joined to an epitope sequence, isopeptide-1 tag, isopeptide-2 tag and isopeptide-3 tag via three or more linkers. Nucleic acid coding sequence and amino acid sequence of the fusion protein are provided in SEQ ID NO: 221 and 222, respectively.

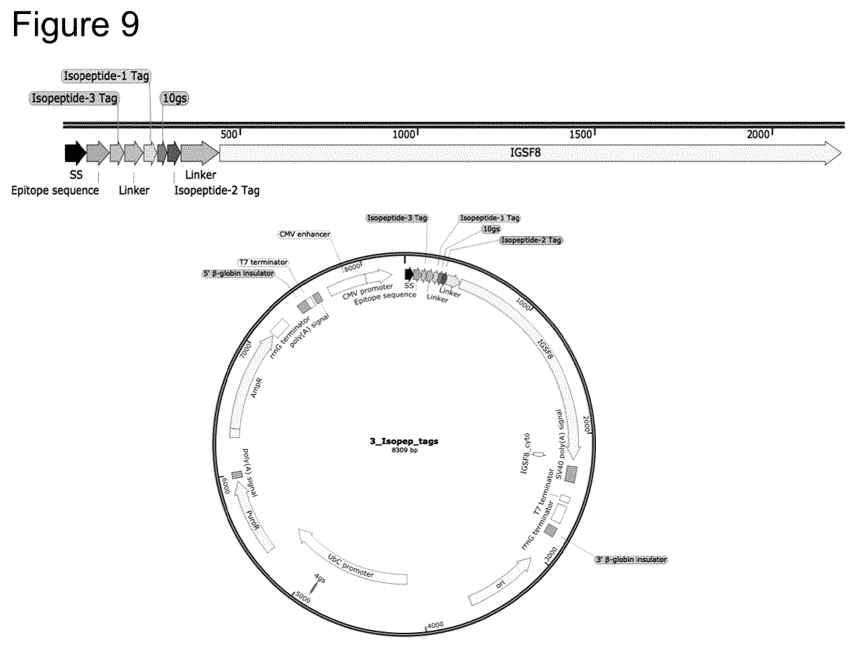

FIG. 10 is a consensus sequence table showing the sequence alignment of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, and 60.

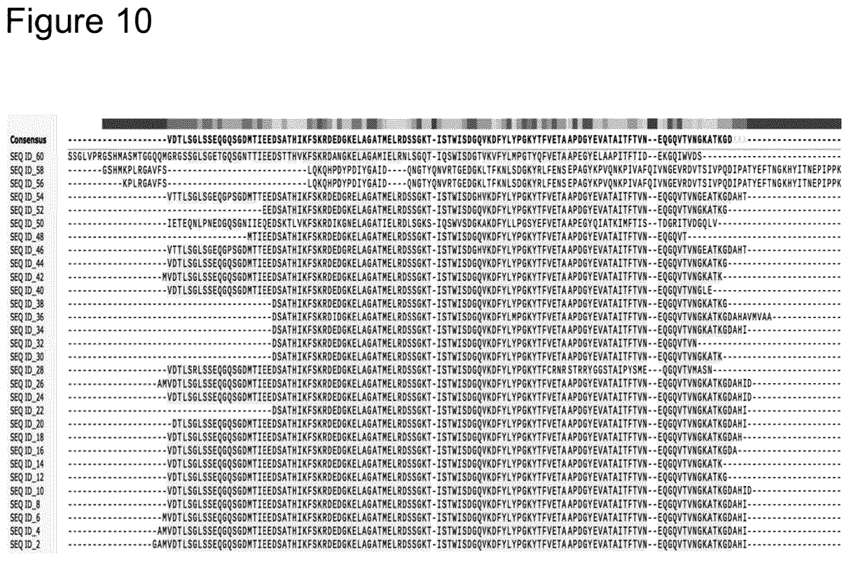

FIG. 11 is Cy3 fluorescence at about 98 kDa range in a SDS-PAGE gel dependent on EVs modified with Tricatcher-1-IGSF8 (of FIG. 4; SEQ ID NO: 204) and Cy3-labeled Isopeptide(1) tag-antisense oligonucleotide (ASO) conjugate demonstrating formation of a covalent bond between isopeptide domain present on Tricatcher-1-IGSF8 EV surface and isopeptide tag of the Isopeptide(1) tag-ASO conjugate consistent with formation of an isopeptide bond.

FIG. 12 is a combined capillary electrophoresis-Western blot analysis (Jess™ Simple Western system) of modified EVs comprising Isopeptide(2) domain-IGSF8 VLM (of FIG. 2; SEQ ID NO: 200; “Iso-2”), Isopeptide(1)_Isopeptide(2) domains-IGSF VLM (of FIG. 3; SEQ ID NO: 202; “Di”) or Tricatcher-1-IGSF8 VLM (of FIG. 4; SEQ ID NO: 204; “Tri”) fusion protein and unmodified EV control (“Un”) incubated with myc-epitope tagged Isopeptide(2) tag (Isopeptide-2 tag; SEQ ID NO: 240 or 242) fusion peptide and detected with anti-myc primary antibody for Isopeptide-2 tag fusion peptide or anti-Flag epitope tag primary antibody for IGSF8 fusion protein. Note that the presence of Isopeptide(2) domain in “Iso-2,” “Di” and “Tri” IGSF VLM fusion protein results in covalent attachment of Isopeptide(2) tag fusion peptide, consistent with the formation of an isopeptide bond. No covalent attachment of the Isopeptide(2) tag fusion peptide to a high molecular weight protein around the location of the IGSF8 VLM fusion protein is seen for the unmodified EV control samples which are not modified with IGSF8 VLM fusion protein.

FIG. 13 is a combined capillary electrophoresis-Western blot analysis (Jess™ Simple Western system) of modified EVs comprising “Iso-2”, “Di” or “Tri” VLM fusion protein as in FIG. 12 (above) and unmodified EV control (“Un”) incubated with S-tag labeled Isopeptide(1) tag fusion peptide (SEQ ID NO: 236 or 238), V5-epitope tagged Isopeptide(3) tag fusion peptide (SEQ ID NO: 244 or 246) or no fusion peptide control, followed by detection with anti-S-tag primary antibody, anti-V5-epitope tag primary antibody or anti-Flag epitope tag primary antibody, the latter to reveal the location of the Flag-epitope tagged VLM fusion protein. Covalent attachment due to isopeptide bond formation between isopeptide domain and isopeptide tag results in detection of the isopeptide tag fusion peptide in the high molecular weight range where the IGSF8 VLM fusion protein migrates.

FIG. 14 is a schematic of a construct map showing the arrangement in an isopeptide domain-VLM fusion protein comprising a signal sequence, epitope sequence, Isopeptide(1) domain, Isopeptide(3) domain and IGSF8 vesicle localization moiety, produced by expression vector 288. The fusion protein additionally comprises a peptide linker which joins Isopeptide-3 and IGSF and a peptide linker which joins Isopeptide-1 and Isopeptide-3. Nucleic acid coding sequence and amino acid sequence of the fusion protein are provided in SEQ ID NO: 205 and 206, respectively.

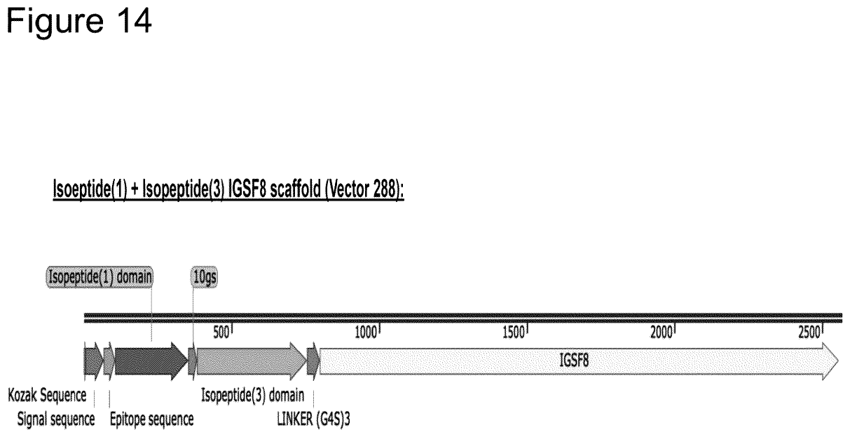

FIG. 15 is a schematic of a construct map showing the arrangement in an isopeptide domain-VLM fusion protein comprising a signal sequence, epitope sequence, Isopeptide(3) domain, Isopeptide(1) domain and IGSF8 vesicle localization moiety, produced by expression vector 289. The fusion protein additionally comprises a peptide linker which joins Isopeptide-1 and IGSF and a peptide linker which joins Isopeptide-3 and Isopeptide-1. Nucleic acid coding sequence and amino acid sequence of the fusion protein are provided in SEQ ID NO: 207 and 208, respectively.

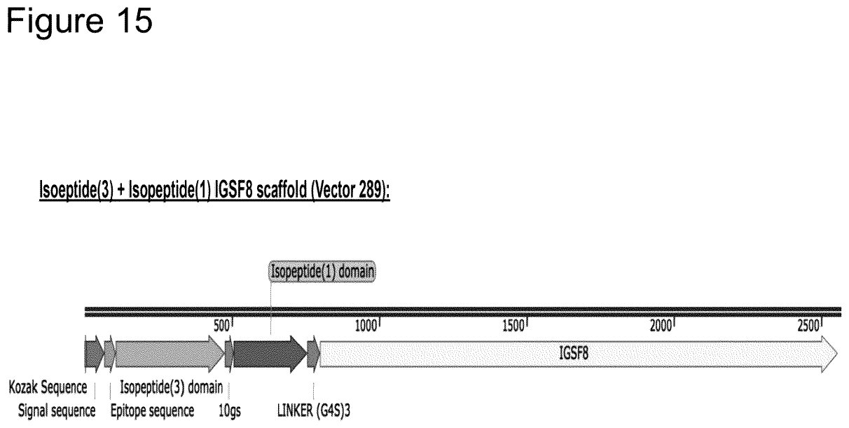

FIG. 16 is a schematic of a construct map showing the arrangement in an isopeptide domain-chimeric VLM fusion protein comprising a signal sequence (mouse Ig Kappa Signal Peptide), epitope sequence, Isopeptide(3) domain, Isopeptide(1) domain and a chimeric VLM comprising Lamp2 surface and transmembrane domains and IL3RA cytosolic domain (Lamp2-IL3RA chimeric VLM) in place of a Lamp2 cytosolic domain, produced by expression vector 290. The fusion protein additionally comprises a peptide linker which joins Isopeptide-1 and Lamp2-IL3RA chimeric VLM and a peptide linker which joins Isopeptide-3 and Isopeptide-1. Nucleic acid coding sequence and amino acid sequence of the fusion protein are provided in SEQ ID NO: 209 and 210, respectively.

FIG. 17 is a schematic of a construct map showing the arrangement in an isopeptide domain-chimeric VLM fusion protein comprising a signal sequence (mouse Ig Kappa Signal Peptide), epitope sequence, Isopeptide(3) domain, Isopeptide(1) domain and a chimeric VLM comprising Lamp2 surface and transmembrane domains and SELPL cytosolic domain (Lamp2-SELPL chimeric VLM) in place of a Lamp2 cytosolic domain, produced by expression vector 291. The fusion protein additionally comprises a peptide linker which joins Isopeptide-1 and Lamp2-SELPL chimeric VLM and a peptide linker which joins Isopeptide-3 and Isopeptide-1. Nucleic acid coding sequence and amino acid sequence of the fusion protein are provided in SEQ ID NO: 211 and 212, respectively.

FIG. 18 is a schematic of a construct map showing the arrangement in an isopeptide domain-chimeric VLM fusion protein comprising a signal sequence, epitope sequence, Isopeptide(3) domain, Isopeptide(1) domain and a chimeric VLM comprising Lamp2 surface and transmembrane domains and PTGFRN cytosolic domain (Lamp2-PTGFRN chimeric VLM) in place of a Lamp2 cytosolic domain, produced by expression vector 293. The fusion protein additionally comprises a peptide linker which joins Isopeptide-1 and Lamp2-PTGFRN chimeric VLM and a peptide linker which joins Isopeptide-3 and Isopeptide-1. Nucleic acid coding sequence and amino acid sequence of the fusion protein are provided in SEQ ID NO: 213 and 214, respectively.

FIG. 19 is a schematic of a construct map showing the arrangement in a targeting moiety fusion protein comprising a signal sequence, epitope sequence, 6A6 scFv, a second epitope tag and Isopeptide(1) tag (Isopeptide tag-1), produced by expression vector 251. The fusion protein additionally comprises one or more peptide linkers which join Isopeptide tag-1 at the C-terminus and 6A6 scFv. Nucleic acid coding sequence and amino acid sequence of the fusion protein are provided in SEQ ID NO: 223 and 224, respectively.

FIG. 20 is a schematic of a construct map showing the arrangement in a targeting moiety fusion protein comprising a signal sequence, Isopeptide tag-1, epitope sequence, 6A6 scFv and a second epitope tag, produced by expression vector 252. The fusion protein additionally comprises one or more peptide linkers which join Isopeptide tag-1 between the signal sequence and 1^{st} epitope tag to 6A6 scFv and one or more peptide linkers which join C-terminal 2^{nd} epitope tag to carboxyl end of the scFv. Nucleic acid coding sequence and amino acid sequence of the fusion protein are provided in SEQ ID NO: 227 and 228, respectively.

FIG. 21 is a schematic of a construct map showing the arrangement in a targeting moiety fusion protein comprising a signal sequence, epitope sequence, alaC scFv, a second epitope tag and Isopeptide tag-1, produced by expression vector 269. The fusion protein additionally comprises one or more peptide linkers which join C-terminal Isopeptide tag-1 to a 2^{nd} epitope tag, one or more peptide linkers which join the 2^{nd} epitope tag to carboxyl end of alaC scFv. Nucleic acid coding sequence and amino acid sequence of the fusion protein are provided in SEQ ID NO: 225 and 226, respectively

### DETAILED DESCRIPTION OF THE INVENTION

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described. References to exemplary nucleic acid and amino acid sequences and, when applicable their respective SEQ ID Nos, are provided in the Tables herein

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 μM, it is intended that the concentration be understood to be at least approximately or about 10 μM.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

As used herein, an “antibody” is defined to be a protein or polypeptides functionally defined as a binding protein and structurally defined as comprising an amino acid sequence that is recognized by one of skill as being derived from the variable region of an immunoglobulin. An antibody can consist of one or more polypeptides substantially encoded by immunoglobulin genes, fragments of immunoglobulin genes, hybrid immunoglobulin genes (made by combining the genetic information from different animals), or synthetic immunoglobulin genes. The recognized, native, immunoglobulin genes include the kappa, lambda, alpha, gamma, delta, epsilon and mu constant region genes, as well as myriad immunoglobulin variable region genes and multiple D-segments and J-segments. Light chains are classified as either kappa or lambda. Heavy chains are classified as gamma, mu, alpha, delta, or epsilon, which in turn define the immunoglobulin classes, IgG, IgM, IgA, IgD and IgE, respectively. Antibodies exist as intact immunoglobulins, as a number of well characterized fragments produced by digestion with various peptidases, or as a variety of fragments made by recombinant DNA technology. Antibodies can derive from many different species (e.g., rabbit, sheep, camel, human, or rodent, such as mouse or rat), or can be synthetic. Antibodies can be chimeric, humanized, or humaneered. Antibodies can be monoclonal or polyclonal, multiple or single chained, fragments or intact immunoglobulins.

As used herein, an “antibody fragment” is defined to be at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either W or Vu), camelid VHH domains, and multi-specific antibodies formed from antibody fragments. The term “scFv” is defined to be a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

As used herein, an “antigen” is defined to be a molecule that provokes an immune response. This immune response may involve either antibody production, or the activation of specific immunologically-competent cells, or both. The skilled artisan will understand that any macromolecule, including, but not limited to, virtually all proteins or peptides, including glycosylated polypeptides, phosphorylated polypeptides, and other post-translation modified polypeptides including polypeptides modified with lipids, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be synthesized or can be derived from a biological sample, or can be a macromolecule besides a polypeptide. Such a biological sample can include, but is not limited to a tissue sample, a tumor sample, a cell or a fluid with other biological components.

As used herein, a “complementary marker” is defined to be a marker on a target cell or in a body that interact with the markers on an EV and/or exosome. For example, complementary markers can interact with markers on the EV and/or exosome to traffic the EV and/or exosome to target, retain the EV and/or exosome at the target, or allow the EV and/or exosome to recognize a target cell.

As used herein, a “delivery vehicle” is defined to be an EV, an exosome, a microvesicle, an ectosome, a microparticle, an apoptotic body, a nanoparticle, an antibody, or other molecule that can carry a payload.

As used herein, an “effective amount” or “therapeutically effective amount” are used interchangeably, and defined to be an amount of a compound, formulation, material, or composition, as described herein effective to achieve a particular biological result.

As used herein, an “epitope” is defined to be the portion of an antigen capable of eliciting an immune response, or the portion of an antigen that binds to an antibody. Epitopes can be a protein sequence or subsequence that is recognized by an antibody.

As used herein, an “expression vector” and an “expression construct” are used interchangeably, and are both defined to be a plasmid, virus, or other nucleic acid designed for modulating protein expression in a cell. The vector or construct is used to introduce a gene into a host cell whereby the vector will interact with polymerases in the cell to express the protein encoded in the vector/construct. The expression vector and/or expression construct may exist in the cell extrachromosomally or integrate into the chromosome. When integrated into the chromosome the nucleic acids comprising the expression vector or expression construct will remain an expression vector or expression construct.

As used herein, an “extracellular vesicle” or “EV” is used interchangeably and is defined to mean cell-derived vesicle having a membrane that surrounds and encloses a central space and is produced by a cell. Membranes of EVs can be composed of a lipid bi-layer having an external surface and internal surface bounding an enclosed volume. The membrane bilayer incorporates proteins and other macromolecules derived from the cell of origin and may comprise phospholipids. The luminal space encapsulates lipids, proteins, organic molecules and macromolecules including nucleic acids and polypeptides. Examples of extracellular vesicles include exosomes, ectosome, microvesicle, microsome or other cell-derived membrane vesicles. Other cell-derived membrane vesicles include a shedding vesicle, a plasma membrane-derived vesicle, and/or an exovesicle.

An extracellular vesicle can have a longest dimension, such as a cross-sectional diameter, of at least about 20, 30, 40, 50, 60, 70, 80, 90, 100, 200, 300, 400, or 500 nm and/or at most about 1000, 500, 400, 300, 200, 100, 90, 80, 70, 60, or 50 nm. In some instances, a longest dimension of a vesicle can range from about 20 nm to about 1000 nm, about 30 nm to about 1000 nm, about 20 nm to about 100 nm, about 30 nm to about 100 nm, about 40 nm to about 100 nm, about 20 nm to about 200 nm, about 30 nm to about 200 nm, about 40 nm to about 200 nm, about 20 nm to about 120 nm, such as about 30 nm to about 120 nm, about 40 nm to about 120 nm, about 20 nm to about 300 nm, about 30 nm to about 300 nm, about 40 nm to about 300 nm, about 50 nm to about 1000 nm, about 100 nm to about 500 nm, about 500 nm to about 1000 nm, and such as about 40 nm to about 500 nm, each range inclusive. When referring to a plurality of vesicles, such ranges can represent the average of all vesicles, including naturally occurring and modified vesicles in the mix.

As used herein, an “exosome” is defined to mean a secreted membrane-enclosed vesicle that originates from the endosome compartment in cells. The exosome can comprise a bilayer membrane, and can comprise various macromolecular cargo either within the internal space, displayed on the external surface of the extracellular vesicle, and/or spanning the membrane. Cargo can comprise nucleic acids, proteins, carbohydrates, lipids, small molecules, and/or combinations thereof. The endosome compartment, or the multi-vesicular body, can fuse with the plasma membrane of the cell, with ensuing release to the extracellular space of their vesicles as exosomes. Cargos such as protein, mRNA, miRNA, tRNA, yRNA, DNA, lipids, and other ingredients derived from the derived cells can be protected from degradation by the external environment and are beneficial to their biological function of active ingredients.

Exosomes may arise as small vesicles within larger membrane structures in the endosome within a cell and have a smaller size, ranging from about 20 nm to about 120 nm, about 30 nm to about 120 nm, about 40 nm to about 120 nm, about 20 nm to about 150 nm, about 30 nm to about 150 nm, about 40 nm to about 150 nm, about 20 nm to about 200 nm, about 30 nm to about 200 nm, about 40 nm to about 200 nm, about 20 nm to about 300 nm, about 30 nm to about 300 nm, or about 40 nm to about 300 nm. Exosomes can range in size from about 20 nm to about 300 nm. Additionally, the exosome may have an average diameter in the range of about 50 nm to about 220 nm. Preferably, in a specific embodiment, the exosome has an average diameter of about 120 nm±20 nm. It has been demonstrated that almost all living cells can secrete exosomes, and exosomes widely exist in various body fluids such as blood, urine, breast milk, or cerebrospinal fluid.

As used herein, the term “average” may be mean, mode or medium for a group of measurements.

As used herein, the term “about” when used before a numerical designation, e.g., diameter, size, temperature, time, amount, concentration, and such other, including a range, indicates approximations which may vary by (+) or (−) 10%, 5% or 1%.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth.

As used herein, a “hematopoietic cell” is defined to be a cell that arises from a hematopoietic stem cell. This includes but is not limited to myeloid progenitor cells, lymphoid progenitor cells, megakaryocytes, erythrocytes, mast cells, myeloblasts, basophils, neutrophils, eosinophils, macrophages, thrombocytes, monocytes, natural killer cells, T lymphocytes, B lymphocytes and plasma cells.

As used herein, “heterologous” is defined to mean the nucleic acid and/or polypeptide are not homologous to the host cell. For example, a construct is heterologous to a host cell if it contains some homologous sequences arranged in a manner not found in the host cell and/or the construct contains some heterologous sequences not found in the host cell.

As used herein, an “isopeptide bond” is defined to be an amide bond between a carboxyl group and an amino group at least one of which is not derived from a protein main chain or alternatively viewed is not part of the protein backbone. An isopeptide bond may form within a single protein or may occur between two peptides or a peptide and a protein. An isopeptide may form intramolecularly within a single protein or intermolecularly i.e. between two peptide/protein molecules. An isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Proteins which are known to form isopeptide bond spontaneously are provided in Table 11 from which isopeptide domain and isopeptide tag may be isolated.

As used herein, an “isopeptide domain” is defined to be a portion of a protein that can form a spontaneous isopeptide bond with a complementary isopeptide tag and upon association with the tag. In one embodiment, the isopeptide domain can be the portion of the full-length protein that remains after excision of the isopeptide tag, where the remaining portion retains the ability to spontaneously form an isopeptide bond upon binding to the isopeptide tag. In a separate embodiment, the isopeptide domain can be the smallest portion of a full-length protein having the ability to spontaneously form an isopeptide bond upon association with a peptide fragment from the same protein. Typically, an isopeptide domain is about 8-14 kDa. Examples of isopeptide domains may be found in Table 1 (e.g., SEQ ID NOS: 1-60).

As used herein, an “isopeptide tag” is defined to be a portion of a protein that can form a spontaneous isopeptide bond with a complementary isopeptide domain and upon association with the domain. The isopeptide tag is a peptide sequence derived from a subdomain of a full-length protein that contains only one of two amino acids which in the contest of the full-length protein form an intra-chain covalent isopeptide bond between the side chains of the two amino acids or between the side chain of one amino acid and main chain amino-group at the N-terminus or carboxyl-group at the C-terminus. In an embodiment, the isopeptide tag may be a minimally sized peptide from a portion of a full-length protein able to form an intra-chain isopeptide bond, where the minimally sized peptide can associate with an isopeptide domain and form an isopeptide both with the isopeptide domain. Typically, an isopeptide tag is about 11-14 amino acids long. Examples of isopeptide tags may be found in Table 1 (e.g., SEQ ID NOS: 61-66).

As used herein, a “marker” is defined to mean a protein, lipid, carbohydrate or other molecule involved in EV trafficking and/or EV interaction with target cells. As such, a marker may be found on an EV and/or a cell. An EV and/or a cell may be characterized in part by the presence of a marker. Preferably, a marker is found on the exterior surface of an EV and/or cell. A marker may be used interchangeable with a “biomarker.”

As used herein, a “polypeptide” may be a protein or a peptide. In an embodiment, polypeptide may be a targeting protein. For example, a polypeptide may be a single chain Fv (scFv). In an embodiment, a polypeptide may be an affinity peptide. For example, an affinity peptide may target a cell surface protein, such as GPC3 (glypican-3) affinity peptide for GPC3 cell surface protein.

As used herein, the term “reporter” or “reporter molecule” refers to a moiety capable of being detected indirectly or directly. Reporters include, without limitation, a chromophore, a fluorophore, a fluorescent protein, a luminescent protein, a receptor, a hapten, an enzyme, and a radioisotope.

As used herein, the term “reporter gene” refers to a polynucleotide that encodes a reporter molecule that can be detected, either directly or indirectly. Exemplary reporter genes encode, among others, enzymes, fluorescent proteins, bioluminescent proteins, receptors, antigenic epitopes, and transporters.

“Surface domain” is a subset of the protein or polypeptide primary sequence that is exposed to the extra-EV environment. The surface domain can be a loop between two transmembrane domains or it can contain one of the termini (amino or carboxy) of the protein. Protein domain topology relative to the membrane bi-layer can be determined empirically by assessing what portions of the protein are digested by an external protease. More recently, characteristic amino acid patterns, such as basic or acidic residues in the juxta-membrane regions of the protein have been used to algorithmically assign probable topologies (extracellular versus cytosolic) to integral membrane proteins. Since EVs have the same membrane topology orientation as the plasma membrane of the whole cell (the outer leaflet of the membrane is the same between cells and EVs), these algorithms can be applied to EV resident proteins as well. As such, the surface domain of an EV localizing transmembrane protein may sometimes be referred to as an extracellular domain due to the same membrane topology of an EV and plasma membrane. For example, the “surface domain” may be a short peptide of approximately 10-15 amino acids. In an embodiment, the “surface domain” may be an unstructured polypeptide. In an embodiment, the “surface domain” is the entire surface domain of an integral membrane protein. In yet another embodiment, the “surface domain” is part or portion of the surface domain of an integral membrane protein. In an embodiment, the surface domain is amino terminal to the transmembrane domain and cytosolic domain. In an embodiment, the surface domain is at the N-terminus of the vesicle localization moiety or the chimeric vesicle localization moiety and is on the external surface of an extracellular vesicle, such as an exosome.

“Transmembrane domain” may be a span of about 18-40 aliphatic, apolar and hydrophobic amino acids that assembles into an alpha-helical secondary structure and spans from one face of a membrane bilayer to the other face, meaning that the N-terminus of the helix extends at least to and in many cases beyond the phospholipid headgroups of one membrane leaflet while the C-terminus extends to the phospholipid headgroups of the other leaflet. In an embodiment, the transmembrane domain connects an amino terminal surface domain with a carboxyl terminal cytosolic domain.

“Cytosolic domain” is a subset of the protein or polypeptide primary sequence that is exposed to the intra-EV or intracellular environment. The cytosolic domain can be a loop between two transmembrane domains or it can contain one of the termini (amino or carboxy) of the protein. Its topology is distinct from that of the transmembrane and the surface domains. In an embodiment, the cytosolic domain is in the cytoplasmic side of a cell. In another embodiment, the cytosolic domain is in the lumen of a vesicle. As such, the cytosolic domain may be also referred to as a lumenal domain or luminal domain. In an embodiment, the cytosolic domain is at the C-terminus of the vesicle localization moiety or the chimeric vesicle localization moiety.

Merely by way of example, sequences corresponding to “surface domain,” “transmembrane domain” and “cytosolic domain” for the proteins disclosed herein may be found within the description under protein accession numbers provided herein. Particularly useful examples are the proteins cataloged within UniProtKB (UniProt Release 2019_11 (11 Dec. 2019)) where under each accession number amino acid sequence along with features and functional domains are provided. For example, topological domains associated with each of the transmembrane vesicle localization moiety provided herein may be found in UniProKB accession number with the description of “extracellular” for the “surface domain,” “helical” for the “transmembrane domain” and “cytoplasmic” for the “cytosolic domain.” Amino acid sequences corresponding to “signal peptide” are also indicated as being processed out of the mature transmembrane protein. In addition, a number of other publicly available databases may also be used to identify the surface (extracellular), transmembrane and cytosolic (lumenal or cytoplasmic) domain, such as Membranome: membrane proteome of single-helix transmembrane proteins (membranome.org; Lomize, A. L. et al. (2017) Membranome: a database for proteome-wide analysis of single-pass membrane proteins. Nucleic Acids Res. 45:D250-D255 and Lomize, A. L. et al. (2018) Membranome 2.0: database for proteome-wide profiling of bitopic proteins and their dimers. Bioinformatics 34:1061-1062) and PDBTM: Protein Data Bank of Transmembrane Proteins (pdbtm.enzim.hu; PDBTM version 2021-01-08) (Kozma, D. et al. (2013) Nucleic Acids Res. 41:D524-D529). Outside of these curated publicly available databases, the classification of transmembrane proteins and identification of surface, transmembrane and cytosolic domains are reviewed in Goder, V. and Spiess, M. (2001) Topogenesis of membrane proteins: determinants and dynamics. FEBS Lett. 504:87-93; Tusnady, G. et al. (2004) Transmembrane proteins in the Protein Data Bank: identification and classification. Bioinformatics 20:2964-2972; Chou, K.-C. and Shen, H.-B. (2007) MemType-2L: A Web server for predicting membrane proteins and their types by incorporating evolution information through Pse-PSSM. Biochem. Biophys. Res. Comm. 360:339-345; Casadio R., Martelli P. L., Bartoli L., Fariselli P. (2010) Topology prediction of membrane proteins: how distantly related homologs come into play. In: Structural Bioinformatics of Membrane Proteins. Springer, Vienna. In a preferred embodiment, a “chimeric vesicle localization moiety” comprises the “surface-and-transmembrane domain” of one vesicle localization moiety and the “cytosolic domain” of a second vesicle localization moiety, wherein the two vesicle localization moieties are different and distinct proteins and are not isoforms. In an embodiment, the “chimeric vesicle localization moiety” comprises the “surface-and-transmembrane domain” of one vesicle localization moiety and the “cytosolic domain” of a second vesicle localization moiety, wherein the two vesicle localization moieties are different and distinct proteins and are not isoforms and wherein the “surface-and-transmembrane domain” may have a mutation. The mutation may be a deletion, insertion or a substitution, so long as the resulting mutant retains at least 80% or at least about 90% of the EV association activity of the unmutated counterpart. In an embodiment, the “chimeric vesicle localization moiety” is derived from combining domains of two proteins encoded by two distinct genes which are not allelic or homologs. In an embodiment, the “chimeric vesicle localization moiety” is derived from combining domains of two proteins encoded by two distinct genes which are not orthologs. In an embodiment, the “chimeric vesicle localization moiety” is derived from combining domains of two proteins encoded by two distinct genes which are not paralogs. In an embodiment, the “chimeric vesicle localization moiety” is derived from combining domains of two proteins encoded by two distinct genes which are paralogs. In an embodiment, the “chimeric vesicle localization moiety” is derived from combining domains of two proteins encoded by two nonhomologous genes. In an embodiment, the “chimeric vesicle localization moiety” is derived from combining domains of two or more proteins encoded by two or more nonhomologous genes. In an embodiment, the “chimeric vesicle localization moiety” is derived from combining domains of two or more proteins encoded by two or more nonhomologous human genes. In an embodiment, the “chimeric vesicle localization moiety” is produced from combining domains of two or more human genes encoding transmembrane proteins. In a preferred embodiment, the “chimeric vesicle localization moiety” is produced from combining two nonhomologous human genes or two human genes not placed within the same gene family, wherein the genes encode transmembrane proteins.

An “isoform” of a protein can be, e.g., a protein resulting from alternative splicing of a gene expressing the protein, a protein resulting from alternative promoter usage of a gene expressing the protein, or a degradation product of the protein.

“Surface-and-transmembrane domain” is a contiguous polypeptide containing both a domain that is exposed to extracellular or extra-EV solvent and a transmembrane domain as described above.

A “linker” may be a peptide or polypeptide with 3 to 1000 amino acids that are generally non-hydrophobic and encode no secondary structural elements such as helices or beta-sheets. Suitable examples include, but are not limited to, any of (Gly)₈, (Gly)₆, (GS)ₙ (n=1-5), (GGS)ₙ (n=1-5), (GGGS)ₙ (n=1-5), (GGGGS)ₙ (n=1-5), (GGGGGS)ₙ (n=1-5)(EAAAK)ₙ (n=1-3), A(EAAAK)₄ALEA(EAAAK)₄A, (GGGGS)ₙ (n=1-4), (Ala-Pro)ₙ (10-34 aa), cleavable linkers such as VSQTSKLTRAETVFPDV, PLGLWA, RVLAEA; EDVVCCSMSY; GGIEGRGS, TRHRQPRGWE, AGNRVRRSVG, RRRRRRRRR, GLFG, and LE.

As used herein “isolated” means a state following one or more purifying steps but does not require absolute purity. “Isolated” extracellular vesicle, exosome or composition thereof means an extracellular vesicle, exosome or composition thereof passed through one or more purifying steps that separate the vesicle, extracellular vesicle, exosome or composition from other molecules, materials or cellular components found in a mixture or outside of the vesicle, extracellular vesicle or exosome or found as part of the composition prior to purification or separation. Isolation and purification may be achieved in accordance with conventional methods of recombinant synthesis or cell free protein synthesis. Separation procedures of interest include affinity chromatography. Affinity chromatography makes use of the highly specific binding sites usually present in biological macromolecules, separating molecules on their ability to bind a particular ligand. For example, covalent bonds attach the ligand to an insoluble, porous support medium in a manner that overtly presents the ligand to the protein sample, thereby using natural biospecific binding of one molecular species to separate and purify a second species from a mixture. Antibodies may be used in affinity chromatography. Preferably a microsphere or matrix is used as the support for affinity chromatography. Such supports are known in the art and are commercially available, and include activated supports that can be combined to the linker molecules. For example, Affi-Gel supports, based on agarose or polyacrylamide are low pressure gels suitable for most laboratory-scale purifications with a peristaltic pump or gravity flow elution. Affi-Prep supports, based on a pressure-stable macroporous polymer, may be suitable for preparative and process scale applications. Isolation may also be performed using methods involving centrifugation, filtration, size exclusion chromatography and vesicle flow cytometry.

As used herein, a “vesicle localization moiety fusion protein” is a fusion protein comprising a vesicle localization moiety and a protein/peptide of interest. In an embodiment, the protein/peptide of interest is an isopeptide domain or an isopeptide tag. In an embodiment, the vesicle localization moiety fusion protein is a fusion protein comprising a vesicle localization moiety and one or more isopeptide domain(s). In another embodiment, the vesicle localization moiety fusion protein is a fusion protein comprising a vesicle localization moiety and one or more isopeptide tag(s). In another embodiment, the vesicle localization moiety fusion protein is a fusion protein comprising a vesicle localization moiety and a combination of one or more isopeptide domain(s) and isopeptide tag(s). In an embodiment, the vesicle localization moiety fusion protein is a chimeric vesicle localization moiety fusion protein. In an embodiment, the chimeric vesicle localization moiety fusion protein is a fusion protein comprising a chimeric vesicle localization moiety and one or more isopeptide domain(s). In another embodiment, the chimeric vesicle localization moiety fusion protein is a fusion protein comprising a chimeric vesicle localization moiety and one or more isopeptide tag(s). In another embodiment, the chimeric vesicle localization moiety fusion protein is a fusion protein comprising a chimeric vesicle localization moiety and a combination of one or more isopeptide domain(s) and isopeptide tag(s).

As used herein, a “protein” is a polypeptide of more than 50 amino acids.

As used herein, a “peptide” is a short polypeptide of about 2 to 50 amino acids.

As used herein, a “vesicle localization moiety” is defined to be a polypeptide that can display an isopeptide domain and/or an isopeptide tag. Vesicle localization moieties can be membrane proteins (integral or peripheral). In an embodiment, the vesicle localization moiety is a chimeric vesicle localization moiety

As used herein, a “targeting moiety” can include, but is not limited to, a small molecule, glycoprotein, polypeptide, peptide, lipid, carbohydrate, nucleic acid, nucleic acid analog (such as, antisense oligonucleotide (ASO, 2′-O-methyl (OMe), 2′-fluoro (F), and 2′-O-methoxyethyl (MOE) RNA, locked nucleic acid (LNA), constrained ethyl (cEt), phosphorodiamidate morpholinos (PMOs), phosphorothioate, and peptide nucleic acid (PNA)), ligand, aptamer, chemical compound, macromolecule or other molecules involved in EV trafficking and/or EV interaction with target cells. The targeting moiety may be displayed inside or on the outside of a vesicle membrane or may span the inner membrane, outer membrane, or both inner and other membranes. For targeting cell surface receptor, ligand, or moiety on the outside of a cell or tissue, the targeting moiety is similarly displayed on the outside of a vesicle membrane, so as to be able to bind to the targeted cell surface receptor, ligand or moiety. In an embodiment, the targeting moiety is an affinity peptide for a cell surface receptor or ligand. Examples of suitable affinity peptides include, but are not limited to, THRPPMWSPVWP (SEQ ID NO.: 194), a targeting moiety(ies) or peptide for transferrin receptor (TfR), and THVSPNQGGLPS (SEQ ID NO.: 196; also called “PEPN”), a targeting moiety(ies) or peptide for glypican-3 (GPC3). Additional examples include CLVSGGMAC (SEQ ID NO.: 158), CLVSGCNTC (SEQ ID NO.: 160), CDLVSGYGC (SEQ ID NO.: 162), CLVSTSATC (SEQ ID NO.: 164), CTALVSQTC (SEQ ID NO.: 166), CWLVSGIGC (SEQ ID NO.: 168), CLVSSVFPC (SEQ ID NO.: 170), CPSLVSSVC (SEQ ID NO.: 172), CGVSLVSTC (SEQ ID NO.: 174), CQLVSGEPC (SEQ ID NO.: 176), CNLVSRRLC (SEQ ID NO.: 178), CLVSWRGSC (SEQ ID NO.: 180), CDHFLVSPC (SEQ ID NO.: 182), CGRGLVSLC (SEQ ID NO.: 184), CFPVALVSC (SEQ ID NO.: 186), CRWSSLVSC (SEQ ID NO.: 188), CWSKSLVSC (SEQ ID NO.: 190) and CPGRSLVSC (SEQ ID NO.: 192).

In another embodiment, the targeting moiety is an antibody, fragment of an antibody or a single chain Fv (scFv). Examples of scFv's include GC33 scFv (SEQ ID NO: 152), 6A6 scFv (SEQ ID NO: 154) and alaC scFv (SEQ ID NO: 156) along with their nucleic acid encoding for the scFv's as provided in Table 6. The targeting moiety may be fused to an isopeptide tag or isopeptide domain and the resulting fusion protein allowed to form an isopeptide bond with its complementary partner (e.g., complementary isopeptide domain-isopeptide tag binding and subsequent spontaneous formation of isopeptide bond between the two partners) displayed on exosomes that are “emptied” of natural cargo, “carry” a naturally occurring cargo or loaded with a payload for delivery to such as target cells or tissues.

As used herein, a “targeting moiety fusion protein” or “a fusion protein of a targeting moiety” or “targeting moiety fusion peptide” or “a fusion peptide of a targeting moiety” is a polypeptide comprising a targeting moiety and an isopeptide domain or isopeptide tag. In an embodiment, such fusion proteins or peptides may be produced by recombinant DNA methods or may be chemically synthesized.

As used herein, a “targeting moiety conjugate” or “a conjugate of a targeting moiety” is a chemical conjugate of a targeting moiety and an isopeptide domain or isopeptide tag. In an embodiment, such conjugates may be produced by chemical crosslinking or photocrosslinking of a targeting moiety and an isopeptide domain or isopeptide tag. In another embodiment, such conjugates may include an isopeptide domain or tag covalently attached to a non-polypeptide targeting moiety.

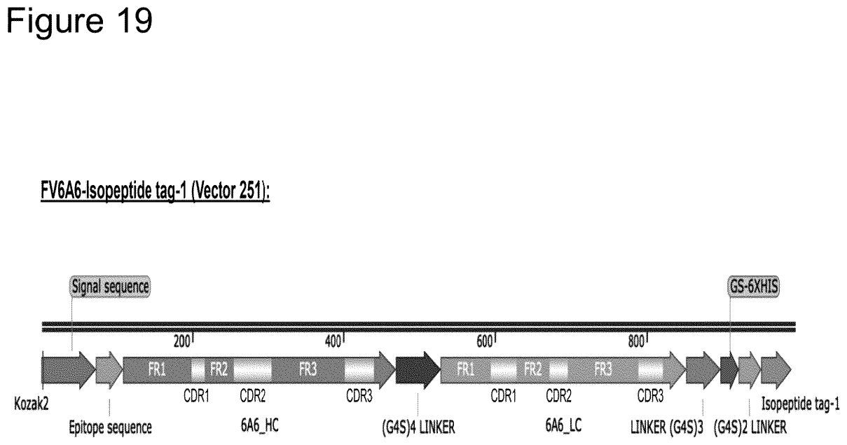

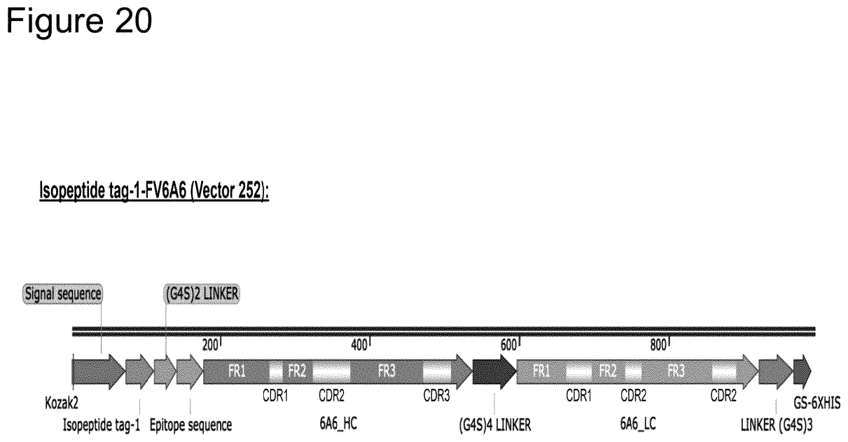

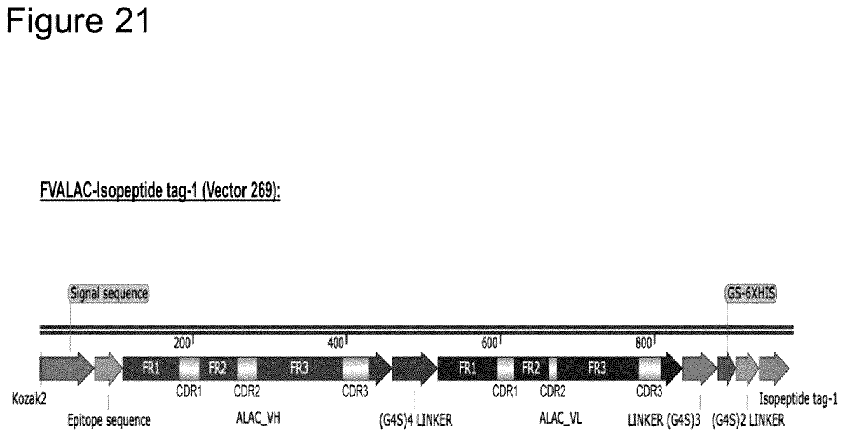

As used herein, a “single chain antibody” (scFv) is defined as an immunoglobulin molecule with function in antigen-binding activities. An antibody in scFv (single chain fragment variable) format consists of variable regions of heavy (V_{H}) and light (V_{L}) chains, which are joined together by a flexible peptide linker. Examples of scFv's as fusion proteins are provided in FIGS. 19-21.

As used herein, “transfected” or “transformed” or “transduced” are defined to be a process by which exogenous nucleic acid is transferred or introduced into a host cell. A “transfected” or “transformed” or “transduced” cell is one which has been transfected, transformed or transduced with exogenous nucleic acid. The cell includes the primary subject cell and its progeny.

### Extracellular Vesicles

The invention provides an isolated extracellular vesicle (e.g., exosome) or composition thereof, which comprises a fusion protein comprising a vesicle localization moiety and one or more isopeptide domain(s). Alternatively, in some embodiments, an extracellular vesicle (e.g., exosome) or composition thereof comprises a fusion protein comprising a vesicle localization moiety and one or more isopeptide tag(s). In some embodiments, an extracellular vesicle (e.g., exosome) or composition comprises a combination of two or more vesicle localization moiety fusion proteins, wherein each vesicle localization moiety fusion protein comprises a vesicle localization moiety and one or more isopeptide domain(s) and wherein at least two different vesicle localization moiety fusion proteins exist in the extracellular vesicle or composition. In some embodiments, an extracellular vesicle (e.g., exosome) or composition comprises a combination of two or more vesicle localization moiety fusion proteins, wherein each vesicle localization moiety fusion protein comprises a vesicle localization moiety and one or more isopeptide tag(s) and wherein at least two different vesicle localization moiety fusion proteins exist in the extracellular vesicle or composition. In an embodiment, the isolated extracellular vesicle (e.g., exosome) or composition comprises a fusion protein comprising a vesicle localization moiety and one or more isopeptide domain(s) and additionally a post-translational modification site in the fusion protein so as to increase stability of the vesicle localization moiety fusion protein. In an embodiment, the post-translational modification site is a glycosylation site in the fusion protein.

In a preferred embodiment, an extracellular vesicle (e.g., exosome) comprises a fusion protein comprising a vesicle localization moiety and one or more isopeptide domain(s).

In some embodiments, an isolated composition comprises an extracellular vesicle or exosome comprising a fusion protein comprising a vesicle localization moiety and one or more isopeptide domain(s). Alternatively, in some embodiments, an isolated composition comprises an extracellular vesicle or exosome comprising a fusion protein comprising a vesicle localization moiety and one or more isopeptide tag(s). In some embodiments, an isolated composition comprises an extracellular vesicle or exosome comprising a combination of two or more vesicle localization moiety fusion proteins, wherein each vesicle localization moiety fusion protein comprises a vesicle localization moiety and one or more isopeptide domain(s) and wherein at least two different vesicle localization moiety fusion proteins exist in the extracellular vesicle or exosome. In some embodiments, an isolated composition comprises an extracellular vesicle or exosome comprising a combination of two or more vesicle localization moiety fusion proteins, wherein each vesicle localization moiety fusion protein comprises a vesicle localization moiety and one or more isopeptide tag(s) and wherein at least two different vesicle localization moiety fusion proteins exist in the extracellular vesicle or exosome.

In a preferred embodiment, an isolated composition comprises an extracellular vesicle or exosome comprising a fusion protein comprising a vesicle localization moiety and one or more isopeptide domain(s).

In some embodiments, an isolated extracellular vesicle (e.g., exosome) or composition thereof comprises a fusion protein comprising a vesicle localization moiety and one or more isopeptide bond(s). In some embodiments, an isolated extracellular vesicle (e.g., exosome) or composition comprises a fusion protein comprising a vesicle localization moiety and one or more isopeptide bond(s) between an isopeptide domain and an isopeptide tag.

In some embodiments, a composition herein comprises an isolated or enriched set of vesicles that selectively target a tissue or cell of interest. Such vesicles can be loaded with a payload to be delivered to the cell or tissue of interest.

An extracellular vesicle (EV) can be a membrane that encloses an internal space. Cell-derived extracellular vesicles can be smaller than the cell from which they are derived and range in diameter from about 20 nm to 1000 nm (e.g., 20 nm to 1000 nm; 20 nm to 200 nm; 90 nm to 150 nm). Such vesicles can be created through the outward budding and fission from plasma membranes, assembled at and released from an endomembrane compartment, or derived from cells or vesiculated organelles having undergone apoptosis, and can contain organelles. They can be produced in an endosome by inward budding into the endosomal lumen resulting in intraluminal vesicles of a multivesicular body (MVB) and released extracellularly as exosomes upon fusion of the multivesicular body (MVB) with the plasma membrane. They can be derived from cells by direct and indirect manipulation that may involve the destruction of said cells. They can also be derived from a living or dead organism, an explanted tissue or organ, and/or a cultured cell.

Examples of extracellular vesicles include exosomes, ectosome, microvesicle, microsome or other cell-derived membrane vesicles. Other cell-derived membrane vesicles include a shedding vesicle, a plasma membrane-derived vesicle, and/or an exovesicle.

EVs may have a cross-sectional diameter smaller than the cell from which they are derived. EVs can have a longest dimension, such as a cross-sectional diameter, of at least about 20, 30, 40, 50, 60, 70, 80, 90, 100, 200, 300, 400, or 500 nm and/or at most about 1000, 500, 400, 300, 200, 100, 90, 80, 70, 60, or 50 nm. In some instances, a longest dimension of a vesicle can range from about 20 nm to about 1000 nm, about 30 nm to about 1000 nm, about 20 nm to about 100 nm, about 30 nm to about 100 nm, about 40 nm to about 100 nm, about 20 nm to about 200 nm, about 30 nm to about 200 nm, about 40 nm to about 200 nm, about 20 nm to about 120 nm, such as about 30 nm to about 120 nm, about 40 nm to about 120 nm, about 20 nm to about 300 nm, about 30 nm to about 300 nm, about 40 nm to about 300 nm, about 50 nm to about 1000 nm, about 500 nm to about 1000 nm, about 100 nm to about 500 nm, about 500 nm to about 1000 nm, or about 40 nm to about 500 nm, each range inclusive. When referring to a plurality of vesicles, such ranges can represent the average (e.g., mean) of all vesicles, including naturally occurring and modified vesicles in the mix.

Exosomes can be secreted membrane-enclosed vesicles that originate from the endosome compartment in cells. The endosome compartment, or the multi-vesicular body, can fuse with the plasma membrane of the cell, with ensuing release to the extracellular space of their vesicles as exosomes. Further, an exosome can comprise a bilayer membrane, and can comprise various macromolecular cargo either within the internal space, displayed on the external surface of the extracellular vesicle, and/or spanning the membrane. Cargo can comprise nucleic acids, proteins, carbohydrates, lipids, small molecules, and/or combinations thereof. Exosomes can range in size from about 20 nm to about 300 nm. Additionally, the exosome may have an average diameter in the range of about 50 nm to about 220 nm. Preferably, in a specific embodiment, the exosome has an average diameter of about 120 nm±20 nm.

In some instances, exosomes and other extracellular vesicles can be characterized and marked based on their protein compositions, such as integrins and tetraspanins. Other protein markers that are used to characterize exosomes and other extracellular vesicles (EVs) include TSG101, ALG-2 interacting protein X (ALIX), flotillin 1, and cell adhesion molecules which are derived from the parent cells in which the exosome and/or EV is formed. Similar to proteins, lipids are major components of exosomes and EVs and can be utilized to characterize them.

Further, naturally occurring exosomes can originate from the endosome and can contain proteins such as heat shock proteins (Hsp70 and Hsp90), membrane transport and fusion proteins (GTPases, Annexins and flotillin), tetraspanins (CD9, CD63, CD81, and CD82) and proteins such as CD47. Among these proteins, heat shock proteins, annexins, and proteins of the Rab family can abundantly be detected in exosomes and can be involved in their intracellular assembly and trafficking. Tetraspanins, a family of transmembrane proteins, can also be detected in exosomes. In a cell, tetraspanins can mediate fusion, cell migration, cell-cell adhesion, and signaling. Other abundant proteins found in exosomes can be the integrins, which can be adhesion molecules that can facilitate cell binding to the extracellular matrix. Integrins can be involved in adhering the vesicles to their target cells. Certain proteins that can be found on the surface of exosomes, such as CD55 and CD59, can protect exosomes from lysis by circulating immune cells, while CD47 on exosomes can act as an anti-phagocytic signal that blocks the uptake of exosomes by immune cells. Other proteins that can be associated with exosomes include thrombospondin, lactadherin, ALIX (also known as PDCD6IP), TSG1012, and SDCB1. Classes of membrane proteins that can naturally occur on the surface of exosomes and other extracellular vesicles include ICAMs, MHC Class I, LAMP2, lactadherin (C1C2 domain), tetraspannins (CD63, CD81, CD82, CD53, and CD37), Tsg101, Rab proteins, integrins, Alix, and lipid raft-associated proteins such as glycosylphosphatidylinositol (GPI)-modified proteins and flotillin.

Besides proteins, exosomes are also rich in lipids, with different types of exosomes containing different types of lipids. The lipid bilayer of exosomes can be constituted of cell plasma membrane types of lipids such as sphingomyelin, phosphatidylcholine, phosphatidylethanolamine, phosphatidylserine, monosialotetrahexosylganglioside (GM3), and phosphatidylinositol. Sphingomyeline and GM3 are responsible for determining the exosomes rigidity while phosphatidylserine is expressed on the plasma membrane of exosomes through different types of phospholipid transportation enzymes. Phosphatidylserine is involved in docking outer proteins, allowing the signaling and fusion of the exosome to the plasma membrane. Other types of lipids that can be found in exosomes are cholesterol, ceramide, and phosphoglycerides, along with saturated fatty-acid chains. Additional optional constituents of exosomes include nucleic acids such as micro RNA (miRNA), messenger RNA (mRNA), and non-coding RNAs. Exosomes can also contain a sugar (e.g. a simple sugar, polysaccharide, or glycan) or other molecules.

### Engineered Extracellular Vesicles

EVs and/or exosomes can be engineered to display markers that provide a desired function and/or property to the EV and/or exosome. In an embodiment, the EV and/or exosome is engineered to have an isopeptide domain on a membrane protein of the EV and/or exosome so that the isopeptide domain is displayed on the outside of the EV and/or exosome where it can react with an isopeptide tag. The isopeptide tag can be fused or conjugated to a variety of different molecules that can impart desired function and/or properties to the EV and/or exosome. For example, the molecule (e.g., targeting moiety) with the isopeptide tag could recognize a target molecule (e.g., a cell and/or tissue marker) on a target cell and/or target tissue. Such molecules could be attached to the EV and/or exosome by the isopeptide tag and so provide the EV and/or exosome with targeting capability for the desired target cell and/or target tissue (e.g., targeting moiety fusion protein or conjugate, wherein the targeting moiety is an affinity peptide or scFv to a marker expressed on a target cell or tissue). Binding of the isopeptide tag by the isopeptide domain results in formation of an isopeptide bond resulting in attachment of the molecule or targeting moiety to the external surface of an EV and/or exosome. Other properties that can be introduced to the EV and/or exosome by the molecule with the isopeptide tag include, for example, therapeutic entities including oligonucleotides, proteins and small molecules or combinations thereof that convey a therapeutic impact to recipient cells and tissues. In an embodiment, the membrane protein of the EV and/or exosome is a VLM or chimeric VLM.

Without being bound by any theory, a “vesicle localization moiety” (also referred to as a vesicle targeting moiety, a scaffold or “VLM”) may be a macromolecule that localizes at an extracellular vesicle. In an embodiment, the vesicle localization moiety is a polypeptide. In an embodiment, the vesicle localization moiety is a protein. In an embodiment, the protein is a single polypeptide chain. In an embodiment, the vesicle localization moiety is a protein that localizes at an extracellular vesicle. In an embodiment, the vesicle localization moiety is a membrane protein. In a preferred embodiment, the vesicle localization moiety is a transmembrane protein comprising a surface domain, a transmembrane domain and a cytosolic domain. Localization of such a transmembrane protein at an extracellular vesicle results in the surface domain at the outer (or external) surface of the vesicle, the transmembrane domain with the lipid bilayer of the vesicle and the cytosolic domain in the lumen (or interior) of the vesicle. Because of topological equivalence, a surface domain may also be referred to as an extracellular domain, since the surface domain on the surface of an exosome shares the same topological state as plasma membrane bound transmembrane protein on the surface of a cell; similarly, a cytosolic domain may be referred to as a lumenal domain, since part of the cytoplasm where the cytosolic domain initially resides is incorporated into the lumen of a vesicle produced by inward budding of an endosomal membrane to eventually produce multiple intraluminal vesicles of a multivesicular body (MVB) prior to secretion of the vesicles as exosomes upon fusion of the MVB with the plasma membrane of an EV producer cell.

In an embodiment, the vesicle localization moiety may be a single pass transmembrane protein. Merely by way of example, the single pass transmembrane protein may comprise an amino-terminal surface domain and a carboxyl-terminal cytosolic domain (lumenal domain) joined by a transmembrane domain. For example, nascent or newly synthesized single pass transmembrane protein may additionally comprise a signal peptide (or signal sequence) preceding the surface domain, which is cleaved by a signal peptidase upon translocation of the nascent protein into a membrane, such as endoplasmic reticulum in eukaryotes or plasma membrane in prokaryotes. In another embodiment, the nascent or newly synthesized transmembrane protein may be processed to a mature transmembrane protein which lacks a signal peptide of the nascent or newly synthesized transmembrane protein.

In one example, the single pass transmembrane protein is a type I transmembrane protein. In an embodiment, the single pass, type I transmembrane protein comprises an amino-terminal surface domain and a carboxyl-terminal cytosolic domain (lumenal domain) joined by a transmembrane domain. In another embodiment, nascent or newly synthesized single pass, type I transmembrane protein additionally comprises a signal peptide preceding the surface domain, which is cleaved by a signal peptidase upon translocation of the nascent protein into a membrane, such as endoplasmic reticulum in eukaryotes or plasma membrane in prokaryotes. In yet another embodiment, the nascent or newly synthesized single pass, type I transmembrane protein is processed to a mature single pass, type I transmembrane protein which lacks a signal peptide of the nascent or newly synthesized single pass, type I transmembrane protein. In a preferred embodiment, the nascent or newly synthesized single pass, type 1 transmembrane protein may be processed to a mature single pass, type I transmembrane protein which lacks a signal peptide of the nascent or newly synthesized single pass, type I transmembrane protein.

The vesicle localization moiety may have a surface domain, a transmembrane domain and a cytosolic domain. Such protein domains are known in the art and are well annotated and defined for the proteins described, herein, in the figures and in annotations associated with Accession Numbers from publicly available databases, referred herein, such as UniProtKB (UniProt Release 2019_11 (11 Dec. 2019); The UniProt Consortium (2019) UniProt: a worldwide hub of protein knowledge. Nucleic Acids Res. 47:D506-515) and Genome Reference Consortium Human Build 38 patch release 13 (GRCh38.p13; GenBank assembly accession GCA_000001405.28 and RefSeq assembly accession GCF_000001405.39. Examples of surface domain (SEQ ID NO: 130 (Lamp2) and 136 (CSTN1)), transmembrane domain (SEQ ID NO: 132 (lamp2) and 138 (CSTN1)) and cytosolic domain (SEQ ID NO: 134 (Lamp2), 140 (CSTN1), 142 (PTGFRN), 144 (ITGA3), 146 (IL3RA), 148 (SELPL) and 150 (ITGB1)) may be found in Table 5 along with their nucleic acid coding sequences.

In an embodiment of the invention, the vesicle localization moiety is produced in a eukaryotic cell, preferably a mammal and most preferably a human.

A “chimeric vesicle localization moiety” or “chimeric VLM” is a vesicle localization moiety which may be produced by substituting one vesicle localization moiety domain with another vesicle localization moiety domain, so as to produce a chimeric vesicle localization moiety or chimeric VLM. A chimeric vesicle localization moiety may be obtained by combining one or more functional domains of one vesicle localization moiety with one or more functional domains of another, different vesicle localization moiety. The combination comprises portion(s) of at least two vesicle localization moieties, so as to obtain a chimeric vesicle localization moiety which is superior in its association with an EV than either of the parental vesicle localization moiety, as quantified by mean recombinant protein density on EV surface and/or fraction (or percent) of total EVs positive for the recombinant protein. In an embodiment, the chimeric vesicle localization moiety comprises a surface domain, a transmembrane domain and a lumenal or cytosolic domain of a transmembrane protein or the two parental transmembrane proteins from which it is derived. In an embodiment, the chimeric vesicle localization moiety has the same arrangement of surface domain, transmembrane domain and lumenal or cytosolic domain as described for the vesicle localization moiety, described above. Merely by way of example, a chimeric vesicle localization moiety comprising a surface-and-transmembrane domain of a first vesicle localization moiety and a cytosolic domain of a second vesicle localization moiety may interact synergistically to increase accumulation at an extracellular vesicle. This not only may improve EV localization but may also change the composition of EVs.

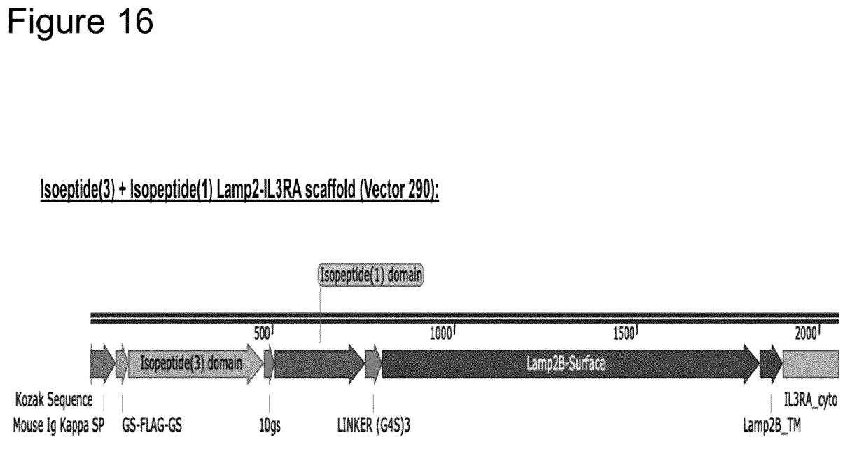

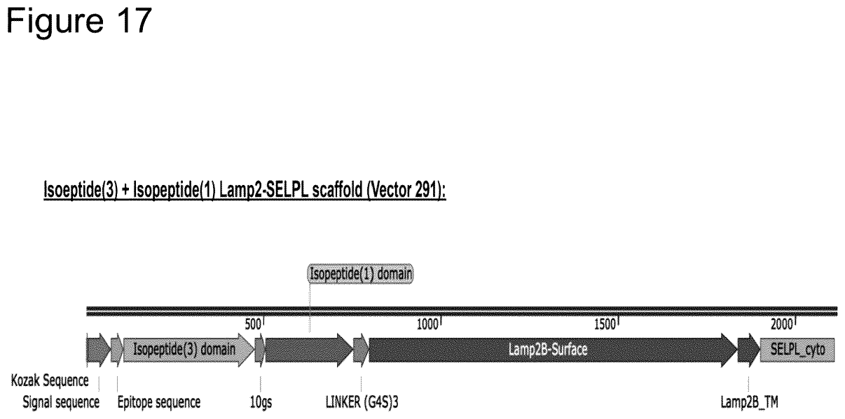

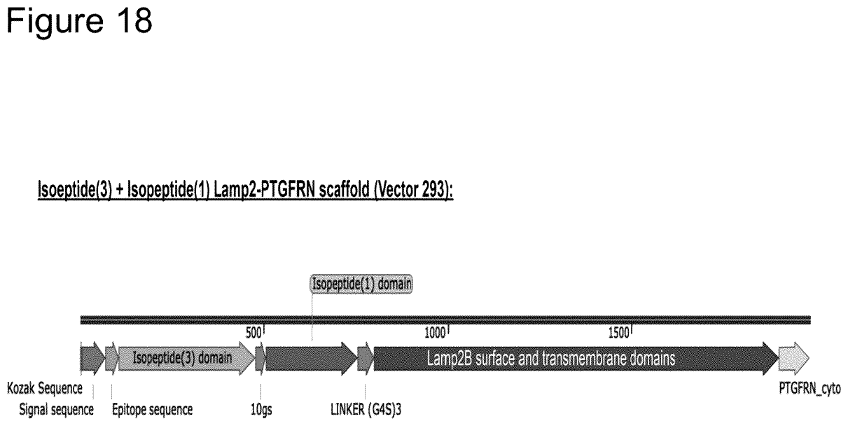

The chimeric vesicle localization moiety can be a single pass transmembrane protein. The chimeric vesicle localization moiety can be a type I transmembrane protein, albeit a chimeric type I transmembrane protein. The chimeric vesicle localization moiety can be a single pass, type I transmembrane protein, albeit a chimeric single pass, type I transmembrane protein. In an embodiment, the chimeric vesicle localization moiety comprises an amino-terminal surface domain and a carboxyl-terminal cytosolic domain (lumenal domain) joined by a transmembrane domain. In an embodiment, nascent or newly synthesized chimeric vesicle localization moiety additionally comprises a signal peptide preceding the surface domain, which is cleaved by a signal peptidase upon translocation of the nascent protein into a membrane, such as endoplasmic reticulum in eukaryotes or plasma membrane in prokaryotes. In an embodiment, the nascent or newly synthesized chimeric vesicle localization moiety is processed to a mature form which lacks a signal peptide of the nascent or newly synthesized transmembrane protein. In an embodiment, the nascent or newly synthesized chimeric vesicle localization moiety is processed to a mature transmembrane protein which lacks a signal peptide of the nascent or newly synthesized transmembrane protein. In an embodiment, the extracellular vesicle comprises a chimeric vesicle localization moiety which has been processed to a mature form lacking a signal peptide of a nascent or newly synthesized chimeric vesicle localization moiety, a transmembrane protein. In an embodiment, the extracellular vesicle comprises a chimeric vesicle localization moiety which has been processed to a mature form lacking a signal peptide of a nascent or newly synthesized chimeric vesicle localization moiety, a transmembrane protein. In an embodiment, the chimeric vesicle localization moiety lacking a signal peptide or mature form may be any of the chimeric vesicle localization moiety as provided in Table 4 (for example, SEQ ID NO: 116, 118, 120, 122, 124 and 126, encoded by nucleic acid SEQ ID NO: 115, 117, 119, 121, 123 and 125, respectively). In an embodiment, nucleic acid sequences provided in Table 4 for chimeric vesicle localization moieties (e.g., Table 4, SEQ ID NO: 115, 117, 119, 121, 123 and 125) may be used to produce polypeptides comprising a chimeric vesicle localization moiety (e.g., Table 7, SEQ ID NO: 210, 212 and 214; see FIGS. 16-18). Furthermore, a nucleic acid comprising a coding sequence for an isopeptide domain or isopeptide tag of interest may be fused in-frame with a coding sequence for a chimeric vesicle localization moiety as provided in Table 7 to encode for a polypeptide comprising an isopeptide domain or tag and a chimeric vesicle localization moiety (e.g., Table 7, SEQ ID NO: 209, 211 and 213). The encoded fusion when expressed in cells may additionally comprise nucleic acid sequence encoding a signal peptide sequence fused inframe at the N-terminus to permit association with cellular membrane and trafficking to form exosomes. In an embodiment, the fusion protein comprises an amino terminal signal peptide sequence, followed by one or more isopeptide domain(s) or isopeptide tag(s) and then by a chimeric vesicle localization moiety or a vesicle localization moiety. In an embodiment, the nucleic acid encodes a fusion protein comprising comprises an amino terminal signal peptide sequence, followed by one or more isopeptide domain(s) or isopeptide tag(s) and then by a chimeric vesicle localization moiety or a vesicle localization moiety.

Suitable examples of the first and/or second vesicle localization moieties may be any of ACE, ADAM10, ADAM15, ADAM9, AGRN, ALCAM, ANPEP, ANTXR2, ATP1A1, ATP1B3, BSG, BTN2A1, CALM1, CANX, CD151, CD19, CD1A, CD1B, CD1C, CD2, CD200, CD200R1, CD226, CD247, CD274, CD276, CD33, CD34, CD36, CD37, CD3E, CD40, CD40LG, CD44, CD47, CD53, CD58, CD63, CD81, CD82, CD84, CD86, CD9, CHMP1A, CHMP1B, CHMP2A, CHMP3, CHMP4A, CHMP4B, CHMP5, CHMP6, CLSTN1, COL6A1, CR1, CSF1R, CXCR4, DDOST, DLL1, DLL4, DSG1, EMB, ENG, EVI2B, F11R, FASN, FCER1G, FCGR2C, FLOT1, FLOT2, FLT3, FN1, GAPDH, GLG1, GRIA2, GRIA3, GYPA, HSPG2, ICAM1, ICAM2, ICAM3, IGSF8, IL1RAP, IL3RA, IL5RA, IST1, ITGA2, ITGA2B, ITGA3, ITGA4, ITGA5, ITGA6, ITGAL, ITGAM, ITGAV, ITGAX, ITGB1, ITGB2, ITGB3, ITGB4, ITGB5, ITGB6, ITGB7, JAG1, JAG2, KIT, LAMP2, LGALS3BP, LILRA6, LILRB1, LILRB2, LILRB3, LILRB4, LMAN2, LRRC25, LY75, M6PR, MFGE8, MMP14, MPL, MRC1, MVB12B, NECTIN1, NOMO1, NOTCH1, NOTCH2, NOTCH3, NOTCH4, NPTN, NRP1, PDCD1, PDCD1LG2, PDCD6IP, PDGFRB, PECAM1, PLXNB2, PLXND1, PROM1, PTGES2, PTGFRN, PTPRA, PTPRC, PTPRJ, PTPRO, RPN1, SDC1, SDC2, SDC3, SDC4, SDCBP, SDCBP2, SELPLG, SIGLEC7, SIGLEC9, SIRPA, SLIT2, SNF8, SPN, STX3, TACSTD2, TFRC, TLR2, TMED10, TNFRSF8, TRAC, TSG101, TSPAN14, TSPAN7, TSPAN8, TYROBP, VPS25, VPS28, VPS36, VPS37A, VPS37B, VPS37C, VPS37D, VPS4A, VPS4B, VTI1A, and VTI1B, or a variant thereof and/or a fragment thereof.

In a preferred embodiment, the cytosolic domain of one vesicle localization moiety is used to replace that of another so as to obtain a chimeric vesicle localization moiety with a surface-and-transmembrane domain of one vesicle localization moiety and a cytosolic domain of a second vesicle localization moiety. Other types of domain swapping between different vesicle localization moieties are contemplated, including chimeric vesicle localization moieties having the arrangement of ABc, AbC, Abc, aBC, aBc and abC, where A, B and C correspond to the surface domain, transmembrane domain and cytosolic domain, respectively, of a first vesicle localization moiety and a, b, and c correspond to the surface domain, transmembrane domain and cytosolic domain, respectively, of a second vesicle localization moiety. Similarly, for any chimeric vesicle localization moiety with surface domain, transmembrane domain and cytosolic domain, obtained by combining domains from about 3 or 4 distinct vesicle localization moieties, the possible number of chimeric vesicle localization moieties contemplated are about 24 and 60, respectively.

While the desired chimeric vesicle localization moieties are ones with superior localization to EVs (over parental vesicle localization moieties contributing to the chimeric vesicle localization moiety), it is also contemplated that some of these chimeric vesicle localization moieties may have desirable qualities other than ability to associate with or be incorporated as part of an EV. In a preferred embodiment, the chimeric vesicle localization moiety comprises a surface-and-transmembrane domain of a first (1^{st}) vesicle localization moiety and a cytosolic domain of a second (2^{nd}) vesicle localization moiety, which is a full-length surface-and-transmembrane domain of the 1^{st} vesicle localization moiety and a full-length cytosolic domain of a 2^{nd} vesicle localization moiety. In a preferred embodiment, the surface domain and transmembrane domain are contiguous derived from a 1^{st} vesicle localization moiety and a cytosolic domain from a 2^{nd} vesicle localization moiety.

In a separate embodiment, the chimeric vesicle localization moiety comprises a surface domain or portion thereof and a transmembrane domain or portion thereof of a 1^{st} vesicle localization moiety and a cytosolic domain or portion thereof of a 2^{nd} vesicle localization moiety. In a separate embodiment, the chimeric vesicle localization moiety comprises a surface domain or portion thereof, a transmembrane domain or portion thereof, and a cytosolic domain or portion thereof, where each domain is chosen from two or more vesicle localization moieties.

In the practice of the invention, a chimeric vesicle localization moiety may be used in place of a vesicle localization moiety, because of advantageous qualities of a chimeric vesicle localization moiety. The advantageous qualities include improved association with an EV or exosome resulting in a greater fraction of the EV or exosomes comprising a chimeric vesicle localization moiety and/or greater concentration of a chimeric VLM at an EV or exosome. Other advantageous qualities, while not limiting, include improved desired composition of EV or exosome composition, improved stability, improved size, improved targeting, improved trafficking and improved isopeptide bond formation. In an embodiment, chimeric VLM or VLM are used in the present invention as fusion proteins to one or more isopeptide domain(s) and/or isopeptide tag(s).

Alternatively, in an embodiment, EV and/or exosome may be engineered to have an isopeptide tag on a membrane protein of the EV and/or exosome so that the isopeptide tag is displayed on the outside of the EV and/or exosome where it can react with an isopeptide domain. The isopeptide domain can be fused or conjugated to a variety of different molecules, such as targeting moieties, that can impart desired function and/or properties to the EV and/or exosome. Such molecules could be attached to the EV and/or exosome by the isopeptide domain and so provide the EV and/or exosome with targeting capability for the desired target cell and/or target tissue (e.g., targeting moiety fusion protein or conjugate, wherein the targeting moiety is an affinity peptide or scFv to a marker expressed on a target cell or tissue). Binding of the isopeptide tag by the isopeptide domain results in formation of an isopeptide bond resulting in attachment of the molecule or targeting moiety to the external surface of an EV and/or exosome. Other properties that can be introduced to the EV and/or exosome by the molecule with the isopeptide domain include, for example, therapeutic entities including oligonucleotides, proteins and small molecules or combinations thereof that convey a therapeutic impact to recipient cells and tissues. In an embodiment, the membrane protein of the EV and/or exosome is a VLM or chimeric VLM.

Alternatively, it is contemplated that an isopeptide tag or isopeptide domain may be fused to other positions within a transmembrane protein (such as a VLM or chimeric VLM). While presence of an isopeptide tag or isopeptide domain N-terminal to a surface domain or transmembrane domain of a modified EV or exosome and isopeptide bond formation to a fusion protein, fusion peptide or conjugate comprising a molecule with a desired functionality or property and a complementary isopeptide domain or isopeptide tag, respectively, can confer a desired functionality or property external to the EV or exosome, the isopeptide tag or domain may be positioned adjacent to the transmembrane domain or within the transmembrane domain to introduce desired functionality or property to the external leaf or internal leaf of a lipid bilayer of the EV or exosome. Similarly, the isopeptide tag or domain may be positioned C-terminal to the transmembrane domain or cytosolic domain of a transmembrane protein (e.g., VLM or chimeric VLM) so as to introduce a desired functionality or property to the lumen of an EV or exosome. Binding to a fusion protein, fusion peptide or conjugate comprising a complementary isopeptide domain or isopeptide tag and a molecule comprising a desired functionality or property and subsequent formation of an isopeptide bond results in introduction of a desired functionality or property to the modified EV or exosome.

In a preferred embodiment, the isopeptide domain or isopeptide tag is positioned N-terminal to a surface domain or transmembrane domain of a transmembrane protein (such as a VLM or chimeric VLM) of an EV or exosome. In a preferred embodiment, the isopeptide domain or isopeptide tag may be fused to a transmembrane protein (such as a VLM or chimeric VLM) of an EV or exosome and is external to the EV or exosome.

Isopeptide bonds are amide bonds formed between carboxyl/carboxamide and amino groups, where at least one of the carboxyl or amino groups is outside of the protein main-chain (the backbone of the protein). Such bonds can be chemically irreversible under biological conditions and they are resistant to most proteases. Bond formation can be enzyme catalyzed, for example by transglutaminase enzymes, where the resulting bonds function to stabilize extracellular matrix structures or to strengthen blood clots, or isopeptide bonds may form spontaneously as has been identified in HK97 bacteriophage capsid formation and Gram-positive bacterial pili. Spontaneous isopeptide bond formation has been proposed to occur after protein folding, through nucleophilic attack of the epsilon-amino group from a lysine on the C-gamma-group of an asparagine, promoted by a nearby glutamate.

Proteins which are capable of spontaneous isopeptide bond formation can be used to make isopeptide tag/isopeptide domain partner pairs which covalently bind to each other and which hence provide irreversible interactions. In this respect, proteins which are capable of spontaneous isopeptide bond formation may be expressed as separate fragments, to give an isopeptide tag and an isopeptide domain partner for the isopeptide tag, where the two fragments are capable of covalently reconstituting by isopeptide bond formation. This covalent reaction through an isopeptide bond makes the peptide-protein interaction stable under conditions where non-covalent interactions would rapidly dissociate—over long times (e.g. weeks), at high temperature (to at least 95° C.), at high force, or with harsh chemical treatment (e.g. pH 2-11, organic solvent, detergents or denaturants). An isopeptide tag may comprise one or more residues involved in the isopeptide bond in the original protein and the isopeptide domain partner may comprise the other residue(s) involved in the isopeptide bond in the original protein. In this way, it is possible to use a peptide tag developed from a protein capable of isopeptide bond formation to engineer a protein of interest using the isopeptide domain with its partner isopeptide tag fused to two molecules that it is desired to fuse together.

An isopeptide tag and isopeptide domain pair may comprise fragments of an isopeptide protein or sequences which are homologous to such fragments e.g. which have at least 50, 60, 70, 80 or 90% identity thereto, which are able to covalently bind to one another e.g. by forming an isopeptide bond. Nucleic acid and amino acid sequence of exemplary isopeptide domains (SEQ ID NO: 1-60) and isopeptide tags (SEQ ID NO: 61-66) are provided in Table 1.

In an embodiment, an isopeptide tag may comprise a peptide that has at least 80% amino acid sequence identity to any of the isopeptide tag sequences provided herein. In an embodiment, an isopeptide domain may comprise a peptide that has at least 80% amino acid sequence identity to any of the isopeptide domain sequences provided herein. In another embodiment, an isopeptide domain may comprise a peptide that has at least 80% amino acid sequence identity to a portion of any of the isopeptide domain sequences provided herein, wherein the portion comprises at least 10 contiguous amino acids inclusive of the reactive amino acid that participates in isopeptide bond formation and forms an isopeptide bond. In another embodiment, an isopeptide domain may comprise a peptide that has at least 80% amino acid sequence identity to a portion of any of the isopeptide domain sequences provided herein, wherein the portion comprises at least 15 contiguous amino acids inclusive of the reactive amino acid that participates in isopeptide bond formation and forms an isopeptide bond. In another embodiment, an isopeptide domain may comprise a peptide that has at least 80% amino acid sequence identity to a portion of any of the isopeptide domain sequences provided herein, wherein the portion comprises between 10 to 20 contiguous amino acids inclusive of the reactive amino acid that participates in isopeptide bond formation and forms an isopeptide bond.

In an embodiment, an isopeptide tag may comprise a peptide that has at least 90% amino acid sequence similarity to any of the isopeptide tag sequences provided herein. In an embodiment, an isopeptide domain may comprise a peptide that has at least 90% amino acid sequence similarity to any of the isopeptide domain sequences provided herein. In another embodiment, an isopeptide domain may comprise a peptide that has at least 90% amino acid sequence similarity to a portion of any of the isopeptide domain sequences provided herein, wherein the portion comprises at least 10 contiguous amino acids inclusive of the reactive amino acid that participates in isopeptide bond formation and forms an isopeptide bond. In another embodiment, an isopeptide domain may comprise a peptide that has at least 90% amino acid sequence similarity to a portion of any of the isopeptide domain sequences provided herein, wherein the portion comprises at least 15 contiguous amino acids inclusive of the reactive amino acid that participates in isopeptide bond formation and forms an isopeptide bond. In another embodiment, an isopeptide domain may comprise a peptide that has at least 90% amino acid sequence similarity to a portion of any of the isopeptide domain sequences provided herein, wherein the portion comprises between 10 to 20 contiguous amino acids inclusive of the reactive amino acid that participates in isopeptide bond formation and forms an isopeptide bond.

Alternatively, an isopeptide domain may be developed from an isopeptide protein and a corresponding isopeptide tag which covalently binds thereto may be identified by screening a peptide library. The isopeptide tag and isopeptide domain fragments can each comprise an amino acid residue from the isopeptide protein which was involved in the spontaneously formed isopeptide bond. Each isopeptide bond generally forms between 2 reactive residues and thus an isopeptide tag and isopeptide domain pair which covalently bind to each other, can each comprise one of the reactive residues involved in the isopeptide bond. In this way, the isopeptide tag and isopeptide domain fragments can bind together by spontaneously forming an isopeptide bond between the reactive residue present in the isopeptide tag and the reactive residue present in the isopeptide domain. The amino acids involved in forming a spontaneous isopeptide bond can be lysine, glutamate and asparagine/aspartate and the isopeptide tag can comprise one of these residues and the isopeptide domain can comprise the other residue. In an embodiment, isopeptide bond is formed between a lysine amino acid and an aspartic amino acid or an asparagine amino acid.

In order for an isopeptide bond to form, the reactive residues e.g. the reactive lysine and asparagine residues (and particularly the relevant atoms thereof; for lysine the C-epsilon atom and for asparagine the C-gamma atom) should be positioned in close proximity to one another in space e.g. in the folded isopeptide protein. The reactive residues, e.g., the lysine and asparagine (and particularly the relevant atoms thereof) are within 4 Angstrom of each other in the folded protein and may be within 3.8, 3.6, 3.4, 3.2, 3.0, 2.8, 2.6, 2.4, 2.2, 2.0, 1.8 or 1.6 Angstrom of each other. The reactive residues (and more particularly their relevant atoms) may be within 1.81, 2.63 or 2.60 Angstrom of each other.

Examples of known proteins capable of spontaneously forming one or more isopeptide bonds include Spy0128 (Kang et al, Science, 2007, 318(5856), 1625-8, which is incorporated by reference in its entirety for all purposes), Spy0125 (Pointon et al, J. Biol. Chem., 2010, 285(44), 33858-66, which is incorporated by reference in its entirety for all purposes) and FbaB (Oke et al, J. Struct Funct Genomics, 2010, 11(2), 167-80, which is incorporated by reference in its entirety for all purposes) from Streptococcus pyogenes, Cna of Staphylococcus aureus (Kang et al, Science, 2007, 318 (5856), 1625-8, which is incorporated by reference in its entirety for all purposes), the ACE19 protein of Enterococcus faecalis (Kang et al, Science, 2007, 318(5856), 1625-8, which is incorporated by reference in its entirety for all purposes), the BcpA pilin from Bacillus cereus (Budzik et al, PNAS USA, 2007, 106(47), 19992-7, which is incorporated by reference in its entirety for all purposes), the minor pilin GBS52 from Streptococcus agalactiae (Kang et al, Science, 2007, 318(5856), 1625-8, which is incorporated by reference in its entirety for all purposes), SpaA from Corynebacterium diphtheriae (Kang et al, PNAS USA, 2009, 106(40), 16967-71, which is incorporated by reference in its entirety for all purposes), SpaP from Streptococcus mutans (Nylander et al, Acta Crystallogr Sect F Struct Biol Cryst Commum., 2011, 67(Pt1), 23-6, which is incorporated by reference in its entirety for all purposes), RrgA (Izore et al, Structure, 2010, 18(1), 106-15), RrgB (El Mortaji et al, J. Biol. Chem., 2010, 285(16), 12405-15, which is incorporated by reference in its entirety for all purposes) and RrgC (El Mortaji et al, J. Biol. Chem., 2010, 285(16), 12405-15, which is incorporated by reference in its entirety for all purposes) from Streptococcus pneumoniae, SspB from Streptococcus gordonii (Forsgren et al, J Mol Biol, 2010, 397(3), 740-51, which is incorporated by reference in its entirety for all purposes). Table 11 provides a list of isopeptide proteins which can spontaneously form an isopeptide bond and which can be used to obtain isopeptide domain and complementary isopeptide tag. Such isopeptide domain and complementary tag may be minimal in amino acid length and may be optimized for efficient isopeptide bond formation. As discussed above, any of these proteins may hence be used as an isopeptide tag/isopeptide domain pair.

Isopeptide domains that can be used herein include, for example, those of SEQ ID NO. 2, 4, 6, 8, 10, 12, 14, 16, 18, 20, 22, 26, 28, 30, 32, 36, 38, 40, 42, 46, 48, 50, 52, 56, 58, 60. Isopeptide tags that can be paired with the isopeptide domains include, for example, those of SEQ ID NO. 62, 64, 66. Pairs of isopeptide domains and isopeptide tags can include, for example, one of the isopeptide domains of SEQ ID NO: 2, 4, 6, 8, 10, 12, 14, 16, 18, 20, 22, 26, 28, 30, 32, 36, 38, 40, 42, 46, 48, 50, 52, paired with one of the isopeptide tags of SEQ ID NO: 62; one of the isopeptide domains of SEQ ID NO: 56, 58 paired with one of the isopeptide tags of SEQ ID NO: 64; and one of the isopeptide domains of SEQ ID NO: 60 paired with one of the isopeptide tags of SEQ ID NO: 66. Examples of isopeptide domains follow (with reactive amino acid participating in isopeptide bond formation underlined).

(SEQ ID NO: 2)
GAMVDTLSGLSSEQGQSGDMTIEEDSATHIKFSKRDEDGKELAGATMEL
RDSSGKTISTWISDGQVKDFYLYPGKYTFVETAAPDGYEVATAITFTVN
EQGQVTVNGKATKGDAHI
(SEQ ID NO: 4)
AMVDTLSGLSSEQGQSGDMTIEEDSATHIKFSKRDEDGKELAGATMELR
DSSGKTISTWISDGQVKDFYLYPGKYTFVETAAPDGYEVATAITFTVNE
QGQVTVNGKATKGDAHI
(SEQ ID NO: 6)
MVDTLSGLSSEQGQSGDMTIEEDSATHIKFSKRDEDGKELAGATMELRD
SSGKTISTWISDGQVKDFYLYPGKYTFVETAAPDGYEVATAITFTVNEQ
GQVTVNGKATKGDAHI
(SEQ ID NO: 8)
VDTLSGLSSEQGQSGDMTIEEDSATHIKFSKRDEDGKELAGATMELRDS
SGKTISTWISDGQVKDFYLYPGKYTFVETAAPDGYEVATAITFTVNEQG
QVTVNGKATKGDAHI
(SEQ ID NO: 10)
VDTLSGLSSEQGQSGDMTIEEDSATHIKFSKRDEDGKELAGATMELRDS
SGKTISTWISDGQVKDFYLYPGKYTFVETAAPDGYEVATAITFTVNEQG
QVTVNGKATKGDAHID
(SEQ ID NO: 12)
VDTLSGLSSEQGQSGDMTIEEDSATHIKFSKRDEDGKELAGATMELRDS
SGKTISTWISDGQVKDFYLYPGKYTFVETAAPDGYEVATAITFTVNEQG
QVTVNGKATKG
(SEQ ID NO: 14)
VDTLSGLSSEQGQSGDMTIEEDSATHIKESKRDEDGKELAGATMELRDS
SGKTISTWISDGQVKDFYLYPGKYTFVETAAPDGYEVATAITFTVNEQG
QVTVNGKATK
(SEQ ID NO: 16)
VDTLSGLSSEQGQSGDMTIEEDSATHIKFSKRDEDGKELAGATMELRDS
SGKTISTWISDGQVKDFYLYPGKYTFVETAAPDGYEVATAITFTVNEQG
QVTVNGKATKGDA
(SEQ ID NO: 18)
VDTLSGLSSEQGQSGDMTIEEDSATHIKFSKRDEDGKELAGATMELRDS
SGKTISTWISDGQVKDFYLYPGKYTFVETAAPDGYEVATAITFTVNEQG
QVTVNGKATKGDAH
(SEQ ID NO: 20)
DTLSGLSSEQGQSGDMTIEEDSATHIKFSKRDEDGKELAGATMELRDSS
GKTISTWISDGQVKDFYLYPGKYTFVETAAPDGYEVATAITFTVNEQGQ
VTVNGKATKGDAHI
(SEQ ID NO: 22)
DSATHIKFSKRDEDGKELAGATMELRDSSGKTISTWISDGQVKDFYLYP
GKYTFVETAAPDGYEVATAITFTVNEQGQVTVNGKATKGDAHI
(SEQ ID NO: 26)
AMVDTLSGLSSEQGQSGDMTIEEDSATHIKFSKRDEDGKELAGATMELR
DSSGKTISTWISDGQVKDFYLYPGKYTFVETAAPDGYEVATAITFTVNE
QGQVTVNGKATKGDAHID
(SEQ ID NO: 28)
VDTLSRLSSEQGQSGDMTIEEDSATHIKFSKRDEDGKELAGATMELRDS
SGKTISTWISDGQVKDFYLYPGKYTFCRNRSTRRYGGSTAIPYSMEQGQ
VTVMASN
(SEQ ID NO: 30)
DSATHIKFSKRDEDGKELAGATMELRDSSGKTISTWISDGQVKDFYLYP
GKYTFVETAAPDGYEVATAITFTVNEQGQVTVNGKATK
(SEQ ID NO: 32)
DSATHIKFSKRDEDGKELAGATMELRDSSGKTISTWISDGQVKDFYLYP
GKYTFVETAAPDGYEVATAITFTVNEQGQVTVNG
(SEQ ID NO: 36)
DSATHIKFSKRDIDGKELAGATMELRDSSGKTISTWISDGQVKDFYLMP
GKYTFVETAAPDGYEVATAITFTVNEQGQVTVNGKATKGDAHAVMVAA
(SEQ ID NO: 38)
DSATHIKFSKRDEDGKELAGATMELRDSSGKTISTWISDGQVKDFYLYP
GKYTFVETAAPDGYEVATAITFTVNEQGQVTVNGKATKG
(SEQ ID NO: 40)
VDTLSGLSSEQGQSGDMTIEEDSATHIKFSKRDEDGKELAGATMELRDS
SGKTISTWISDGQVKDFYLYPGKYTFVETAAPDGYEVATAITFTVNEQG
QVTVNGLE
(SEQ ID NO: 42)
MVDTLSGLSSEQGQSGDMTIEEDSATHIKFSKRDEDGKELAGATMELRD
SSGKTISTWISDGQVKDFYLYPGKYTFVETAAPDGYEVATAITFTVNEQ
GQVTVNGKATK
(SEQ ID NO: 46)
VTTLSGLSGEQGPSGDMTTEEDSATHIKFSKRDEDGRELAGATMELRDS
SGKTISTWISDGHVKDFYLYPGKYTFVETAAPDGYEVATAITFTVNEQG
QVTVNGEATKGDAHT
(SEQ ID NO: 48)
MTIEEDSATHIKFSKRDEDGKELAGATMELRDSSGKTISTWISDGQVKD
FYLYPGKYTFVETAAPDGYEVATAITFTVNEQGQVT
(SEQ ID NO: 50)
IETEQNLPNEDGQSGNIIEQEDSKTLVKFSKRDIKGNELAGATIELRDL
SGKSIQSWVSDGKAKDFYLLPGSYEFVETAAPEGYQIATKIMFTISTDG
RITVDGQLV
(SEQ ID NO: 52)
EEDSATHIKFSKRDEDGKELAGATMELRDSSGKTISTWISDGQVKDFYL
YPGKYTFVETAAPDGYEVATAITFTVNEQGQVTVNGKATKG
(SEQ ID NO: 56)
KPLRGAVFSLQKQHPDYPDIYGAIDQNGTYQNVRTGEDGKLTFKNLSDG
KYRLFENSEPAGYKPVQNKPIVAFQIVNGEVRDVTSIVPQDIPATYEFT
NGKHYITNEPIPPK
(SEQ ID NO: 58)
GSHMKPLRGAVFSLQKQHPDYPDIYGAIDQNGTYQNVRTGEDGKLTFKN
LSDGKYRLFENSEPAGYKPVQNKPIVAFQIVNGEVRDVTSIVPQDIPAT
YEFTNGKHYITNEPIPPK
(SEQ ID NO: 60)
SSGLVPRGSHMASMTGGQQMGRGSSGLSGETGQSGNTTIEEDSTTHVKF
SKRDANGKELAGAMIELRNLSGQTIQSWISDGTVKVFYLMPGTYQFVET
AAPEGYELAAPITFTIDEKGQIWVDS.

Additional isopeptide domains include the following present as a fusion to a targeting moiety. In one example, the targeting moiety is a single chain Fv (scFv) antibody, such as a GC33 single chain Fv (scFv) antibody fragment directed against glypican-3 (GPC3) cell surface protein. In this specific example, sequences which are in bold corresponds to a heavy chain variable region of an antibody comprising complementary determining region (CDR) and framework sequences (FR) found outside the CDR sequences in the order: FR1-CDR1-FR2-CDR2-FR3-CDR3-FR4. The underlined sequences signify a linker. The sequences which are both in bold and italics signify a light chain variable region of an antibody comprising complementary determining region (CDR) and framework sequences (FR) found outside the CDR sequences in the order: FR1-CDR1-FR2-CDR2-FR3-CDR3-FR4. Sequences which are in italics signify any of an isopeptide-1, -2 or -3 domain. Sequences in lowercase letters signify an epitope sequence.

### GC33+Isopeptide Domain (Isopeptide-1)

(SEQ ID NO: 248)
QVQLVQSGAEVKKPGASVKVSCKASGYTFTDYEMHWVRQAPGQGLEWMGALDP
KTGDTAYSQKFKGRVTLTADKSTSTAYMELSSLTSEDTAVYYCTRFYSYTYWGQG
TLVTVSS
SSGGSSRSSSSGGGGSGGGG
DVVMTQSPLSLPVTPGEPASISCRSSQSLVHSNGNTYLHWYLQKPGQSPOLLIYKVSNRF
SGVPDRFSGSGSGTDETLKISRVEAEDYGVYYCSQNTHVPPTFGQGTKLEIKSGGGGSG
GGGketaaakferqhmdsDSATHIKFSKRDEDGKELAGATMELRDSSGKTISTWISDGQVKDFYLY
PGKYTFVETAAPDGYEVATAITFTVNEQGQVTVNG

The isopeptide domain (isopeptide-1) binds to Isopeptide(1) tag:

(SEQ ID NO: 62)
AHIVMVDAYKPTK.

### GC33+Isopeptide Domain (Isopeptide-2)

(SEQ ID NO: 250)
QVQLVQSGAEVKKPGASVKVSCKASGYTFTDYEMHWVRQAPGQGLEWMGALDP
KTGDTAYSQKFKGRVTLTADKSTSTAYMELSSLTSEDTAVYYCTRFYSYTYWGQG
TLVTVSS
SSGGSSRSSSSGGGGSGGGG
DVVMTQSPLSLPVTPGEPASISCRSSQSLVHSNGNTYLHWYLQKPGQSPQLLIYKVSNRF
SGVPDRFSGSGSGTDFTLKISRVEAEDVGVYYCSQNTHVPPTFGQGTKLEIKSGGGGSG
GGGeqkliseedlGSHMKPLRGAVFSLQKQHPDYPDIYGAIDQNGTYQNVRTGEDGKLTFKN
LSDGKYRLFENSEPAGYKPVQNKPIVAFQIVNGEVRDVTSIVPQDIPATYEFTNGKHYIT
NEPIPPK

The isopeptide domain (isopeptide-2) binds to isopeptide(2) tag:

(SEQ ID NO: 64)
KLGDIEFIKVNK.

### GC33+Isopeptide Domain (Isopeptide-3)

(SEQ ID NO: 252)
QVQLVQSGAEVKKPGASVKVSCKASGYTFTDYEMHWVRQAPGQGLEWMGALDP
KTGDTAYSQKFKGRVTLTADKSTSTAYMELSSLTSEDTAVYYCTRFYSYTYWGQG
TLVTVSS
SSGGSSRSSSSGGGGSGGGG
DVVMTQSPLSLPVTPGEPASISCRSSQSLVHSNGNTYLHWYLQKPGQSPQLLIYKVSNRF
SGVPDRFSGSGSGTDFTLKISRVEAEDVGVYYCSQNTHYPPTFGQGTKLEIKSGGGGSG
GGGgkpipnpllgldstSSGLVPRGSHMASMTGGQQMGRGSSGLSGETGQSGNTTIEEDSTTH
VKFSKRDANGKELAGAMIELRNLSGQTIQSWISDGTVKVFYLMPGTYQFVETAAPEGYE
LAAPITFTIDEKGQIWVDS

The isopeptide domain (isopeptide 3) binds isopeptide(3) tag: DPIVMIDNDKPIT (SEQ ID NO: 66).

Examples of isopeptide tags follow (with reactive amino acid participating in isopeptide bond formation underlined):

(SEQ ID NO: 62)
AHIVMVDAYKPTK
(SEQ ID NO: 64)
KLGDIEFIKVNK
(SEQ ID NO: 66)
DPIVMIDNDKPIT

The isopeptide tag and/or isopeptide domain may be fused to molecules that provide the EV and/or exosome with a desired function and/or property (a molecule so fused may be referred to in the application as a “molecule tag;” while a preferred embodiment is to fuse the molecule with an isopeptide tag, the molecule may also be fused to an isopeptide domain depending on the partner polypeptide which will participate in isopeptide bond formation). The molecule can be a polypeptide (such as, for example, a targeting protein, e.g., single chain Fv (scFv)), a peptide or affinity peptide (such as, for example, THVSPNQGGLPS (SEQ ID NO: 196) affinity peptide for GPC3), lipid, carbohydrate, nucleic acid, ligand, aptamer, polymer, small molecule drug, chemical compound, or other macromolecule with a desired biochemical or biophysical property. When the molecule is a protein or polypeptide, the isopeptide tag can be fused, for example, at the N- or C-terminus of such proteins or polypeptides or in an internal loop. Optionally, a linker may flank the isopeptide tag or isopeptide domain, e.g. a glycine/serine rich linker, in order to enhance accessibility for reaction. The linker may include a site for cleavage.

The isopeptide domain can be fused with a vesicle localization moiety that is present on an EV and/or exosome. Examples of vesicle localization moieties that can be fused with the isopeptide domain include, for example, ACE, ADAM10, ADAM15, ADAM9, AGRN, ALCAM, ANPEP, ANTXR2, ATP1A1, ATP1B3, BSG, BTN2A1, CALM1, CANX, CD151, CD19, CD1A, CD1B, CD1C, CD2, CD200, CD200R1, CD226, CD247, CD274, CD276, CD33, CD34, CD36, CD37, CD3E, CD40, CD40LG, CD44, CD47, CD53, CD58, CD63, CD81, CD82, CD84, CD86, CD9, CHMP1A, CHMP1B, CHMP2A, CHMP3, CHMP4A, CHMP4B, CHMP5, CHMP6, CLSTN1, COL6A1, CR1, CSF1R, CXCR4, DDOST, DLL1, DLL4, DSG1, EMB, ENG, EVI2B, F11R, FASN, FCER1G, FCGR2C, FLOT1, FLOT2, FLT3, FN1, GAPDH, GLG1, GRIA2, GRIA3, GYPA, HSPG2, ICAM1, ICAM2, ICAM3, IGSF8, IL1RAP, IL3RA, IL5RA, IST1, ITGA2, ITGA2B, ITGA3, ITGA4, ITGA5, ITGA6, ITGAL, ITGAM, ITGAV, ITGAX, ITGB1, ITGB2, ITGB3, ITGB4, ITGB5, ITGB6, ITGB7, JAG1, JAG2, KIT, LAMP2, LGALS3BP, LILRA6, LILRB1, LILRB2, LILRB3, LILRB4, LMAN2, LRRC25, LY75, M6PR, MFGE8, MMP14, MPL, MRC1, MVB12B, NECTIN1, NOMO1, NOTCH1, NOTCH2, NOTCH3, NOTCH4, NPTN, NRP1, PDCD1, PDCD1LG2, PDCD6IP, PDGFRB, PECAM1, PLXNB2, PLXND1, PROM1, PTGES2, PTGFRN, PTPRA, PTPRC, PTPRJ, PTPRO, RPN1, SDC1, SDC2, SDC3, SDC4, SDCBP, SDCBP2, SELPLG, SIGLEC7, SIGLEC9, SIRPA, SLIT2, SNF8, SPN, STX3, TACSTD2, TFRC, TLR2, TMED10, TNFRSF8, TRAC, TSG101, TSPAN14, TSPAN7, TSPAN8, TYROBP, VPS25, VPS28, VPS36, VPS37A, VPS37B, VPS37C, VPS37D, VPS4A, VPS4B, VTI1A and VTI1B or an isoform thereof, a homologue thereof, a variant thereof or a functional fragment thereof, or an exosomal polypeptide. These exemplary vesicle localization moieties are single pass membrane proteins and the isopeptide domain can be fused with or without a linker to the N-terminal portion of the vesicle localization moieties. The isopeptide domain can be at the N-terminus or at any position on the vesicle localization moiety that is outside the cell. For example, the isopeptide domain could be fused between the surface and transmembrane domains of the vesicle localization moiety, or the isopeptide domain could be fused to the vesicle localization moiety so that the isopeptide domain is displayed on a surface domain of the vesicle localization moiety in a desired manner. When the isopeptide domain may be fused to the vesicle localization moiety, linkers may be used at one or both sides of the isopeptide domain sequence.

Further, the vesicle localization moiety may be a chimeric vesicle localization moiety comprising a portion of a first vesicle localization moiety selected from the list above joined to a portion of a second vesicle localization moiety different from the first and also selected from the list above. In one embodiment, the chimeric vesicle localization moiety comprises the extracellular domain and transmembrane of a first vesicle localization moiety and the cytosolic domain of a second vesicle localization moiety. In another embodiment, the chimeric vesicle localization moiety comprises a portion of the N-terminal fragment of the first vesicle localization moiety and a portion of the C-terminal fragment of the second vesicle localization moiety, wherein the chimeric vesicle localization moiety is incorporated into extracellular vesicles and comprises an extracellular domain and a transmembrane domain. In another embodiment, the chimeric vesicle localization moiety comprises a portion of the N-terminal fragment of the first vesicle localization moiety and a portion of the C-terminal fragment of the second vesicle localization moiety, wherein the chimeric vesicle localization moiety is incorporated into extracellular vesicles and comprises an extracellular domain, a transmembrane domain and a cytosolic domain.

Alternatively, the vesicle localization moiety can be a multipass membrane protein and the isopeptide domain may be fused with one of the surface loops or domains of the multipass vesicle localization moiety. When the isopeptide domain may be fused to the vesicle localization moiety, linkers may be used at one or both sides of the isopeptide domain sequence.

In an embodiment, an EV may have or comprise a vesicle localization moiety of the invention. In a separate embodiment, an EV may have or comprise a combination of vesicle localization moieties of the invention. In a separate embodiment, an EV may have or comprise a combination of two or more vesicle localization moieties of the invention. In another embodiment, an EV may have or comprise a combination of three or more vesicle localization moieties of the invention. In another embodiment, an EV may have or comprise a combination of four or more vesicle localization moieties of the invention. In another embodiment, an EV may have or comprise a combination of five or more vesicle localization moieties of the invention. In an embodiment, an EV may have many copies of a vesicle localization moiety of the invention. In another embodiment, an EV may have many copies of two or more vesicle localization moiety of the invention.

EVs and/or exosomes, and/or molecule-vesicle localization moiety fusion protein can also be engineered to increase their serum half-life and reduce their immunogenicity. As used herein, a “vesicle localization moiety fusion protein” (also called “VLM fusion protein”) is a fusion protein comprising a vesicle localization moiety and a polypeptide (e.g., protein or peptide) that can participate and form an isopeptide bond. A “chimeric vesicle localization moiety fusion protein” (also called “chimeric VLM fusion protein”) is a fusion protein comprising a chimeric vesicle localization moiety and a polypeptide (e.g., protein or peptide) that can participate and form an isopeptide bond. The polypeptide that can participate and form an isopeptide bond may be an isopeptide domain or an isopeptide tag. As used herein, a “molecule tag” (may also be called “targeting moiety fusion protein” or “targeting moiety conjugate” or “molecule of interest fusion protein” or “molecule of interest conjugate” depending on context) is an isopeptide domain or isopeptide tag covalently coupled to a molecule of interest (i.e., desired molecule) wherein the molecule of interest may be a polypeptide (e.g., protein (such as, a targeting protein (e.g., scFv) or peptide or affinity peptide (such as, a GPC affinity peptide)), lipid, carbohydrate, nucleic acid, ligand, aptamer, small molecules, chemical compound or macromolecules. A targeting moiety fusion protein (or targeting moiety fusion peptide) or molecule of interest fusion protein (or molecule of interest fusion peptide) can be a single polypeptide derived from two separate polypeptides or portions of two separate polypeptides, wherein one of the two polypeptides or portion thereof is a targeting moiety of interest or a molecule of interest and the other polypeptide or portion thereof is an isopeptide domain or isopeptide tag. A targeting moiety conjugate or molecule of interest conjugate can be an isopeptide domain or isopeptide tag covalent linked to a lipid, carbohydrate, nucleic acid, nucleic acid analog, ligand, aptamer, small molecules, chemical compound or macromolecules. Such covalent linkage often requires chemical crosslinking or photocrosslinking in order to form conjugates.

In a preferred embodiment, the molecule tag may be a fusion of an isopeptide tag and a polypeptide (such as a scFv antibody or affinity peptide). As used herein, a “molecule-VLM fusion protein” is a fusion protein comprising a VLM fusion protein and a molecule tag, so as to permit an EV to display a targeting moiety(ies) (e.g., a desired peptide or protein) on its surface in which the desired molecule is coupled to the surface of the EV through an isopeptide bond covalently linking the molecule to a vesicle localization moiety. As such, the “molecule-vesicle localization moiety fusion protein” is a fusion protein comprising a fusion protein of a vesicle localization moiety and a polypeptide that can participate and form an isopeptide bond covalent linked via an isopeptide bond to a molecule tag comprising a molecule of interest (for example, a targeting peptide or polypeptide) fused to a polypeptide partner that can participate and form an isopeptide bond. For example, in a preferred embodiment, the vesicle localization moiety may be fused to an isopeptide domain while the molecule, a desired moiety such as, for example, a desired peptide or protein to target a cell or a specific cell type, may be fused to an isopeptide tag so that the isopeptide domain and isopeptide tag may participate in formation of an isopeptide bond thereby covalently linking the vesicle localization moiety and the molecule (of interest). In a separate embodiment, the vesicle localization moiety may be fused to an isopeptide tag while the molecule may be fused to an isopeptide domain. Further, the isopeptide domain may be fragmented to produce a catalytic domain and a peptide fragment; the latter (or its derivative) may still participate in isopeptide bond formation catalyzed by the split catalytic domain.

As such, in an embodiment, the vesicle localization moiety may be fused to an isopeptide tag (vesicle localization moiety fusion protein), the molecule (e.g., a peptide or protein of interest) may be fused to a second isopeptide tag (molecule tag), and an isopeptide bond between the vesicle localization moiety fusion protein and the molecule tag may be catalyzed by a split catalytic domain (a ligase) derived from fragmenting an isopeptide domain into a catalytic component and a second isopeptide tag. Further, the EV and/or exosome can be modified with a protracting moiety that can be made of 1, 2, 3, 4, 5 or more moieties of a synthetic polymer. The synthetic polymer can be biodegradable or non-biodegradable. Biodegradable polymers useful as protracting moieties include, but are not limited to, poly(2-methacryloyloxyethyl phosphorylcholine) (PMPC) and poly[oligo(ethylene glycol) methyl ether methacrylate] (POEGMA). Non-biodegradable polymers useful as protracting moieties include without limitation poly(ethylene glycol)(PEG), polyglycerol, poly(N-(2-hydroxypropyl)methacrylamide)(PHPMA), polyoxazolines and poly(N-vinylpyrrolidone)(PVP).

The synthetic polymer can include or be a PEG. Conjugation of one or more PEG moieties to a protein increases its half-life by increasing its MW, hydrodynamic radius/volume and overall size and thus reducing its renal clearance. PEGylation can increase the solubility, reduce the aggregation and immunogenicity, and avoid phagocytosis of the protein. PEG is flexible, uncharged and non-biodegradable, has relatively low immunogenicity, and has been approved by the US Food and Drug Administration as GRAS (generally recognized as safe). The one or more PEG moieties independently can be linear or branched. Furthermore, the one or more PEG moieties independently can terminate in a hydroxyl group, a methoxy group (mPEG) or another capped group.

In some embodiments, the individual mass (e.g., average molecular weight), or the total mass, of the one or more synthetic polymer moieties is about 10-50, 10-20, 20-30, 30-40 or 40-50 kDa, or about 10, 20, 30, 40 or 50 kDa. The individual mass (e.g., average MW), or the total mass, of the one or more synthetic polymer moieties can also be greater than about 50 kDa, such as about 50-100, 50-60, 60-70, 70-80, 80-90 or 90-100 kDa, or about 60, 70, 80, 90 or 100 kDa. Moreover, the mass (e.g., average MW) of an individual synthetic polymer moiety can be less than about 10 kDa, such as about 1-5 or 5-10 kDa, or about 5 kDa. In certain embodiments, the individual mass (e.g., average MW), or the total mass, of the one or more synthetic polymer (e.g., PEG) moieties is about 20 or 40 kDa. The half-life of the modified protein can be tuned based on the length of the synthetic polymer, with a longer polymer generally conferring a longer half-life.

EVs and/or exosomes, and/or molecule-vesicle localization moiety fusion protein can be engineered to include post translation modifications that reduce the immunogenicity of the EV and/or exosome, and/or the molecule-vesicle localization moiety fusion protein. Such post translational modifications can include, for example, glycosylation (e.g., N-linked glycosylation and O-linked glycosylation), lipidation, phosphorylation, sulfation, acetylation (e.g., acetylation of the N-terminus), amidation (e.g., amidation of the C-terminus), hydroxylation, methylation, formation of an intramolecular or intermolecular disulfide bond, formation of a lactam between two side chains, formation of pyroglutamate, and ubiquitination.

### Targeting Moieties of Interest

Any of the extracellular vesicles disclosed herein may include one or more targeting moieties of interest. They can be embedded in or displayed on vesicle membranes. The extracellular vesicle can be an exosome, and the targeting moiety can be displayed on the outer surface of the exosome. For example, the targeting moiety may be displayed/joined/attached to the surface domain of the chimeric localization moiety.

In a preferred example, the invention provides an extracellular vesicle of the invention comprising a fusion protein comprising (1) a chimeric vesicle localization moiety comprising a surface and transmembrane domain of a first vesicle localization moiety and a cytosolic domain of a second vesicle localization moiety and (2) one or more isopeptide domain(s) and/or isopeptide tag(s), wherein one or more targeting moiety fusion protein(s), peptide(s) or conjugate(s) comprising a complementary isopeptide tag or isopeptide domain and a targeting moiety is covalently attached via an isopeptide bond formed between an isopeptide domain and an isopeptide tag. In a preferred embodiment, the cytosolic domain of the second (2^{nd}) VLM replaces cytosolic domain of the first (1^{st}) VLM in the chimeric VLM. Further, herein, other chimeric vesicle localization moieties are contemplated having the arrangement of ABc, AbC, Abc, aBC, aBc and abC, where A, B and C correspond to the surface domain, transmembrane domain and cytosolic domain, respectively, of a first vesicle localization moiety and a, b, and c correspond to the surface domain, transmembrane domain and cytosolic domain, respectively, of a second vesicle localization moiety. In a preferred embodiment, the targeting moiety(ies) is/are similarly displayed on the external surface of these engineered EVs and/or exosomes.

Targeting moieties (such as tissue specific targeting moieties) can comprise a small molecule, glycoprotein, polypeptides, peptide, oligopeptide, protein, lipid, carbohydrate, nucleic acid, polysaccharides, ligand, aptamer, small molecules, chemical compound or macromolecules, therapeutic drugs, imaging moieties or other molecules that facilitates the targeting of the vesicle to a cell or tissue of interest. The term “polypeptide,” “peptide,” “oligopeptide,” and “protein,” are used interchangeably herein, and refer to a polymeric form of amino acids of any length, which can include coded and non-coded amino acids, chemically, or biochemically modified or derivatized amino acids, and polypeptides having modified peptide backbones. Targeting moieties can comprise a nucleic acid analog, such as, antisense oligonucleotide (ASO, 2′-O-methyl (OMe), 2′-fluoro (F), and 2′-O-methoxyethyl (MOE) RNA, locked nucleic acid (LNA), constrained ethyl (cEt), phosphorodiamidate morpholinos (PMOs), phosphorothioate, and peptide nucleic acid (PNA)).

In one embodiment of the invention, a targeting moiety may be an antibody, a ligand or a functional epitope thereof that binds to a cell or tissue marker, for example, a cell surface receptor.

As used herein, the term antibody can be a protein or polypeptide functionally defined as a binding protein and structurally defined as comprising an amino acid sequence that is recognized by one of skill in the art as being derived from a variable region of an immunoglobulin. An antibody can comprise one or more polypeptides substantially encoded by immunoglobulin genes, fragments of immunoglobulin genes, hybrid immunoglobulin genes (made by combining the genetic information from different animals), or synthetic immunoglobulin genes. The recognized, native, immunoglobulin genes can include the kappa, lambda, alpha, gamma, delta, epsilon and mu constant region genes, as well as myriad immunoglobulin variable region genes and multiple D-segments and J-segments. Light chains can be classified as either kappa or lambda. Heavy chains can be classified as gamma, mu, alpha, delta, or epsilon, which in turn define the immunoglobulin classes, IgG, IgM, IgA, IgD and IgE, respectively.

Antibodies may exist as intact immunoglobulins, as a number of well characterized fragments produced by digestion with various peptidases to produce, for example, antigen-binding fragments F(ab′)₂, Fab and Fab′, or as a variety of fragments made by recombinant DNA technology, such as variable fragment (Fv), single chain variable fragment (scFv), diabodies, tascFv, bis-scFv, nanobody (e.g., V_{H}H or V_{NAR} fragment), and miniaturized “3G” fragment (Nelson, A. L. (2010) Antibody fragments. MAbs 2: 77-83; and Muyldermans, S. (2103) Nanobodies: natural single-domain antibodies. Ann. Rev. Biochem. 82:775-797). Antibodies can derive from many different species (e.g., rabbit, sheep, camel, human, or rodent, such as mouse or rat), or can be synthetic. Antibodies can be chimeric, humanized, or human. Antibodies can be monoclonal or polyclonal, multiple or single chained, fragments or intact immunoglobulins. In a preferred embodiment, the antibody is a scFv. Examples of scFv as targeting moieties are provided in Table 6 under SEQ ID NO: 151-156 for both nucleic acid coding sequence as well as amino acid sequence.

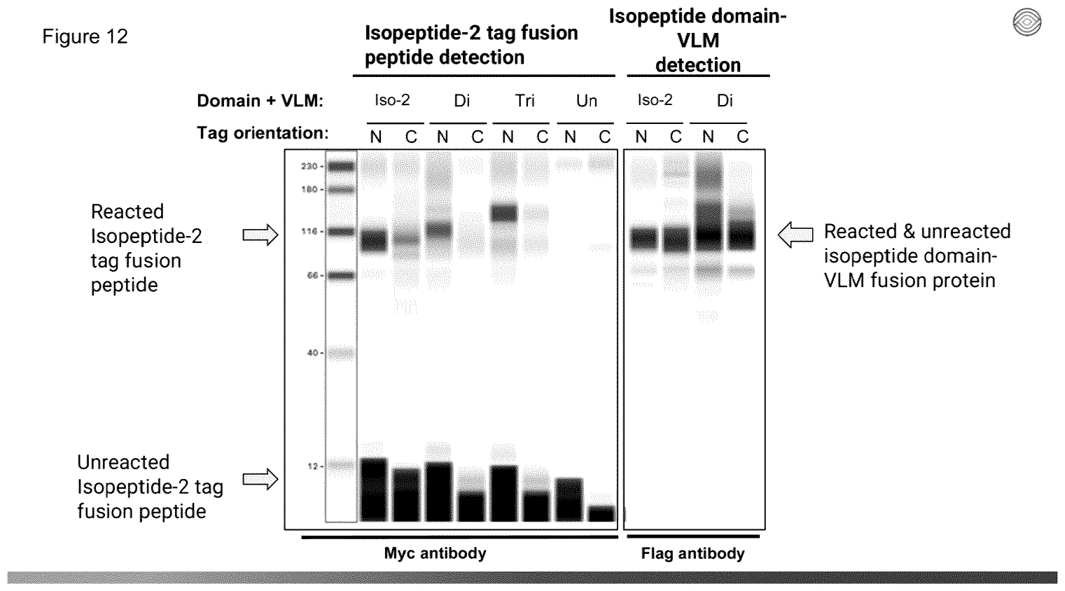

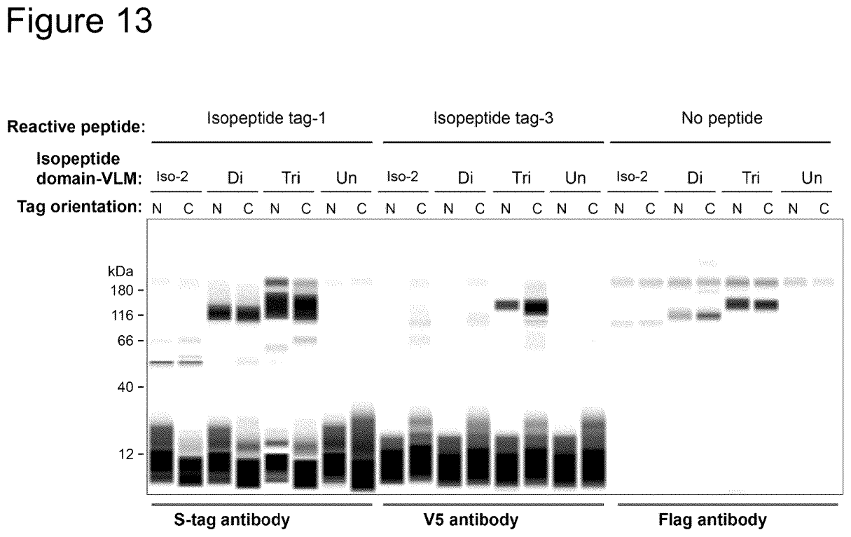

In an embodiment of the invention, the targeting moiety is a peptide (e.g., an affinity peptide). Examples of affinity peptides are provided in Table 6 under SEQ ID NO: 157-196 for both nucleic acid coding sequence as well as amino acid sequence and additionally in FIGS. 12 and 13 and Examples 11 and 12.

In another embodiment, the targeting moiety may be an antibody fragment. In yet another embodiment, the antibody fragment may be any of F(ab′)₂, Fab, Fab′, Fv, scFv, diabodies, tascFv, bis-scFv, nanobody and miniaturized “3G” fragment. In a preferred embodiment, the antibody fragment is single chain Fv (scFv), wherein variable region of heavy chain (V_{H}) and variable region of light chain (V_{L}) are joined together by a flexible linker. The variable region of heavy chain fragment can precede the variable region of light chain fragment, or vice versa. The flexible linker is often glycine-serine rich, such as a (GGGGS)₄ linker. In one embodiment, the scFv binds a target on the surface of a cell or tissue. In a preferred embodiment, the scFv is attached to a chimeric vesicle localization moiety incorporated in an extracellular vesicle (such as an exosome) and displayed outside the extracellular vesicle (e.g., exosome). In a more preferred embodiment, the scFv is attached to a chimeric vesicle localization moiety and displayed outside an extracellular vesicle (e.g., exosome) preferentially or selectively targets a specific cell type or tissue. Merely by way of example, the antibody fragment may be monospecific or bispecific. In an embodiment of the invention, the antibody fragment may be multivalent.

Examples of suitable antibodies particularly single chain Fv antibodies; and fragments, include antibodies directed against any of Thy1, MHC class II, C3d-binding region of complement receptor type 2 (CR2), VCAM-1, E-selectin, alpha 8 integrin, integrin alpha-M (CD11b) and CD163. Exemplary antibodies from which Fab and/or scFv antibodies may be prepared include OX7 antibody against Thy1 protein (Suana, A. J. et al., J. Pharmacol. Exp. Ther. 2011; 337:411-422; RT1 antibody against MHC class II protein (Hultman, K. L. et al., ACS Nano. 2008; 2:477-484); monoclonal antibody to C3d binding region of CR2 (Serkova, N. J. et al., Radiology. 2010; 255:517-526); monoclonal antibody to VCAM-1 (clone M/K2, Cambridge Bioscience) (Akhtar, A. M., PLoS One. 2010; 5:e12800); monoclonal antibody, MES-1, directed to E-selectin (Asgeirsdottir, S. A. et al., Mol. Pharmacol. 2007; 72:121-131); anti-α8 integrin antibody (Santa Cruz Biotechnologies) (Scindia, Y. et al., Arthritis Rheum. 2008; 58:3884-3891); monoclonal antibody against CD11b (Shirai, T. et al., Drug Targeting. 2012; 20:535-543); and anti-CD163 monoclonal antibody (ED2; sc-58965, Santa Cruz Biotechnology) (Sawano, T. et al. 2015. Oncology reports. 33: 2151-60). Suitable examples of scFv as targeting moieties are provided in Table 6 under SEQ ID NO: 151-156 for both nucleic acid coding sequence as well as amino acid sequence and in FIGS. 19-21.

Any of the targeting moieties described herein can enhance the selectivity of the vesicles towards the target cell of interest as compared to one or more other tissues or cells. The one or more selective targeting moieties can be expressed on modified vesicles in a way that allows such modified vesicles to bind to intended targets. The one or more targeting moieties can expose sufficient amount of amino acids to allow such binding.

The modified vesicles provided herein can comprise one or more targeting moieties that selectively target the vesicles to cells or tissue of interest by binding or physically interacting with markers expressed on such cells.

The term “selective” or “selectively” as used herein in the context of selective targeting or selective binding or selective interaction can refer to a preferential targeting, binding or interaction to a cell, tissue, or organ of interest as compared to at least one other type of cell, tissue or organ.

A “functional fragment” of a protein can mean a fragment of the protein which retains a function of a full-length protein from which it is derived, e.g., a targeting or binding function identical or similar to that of the full-length protein. A “functional fragment” of an antibody can be its antigen binding portion or fragment, which confers binding specificity for the intact antibody. A function can be similar to a function of a full-length protein if it retains at least 75%, 80%, 85%, 90%, 95%, 99%, or 100% of that function of the full-length protein. The function can be measured e.g., using an assay, e.g., an in vivo binding assay, a binding assay in a cell, or an in vitro binding assay.

In general, “sequence identity” or “sequence homology”, refer to a nucleotide-to-nucleotide or amino acid-to-amino acid correspondence of two polynucleotides or polypeptide sequences, respectively. As used herein, “sequence identity” or “identity” refers, in the context of two nucleic acid sequences or amino acid sequences, to the residues in the two sequences that are the same when aligned for maximum correspondence over a specified comparison window.

As used herein, “percent sequence identity” means the value determined by comparing two optimally aligned sequences over a comparison window, wherein (the portion of the polynucleotide or polypeptide sequence in the comparison window may comprise additions or deletions (i.e., gaps) compared to the reference sequence which does not comprise additions or deletions comprises) can for optimal alignment of the two sequences. The percentage can be calculated by determining the number of positions at which the identical nucleotide or amino acid occurs in both sequences to yield the number of matched positions, dividing the number of matched positions by the total number of positions in the comparison window and multiplying the result by 100 to determine the percentage of sequence identity.

Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings; Needleman, S. B. and Wunsch, C. D. (1970) A general method applicable to the search for similarities in the amino acid sequence of two proteins. J. Mol. Biol. 48:443-53), the BLAST algorithm (see, e.g., the BLAST alignment tool available at blast.ncbi.nlm.nih.gov/Blast.cgi, optionally with default settings; Altschul, S. F. et al. (1990) Basic local alignment search tool. J. Mol. Biol. 215:403-410; and Altschul, S. F. et al. (1997) Gapped BLAST and PSI-BLAST: a new generation of protein database search programs. Nucleic Acids Res. 25:3389-3402), and the Smith-Waterman algorithm (see, e.g., the EMBOSS Water aligner available at www.ebi.ac.uk/Tools/psa/emboss water/, optionally with default settings; Smith, T. F. and Waterman, M. S. (1981) Identification of common molecular subsequences. J. Mol. Biol. 147:195-7). Optimal alignment may be assessed using any suitable parameters of a chosen algorithm, including default parameters.

The “percent identity” between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program can be based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program can define identity as the number of identical aligned symbols (i.e., nucleotides or amino acids), divided by the total number of symbols in the shorter of the two sequences. The program may be used to determine percent identity over the entire length of the sequences being compared. Default parameters can be provided to optimize searches with short query sequences, for example, with the BLASTP program. The program can also allow use of an SEG filter to mask-off segments of the query sequences as determined by the SEG program of Wootton, J. C. and Federhen, S. (1993) Computers Chem. 17:149-163. High sequence identity can include sequence identity in ranges of sequence identity of approximately 80% to 99% and integer values there between.

A “homolog” or “homologue” can refer to any sequence that has at least about 90%, 95%, 96%, 97%, 98%, 99%, or 99.5% sequence homology to another sequence. Preferably, a homolog or homologue refers to any sequence that has at least about 98%, 99%, or 99.5% sequence homology to another sequence. In some cases, the homolog can have a functional or structural equivalence with the native or naturally occurring sequence. In some cases, the homolog can have a functional or structural equivalence with a domain, a motif or a part of the protein, that is encoded by the native sequence or naturally occurring sequence.

Homology comparisons may be conducted with sequence comparison programs. Computer programs may calculate percent (%) homology between two or more sequences and may also calculate the sequence identity shared by two or more amino acid or nucleic acid sequences. Sequence homologies may be generated by any of a number of computer programs, for example BLAST or FASTA, etc. A suitable computer program for carrying out such an alignment is the GCG Wisconsin Bestfit package (University of Wisconsin, U.S.A; Devereux, J. et al. (1984) Nucleic Acids Res. 12:387). Examples of other software than may perform sequence comparisons include, but are not limited to, the BLAST package (see Ausubel, F. M. et al. (1999) Short Protocols in Molecular Biology, 4^{th} Ed.—Chapter 18), FASTA (Atschul, S. F. et al. (1990) J. Mol. Biol. 215:403-410) and the GENEWORKS suite of comparison tools.

Percent homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments can be performed over a relatively short number of residues.

In an otherwise identical pair of sequences, one insertion or deletion may cause the following amino acid or nucleotide residues to be put out of alignment, thus potentially resulting in a large reduction in % homology when a global alignment is performed. Consequently, the sequence comparison method can be designed to produce optimal alignments that take into consideration possible insertions and deletions without unduly penalizing the overall homology or identity score. This can be achieved by inserting “gaps” in the sequence alignment to try to maximize local homology or identity.

BLAST 2 Sequences is another tool that can be used for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health).

Homologous sequences can also have deletions, insertions or substitutions of amino acid residues which result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone.

Substantially homologous sequences of the present invention include variants of the disclosed sequences, e.g., those resulting from site-directed mutagenesis, as well as synthetically generated sequences. In some cases, the variants may be allelic variants due to different alleles. In some cases, the variants may be derived from the same gene or allele due to alternative transcription start site or alternative splicing, resulting in variants which are isoforms.

An extracellular vesicle of the present disclosure can be one that comprises (e.g., on its surface) one or more targeting moiety(ies) to a marker (also referred to herein as a marker of interest). A marker of interest may be a cell surface marker of a target cell of interest to which an EV of the present invention is intended to target or bind. In some embodiments, an EV of the present disclosure is one that comprises (e.g., on its surface) targeting moiety(ies) to a marker of interest or a homologue(s) of a marker of interest. In some instance, an EV comprises at least 2, 3, 4, 5, 6, 7, 8, 9 or 10 different targeting moiety(ies). In an embodiment, an EV comprises a chimeric vesicle localization moiety attached to one or more targeting moiety(ies) to a marker of interest. The marker of interest may be a cell surface marker. In an embodiment, an EV comprises two or more chimeric vesicle localization moieties, wherein each chimeric vesicle localization moiety comprises a different targeting moiety(ies) targeted to the same marker of interest. In an embodiment, an EV comprises two or more chimeric vesicle localization moieties, wherein each chimeric vesicle localization moiety comprises a different targeting moiety(ies) targeted to the different markers of interest. In an embodiment, an EV comprises two or more chimeric vesicle localization moieties, wherein each chimeric vesicle localization moiety comprises a different targeting moiety(ies) targeted to the different markers of interest present on the same cell. In an embodiment, an EV comprises two or more chimeric vesicle localization moieties, wherein each chimeric vesicle localization moiety comprises a different targeting moiety(ies) targeted to the different markers of interest present on different cell types. In an embodiment, a vesicle comprises two or more chimeric vesicle localization moieties, wherein each chimeric vesicle localization moiety comprises a different targeting moiety(ies) targeted to the different markers of interest present in a tissue. In an embodiment, a vesicle comprises two or more chimeric vesicle localization moieties, wherein each chimeric vesicle localization moiety comprises a different targeting moiety(ies) targeted to the different markers of interest present in different tissues. In some instance, a vesicle comprises a sufficient number of targeting moiety(ies) to selectively target cells of interest over other cells. In some instance, a vesicle comprises a sufficient number of targeting moiety(ies) to selectively target a tissue of interest over other tissues.

In some cases, the vesicle comprises a concentration of a targeting moiety of interest that is 2, 3, 4, 5, 6, 8, 10, 12, 14, 17, 18, 20, 22, 25, 28, 30, 33, 35, 38, 40, 43, 44, 46, 48, 50, 52, 55, 57, 59, 62, 65, 68, 70, 72, 75, 78, 80, 82, 85, 89, 91, 92, 95, 100, 110, 120, 125, 130, 135, 145, 150, 155, 160, 170, 180, 185, 200, 210, 220, 230, 250, 270, 280, 290, 300, 310, 320, 330, 340, 350, 380, 400, 410, 430, 440, 450, 470, 490, 500, 510, 525, 540, 560, 580, 590, 600, 620, 650, 670, 680, 690, 700, 720, 740, 760, 780, 800, 820, 840, 860, 880, 890, 900, 920, 940, 960, 980 or 1000 times higher than the concentration of the targeting moiety on the surface of a naturally occurring vesicle. In some cases, the vesicle comprises a targeting moiety which is not naturally associated with a vesicle or an extracellular vesicle. In a preferred embodiment, the vesicle comprises a targeting moiety of interest fused to a chimeric vesicle localization moiety. In a separate preferred embodiment, the vesicle comprises two or more targeting moiety of interest fused to one or more chimeric vesicle localization moiety.

### Fusion Proteins

The “fusion protein” can be a single polypeptide derived from two separate polypeptides or portions of two separate polypeptides. As such, a chimeric vesicle localization may be considered a fusion protein. Similarly, a single polypeptide comprising (1) an isopeptide domain or an isopeptide tag and (2) a vesicle localization domain may also be considered a fusion protein. Other example of a fusion protein is a single polypeptide comprising (1) an isopeptide domain or an isopeptide tag and (2) a protein or peptide targeting moiety. Further fusion protein may be made from one or more isopeptide tag and/or isopeptide domain and a chimeric vesicle localization moiety. In an embodiment, two fusion proteins with complementary isopeptide tag and isopeptide domain may be covalently attached to each other through an isopeptide bond, such that, for example a targeting moiety fusion protein (to either an isopeptide tag or isopeptide domain) may be covalently linked to a vesicle localization moiety fusion protein or a chimeric vesicle localization moiety fusion protein (with a complementary isopeptide domain or isopeptide tag, respectively). In an embodiment of the invention a fusion protein (or peptide or conjugate) of the invention which comprises a isopeptide domain or isopeptide tag and a VLM or chimeric VLM may be paired or matched with any other fusion protein (or peptide or conjugate) described herein comprising a complementary isopeptide tag or domain and a targeting moiety, so long as the isopeptide domain binds to its complementary isopeptide tag and forms an isopeptide bond. For example, any fusion protein (or peptide or conjugates) comprising isopeptide domain having SEQ ID NO: 2, 4, 6, 8, 10, 12, 14, 16, 18, 20, 22, 24, 26, 28, 30, 32, 34, 36, 38, 40, 42, 44, 46, 48, 50, 52 or 54 and a VLM may be paired with any fusion protein (or peptide or conjugate) comprising its complementary isopeptide tag having e.g., SEQ ID NO: 62, so as to form an isopeptide bond covalently linking one fusion protein (or peptide or conjugate) to another fusion protein (or peptide or conjugate).

In an embodiment, the fusion protein comprising a chimeric vesicle localization moiety (or a vesicle localization moiety) additionally comprises a signal peptide. In an embodiment, the nascent or newly synthesized polypeptide of the chimeric vesicle localization moiety fusion protein comprises a signal peptide sequence at the N-terminus. Fusion protein of the chimeric vesicle localization (or a vesicle localization moiety) of interest comprises one or more isopeptide domain(s) or isopeptide tag(s). In an embodiment, the nascent polypeptide or newly synthesized polypeptide is a polypeptide being produced or initially produced by ribosome translation of an mRNA encoding the chimeric vesicle localization moiety fusion protein. In an embodiment, the nascent or newly synthesized polypeptide of the chimeric vesicle localization moiety fusion protein comprises from amino-to-carboxyl terminus in the order: signal peptide, one or more isopeptide domain(s) or isopeptide tag(s), surface domain, transmembrane domain and cytosolic domain. In an embodiment, the nascent or newly synthesized polypeptide of the chimeric vesicle localization moiety fusion protein may additionally comprise any one or more linkers, epitope tags and/or glycosylation sites. In an embodiment, the signal peptide sequence may be a naturally occurring sequence or an engineered (not naturally occurring) sequence. The naturally occurring sequence may be any of the signal peptide sequence associated with a naturally occurring vesicle localization moiety listed in Tables 2 and 3, in addition to other naturally occurring signal peptide sequences. In an embodiment, the engineered signal peptide sequence may be an artificial signal peptide sequence which directs strong protein secretion and expression in human cells. In an embodiment, the engineered signal peptide may be MWWRLWWLLLLLLLLWPMVWA (SEQ ID NO: 279). In a separate embodiment, the signal peptide sequence may be any of those listed in Table 12.

Examples of suitable linkers include, but are not limited to, any of (Gly)₈, (Gly)₆, (GS)ₙ (n=1-5), (GGS)ₙ (n=1-5), (GGGS)ₙ (n=1-5), (GGGGS)ₙ (n=1-5), (GGGGGS)ₙ (n=1-5) (EAAAK)ₙ (n=1-3), A(EAAAK)₄ALEA(EAAAK)₄A, (GGGGS)ₙ (n=1-4), (Ala-Pro)ₙ (10-34 aa), cleavable linkers such as VSQTSKLTRAETVFPDV, PLGLWA, RVLAEA; EDVVCCSMSY; GGIEGRGS, TRHRQPRGWE, AGNRVRRSVG, RRRRRRRRR, GLFG, and LE. Examples of suitable epitope tags include, but are not limited to, FLAG tags such as single or 3×FLAG tags, Myc tags, V5 tags, S-tags, HA tags, 6×His tag, or a combination thereof.

In a separate embodiment, the chimeric vesicle localization moiety lacks a signal peptide. In an embodiment, the chimeric vesicle localization moiety fusion protein is a mature or processed polypeptide. In an embodiment, the mature or processed polypeptide lacks the signal peptide sequence of the nascent polypeptide. In an embodiment, the mature or processed polypeptide comprises a glycosylation site. In an embodiment, the mature or processed polypeptide is a glycoprotein. In an embodiment, the glycoprotein comprises glycans. In an embodiment, the glycoprotein comprises N-linked glycan, O-linked glycan, phosphoglycan, C-linked glycan and/or GPI anchor. In an embodiment, the chimeric vesicle localization moiety is a mature or processed vesicle localizing polypeptide found in association or incorporated by an EV and lacks a signal peptide sequence present in the nascent polypeptide prior to maturation or processing. In an embodiment, it may be advantageous for the chimeric vesicle localization moiety (or vesicle localization moiety) fusion protein to additionally comprise a signal peptide sequence at its amino terminus when such fusion protein is expressed in cells and incorporated into exosomes; it may be desirable to also produce chimeric vesicle localization moiety fusion protein lacking signal peptide sequence when such fusion protein might be used to directly incorporate into an EV or exosome isolated from a cell.

The one or more targeting moieties of interest attached to an isopeptide tag or isopeptide domain as a fusion protein or a conjugate may be linked to a fusion protein comprising a vesicle localization domain or a chimeric vesicle localization domain and an isopeptide domain or isopeptide tag, respectively, wherein the isopeptide domain or isopeptide tag of the VLM or chimeric VLM is displayed on the surface of an EV or exosome. In an embodiment, the EV or exosome comprises on its outer surface one or more targeting moieties of interest linked through an isopeptide bond between an isopeptide tag and an isopeptide domain to a VLM or chimeric VLM incorporated within the lipid bilayer of the EV or exosome. In an embodiment, the one or more targeting moieties of interest can be a fusion protein, wherein the targeting moiety fusion protein comprises (1) a polypeptide or peptide that binds to a cell or tissue marker, cell or tissue surface receptor, cell or tissue ligand, a cell or tissue membrane protein or a molecule present on the outside facing surface or external to the cell surface and (2) an isopeptide domain or isopeptide tag. In an embodiment, the one or more targeting moieties of interest can be a targeting moiety conjugate, wherein the conjugate comprises (1) a molecule that targets a cell or tissue, and (2) an isopeptide domain or isopeptide tag. In an embodiment, an EV or exosome is an engineered or modified EV or exosome comprising a fusion protein comprising a VLM and an isopeptide domain or isopeptide tag. In a preferred embodiment, an EV or exosome is an engineered or modified EV or exosome comprising a fusion protein comprising a chimeric VLM and an isopeptide domain or isopeptide tag.

Examples of vesicle localization moieties from which chimeric vesicle localization moieties may be produced by domain swapping include any of the following: ACE, ADAM10, ADAM15, ADAM9, AGRN, ALCAM, ANPEP, ANTXR2, ATP1A1, ATP1B3, BSG, BTN2A1, CALM1, CANX, CD151, CD19, CD1A, CD1B, CD1C, CD2, CD200, CD200R1, CD226, CD247, CD274, CD276, CD33, CD34, CD36, CD37, CD3E, CD40, CD40LG, CD44, CD47, CD53, CD58, CD63, CD81, CD82, CD84, CD86, CD9, CHMP1A, CHMP1B, CHMP2A, CHMP3, CHMP4A, CHMP4B, CHMP5, CHMP6, CLSTN1, COL6A1, CR1, CSF1R, CXCR4, DDOST, DLL1, DLL4, DSG1, EMB, ENG, EVI2B, F11R, FASN, FCER1G, FCGR2C, FLOT1, FLOT2, FLT3, FN1, GAPDH, GLG1, GRIA2, GRIA3, GYPA, HSPG2, ICAM1, ICAM2, ICAM3, IGSF8, IL1RAP, IL3RA, IL5RA, IST1, ITGA2, ITGA2B, ITGA3, ITGA4, ITGA5, ITGA6, ITGAL, ITGAM, ITGAV, ITGAX, ITGB1, ITGB2, ITGB3, ITGB4, ITGB5, ITGB6, ITGB7, JAG1, JAG2, KIT, LAMP2, LGALS3BP, LILRA6, LILRB1, LILRB2, LILRB3, LILRB4, LMAN2, LRRC25, LY75, M6PR, MFGE8, MMP14, MPL, MRC1, MVB12B, NECTIN1, NOMO1, NOTCH1, NOTCH2, NOTCH3, NOTCH4, NPTN, NRP1, PDCD1, PDCD1LG2, PDCD6IP, PDGFRB, PECAM1, PLXNB2, PLXND1, PROM1, PTGES2, PTGFRN, PTPRA, PTPRC, PTPRJ, PTPRO, RPN1, SDC1, SDC2, SDC3, SDC4, SDCBP, SDCBP2, SELPLG, SIGLEC7, SIGLEC9, SIRPA, SLIT2, SNF8, SPN, STX3, TACSTD2, TFRC, TLR2, TMED10, TNFRSF8, TRAC, TSG101, TSPAN14, TSPAN7, TSPAN8, TYROBP, VPS25, VPS28, VPS36, VPS37A, VPS37B, VPS37C, VPS37D, VPS4A, VPS4B, VTI1A or VTI1B or an isoform thereof, or a homologue thereof, or a variant, or a functional fragment thereof, or an exosomal polypeptide. In a preferred embodiment, the chimeric vesicle localization moieties may be produced by domain swapping include any of the following: ADAM10, ALCAM, CLSTN1, IGSF8, IL3RA, ITGA3, ITGB1, LAMP2, LILRB4, PTGFRN, or SELPLG or an isoform thereof, or a homologue thereof, or a functional fragment thereof. Domain swapping is most easily achieved through recombinant DNA methods using coding sequence provided or referred to in Tables 2 and 3 to precisely dissect and fuse two different coding sequences inframe with each other to obtain a single nucleic acid encoding a chimeric vesicle localization moiety. Nucleic acid sequences encoding exemplary chimeric vesicle localization moieties may be obtained in Table 4 (for example, see SEQ ID NO: 115, 117, 119, 121, 123 and 125).

In an embodiment, a chimeric vesicle localization moiety may be produced by domain swapping two non-homologous vesicle localization moieties. In an embodiment, a chimeric vesicle localization moiety may be produced by domain swapping two vesicle localization moieties which are not orthologs. In an embodiment, a chimeric vesicle localization moiety may be produced by domain swapping two vesicle localization moieties which are not paralogs. In an embodiment, a chimeric vesicle localization moiety may be produced by domain swapping two vesicle localization moieties which are paralogs. In an embodiment, a chimeric vesicle localization moiety may be produced by domain swapping two vesicle localization moieties which are not allelic variants. In an embodiment, a chimeric vesicle localization moiety may be produced by domain swapping two vesicle localization moieties which are not isoforms. In an embodiment, a chimeric vesicle localization moiety may be produced by domain swapping two vesicle localization moieties which are not related by an ancestral gene or gene duplication. In an embodiment, a chimeric vesicle localization moiety may be produced by domain swapping two vesicle localization moieties which are related by gene duplication and have evolved to be paralogs encoded by homologous genes at a different genetic locus (not allelic). In an embodiment, a chimeric vesicle localization moiety may be produced by domain swapping two vesicle localization moieties which are distinct and non-homologous proteins. In an embodiment, a chimeric vesicle localization moiety may be produced by domain swapping two vesicle localization moieties, wherein the domains being swapped share less than about 95%, 90%, 70%, 50% or preferably less than about 30% amino acid sequence identity with gaps allowed in the sequence alignment to maximize sequence identity. In an embodiment, a chimeric vesicle localization moiety may be produced by domain swapping two vesicle localization moieties, wherein the domains being swapped differ in the length of the primary amino acid sequence by more than about 1.3-fold, 1.5-fold, 1.7-fold, 1.9-fold, 2.3-fold, 2.7-fold or more preferably about 3-fold compared to the shorter domain. The domains of a vesicle localization moiety may be determined in relation to membrane of a vesicle and may be described as surface domain (outside of the vesicle; also referred to sometimes as extracellular domain, which is topologically equivalent), transmembrane domain (spanning the lipid bilayer of the vesicle) and lumenal domain (in the interior of the vesicle; also referred to as a cytosolic domain prior to formation of a vesicle, which is topologically equivalent). In an embodiment, the three domains present in a vesicle localization moiety may be swapped with one or more domains from one or more other vesicle localization moiety. In a preferred embodiment, the cytosolic domain or lumenal domain of a vesicle localization moiety is swapped with a cytosolic domain or lumenal domain of a second vesicle localization moiety so as to produce a chimeric vesicle localization moiety with a surface-and-transmembrane domain of a 1st vesicle localization moiety and a cytosolic domain of a 2nd vesicle localization moiety.

Methods for making such fusion proteins and for localizing fusion proteins to exosomes can be as described, e.g., in Limoni S K, et al. Appl Biochem Biotechnol. 2018 Jun. 28. doi: 10.1007/s12010-018-2813-4.

### Nucleic Acids

The production of engineered vesicles can involve generation of nucleic acids that encode, at least, in part, one or more of the cell-type specific or selective targeting moieties described herein, one or more of the targeting moiety(ies) described herein, one or more of the vesicle localization moieties including chimeric vesicle localization moieties described herein, one or more fusion proteins described herein, or a combination thereof.

The disclosure includes vectors. Methods which are well known to those skilled in the art can be used to construct expression vectors containing coding sequences and appropriate transcriptional/translational control signals. Generally, expression vectors include transcriptional and translational regulatory nucleic acid operably linked to the nucleic acid encoding the protein. The term “control sequences” refers to DNA sequences necessary for the expression of an operably linked coding sequence in a particular expression system, e.g. mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

These methods include, for example, in vitro recombinant DNA techniques, synthetic techniques and in vivo recombination/genetic recombination. Alternatively, RNA capable of encoding the polypeptides of interest may be chemically synthesized. One of skill in the art can readily utilize well-known codon usage tables and synthetic methods to provide a suitable coding sequence for any of the polypeptides of the invention.

In some embodiments, a vector comprises nucleic acids encoding one or more cell-type specific or selective targeting moieties operably linked to nucleic acids that encode one or more isopeptide tags or isopeptide domains. In some embodiments, a vector comprises nucleic acids encoding one or more vesicle localization moieties, preferably chimeric vesicle localization moieties operably linked to nucleic acids that encode one or more isopeptide tags or isopeptide domains. A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate the initiation of translation. Generally, “operably linked” means that the DNA sequences being linked are contiguous, and, in the case of a secretory leader, contiguous and in reading phase. Linking is accomplished by ligation or through amplification reactions. Synthetic oligonucleotide adaptors or linkers may be used for linking sequences in accordance with conventional practice.

In some embodiments, a vector comprises nucleic acids encoding the amino acid sequences or portion thereof set forth in Table 2 or Table 3, the latter through the accession number which provides the amino acid sequence for the VLMs so listed. In an embodiment, a vector comprises nucleic acids encoding the chimeric vesicle localization moiety produced from the vesicle localization moieties disclosed herein or in Table 4 (for example, see SEQ ID NO: 115, 117, 119, 121, 123 and 125). In one example, a vector comprises nucleic acids encoding an IGSF8 vesicle localization moiety operably linked to nucleic acids encoding any one or more of an isopeptide domain or an isopeptide tag, as disclosed herein. In one example, a vector comprises nucleic acids encoding a chimeric vesicle localization moiety operably linked to nucleic acids encoding any one or more of an isopeptide domain or isopeptide tag. In one example, a vector comprises nucleic acids encoding an isopeptide domain or isopeptide tag operably linked to nucleic acids encoding any one or more of a targeting moiety(ies) of interest or cell-type specific or selective targeting moieties. In an embodiment, a cell-type specific or selective targeting moiety is a peptide. In an embodiment, a cell-type specific or selective targeting moiety is an antibody or an antibody fragment. In an embodiment, a cell-type specific or selective targeting moiety is an F(ab′)₂, Fab or Fab′. In a preferred embodiment, a cell-type specific or selective targeting moiety is a scFv.

The nucleic acids may be natural, synthetic or a combination thereof. The nucleic acids may be RNA, mRNA, DNA or cDNA. Nucleic acid encoding the protein may be produced using known synthetic techniques, incorporated into a suitable expression vector using well established methods to form a protein-encoding expression vector which is introduced into a cell for protein expression using known techniques, such as transfection, lipofection, transduction and electroporation. The nucleic acids may be isolated and obtained in substantial purity. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

Expression of the nucleic acids can be regulated by their own or by other regulatory sequences known in the art. The nucleic acids of the invention can be introduced into suitable host cells using a variety of techniques available in the art. The expressed protein may localize or form an exosome or extracellular vesicle and released from the producing cell. Such exosomes or extracellular vesicles may be harvested from the culture medium. Similarly, the selected protein may be produced using recombinant techniques, or may be otherwise obtained, and then may be introduced directly into isolated exosomes by electroporation or transfection e.g. electroporation, transfection using cationic lipid-based transfection reagents, and the like.

The nucleic acids can also include expression vectors, such as plasmids, or viral vectors, or linear vectors, or vectors that integrate into chromosomal DNA. Expression vectors can contain a nucleic acid sequence that enables the vector to replicate in one or more selected host cells. Such sequences are well known for a variety of cells. The origin of replication from the plasmid pBR322 is suitable for most Gram-negative bacteria. In eukaryotic host cells, e.g., mammalian cells, the expression vector can be integrated into the host cell chromosome and then replicate with the host chromosome or the expression vector may be an episome and replicate autonomously independent of the host chromosome.

Expression vectors also can contain a selection gene, also termed a selectable marker. The selection gene can encode a protein necessary for the survival or growth of transformed host cells grown in a selective culture medium. Host cells not transformed with the vector containing the selection gene will not survive in the selective culture medium. Selection genes can encode proteins that (a) confer resistance to antibiotics or other toxins, e.g., ampicillin, neomycin, G418, puromycin, hygromycin, methotrexate, or tetracycline, (b) complement auxotrophic deficiencies, or (c) supply critical nutrients not available from complex media, e.g., the gene encoding D-alanine racemase for Bacilli. An exemplary selection scheme can utilize a drug to arrest growth of a host cell. Those cells that are successfully transformed with a heterologous gene can produce a protein conferring drug resistance and thus survive the selection regimen. Other selectable markers for use in bacterial or eukaryotic (including mammalian) systems are well-known in the art.

An example of a promoter that is capable of expressing a transgene in a mammalian nervous system cell is the EF1a promoter. Another example of a promoter is the immediate early cytomegalovirus (CMV) promoter sequence. Other constitutive promoter sequences may also be used, including, but not limited to the simian virus 40 (SV40) early promoter, mouse mammary tumor virus promoter (MMTV), human immunodeficiency virus (HIV) long terminal repeat (LTR) promoter, MoMuLV promoter, phosphoglycerate kinase (PGK) promoter, MND promoter (a synthetic promoter that contains the U3 region of a modified MoMuLV LTR with myeloproliferative sarcoma virus enhancer, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. The promoter can be a non-constitutive promoter.

Inducible or repressible promoters are also contemplated for use in this disclosure. Examples of inducible promoters include a metallothionein promoter, a glucocorticoid promoter, a progesterone promoter, a tetracycline promoter, a c-fos promoter, the T-REx system of ThermoFisher which places expression from the human cytomegalovirus immediate-early promoter under the control of tetracycline operator(s), and RheoSwitch promoters of Intrexon.

Expression vectors typically have promoter elements, e.g., enhancers, to regulate the frequency of transcriptional initiation. These can be located in the region 30-110 bp upstream of the start site, although a number of promoters have been shown to contain functional elements downstream of the start site as well. The spacing between promoter elements can frequently be flexible, so that promoter function can be preserved when elements are inverted or moved relative to one another. The expression vector may be a mono-cistronic construct, a bi-cistronic construct or multiple cistronic construct. For a bi-cistronic construct, the two cistrons can be oriented in opposite directions with the control regions for the cistrons located in between the two cistrons. When the construct has more than two cistrons, the cistrons can be arranged in two groups with the two groups oriented in opposite directions for transcription.

It can be desirable to modify the polypeptides described herein. There can be many ways of generating alterations in a given nucleic acid construct to generate variant polypeptides. Such methods can include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other techniques (see, e.g., Gillam and Smith, Gene 8:81-97, 1979; Roberts et al., Nature 328:731-734, 1987, which is incorporated by reference in its entirety for all purposes). The recombinant nucleic acids encoding the polypeptides described herein can be modified to provide preferred codons which can enhance translation of the nucleic acid in a selected organism or cell line.

The polynucleotides can also include nucleotide sequences that are substantially equivalent (homologues) to other polynucleotides described herein. Polynucleotides can have at least about 80%, more typically at least about 90%, and even more typically at least about 95%, sequence identity to another polynucleotide. In an embodiment, a polynucleotide encoding a protein may be considered equivalent to a second polynucleotide encoding the same protein due to degeneracy of the genetic codon. Such polynucleotides are anticipated herein.

The nucleic acids can also provide the complement of the polynucleotides including a nucleotide sequence that has at least about 80%, more typically at least about 90%, and even more typically at least about 95%, sequence identity to a polynucleotide encoding a polypeptide recited herein. The polynucleotide can be DNA (genomic, cDNA, amplified, or synthetic) or RNA. Nucleic acids which encode protein analogs or variants (i.e., wherein one or more amino acids are designed to differ from the wild type polypeptide) may be produced using site directed mutagenesis or PCR amplification in which the primer(s) have the desired point mutations. For a detailed description of suitable mutagenesis techniques, see Sambrook et al., Molecular Cloning: A Laboratory Manual, Cold Spring Harbor Laboratory Press, Cold Spring Harbor, N.Y. (1989) and/or Current Protocols in Molecular Biology, Ausubel et al., eds, Green Publishers Inc. and Wiley and Sons, N.Y (1994), each of which is incorporated by reference in its entirety for all purposes. Chemical synthesis using methods well known in the art, such as that described by Engels et al., Angew Cher Intl Ed. 28:716-34, 1989 (which is incorporated by reference in its entirety for all purposes), may also be used to prepare such nucleic acids.

Amino acid “substitutions” for creating variants can result from replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

When the nucleic acid is introduced into a cell ex vivo, the nucleic acid may be combined with a substance that promotes transference of a nucleic acid into a cell, for example, a reagent for introducing a nucleic acid such as a liposome or a cationic lipid, in addition to any additional excipients. Electroporation applying voltages in the range of about 20-1000 V/cm may be used to introduce nucleic acid or protein into exosomes. Transfection using cationic lipid-based transfection reagents such as, but not limited to, Lipofectamine® MessengerMAX™ Transfection Reagent, Lipofectamine® RNAiMAX Transfection Reagent, Lipofectamine® 3000 Transfection Reagent, or Lipofectamine® LTX Reagent with PLUS™ Reagent, may also be used. The amount of transfection reagent used may vary with the reagent, the sample and the cargo to be introduced. Alternatively, a vector carrying the nucleic acid of the present invention can also be used. Particularly, a composition in a form suitable for administration to a living body which contains the nucleic acid of the present invention carried by a suitable vector can be suitable for in vivo gene therapy.

The nucleic acid constructs can include linker peptides. The linker peptides can adopt a helical, β-strand, coil-bend or turn conformations. The linker motifs can be flexible linkers, rigid linkers or cleavable linkers. The linker peptides can be used for increasing the stability or folding of the peptide, avoid steric clash, increase expression, improve biological activity, enable targeting to specific sites in vivo, or alter the pharmacokinetics of the resulting fusion peptide by increasing the binding affinity of the targeting domain for its receptor. Folding, as used herein, refers to the process of forming the three-dimensional structure of polypeptides and proteins, where interactions between amino acid residues act to stabilize the structure. Non-covalent interactions are important in determining structure, and the effect of membrane contacts with the protein may be important for the correct structure. For naturally occurring proteins and polypeptides or derivatives and variants thereof, the result of proper folding is typically the arrangement that results in optimal biological activity, and can conveniently be monitored by assays for activity, e.g. ligand binding, enzymatic activity, etc.

The linker peptides can generally be composed of small non-polar (Gly) or non-polar (Ser) amino acids. The linker peptides can have sequences consisting primarily of stretches of glycine and/or serine residues. But can contain additional amino acids, such as Thr and Ala to maintain flexibility, as well as polar amino acids, such as Lys and Glu to improve solubility. In other cases, rigid linkers can have a Proline-rich sequence, such as (XP)ₙ, with X designating any amino acid, preferably Ala, Lys or Glu. In other cases, cleavable linkers can be used susceptible to reductive or enzymatic cleavage, such as disulfide or protease sensitive sequences, respectively. In some cases, the linker peptides can be linked to a reporter moiety, such as a fluorescent protein. Examples of linker sequences include but are not limited to, any of (Gly)₈, (Gly)₆, (GS)ₙ (n=1-5), (GGS)ₙ (n=1-5), (GGGS)ₙ (n=1-5), (GGGGS)ₙ (n=1-5), (GGGGGS)ₙ (n=1-5) (EAAAK)ₙ (n=1-3), A(EAAAK)₄ALEA(EAAAK)₄A, (GGGGS)ₙ (n=1-4), (Ala-Pro)ₙ (10-34 aa), cleavable linkers such as VSQTSKLTRAETVFPDV, PLGLWA, RVLAEA; EDVVCCSMSY; GGIEGRGS, TRHRQPRGWE, AGNRVRRSVG, RRRRRRRRR, GLFG, and LE.

The nucleic acid sequence can also contain signal sequences that encode for signal peptides that function as recognition sequences for sorting of the resulting fusion protein to the vesicular surface. The signal sequence can comprise a tyrosine-based sorting signal and can contain the NPXY where N stands for asparagine, P stands for proline, Y stands for tyrosine and X stands for any amino acid (alanine, cysteine, aspartic acid, glutamic acid, phenylalanine, glycine, histidine, isoleucine, lysine, leucine, methionine, asparagine, proline, glutamine, arginine, serine, threonine, valine, tryptophan or tyrosine). In some cases, the signal sorting motif can comprise a YXXO consensus motif, where O stands for an amino acid residue with a bulky hydrophobic side chain. In some cases, the sorting signal can comprise a (DE)XXXL(LI) consensus motif where D stands for aspartic acid, E stands for glutamic acid, X stands for any amino acid, L stands for leucine and I stands for isoleucine. In some cases, the signal sequence can comprise a di-leucine-based signal sequence motif such as (DE)XXXL(LI) or DXXLL consensus motifs, where D stands for aspartic acid, E stands for glutamic acid, X stands for any amino acid, L stands for leucine and I stands for isoleucine. In some cases, the signal peptic can comprise an acidic cluster. In some cases, the signal peptide can comprise a FW-rich consensus motif, where F stands for phenylalanine and W stands for tryptophan. In some cases, the signal peptide can comprise a proline-rich domain. In some cases, the sorting signal comprises the consensus motif NPFX (1,2) D, where N stands for asparagine, P stands for proline, F stands for phenylalanine, D stands for aspartic acid and X stands for any amino acids. In some cases, the encoded signal peptides can be recognized by adaptor protein complexes AP-1, AP-2, AP-3 and AP-4. In some cases, the DXXLL signals are recognized by another family of adaptors known as GGAs. In some cases, the signal peptides can be ubiquitinated. In an embodiment of the invention, the signal peptide is an immunoglobulin κ-chain signal peptide sequence, METDTLLLWVLLLWVPGSTGD (SEQ ID NO: 281). In another embodiment, the signal peptide is a human signal sequence. In a preferred embodiment, the signal peptide is a computationally designed signal peptide. In a preferred embodiment, the signal peptide sequence is MWWRLWWLLLLLLLLWPMVWA (SEQ ID NO: 279). Other signal peptides along with coding sequences are provided in Table 12.

### Production of Extracellular Vesicles

Any of the nucleic acids herein can be used for heterologous expression in a cell of a fusion protein comprising one or more chimeric vesicle localization moiety (or a VLM) and one or more isopeptide domains or isopeptide tags, wherein the fusion protein localizes or is an integral part of an extracellular vesicle produced by the cell. In an embodiment, any of the nucleic acids herein can be used for heterologous expression in a cell of a fusion protein comprising one or more a targeting moieties of interest and one or more isopeptide domains or isopeptide tags. Alternatively, a targeting moiety conjugate may be prepared wherein the targeting moiety conjugate comprises a targeting moiety of interest and an isopeptide domain or isopeptide tag. In an embodiment, such targeting moiety conjugates are prepared by chemical coupling or crosslinking. In an embodiment, such targeting moiety conjugates are prepared by chemical synthesis or a combination of chemical synthesis and recombinant DNA methods. In an embodiment, EVs and/or exosomes comprising a VLM or chimeric VLM fusion protein comprising (1) a VLM or chimeric VLM and (2) an isopeptide domain or isopeptide tag is covalently linked to a targeting moiety fusion protein comprising (1) a targeting moiety and (2) an isopeptide domain or isopeptide tag. In an embodiment, a cell or tissue-targeting EV or exosome comprises a VLM or chimeric VLM fusion protein comprising (1) a VLM or chimeric VLM and (2) an isopeptide domain or isopeptide tag; a targeting moiety fusion protein comprising (1) a targeting moiety and (2) an isopeptide domain or isopeptide tag; and an isopeptide bond between an isopeptide domain and an isopeptide tag between the VLM or chimeric VLM fusion protein and the targeting moiety fusion protein. In an embodiment, EVs and/or exosomes comprising a VLM or chimeric VLM fusion protein comprising (1) a VLM or chimeric VLM and (2) an isopeptide domain or isopeptide tag is covalently linked to a targeting moiety conjugate comprising (1) a targeting moiety and (2) an isopeptide domain or isopeptide tag. In an embodiment, a cell or tissue-targeting EV or exosome comprises a VLM or chimeric VLM fusion protein comprising (1) a VLM or chimeric VLM and (2) an isopeptide domain or isopeptide tag; a targeting moiety conjugate comprising (1) a targeting moiety and (2) an isopeptide domain or isopeptide tag; and an isopeptide bond between an isopeptide domain and an isopeptide tag between the VLM or chimeric VLM fusion protein and the targeting moiety conjugate.

Common GMP-grade cells used in such heterologous expression and from which vesicles may be isolated, including extracellular vesicles and exosomes, include HEK293 (human embryonic kidney cell line), variants of HEK293, such as HEK293T, HEK 293-F, HEK 293T, and HEK 293-H, dendritic cells, mesenchymal stem cell (MSCs), HT-1080, PER.C6, HeLa, C127, BHK, Sp2/0, NS0, Epi293, Expi293F, and any variants thereof, and any of the following types of allogeneic stem cell lines: Hematopoietic Stem Cells, such as bone marrow HSC, Mesenchymal Stem Cells, such as bone marrow MSC or placenta MSC, human Embryonic Stem Cells or its more differentiated progeny, such as hESC-derived dendritic cell or hESC-derived oligodendrocyte progenitor cell, Neural Stem Cells (NSCs), endothelial progenitor cells (EPCs), or induced Pluripotent Stem Cells (iPSCs).

In an embodiment, any of the cells used for heterologous expression may serve as a source for vesicles, especially extracellular vesicles comprising one or more chimeric vesicle localization moiety(ies)(or VLM) operably linked to one or more isopeptide domains or isopeptide tags. In a preferred embodiment, any of the cell used for heterologous expression may serve as a source for vesicles, especially extracellular vesicles comprising one or more chimeric vesicle localization moieties (or VLM) covalently linked to one or more isopeptide domains or isopeptide tags or a fusion protein comprising one or more chimeric vesicle localization moieties and to one or more isopeptide domains or isopeptide tags.

Any of the polypeptides herein can be produced by a cell (or cell line) generating vesicles which contain the polypeptide. Alternatively, the targeting moiety can be heterologously expressed by the cell producing the vesicle. In an embodiment, the cell producing the vesicle expresses a chimeric vesicle localization moiety (or a VLM) fusion protein and targeting moiety fusion protein, wherein the chimeric VLM (or VLM) fusion protein comprises a chimeric VLM (or VLM) and an isopeptide domain or isopeptide tag, wherein the targeting moiety fusion protein comprises a targeting moiety of interest and an isopeptide domain or isopeptide tag, and wherein the targeting moiety fusion protein associates with the chimeric vesicle localization moiety (or VLM) fusion protein on the surface of an EV or exosome by an isopeptide bond between the isopeptide domain and the isopeptide tag.

In a preferred embodiment, the cell producing the vesicle also expresses a fusion protein comprising a chimeric vesicle localization moiety and an isopeptide domain or isopeptide tag (i.e., a chimeric VLM fusion protein), which are covalently linked in a single polypeptide incorporated into a vesicle, preferably an extracellular vesicle or exosome, produced by the cell. In an embodiment, an extracellular vesicle or exosome producing cell may be considered a producer cell (for an EV or exosome). In an embodiment, more than one targeting moieties may be attached to a single chimeric vesicle localization moiety though one or more isopeptide bonds. In a separate embodiment, a chimeric vesicle localization moiety (or VLM) fusion protein covalently linked to one or more targeting moiety fusion proteins may be present at or are associated with a vesicle, wherein the chimeric vesicle localization moiety (or VLM) fusion protein comprises a chimeric VLM (or VLM) and one or more isopeptide domain(s) and/or isopeptide tag(s), wherein the chimeric VLM (or VLM) fusion protein is covalently linked to one or more targeting moiety fusion proteins through an isopeptide bond formed between an isopeptide domain and an isopeptide tag, and wherein the targeting moiety fusion protein comprise a targeting moiety and an isopeptide domain or isopeptide tag. In a separate embodiment, more than one type of chimeric vesicle localization moiety covalently linked to one or more targeting moieties may be present at or are associated with a vesicle, wherein each type of chimeric vesicle localization moiety differs by at least one amino acid. In an embodiment, the targeting moiety fusion protein is coupled to the vesicle by the producing cell, during vesicle biogenesis or prior to vesicle secretion or isolation through an isopeptide bond. In a different embodiment, the targeting moiety is coupled to the vesicle through an isopeptide bond after the vesicles are produced and/or isolated.

Modified extracellular vesicles can be obtained from a subject, from primary cell culture cells obtained from a subject, from cell lines (e.g., immortalized cell lines), and other cell sources. One can make modified extracellular vesicles with specific markers in several ways. One such method includes engineering cells directly in culture to express VLM or chimeric VLM fusion proteins that are then incorporated into the modified extracellular vesicles harvested as delivery vehicles from these engineered cells. Cells which are used for modified extracellular vesicle production are not necessarily related to or derived from the cell targets of interest. Once derived, vesicles may be isolated based on their size, biochemical parameters, or a combination thereof. Another method that can be used in conjunction with or independent of the direct cell engineering is physical isolation of particular subpopulations (subtypes) of modified vesicles with desired targeting moieties or desired characteristics from the broad, general set of all vesicles produced by a subject. Another method that can be used in conjunction with the previously described two methods or independently is direct incorporation of desired VLM or chimeric VLM fusion proteins on the vesicles surface. In this method, a general population of extracellular vesicles or a specific population of extracellular vesicles are isolated from cell culture. The isolated EVs may be then treated to incorporate desired VLM or chimeric VLM fusion protein into the vesicles (e.g., liposomal fusion) to generate modified vesicles. It is noted that these methods can be combined in different ways. Finally, the modified or engineered EVs or exosomes may be used to attach targeting moiety fusion proteins or conjugates of interest through the interaction between complementary isopeptide domain and isopeptide tag resulting in an isopeptide bond (i.e., a covalent bond) between the targeting moiety fusion protein or conjugate and VLM or chimeric VLM fusion protein. In this manner, desired EVs and/or exosomes targeting different markers, macromolecules, cell types or tissue types may be readily produced from a stock of engineered or modified EVs and/or exosomes and a collection of targeting moiety conjugates or fusion proteins, wherein the collection comprises targeting moiety conjugates or fusion proteins that differ in their ability to target specific markers, macromolecules, cell types or tissue types.

For example, the process can be direct engineering of cells for modified vesicles production followed by isolating modified vesicles, as described in Examples 2 and 8.

The modified vesicles can be incorporated with the targeting moieties directly with or without cholesterol or other phospholipids. The modified vesicle protein mixture can be created via gentle mixing and incubation or several cycles of freezing and thawing.

The modified vesicles can be derived from eukaryotic cells that can be obtained from a subject (autologous) or from allogeneic cell lines. The subject may be any living organism. Examples of subjects include humans, dogs, cats, mice, rats, and transgenic species thereof. Vesicles can be concentrated and separated from the circulatory cells using centrifugation, filtration, or affinity chromatography columns.

### EV Payloads

EVs, including exosomes, engineered to include vesicle localization moieties with isopeptide domains or isopeptide tags or both bound to a targeting moiety through an isopeptide bond, can be used to deliver payloads to cells targeted by the EV and/or exosome. In some instances, the payload is embedded in the vesicle, e.g., the lipid bilayer. Alternatively, or additionally, the payload can be surrounded by the vesicle or lipid bilayer.

As described above, molecules (e.g., a targeting moiety) bound to the vesicle localization moiety fusion protein (e.g., chimeric VLM fusion protein) through isopeptide bonds can traffic the EV and/or exosome in the body to target cells, and the molecules (e.g., a targeting moiety) bound by an isopeptide bond to the vesicle localization moiety fusion protein can also be involved in target cell recognition, interaction, and/or internalization. These molecule-vesicle localization moieties fusion protein (or chimeric VLM fusion protein) can also be used with nanoparticles and other delivery vehicles to be directed to a target cell. See György, Bence, et al. Biomaterials 35 (2014) 26:7598-7609. EV's with these molecule-vesicle localization moiety fusion proteins can also be fused with liposomes or encapsulate other delivery vehicles, such as adeno-associated viral vectors, gene therapy viral vectors, adeno-associated viruses or oncolytic viruses, to enhance their delivery to target cell(s). EV's, exosomes, microparticles, nanoparticles, etc. can carry a payload that is to be delivered to the target cell. Molecule-vesicle localization moiety (e.g., fusion proteins) on the EV and/or exosome can be used in combination with protein that are fused onto the EV and/or exosome surface to improve targeting. Examples of such proteins include apolipoproteins (Apo) A and E, receptor-associated protein (RAP), transferrin (Tf), lactotransferrin, melanotransferrin (p97), leptin, wheat germ agglutinin, non-toxic mutant of diptheria toxin (CRM197), rabies virus glycoprotein (RVG29), Angiopep-2, glutathione (GSH), THR, G23, and others. See Oller-Salvia et al., Chem Soc Rev 45:4690-4707 (2016).

Payloads can be, for example, a small molecule, polypeptide, nucleic acid, lipid, carbohydrate, ligand, receptor, reporter, drug, or combination of the foregoing (e.g., two or more drugs, or one or more drugs combined with a lipid, etc.). Examples of payloads, include, for example pharmaceuticals (e.g., small molecules), biologics (e.g., antibodies, recombinant proteins, or monoclonal antibodies), RNA (siRNA, shRNA, miRNA, antisense RNA, mRNA, noncoding RNA, tRNA, rRNA, other RNAs), reporters, lipids, carbohydrates, nucleic acid constructs (e.g., viral vectors, plasmids, lentivirus, expression constructs, other constructs), oligonucleotides, aptamers, cytotoxic agents, anti-inflammatory agents, antigenic peptides, small molecules, nucleic acid analogs (e.g., antisense oligonucleotide (ASO, 2′-O-methyl (OMe), 2′-fluoro (F), and 2′-O-methoxyethyl (MOE) RNA, locked nucleic acid (LNA), constrained ethyl (cEt), phosphorodiamidate morpholinos (PMOs), phosphorothioate, and peptide nucleic acid (PNA)), and nucleic acids and polypeptides for gene therapy. Payloads can also be complex molecular structures such as viral nucleic acid constructs (encoding transgenes) with accessory proteins for delivery to target cells where the nucleic acid construct can be (if needed) reverse transcribed, delivered to the nucleus, and integrated (or maintained extrachromosomally). Optionally, the construct with a desired transgene(s) can be specifically targeted to a site in the chromosome of the target cell using CRISPR/CAS (e.g., CAS9, CAS13a, CAS13b) and appropriate guide RNAs. Payloads may be loaded into the extracellular vesicle internal membrane space, displayed on, or partially or fully embedded in the lipid bi-layer surface of the extracellular vesicle or some combination thereof.

Examples of pharmaceutical and biologic payloads include drugs for treating diseases and syndromes, cytotoxic agents, and anti-inflammatory drugs. In some cases, the payloads can be fenretinide, sunitinib (e.g., sunitinib malate), sorafenib, Doxorubicin, Mertansine (i.e. DM1) or Imatinib (i.e. Gleevec, STI-571) or any combination thereof.

Examples of RNA payloads include siRNAs, miRNAs, shRNA, antisense RNAs, small nuclear RNA (snRNA), small nucleolar RNA (snoRNA), long intergenic noncoding RNA (lincRNA), piwi interacting RNA (piRNA), ribosomal RNA (rRNA), tRNA, yRNA, and rRNA.

Examples of noncoding RNA payloads include microRNA (miRNA), long non-coding RNA (lncRNA), small nuclear RNA (snRNA), small nucleolar RNA (snoRNA), long intergenic non-coding RNA (lincRNA), piwi-interacting RNA (piRNA), ribosomal RNA (rRNA), yRNA and transfer RNA (tRNA). miRNAs and incRNAs in particular are powerful regulators of homeostasis and cell signaling pathways, and delivery of such RNAs by an EV can impact the target cell. LncRNAs exceed 200 nucleotides in length and act concurrently with DNA-binding proteins and other elements to epigenetically regulate DNA transcription. lncRNAs can regulate gene expression and are involved in stem cell differentiation, and control of cellular activities.

Treatment payloads carried by the modified vesicles can include, for example nucleic acids such as miRNAs, mRNAs, siRNAs, anti-sense oligonucleotides (ASOs), DNA aptamers, CRISPR/Cas9 therapies that inhibit oncogenes, Cytotoxic transgene therapy to induce conditional toxicity, splice switching oligonucleotides or transgenes encoding toxic proteins. In some examples, the payload can be a nucleic acid payload listed in Table 13.

In some cases, a payload can be a reporter moiety. Reporters are moieties capable of being detected indirectly or directly. Reporters include, without limitation, a chromophore, a fluorophore, a fluorescent protein, a luminescent protein, a receptor, a hapten, an enzyme, and a radioisotope.

Examples of reporters include one or more of a fluorescent reporter, a bioluminescent reporter, an enzyme, and an ion channel. Examples of fluorescent reporters include, for example, green fluorescent protein from Aequorea victoria or Renilla reniformis, and active variants thereof (e.g., blue fluorescent protein, yellow fluorescent protein, cyan fluorescent protein, etc.); fluorescent proteins from Hydroid jellyfishes, Copepod, Ctenophora, Anthrozoas, and Entacmaea quadricolor, and active variants thereof; and phycobiliproteins and active variants thereof. Chemiluminescent reporters include, for example, placental alkaline phosphatase (PLAP) and secreted placental alkaline phosphatase (SEAP) based on small molecule substrates such as CPSD (Disodium 3-(4-methoxyspiro {1,2-dioxetane-3,2′-(5′-chloro)tricyclo [3.3.1.13,7]decan}-4-yl)phenyl phosphate, β-galactosidase based on 1,2-dioxetane substrates, neuraminidase based on NA-Star® substrate, all of which are commercially available from ThermoFisher Scientific. Bioluminescent reporters include, for example, aequorin (and other Ca+2 regulated photoproteins), luciferase based on luciferin substrate, luciferase based on Coelenterazine substrate (e.g., Renilla, Gaussia, and Metridina), and luciferase from Cypridina, and active variants thereof. In some embodiments, the bioluminescent reporter include, for example, North American firefly luciferase, Japanese firefly luciferase, Italian firefly luciferase, East European firefly luciferase, Pennsylvania firefly luciferase, Click beetle luciferase, railroad worm luciferase, Renilla luciferase, Gaussia luciferase, Cypridina luciferase, Metrida luciferase, OLuc, and red firefly luciferase, all of which are commercially available from ThermoFisher Scientific and/or Promega. Enzyme reporters include, for example, β-galactosidase, chloramphenicol acetyltransferase, horseradish peroxidase, alkaline phosphatase, acetylcholinesterase, and catalase. Ion channel reporters, include, for example, cAMP activated cation channels. The reporter or reporters may also include a Positron Emission Tomography (PET) reporter, a Single Photon Emission Computed Tomography (SPECT) reporter, a photoacoustic reporter, an X-ray reporter, and an ultrasound reporter.

Nucleic acid payloads can be oligonucleotides, recombinant polynucleotides, DNA, RNA, or otherwise synthetic nucleic acids. The nucleic acids can cause splice switching of RNAs in the target cell, turn off aberrant gene expression in the target cell, replace aberrant (mutated) genes in the chromosome of the target cell with genes encoding a desired sequence. The replacement nucleic acids can be an entire transgene or can be short segments of the mutated/aberrant gene that replaces the mutated sequence with a desired sequence (e.g., a wild-type sequence). Alternatively, the nucleic acid payloads can alter a wild-type gene sequence in the target cell to a desired sequence to produce a desired result. The payload nucleic acids can also introduce a transgene into the target cell that is not normally expressed. The payload nucleic acids can also cause desired deletions of nucleic acids from the genome of the target cell. Examples of nucleic acid payloads include, but are not limited to, those listed in Table 13.

Appropriate genome editing systems can be used with the payload nucleic acids such as CRISPR, TALEN, or Zinc-Finger nucleases. The efficiency of homologous and non-homologous recombination can be facilitated by genome editing technologies that introduce targeted double-stranded breaks (DSB). Examples of DSB-generating technologies are CRISPR/Cas9, TALEN, Zinc-Finger Nuclease, or equivalent systems. See, e.g., Cong et al. Science 339.6121 (2013): 819-823, Li et al. Nucl. Acids Res (2011): gkr188, Gaj et al. Trends in Biotechnology 31.7 (2013): 397405, all of which are incorporated by reference in their entirety for all purposes. Payload nucleic acids can be integrated into desired sites in the genome (e.g., to repair or replace nucleic acids in the chromosome of the target cell), or transgenes can be integrated at desired sites in the genome including, for example, genomic safe harbor site, such as, for example, the CCR5, AAVS1, human ROSA26, or PSIP1 loci. Sadelain et al., Nature Rev. 12:51-58 (2012); Fadel et al., J. Virol. 88(17):9704-9717 (2014); Ye et al., PNAS 111(26):9591-9596 (2014), all of which are incorporated by reference in their entirety for all purposes. When a CRISPR system is used, Cas9 in the target cell may be derived from a plasmid encoding Cas9, an exogenous mRNA encoding Cas9, or recombinant Cas9 polypeptide alone or in a ribonucleoprotein complex. Kim et al (2014) Genome 1012-19. doi:10.1101/gr.171322.113; Wang et al (2013) Cell 153 (4). Elsevier Inc.: 910-18. doi:10.1016/j.cell.2013.04.025, both of which are incorporated by reference in their entirety for all purposes.

### Introducing Payloads

Payloads can be incorporated into vesicles through several methods involving physical manipulation. Physical manipulation methods include but are not limited to, electroporation, sonication, mechanical vibration, extrusion through porous membranes, electric current and combinations thereof, which cause disruption of vesicle membrane. Loading of cargo to vesicles described herein may involve passive loading processes such as mixing, co-incubation, or active loading processes such as electroporation, sonication, mechanical vibration, extrusion through porous membranes, electric current and combinations thereof. In some embodiments, said loading can be done concomitantly with vesicle assembly.

Payloads of interest can be passively loaded into vesicles by incubation with payloads to allow diffusion into the vesicles along the concentration gradient. The hydrophobicity of the drug molecules can affect the loading efficiency. Hydrophobic drugs can interact with the lipid layers of the vesicle membrane and enable stable packaging of the drug in the vesicle's lipid bilayer. In some embodiments, purified exosome solution suspended in buffer solution can be incubated with payload. In some preferred embodiments, the payload is dissolved in a solvent mixture that can include DMSO, to allow passive diffusion into exosomes. Following this, the payload-exosomes mixture is made free from un-encapsulated payload. In preferred embodiments, centrifugation or size-exclusion columns are used to remove precipitates from the supernatant. LC/MS methods can be used for the measurement and characterization of payload in the exosome-payload formulation, following lysis and removal of the exosome fraction.

Nucleic acids of interest can be incubated with purified exosomes to allow transfection of purified exosomes in the presence of a suitable lipid-based transfection reagent. Centrifugation can be used to purify the suspension and isolate the transfected exosome population. Transfected exosomes can then be added to target cells or used in vivo.

Payload can be diffused into cells by incubation with cells that then produce exosomes that carry the payload. For example, cells treated with a drug can secrete exosomes loaded with the drug. In a previous example, Pascucci et al., have treated SR4987 mesenchymal stroma cells with a low dose of paclitaxel for 24 h, then washed the cells and reseeded them in a new flask with fresh medium. After 48 h of culture, the cell conditioned medium was collected, and exosomes were isolated. The paclitaxel-loaded exosomes from the treated cells had significant, strong anti-proliferative activities against CFPAC-1 human pancreatic cells in vitro, as compared with the exosomes from untreated cells (Pascucci, L. et al., Journal of Controlled Release, 192 (2014): 262-270.

Extracellular vesicles secreted from cells can be mixed with payloads and subsequently sonicated by using a homogenizer probe. The mechanical shear force from the sonicator probe can compromise the membrane integrity of the exosomes and subsequently allow the drug to diffuse into the exosomes during this membrane deformation, especially, a hydrophilic drug.

In another embodiment, extracellular vesicles from cells can be mixed with a payload, and the mixture can be loaded into a syringe-based lipid extruder with 100-400 nm porous membranes under a controlled temperature. The exosome membrane can be disrupted during the extrusion process can allow vigorous mixing with the drug. In some examples, the number of effective extrusions can vary from 1-10 to effectively deliver drugs into exosomes.

Payload of interest can be incubated with exosomes at room temperature for a fixed amount of time. Repeated freeze-thaw cycles are then performed to ensure drug encapsulation. The method can result in a broad distribution of size ranges for the resulting exosomes, and then, the mixture is rapidly frozen at −80° C. or in liquid nitrogen and thawed at room temperature. The number of effective freeze-thaw cycle may vary from 2-7 for effective encapsulation. In another embodiment, membrane fusion between exosomes and liposomes can be initiated through freeze-thaw cycles to create exosome-mimetic particles.

In another cases, small pores can be created in exosomes membrane through application of an electrical field to exosomes suspended in a conductive solution. The phospholipid bilayer of the exosomes can be disturbed by the electrical current. Payloads can subsequently diffuse into the interior of the exosomes via the pores. The integrity of the exosome membrane can then be recovered after the drug loading process. In some examples, nucleic acids, e.g., mRNA, siRNA or miRNA can be loaded into exosomes using this method.

In some cases, electroporation can be conducted in an optimized buffer such as trehalose disaccharide to aid in maintaining structural integrity and can inhibit the aggregation of exosomes.

Membrane permeabilization can be initiated through incubation with surfactants, such as, saponin. In some examples, hydrophilic molecules can be assisted in exosome encapsulation by this process.

Chemistry based approaches can also be used to directly attach molecules to the surfaces of exosomes via covalent bonds. In some examples, copper-catalyzed azide alkyne cycloaddition can be used for the bioconjugation of small molecules and macromolecules to the surfaces of exosomes as shown in Wang et al., 2015 and Hood et al., 2016—the references incorporated in their entirety.

In another embodiment, fluorophores and microbeads conjugated to highly specific antibodies can bind a particular antigen on the cell surface. Specific antigen-conjugated microbeads can be used for exosome isolation and tracking in vivo.

Introducing Nucleic Acid Payloads into EVs, Exosomes and Eukaryotic Cells

A process for introducing a desired nucleic acid (e.g., a transgene payload) to a cell or an EV includes a step of introducing the nucleic acid into a eukaryotic cell, EV or exosome. This step can be carried out ex vivo. For example, a cell, EV, or exosome can be transformed ex vivo with a virus vector or a non-virus vector carrying a desired nucleic acid.

In a process, a eukaryotic cell, EV, or exosome can be used. The eukaryotic cell, EV, or exosome can be derived from a mammal, for example, a human cell, or a cell derived from a non-human mammal such as a monkey, a mouse, a rat, a pig, a horse, or a dog can be used. The cell used in the process is not particularly limited, and any cell, EV or exosome can be used. The aforementioned cells, EVs, or exosomes may be collected from a living body, obtained by expansion culture of a cell collected from a living body, or established as a cell strain. When the EV or exosome is to be used in a living body the nucleic acids can be introduced into a cell collected from the living body itself.

The nucleic acids can be introduced to the eukaryotic cell by transfection (e.g., Gorman, et al. Proc. Natl. Acad. Sci. 79.22 (1982): 6777-6781, which is incorporated by reference in its entirety for all purposes), transduction (e.g., Cepko and Pear (2001) Current Protocols in Molecular Biology unit 9.9; DOI: 10.1002/0471142727.mb0909s36, which is incorporated by reference in its entirety for all purposes), calcium phosphate transformation (e.g., Kingston, Chen and Okayama (2001) Current Protocols in Molecular Biology Appendix 1C; DOI: 10.1002/0471142301.nsa01cs01, which is incorporated by reference in its entirety for all purposes), cell-penetrating peptides (e.g., Copolovici, Langel, Eriste, and Langel (2014) ACS Nano 2014 8 (3), 1972-1994; DOI: 10.1021/nn4057269, which is incorporated by reference in its entirety for all purposes), electroporation (e.g. Potter (2001) Current Protocols in Molecular Biology unit 10.15; DOI: 10.1002/0471142735.im1015s03 and Kim et al (2014) Genome 1012-19. doi:10.1101/gr.171322.113, Kim et al. 2014 describe the Amaza Nucleofector, an optimized electroporation system, both of these references are incorporated by reference in their entirety for all purposes), microinjection (e.g., McNeil (2001) Current Protocols in Cell Biology unit 20.1; DOI: 10.1002/0471143030.cb2001s18, which is incorporated by reference in its entirety for all purposes), liposome or cell fusion (e.g., Hawley-Nelson and Ciccarone (2001) Current Protocols in Neuroscience Appendix IF; DOI: 10.1002/0471142301.nsa01fs10, which is incorporated by reference in its entirety for all purposes), mechanical manipulation (e.g. Sharon et al. (2013) PNAS 2013 110(6); DOI: 10.1073/pnas.1218705110, which is incorporated by reference in its entirety for all purposes) or other well-known technique for delivery of nucleic acids to eukaryotic cells.

Once introduced, the nucleic acid can exist episomally, or can be integrated into the genome of the eukaryotic cell using well known techniques such as recombination (e.g., Lisby and Rothstein (2015) Cold Spring Harb Perspect Biol. March 2; 7(3). pii: a016535. doi: 10.1101/cshperspect.a016535, which is incorporated by reference in its entirety for all purposes), or non-homologous integration (e.g., Deyle and Russell (2009) Curr Opin Mol Ther. 2009 August; 11(4):442-7, which is incorporated by reference in its entirety for all purposes). The efficiency of homologous and non-homologous recombination can be facilitated by genome editing technologies that introduce targeted double-stranded breaks (DSB). Examples of DSB-generating technologies are CRISPR/Cas9, TALEN, Zinc-Finger Nuclease, or equivalent systems (e.g., Cong et al. Science 339.6121 (2013): 819-823, Li et al. Nucl. Acids Res (2011): gkr188, Gaj et al. Trends in Biotechnology 31.7 (2013): 397-405, all of which are incorporated by reference in their entirety for all purposes), transposons such as Sleeping Beauty (e.g., Singh et al (2014) Immunol Rev. 2014 January; 257(1):181-90. doi: 10.1111/imr.12137, which is incorporated by reference in its entirety for all purposes), targeted recombination using, for example, FLP recombinase (e.g., O'Gorman, Fox and Wahl Science (1991) 15:251(4999):1351-1355, which is incorporated by reference in its entirety for all purposes), CRE-LOX (e.g., Sauer and Henderson PNAS (1988): 85; 5166-5170), or equivalent systems, or other techniques known in the art for integrating the nucleic acid into the eukaryotic cell genome.

The nucleic acids can be integrated into a chromosome of the eukaryotic cell or can be present in the eukaryotic cell extra-chromosomally. The nucleic acids can be integrated using a genome editing enzyme (CRISPR, TALEN, Zinc-Finger nuclease), and appropriate nucleic acids. The nucleic acids can encode a transgene which can be integrated into the eukaryotic cell chromosome at a genomic safe harbor site, such as, for example, the CCR5, AAVS1, human ROSA26, or PSIP1 loci. The integration of the nucleic acid encoding the transgene at the CCR5, PSIP1, or TRAC locus (T-cell receptor α constant locus) can be done using a gene editing system, such as, for example, CRISPR, TALEN, Sleeping Beauty Transposase, PiggyBac transposase, or Zinc-Finger nuclease systems. Eyquem et al., Nature 543:113-117 (2017), which is incorporated by reference in its entirety for all purposes. The eukaryotic cell can be a human and a CRISPR system can be used to integrate the transgene at the CCR5 or PSIP1 locus. Integration of the nucleic acid at CCR5, PSIP1, or TRAC locus using the CRISPR system also may delete a portion, or all, of the CCR5 gene, PSIP1 gene, or TRAC locus. Cas9 in the eukaryotic cell may be derived from a plasmid encoding Cas9, an exogenous mRNA encoding Cas9, or recombinant Cas9 polypeptide alone or in a ribonucleoprotein complex. Kim et al (2014) Genome 1012-19. doi:10.1101/gr.171322.113; Wang et al (2013) Cell 153 (4). Elsevier Inc.: 910-18. doi:10.1016/j.cell.2013.04.025, both of which are incorporated by reference in their entirety for all purposes.

Chemical means for introducing a polynucleotide into a eukaryotic cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system. Nucleic acids can also be loaded into EVs, liposomes or other vesicles using the techniques of Haraszhi et al., Bio. Protocol. 7:e2338, DOI: 10.21769/BioProtoc.2338 (2017), which is incorporated by reference in its entirety for all purposes.

Transduction can be done with a virus vector such as a retrovirus vector (including an oncoretrovirus vector, a lentivirus vector, and a pseudo type vector), an adenovirus vector, an adeno-associated virus (AAV) vector, a simian virus vector, a vaccinia virus vector or a sendai virus vector, an Epstein-Barr virus (EBV) vector, and a HSV vector can be used. As the virus vector, a virus vector lacking the replicating ability so as not to self-replicate in an infected cell is preferably used.

When a retrovirus vector is used to transduce the host cell, the process can be carried out by selecting a suitable packaging cell based on an LTR sequence and a packaging signal sequence possessed by the vector and preparing a retrovirus particle using the packaging cell. Examples of the packaging cell include PG13 (ATCC CRL-10686), PA317 (ATCC CRL-9078), GP+E-86 and GP+envAm-12 (U.S. Pat. No. 5,278,056, which is incorporated by reference in its entirety for all purposes), and Psi-Crip (Proceedings of the National Academy of Sciences of the United States of America, vol. 85, pp. 6460-6464 (1988), which is incorporated by reference in its entirety for all purposes). A retrovirus particle can also be prepared using a 293 cell or a T cell having high transfection efficiency. Many kinds of retrovirus vectors produced based on retroviruses and packaging cells that can be used for packaging of the retrovirus vectors are widely commercially available from many companies.

A number of viral based systems have been developed for gene transfer into mammalian cells. A desired nucleic acid (can encode one or multiple genes, or functional RNAs, or other nucleic acids) can be inserted into a vector and packaged in viral particles using techniques known in the art. The recombinant virus can then be isolated and delivered to cells of the subject either in vivo or ex vivo. A number of viral systems are known in the art. Adenovirus vectors can be used. A number of adenovirus vectors are known in the art and can be used. In addition, lentivirus vectors can be used.

An expression construct can be used in combination with a liposome (or an EV) and a condensing agent such as a cationic lipid as described in WO 96/10038, WO 97/18185, WO 97/25329, WO 97/30170 and WO 97/31934 (which are incorporated herein by reference in their entirety for all purposes).

Chemical structures with the ability to promote stability and/or translation efficiency can be used. The RNA preferably has 5′ and 3′ UTRs. The 5′ UTR can be between one and 3000 nucleotides in length. The length of 5′ and 3′ UTR sequences to be added to the coding region can be altered by different methods, including, but not limited to, designing primers for PCR that anneal to different regions of the UTRs. Using this approach, the 5′ and 3′ UTR lengths can be modified to achieve optimal translation efficiency following transfection of the transcribed RNA. The 5′ and 3′ UTRs can be the naturally occurring, endogenous 5′ and 3′ UTRs for the nucleic acid of interest. The UTR sequences that are not endogenous to the nucleic acid of interest can be added by incorporating the UTR sequences into the forward and reverse primers or by other modification techniques applied to the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

The mRNA may have both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

In the step of introducing a nucleic acid into a cell, a functional substance for improving the introduction efficiency can also be used (e.g. WO 95/26200 and WO 00/01836, which are incorporated herein by reference in their entirety for all purposes). Examples of the substance for improving the introduction efficiency include a substance having ability to bind to a virus vector, for example, fibronectin and a fibronectin fragment. A fibronectin fragment can have a heparin binding site, for example, a fragment commercially available as RetroNetcin (registered trademark, CH-296, manufactured by TAKARA BIO INC.) can be used. Also, polybrene which is a synthetic polycation having an effect of improving the efficiency of infection of a retrovirus into a cell, a fibroblast growth factor, V type collagen, polylysine or DEAE-dextran can be used. The functional substance can be immobilized on a suitable solid phase, for example, a container used for cell culture (plate, petri dish, flask or bag) or a carrier (microbeads etc.).

Packaging of polypeptides, nucleic acids, and/or drugs placed inside EVs and exosomes can be conducted via incubation in cell culture in a similar manner to the methods described to engineer EVs with certain surface proteins. See above. For example, methods can be used such as those described in McNaughton et al., Proc. Natl Acad Sci 106:6111-16 (2009), or Kotmakci et al., J. Pharm. Pharm. Sci. 18:396-413 (2015), both of which are incorporated by reference in their entirety for all purposes. Specifically, the parental EV or EV subpopulation produced from regular flask/dish culture or bioreactor culture of transfected cells or non-transfected cells can be directly incorporated with the polypeptide, nucleic acid or small molecule via electroporation of the EV and polypeptide, nucleic acid or small molecule. The controlled electric pulse creates the permeabilized area on the EV surface membrane for polypeptide, nucleic acid or small molecule insertion/incorporation. Electroporation requires an incubation period after the electroporation process in order to ensure that the membrane is recovered. Additionally, drug loading after EV isolation may be achieved by simple incubation of the drug of interest with isolated exosomes. This allows loading of lipophilic molecules in the lipid bilayer of the EVs. Other methods for polypeptides, nucleic acids, and/or drugs include lipofectamine and packaging the payloads during biogenesis in transfected cells.

### Target Cells

The vesicles described herein can be used to selectively target a cell, tissue, or organ of interest. The target may be on a cell, in a cell (e.g. in the cell nucleus for nucleus targeting) or in an extracellular matrix.

In some embodiments, the target cell is an eukaryotic cell. A target cell can be a cell from an animal such as a mouse, rat, rabbit, hamster, porcine, bovine, feline, or canine. The target cells can be mammalian cells, such as mouse, rat, rabbit, hamster, porcine, bovine, feline, or canine. The mammalian cells can be cells of primates, including but not limited to, monkeys, chimpanzees, gorillas, and humans. The mammalians cells can be mouse cells, as mice routinely function as a model for other mammals, most particularly for humans. See, e.g., Hanna, J. et al., Science 318:1920-23, 2007; Holtzman, D. M. et al., J Clin Invest. 103(6):R15-R21, 1999; Warren, R. S. et al., J Clin Invest. 95: 1789-1797, 1995; each publication is incorporated by reference in its entirety for all purposes. Animal cells include, for example, fibroblasts, epithelial cells (e.g., renal, mammary, prostate, lung), keratinocytes, hepatocytes, adipocytes, endothelial cells, and hematopoietic cells. The animal cells can be adult cells (e.g., terminally differentiated, dividing or non-dividing) or embryonic cells (e.g., blastocyst cells, etc.) or stem cells.

The target cell also can be a cell line derived from an animal or other source. Examples of specific cell lines include HEK293 and variants of HEK293 such as HEK293T, ARPE19, NS0, NS1 (mice cell lines), CHO-K1 (general CHO), GS-CHO, CHO-DG44 (Chinese hamster ovary, HeLa, PER.C6, Epi293, Expi293F (ThermoFisher, Catalog No. A14527) and hTERT.

The target cells can be stem cells. A variety of stem cells types are known in the art and can be used as the target cell, including for example, embryonic stem cells, inducible pluripotent stem cells, hematopoietic stem cells, neural stem cells, epidermal neural crest stem cells, mammary stem cells, intestinal stem cells, mesenchymal stem cells, olfactory adult stem cells, testicular cells, and progenitor cells (e.g., neural, angioblast, osteoblast, chondroblast, pancreatic, epidermal, etc.). The stem cells can be stem cell lines derived from cells taken from a subject.

Target cells can also be any of musculoskeletal cells, kidney cells, neural cells, brain cells, blood-brain barrier cells, cardiac muscle cells, and liver cells.

### Pharmaceutical Compositions

Pharmaceutical compositions disclosed herein may comprise modified extracellular vesicles of the invention and/or liposomes with (or without) a payload, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions are in one aspect formulated for intravenous administration or intracranial administration or intranasal administration to the central nervous system. Compositions described herein may include lyophilized EVs (e.g., exosomes). In a preferred embodiment, composition comprises an EV or exosome and a pharmaceutically acceptable excipient.

In some embodiments, a composition herein comprises an isolated or enriched set of vesicles that selectively target a tissue or cell of interest. Such vesicles can be loaded with a payload as described herein to be delivered to the cell or tissue of interest. In an embodiment, such vesicles that selectively target a tissue or cell of interest may be an EV or exosome comprising: (1) a VLM or chimeric VLM fusion protein comprising one or more isopeptide domain(s) and a VLM or chimeric VLM; and (2) one or more cell or tissue targeting moiety(ies) of interest linked to an isopeptide tag; wherein (1) and (2) are covalently attached through one or more isopeptide bond(s). In another embodiment, such vesicles that selectively target a tissue or cell of interest may be an EV or exosome comprising: (1) a VLM or chimeric VLM fusion protein comprising one or more isopeptide tag(s) and a VLM or chimeric VLM; and (2) one or more cell or tissue targeting moiety(ies) of interest linked to an isopeptide domain; wherein (1) and (2) are covalently attached through one or more isopeptide bond(s). In another embodiment, such vesicles that selectively target a tissue or cell of interest may be an EV or exosome comprising: (1) a VLM or chimeric VLM fusion protein comprising one or more isopeptide tag(s) and/or isopeptide domain(s) and a VLM or chimeric VLM; and (2) one or more cell or tissue targeting moiety(ies) of interest linked to an isopeptide domain or isopeptide tag; wherein (1) and (2) are covalently attached through one or more isopeptide bond(s). In an embodiment, cell targeting moiety(ies) may be a polypeptide, peptide, nucleic acid, nucleic acid analogs, carbohydrate, lipid, ligand, aptamer, chemical compound, macromolecule or other molecules.

In one embodiment of the invention, the chimeric vesicle localization moiety may comprise a surface-and-transmembrane domain of a first vesicle localization moiety and a cytosolic domain of a second vesicle localization moiety. In a preferred embodiment, the first and second vesicle localization moieties are distinct/different proteins and not isoforms. In a preferred embodiment, the first and second vesicle localization moieties are distinct/different proteins and not an allelic variant. In a preferred embodiment, the first and second vesicle localization moieties are distinct/different proteins and not a homolog. In a preferred embodiment, the first and second vesicle localization moieties are distinct/different proteins and not an ortholog. In an embodiment, the first and second vesicle localization moieties are distinct/different proteins but are paralogs. In a preferred embodiment, the first and second vesicle localization moieties are distinct/different proteins and are not paralogs.

In an embodiment, the first and second vesicle localization moieties are distinct/different proteins from a eukaryote or of eukaryotic origin. The eukaryote may include any of animal, plant, fungi, and protist. In an embodiment, the first and second vesicle localization moieties are distinct/different proteins from a mammal or of mammalian origin. The mammal may include, but is not limited to, a human, monkey, chimpanzee, ape, gorilla, cattle, pig, sheep, horse, donkey, kangaroo, rat, mouse, guinea pig, hamster, cat, dog, rabbit and squirrel. In an embodiment, the first and second vesicle localization moieties are distinct/different proteins from a human or of human origin.

In an embodiment, the chimeric vesicle localization moiety is obtained using recombinant DNA methods. The chimeric vesicle localization moiety can be produced from expression of a nucleic acid encoding amino acid sequence of the 1^{st} vesicle localization moiety and the 2^{nd} vesicle localization moiety. The nucleic acid encoding the chimeric vesicle localization moiety can be introduced into an expression vector or system. Examples of nucleic acid sequences are provided in the Tables herein and the Sequence Listing provided herewith. The expression vector or system may be introduced into a cell which expresses the chimeric vesicle localization moiety (or a vesicle localization moiety) as a polypeptide or fusion protein, optionally with a signal peptide sequence at its amino terminus. In an embodiment, preferably the cell is a mammalian cell, more preferably a human cell. In an embodiment, the expression vector or system may be introduced into a producer cell, which produces extracellular vesicles, preferably exosomes. In the case of VLM or chimeric VLM fusion protein produced from an expression vector introduced into a producer cell, the nucleic acid encoding the VLM or chimeric VLM fusion protein additionally comprises a sequence for a signal peptide at the start (5′ end) of the coding sequence. In an embodiment, the producer cell is a mammalian cell. In a preferred embodiment, the producer cell is a human cell. Alternatively, the expression vector or system may be used in an in vitro transcription and translation system to produce a chimeric vesicle localization moiety fusion protein as a polypeptide. In an embodiment, the in vitro produced chimeric vesicle localization moiety fusion protein may be isolated. In an embodiment, an isolated chimeric vesicle localization moiety fusion protein may be introduced into an extracellular vesicle or exosome isolated from cells. In the case of VLM or chimeric VLM produced from an expression vector using an in vitro transcription and translation system, the nucleic acid encoding the VLM or chimeric VLM fusion protein preferably lacks a sequence for a signal peptide at the start (5′ end) of the coding sequence.

Examples of suitable and preferred first and second vesicle localization moieties include, but are not limited to, ADAM10, ALCAM, CLSTN1, IGSF8, IL3RA, ITGA3, ITGB1, LAMP2, LILRB4, PTGFRN, and SELPLG. Examples of some resulting chimeric vesicle localization moieties may be seen in Table 4 (e.g., SEQ ID NO: 116, 118, 120, 122, 124 and 126, encoded by nucleic acid SEQ ID NO: 115, 117, 119, 121, 123 and 125, respectively). Further examples of suitable vesicle localization moieties may include, but are not limited to, a growth factor receptor, Fc receptor, interleukin receptor, immunoglobulin, MHC-I or MHC-II component, CD antigen, and escort protein. Examples of suitable second vesicle localization moieties include, but are not limited to, the same examples as described for the first vesicle localization moieties.

The vesicle-localization moiety may further comprise a peptide or protein with a modified amino acid. The modified amino acid may result from an attachment of a hydrophobic group. The attachment of a hydrophobic group may be myristoylation for attachment of myristate, palmitoylation for attachment of palmitate, prenylation for attachment of a prenyl group, farnesylation for attachment of a farnesyl group, geranylgeranylation for attachment of a geranylgeranyl group or glycosylphosphatidylinositol (GPI) anchor formation for attachment of a glycosylphosphatidylinositol comprising a phosphoethanolamine linker, glycan core and phospholipid tail. The attachment of a hydrophobic group may be performed by chemical synthesis in vitro or is performed enzymatically in a post-translational modification reaction.

Examples of the first and second vesicle localization moieties include, but are not limited to, any of ACE, ADAM10, ADAM15, ADAM9, AGRN, ALCAM, ANPEP, ANTXR2, ATP1A1, ATP1B3, BSG, BTN2A1, CALM1, CANX, CD151, CD19, CD1A, CD1B, CD1C, CD2, CD200, CD200R1, CD226, CD247, CD274, CD276, CD33, CD34, CD36, CD37, CD3E, CD40, CD40LG, CD44, CD47, CD53, CD58, CD63, CD81, CD82, CD84, CD86, CD9, CHMP1A, CHMP1B, CHMP2A, CHMP3, CHMP4A, CHMP48, CHMP5, CHMP6, CLSTN1, COL6A1, CR1, CSF1R, CXCR4, DDOST, DLL1, DLL4, DSG1, EMB, ENG, EVI2B, F11R, FASN, FCER1G, FCGR2C, FLOT1, FLOT2, FLT3, FN1, GAPDH, GLG1, GRIA2, GRIA3, GYPA, HSPG2, ICAM1, ICAM2, ICAM3, IGSF8, IL1RAP, IL3RA, IL5RA, IST1, ITGA2, ITGA2B, ITGA3, ITGA4, ITGA5, ITGA6, ITGAL, ITGAM, ITGAV, ITGAX, ITGB1, ITGB2, ITGB3, ITGB4, ITGB5, ITGB6, ITGB7, JAG1, JAG2, KIT, LAMP2, LGALS3BP, LILRA6, LILRB1, LILRB2, LILRB3, LILRB4, LMAN2, LRRC25, LY75, M6PR, MFGE8, MMP14, MPL, MRC1, MVB12B, NECTIN1, NOMO1, NOTCH1, NOTCH2, NOTCH3, NOTCH4, NPTN, NRP1, PDCD1, PDCD1LG2, PDCD6IP, PDGFRB, PECAM1, PLXNB2, PLXND1, PROM1, PTGES2, PTGFRN, PTPRA, PTPRC, PTPRJ, PTPRO, RPN1, SDC1, SDC2, SDC3, SDC4, SDCBP, SDCBP2, SELPLG, SIGLEC7, SIGLEC9, SIRPA, SLIT2, SNF8, SPN, STX3, TACSTD2, TFRC, TLR2, TMED10, TNFRSF8, TRAC, TSG101, TSPAN14, TSPAN7, TSPAN8, TYROBP, VPS25, VPS28, VPS36, VPS37A, VPS37B, VPS37C, VPS37D, VPS4A, VPS4B, VTI1A, or VTI1B or a homologue thereof; or variant thereof; or a combination thereof. Amino acid sequences and associated nucleic acid encoding sequences for the vesicle localization moieties (above) may be obtained in Tables 2 and 3; where the sequences are not directly provided in the table, the sequences may be obtained from provided Accession Number and database referred to in the tables.

In an embodiment, the first and second vesicle localization moieties from which a chimeric vesicle localization moiety is derived may be from any of the transmembrane proteins listed in Table 2 or 3 or a homologue thereof. In an embodiment, the chimeric vesicle localization moiety comprises a surface-and-transmembrane domain of a 1^{st} vesicle localization moiety selected from any of the transmembrane protein listed in Table 2 or a homologue thereof and a cytosolic domain of a 2^{nd} vesicle localization moiety selected from any of the transmembrane protein listed in Table 3 or a homologue thereof. In a separate embodiment, the chimeric vesicle localization moiety comprises a surface-and-transmembrane domain of a 1^{st} vesicle localization moiety selected from any of the transmembrane protein listed in Table 3 or a homologue thereof and a cytosolic domain of a 2^{nd} vesicle localization moiety selected from any of the transmembrane protein listed in Table 2 or a homologue thereof. In a preferred embodiment, the chimeric vesicle localization moiety comprises a surface-and-transmembrane domain of a 1^{st} vesicle localization moiety selected from any of the transmembrane protein listed in Table 2 or a homologue thereof and a cytosolic domain of a 2^{nd} vesicle localization moiety from any of the transmembrane protein listed in Table 2 or a homologue thereof, but not selected for the 1^{st} vesicle localization domain.

In an embodiment, nucleic acid sequences as provided in Tables 2 or through the accession number in Table 3 may be used to produce a chimeric vesicle localization moiety through recombinant DNA method. In an embodiment, the next adjacent amino acid of a surface domain is followed and joined to first amino acid of a transmembrane domain and the last amino acid of the transmembrane domain is joined to the first amino acid of a cytosolic domain. In an embodiment, a vesicle localization moiety in Tables 2 and 3 comprises a transmembrane protein in which from amino-to-carboxyl terminal direction, last amino acid of a surface domain is joined to first amino acid of a transmembrane domain, and further, last amino acid of the transmembrane domain is joined to first amino acid of a cytosolic domain. Note additional presence of a signal peptide sequence with its last amino acid joined to the first amino acid of the surface domain for the amino acid sequences in Table 2 and the nucleic acid sequences in Table 2 or the vesicle localization moiety coding sequences associated with each ENST number in Table 3. During cellular expression, the signal peptide is cleaved from the nascent protein to produce a mature vesicle localization moiety found associated with an EV. For example, the full length vesicle localization moiety for Lamp2 with its native signal sequence (SEQ ID NO: 94) following processing results in a mature Lamp2 (SEQ ID NO: 112) lacking the first 28 amino acids which make up the Lamp2 signal sequence; similarly, CLSTN1 with its signal sequence (SEQ ID NO: 76) following processing results in a mature CLSTN1 (SEQ ID NO: 114) lacking first 28 amino acids which make up the CLSTN1 signal sequence, and IGSF8 with its signal sequence (SEQ ID NO: 78) results in a mature IGSF8 (SEQ ID NO: 128) lacking the first 27 amino acid sequence which makes up the IGSF8 signal sequence. Tables 2 and 3 provide full-length vesicle localization moieties with signal peptides and nucleic acid coding sequences. Amino acid sequences of vesicle localization moieties and amino acid sequences for signal peptide, surface domain, transmembrane domain and cytosolic domain along with nucleic acid coding sequences may additionally be accessed through accession numbers associated with the UniProtKB and Ensembl ENSP and ENST identifiers.

In an embodiment, the chimeric vesicle localization moiety comprises a surface-and-transmembrane domain of a 1^{st} vesicle localization moiety and a cytosolic domain of a 2^{nd} vesicle localization moiety. The 1^{st} vesicle localization moiety may include any of ADAM10, ALCAM, CLSTN1, IGSF8, IL3RA, ITGA3, ITGB1, LAMP2, LILRB4, PTGFRN or SELPLG or a homologue thereof or variant thereof. The 2^{nd} vesicle localization moiety may be selected from the same group of transmembrane proteins so long as the first and second vesicle localization moieties are from different or non-homologous proteins. Amino acid sequences and nucleic acid sequences encoding ADAM10, ALCAM, CLSTN1, IGSF8, IL3RA, ITGA3, ITGB1, LAMP2, LILRB4, PTGFRN, and SELPLG are provided in Table 2 along with Ensembl ENSP and ENST identifiers (Hunt, S. E. et al. (2018) Database, 2018, 1-12; doi: 10.1093/database/bay119; Yates, A. D. et al., (2019) Nucleic Acids Res. 48:D682-D688).

In a preferred embodiment, the chimeric vesicle localization moiety comprises a LAMP2 surface-and-transmembrane domain (amino acid sequence and nucleic acid sequence for LAMP2 may be obtained under Accession Number ENSP00000360386 encoded by Transcript ID ENST00000371335 from Gene ID ENSG00000005893, based on assembled sequence in Genome Reference Consortium Human Build 38 patch release 13 (GRCh38.p13; GenBank assembly accession GCA_000001405.28 and RefSeq assembly accession GCF_000001405.39)). In a preferred embodiment, LAMP2 protein with Accession Number ENSP00000360386 encoded by Transcript ID ENST00000371335 is LAMP2B. The chimeric vesicle localization moiety comprising a LAMP2 surface-and-transmembrane domain additionally comprises a cytosolic domain of ADAM10, ALCAM, CLSTN1, IGSF8, IL3RA, ITGA3, ITGB1, LILRB4, PTGFRN, or SELPLG or a homologue or portion thereof. In a preferred embodiment, the chimeric vesicle localization moiety comprising LAMP2 surface-and-transmembrane domains additionally comprises a cytosolic domain of PTGFRN, ITGA3, IL3RA, SELPLG, ITGB1, or CLSTN1 or a homologue or portion thereof, but lacks the LAMP2 cytosolic domain.

In one embodiment, the homologue or portion may retain at least about 80% or at least about 90% of cytosolic domain activity of PTGFRN, ITGA3, IL3RA, SELPLG, ITGB1, or CLSTN1 which may be determined by detecting its accumulation at an extracellular vesicle. Accumulation may be assessed for a chimeric vesicle localization moiety on the basis of the percent of extracellular vesicle positive for the chimeric vesicle localization moiety, and/or the mean abundance of localization moiety in an extracellular vesicle positive for the localization moiety and ignoring extracellular vesicles lacking the localization moiety, as measured by vesicle flow cytometry. The mean abundance of localization moiety in an extracellular vesicle may be the mean concentration, density or amount of localization moiety in an extracellular vesicle positive for the localization moiety. In an embodiment, an alternative measure can also be used, including total number of extracellular vesicles positive for the localization moiety.

In an embodiment, a homologue is an ortholog derived from a common ancestral gene and encodes a protein with the same function in different species. In an embodiment, a homologue is a paralog derived from a homologous gene that has evolved by gene duplication and encodes for a protein with similar but not identical function. Homologous proteins, including orthologs and paralogs, may be identified based on amino acid sequences, curated, grouped and aligned in publicly available databases, such as HomoloGene at the National Center for Biotechnology Information of the National Institutes of Health (NCBI Resource Coordinators (2016) Database resources of the National Center for Biotechnology Information. Nucleic Acids Res. 44:D7-D9), OrthoDB (Waterhouse, R. M. et al. (2011) OrthoDB: the hierarchical catalog of eukaryotic orthologs in 2011. Nucleic Acids Res. 39:D283-8), HOGENOM (Penel, S. et al. (2009) Databases of homologous gene families for comparative genomics. BMC Bioinformatics 10:53), TreeFam (Ruan, J. et al. (2008) TreeFam: 2008 Update. Nucleic Acids Res. 36: D735-D740), Gene Sorter (Kent, W. J. et al. (2005) Exploring relationships and mining data with the UCSC Gene Sorter. Genome Res. 15:737-41), and InParanoid (Sonnhammer, E. L. L. and Östlund, G. (2015) InParanoid 8: orthology analysis between 273 proteomes, mostly eukaryotic. Nucleic Acids Res. 43:D234-D239).

Pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

Suitable pharmaceutically acceptable excipients are well known to a person skilled in the art. Merely by way of example, excipients include, but are not limited to, surfactants, lipophilic vehicles, hydrophobic vehicles, sodium citrate, calcium carbonate, and dicalcium phosphate. Examples of pharmaceutically acceptable excipients include phosphate buffered saline (e.g. 0.01 M phosphate, 0.138 M NaCl, 0.0027 M KCl, pH 7.4), an aqueous solution containing a mineral acid salt such as a hydrochloride, a hydrobromide, a phosphate, or a sulfate, saline, a solution of glycol or ethanol, and a salt of an organic acid such as an acetate, a propionate, a malonate or a benzoate. An adjuvant such as a wetting agent or an emulsifier, and a pH buffering agent can also be used. The pharmaceutically acceptable excipients described in Remington's Pharmaceutical Sciences (Mack Pub. Co., N.J. 1991)(which is incorporated herein by reference in its entirety for all purposes) can be appropriately used. The composition can be formulated into a known form suitable for parenteral administration, for example, injection or infusion. The composition may comprise formulation additives such as a suspending agent, a preservative, a stabilizer and/or a dispersant, and a preservation agent for extending a validity term during storage.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, peritumorally, intrathecally, via intraventricular delivery, intrasternal delivery, intranodally, intramedullary, intramuscularly, intranasally, intraarterially, into an afferent lymph vessel, by intravenous (i.v.) injection, intracranial injection, intramuscular injection, subcutaneous injection, intradermal injection, or intraperitoneally. In one aspect, the compositions of the present invention are administered to a patient by intradermal or subcutaneous injection. In one aspect, the modified vesicles compositions described herein are administered by i.v. injection. Compositions can be administered in a way which allows them to cross the blood-brain barrier, vascular barrier, or other epithelial barrier

When “an immunologically effective amount” or “therapeutic amount” is indicated, the precise amount of the compositions of the present invention to be administered can be determined by a physician with consideration of individual differences in age, weight, tumor size, extent of infection or metastasis, disease condition or condition to be treated, and condition of the patient (subject). As used herein, a “subject” means a mammal. The mammal can be a human or an animal such as a non-human primate, mouse, rat, dog, cat, horse, monkey, ape, rabbit or cow, but are not limited to these examples. Mammals, other than humans, can be advantageously used as subjects that represent animal models of disorders associated with, e.g., cancer. In addition, the methods and compositions described herein can be used to treat domesticated animals and/or pets. The terms, “patient” and “subject” are used interchangeably. A subject can be male or female.

A pharmaceutical composition comprising the modified or engineered EVs or exosomes described herein may be administered at a dosage of 10⁴ to 10¹² EV/kg body weight, or 10⁴ to 10⁹ EV/kg body weight, or 10⁶ to 10⁹ EV/kg bodyweight, in some instances 1 ug to 1 mg exosomal proteins per dose, including all integer values within those ranges. An EV and/or exosome composition may also be administered multiple times at these dosages. EVs and/or exosomes can also be administered by using infusion techniques that are commonly known.

### Uses of EVs of the Invention

EVs of the invention have many of the desirable features of an ideal drug delivery system, such as a long circulating half-life, the intrinsic ability to target tissues, biocompatibility, and minimal or no inherent toxicity issues. Diseases that can be treated with the engineered EVs and/or exosomes described herein using an effective amount thereof include, for example, skeletal muscle disorders, renal diseases, neurodegenerative disorders, cancers (e.g. a hepatocarcinoma), cardiovascular disease, and liver diseases. In an embodiment, the engineered EV and/or exosome comprises a targeting moiety, a VLM and an isopeptide bond that operationally links a targeting moiety to a VLM and optionally a payload. In a preferred embodiment, the engineered EV and/or exosome comprises a targeting moiety, a chimeric VLM and an isopeptide bond that operationally links a targeting moiety to a chimeric VLM and optionally a payload.

The desired amount of molecule-vesicle localization moiety fusion protein or targeting moiety fusion protein (or conjugate)-VLM (or chimeric VLM) fusion protein on an engineered EV and/or exosome, nanoparticle, or other delivery vehicle (collectively “delivery vehicles”) may consider the target cell concentration, density of complementary markers on the target cell, whether target cells are associated with other target cells (e.g., in a tumor or a biofilm), target cells' local microenvironment, the binding affinity (K_{d}) of a marker for a complementary marker on the target cell, and the concentration of delivery vehicle. These parameters can be used to arrive at a desired density of molecule-vesicle localization moiety fusion protein or targeting moiety fusion protein (or conjugate)-VLM (or chimeric VLM) fusion protein on the delivery vehicle. The following equation can be used, at least in part, to arrive at the desired amount of marker expressed on the surface of the delivery vehicle: [molecule-vesicle localization moiety fusion]=[target cell][target marker density][K_{d}][delivery vehicle]⁻¹ Eq. I (similarly also for targeting moiety fusion protein (or conjugate)-VLM (or chimeric VLM) fusion protein in place of molecule-vesicle localization moiety fusion)

The desired amount of molecule-vesicle localization moiety fusion protein on the delivery vehicle can produce 1-100,000 molecule-vesicle localization moiety fusion proteins, or 1-1,000 molecule-vesicle localization moiety fusion proteins, or 1-300 molecule-vesicle localization moiety fusion proteins on the surface of the delivery vehicle. The molecule-vesicle localization moiety fusion proteins can bind to complementary markers with an affinity in the micromolar (μM) range and the desired number of molecule-vesicle localization moiety fusion proteins on the surface delivery vehicle can be 1-10,000, or 50-1,000, or 100-1,000. The molecule-vesicle localization moiety fusion protein can bind to complementary markers with an affinity in the nanomolar (nM) range and the desired number of molecule-vesicle localization moiety fusion proteins on the surface of the delivery vehicle can be 1-1,000, or 1-500, or 1-300, or 10-100. Similarly, also for targeting moiety fusion protein (or conjugate)-VLM (or chimeric VLM) fusion protein in place of molecule-vesicle localization moiety fusion protein.

The desired number of a molecule-vesicle localization moiety fusion protein on a delivery vehicle can be 2-1,000, 10-1,000, 10-5,000, 10-10,000, 10-50,000, 10-100,000, 10-500,000, or 10-1,000,000. The desired number of a molecule-vesicle localization moiety fusion protein on a delivery vehicle can be 100-1,000, 100-5,000, 100-10,000, 100-50,000, 100-100,000, 100-500,000, 100-1,000,000, 1,000-5,000, 1,000-10,000, 1,000-50,000, 1,000-100,000, 1,000-500,000, 1,000-1,000,000, 10,000-50,000, 10,000-100,000, 10,000-500,000, or 10,000-1,000,000. The desired number of a molecule-vesicle localization moiety fusion protein on a delivery vehicle can be at least 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, 500, 550, 600, 650, 700, 750, 800, 850, 900, 950, 1000, 2000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, 50,000, 51,000, 52,000, 53,000, 54,000, 55,000, 60,000, 65,000, 70,000, 75,000, 80,000, 85,000, 90,000, 95,000 or 100,000. The desired number of a molecule-vesicle localization moiety fusion protein on a delivery vehicle can be fewer than 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, 500, 550, 600, 650, 700, 750, 800, 850, 900, 950, 1000, 2000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23.000, 24,000, 25,000, 26.000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, 50,000, 51,000, 52,000, 53,000, 54,000, 55,000, 60,000, 65,000, 70,000, 75,000, 80,000, 85,000, 90,000, 95,000 or 100,000. The desired number of a molecule-vesicle localization moiety fusion protein on a delivery vehicle can be 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, 500, 550, 600, 650, 700, 750, 800, 850, 900, 950, 1000, 2000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, 50,000, 51,000, 52,000, 53,000, 54,000, 55,000, 60,000, 65,000, 70,000, 75,000, 80,000, 85,000, 90,000, 95,000 or 100,000. The delivery vehicle can be an EV or an exosome and the number of molecule-vesicle localization moiety fusion proteins on the surface of the EV or exosome can be 100-100,000. The molecule-vesicle localization moiety fusion protein can bind to a complementary marker on the target with an affinity in the micromolar (μM) range (e.g., 1-500 μM) and the desired number of molecule-vesicle localization moiety fusion proteins on the surface of the delivery vehicle can be 100-100,000. The molecule-vesicle localization moiety fusion protein can bind to a complementary marker on the target with an affinity in the micromolar (μM) range and the desired number of molecule-vesicle localization moiety fusion proteins on the surface of the delivery vehicle can be 100-1,000, 100-5,000, 100-10,000, 100-50,000, 100-100,000, 100-500,000, 100-1,000,000, 1,000-5,000, 1,000-10,000, 1,000-50,000, 1,000-100,000, 1,000-500,000, 1,000-1,000,000, 10,000-50,000, 10,000-100,000, 10,000-500,000, or 10,000-1,000,000. The molecule-vesicle localization moiety fusion protein can bind to a complementary marker on the target with an affinity in the nanomolar (nM) range to sub-nanomolar range and the desired number of molecule-vesicle localization moiety fusion proteins on the surface of the delivery vehicle can be 10-100,000. The molecule-vesicle localization moiety fusion protein can bind to a complementary marker on the target with an affinity in the nanomolar (nM) range (e.g., 1-500 nM) to sub-nanomolar range and the desired number of molecule-vesicle localization moiety fusion protein on the surface of the delivery vehicle can be 100-1,000, 100-5,000, 100-10,000, 100-50,000, 100-100,000, 100-500,000, 100-1,000,000, 1,000-5,000, 1,000-10,000, 1,000-50,000, 1,000-100,000, 1,000-500,000, 1,000-1,000,000, 10,000-50,000, 10,000-100,000, 10,000-500,000, or 10,000-1,000,000. Similarly, also for targeting moiety fusion protein (or conjugate)-VLM (or chimeric VLM) fusion protein in place of molecule-vesicle localization moiety fusion protein.

The effective (desired) amount of molecule-vesicle localization moiety fusion protein can be an amount which gives a desired amount of area under the curve for a desired activity (e.g., binding to target or target cell killing). The effective amount of molecule-vesicle localization moiety fusion protein can be the amount which gives the maximal amount of area under the curve for a desired activity (e.g., binding to target or target cell killing). The effective amount of molecule-vesicle localization moiety fusion protein can be the amount which gives the optimal amount of area under the curve for a desired activity (e.g., binding to target or target cell killing). The effective amount of molecule-vesicle localization moiety fusion protein can be the amount which gives the desired activity rate maximum (analogous to Cₘₐₓ) for a desired activity (e.g., binding to target or target cell killing). The effective amount of molecule-vesicle localization moiety fusion protein can be the amount which gives the maximal activity rate for a desired activity (e.g., binding to target or target cell killing). The effective amount of molecule-vesicle localization moiety fusion protein can be the amount which gives the optimal activity rate for a desired activity (e.g., binding to target or target cell killing). EV, exosome, nanoparticle, or other delivery vehicle activities that may be customized include, for example, any activities useful in the treatment of disease, including, for example, binding of target, target cell killing, differentiation of target cell, expression of transgene in target cell, etc. Similarly, also for targeting moiety fusion protein (or conjugate)-VLM (or chimeric VLM) fusion protein in place of molecule-vesicle localization moiety fusion protein.

### Kits of the Invention

According to another aspect of the invention, kits are provided. Kits according to the invention include package(s) comprising any of the compositions of the invention (including the extracellular vesicles of the invention, chimerical vesicle localization moieties, fusion proteins, and nucleic acids).

The phrase “package” means any vessel containing compositions presented herein. In preferred embodiments, the package can be a box or wrapping. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes (including pre-filled syringes), bottles, and any packaging material suitable for a selected formulation and intended mode of administration and treatment.

The kit can also contain items that are not contained within the package but are attached to the outside of the package, for example, pipettes.

Kits may optionally contain instructions for administering compositions of the present invention to a subject having a condition in need of treatment. Kits may also comprise instructions for approved uses of components of the composition herein by regulatory agencies, such as the United States Food and Drug Administration. Kits may optionally contain labeling or product inserts for the present compositions. The package(s) and/or any product insert(s) may themselves be approved by regulatory agencies. The kits can include compositions in the solid phase or in a liquid phase (such as buffers provided) in a package. The kits also can include buffers for preparing solutions for conducting the methods, and pipettes for transferring liquids from one container to another.

The kit may optionally also contain one or more other compositions for use in combination therapies as described herein. In certain embodiments, the package(s) is a container for any of the means for administration such as intratumoral delivery, peritumoral delivery, intraperitoneal delivery, intrathecal delivery, intramuscular injection, subcutaneous injection, intravenous delivery, intra-arterial delivery, intraventricular delivery, intrasternal delivery, intracranial delivery, or intradermal injection.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1. A Vesicle Localization Moiety with One or More Isopeptide Domain(s) or Isopeptide Tag(s)

The vesicle localization moiety protein, such as IGSF8, is used in this example. However, any VLM disclosed herein, including chimeric VLM may be used in place of the IGSF8. Further, preferred VLMs are single pass transmembrane proteins, especially type I single pass transmembrane proteins provided in Tables 2 and 3. In a preferred embodiment, chimeric VLMs comprising amino-terminal surface-and-transmembrane domain of a type I single pass transmembrane protein as a VLM followed by cytosolic/lumenal domain of a 2^{nd} type I single pass transmembrane protein as a 2^{nd} VLM is covalently linked as in a fusion protein to one or more isopeptide domain(s) or isopeptide tag(s). The VLM and chimeric VLM can have a signal peptide sequence prior to insertion into lipid bilayer, and following insertion, the signal peptide sequence can be cleaved and lost from the VLM or chimeric VLM. In a preferred embodiment, the fusion protein comprises one or more isopeptide domain(s) or tag(s) upstream of a VLM or chimeric VLM with optionally an amino terminal signal peptide sequence which is cleaved following incorporation of the fusion protein into an EV or exosome. Non-limiting examples of fusion proteins of IGSF8 as a VLM and one or more isopeptide domain(s) or tag(s) follows.

The extracellular portion of IGSF8 may be fused with an isopeptide domain to make a vesicle localization moiety fusion protein:

(SEQ ID NO: 198)
dykdhdgdykdhdidykddddkGSGDSATHIKFSKRDEDGKELAGATME
LRDSSGKTISTWISDGQVKDFYLYPGKYTFVETAAPDGYEVATAITFTV
NEQGQVTVNGGSPANLKALEAQKQKEQRQAAEELANAKKLKEQLEKREV
LVPEGPLYRVAGTAVSISCNVTGYEGPAQQNFEWFLYRPEAPDTALGIV
STKDTQFSYAVFKSRVVAGEVQVQRLQGDAVVLKIARLQAQDAGIYECH
TPSTDTRYLGSYSGKVELRVLPDVLQVSAAPPGPRGRQAPTSPPRMTVH
EGQELALGCLARTSTQKHTHLAVSFGRSVPEAPVGRSTLQEVVGIRSDL
AVEAGAPYAERLAAGELRLGKEGTDRYRMVVGGAQAGDAGTYHCTAAEW
IQDPDGSWAQIAEKRAVLAHVDVQTLSSQLAVTVGPGERRIGPGEPLEL
LCNVSGALPPAGRHAAYSVGWEMAPAGAPGPGRLVAQLDTEGVGSLGPG
YEGRHIAMEKVASRTYRLRLEAARPGDAGTYRCLAKAYVRGSGTRLREA
ASARSRPLPVHVREEGVVLEAVAWLAGGTVYRGETASLLCNISVRGGPP
GLRLAASWWVERPEDGELSSVPAQLVGGVGQDGVAELGVRPGGGPVSVE
LVGPRSHRLRLHSLGPEDEGVYHCAPSAWVQHADYSWYQAGSARSGPVT
VYPYMHALDTLFVPLLVGTGVALVTGATVLGTITCCFMKRLRKR.

The sequence in lower case and italicized is an epitope tag. The sequence in bold is the isopeptide domain. The sequences in underline are linkers. The sequence in caps is IGSF8. An alternative IGSF8 vesicle localization moiety-isopeptide domain fusion protein is

(SEQ ID NO: 200)
dykdhdgdykdhdidykddddkGSGGSHMKPERGAVESLQKQHPDYPDI
YGAIDQNGTYQNVRTGEDGKLTFKNLSDGKYRLFENSEPAGYKPVQNKP
IVAFQIVNGEVRDVTSIVPQDIPATYEFTNGKHYITNEPIPPKGSPANL
KALEAQKQKEQRQAAEELANAKKLKEQLEKREVLVPEGPLYRVAGTAVS
ISCNVTGYEGPAQQNFEWFLYRPEAPDTALGIVSTKDTQFSYAVFKSRV
VAGEVQVQRLQGDAVVLKIARLQAQDAGIYECHTPSTDTRYLGSYSGKV
ELRVLPDVLQVSAAPPGPRGRQAPTSPPRMTVHEGQELALGCLARTSTQ
KHTHLAVSFGRSVPEAPVGRSTLQEVVGIRSDLAVEAGAPYAERLAAGE
LRLGKEGTDRYRMVVGGAQAGDAGTYHCTAAEWIQDPDGSWAQIAEKRA
VLAHVDVQTLSSQLAVTVGPGERRIGPGEPLELLCNVSGALPPAGRHAA
YSVGWEMAPAGAPGPGRLVAQLDTEGVGSLGPGYEGRHIAMEKVASRTY
RLRLEAARPGDAGTYRCLAKAYVRGSGTRLREAASARSRPLPVHVREEG
VVLEAVAWLAGGTVYRGETASLLCNISVRGGPPGLRLAASWWVERPEDG
ELSSVPAQLVGGVGQDGVAELGVRPGGGPVSVELVGPRSHRLRLHSLGP
EDEGVYHCAPSAWVQHADYSWYQAGSARSGPVTVYPYMHALDTLFVPLL
VGTGVALVTGATVLGTITCCFMKRLRKR.

An IGSF8 vesicle localization moiety fusion protein with two isopeptide domains is:

(SEQ ID NO: 202)
dykdhdgdykdhdidykddddkGSGDSATHIKFSKRDEDGKELAGATME
LRDSSGKTISTWISDGQVKDFYLYPGKYTFVETAAPDGYEVATAITFTV
NEQGQVTVNGGGGGSGGGGSGSHMKPLRGAVESLQKQHPDYPDIYGAID
QNGTYQNVRTGEDGKLTFKNLSDGKYRLFENSEPAGYKPVQNKPIVAFQ
IVNGEVRDVTSIVPQDIPATYEFTNGKHYITNEPIPPKGSPANLKALEA
QKQKEQRQAAEELANAKKLKEQLEKREVLVPEGPLYRVAGTAVSISCNV
TGYEGPAQQNFEWFLYRPEAPDTALGIVSTKDTQFSYAVFKSRVVAGEV
QVQRLQGDAVVLKIARLQAQDAGIYECHTPSTDTRYLGSYSGKVELRVL
PDVLQVSAAPPGPRGRQAPTSPPRMTVHEGQELALGCLARTSTQKHTHL
AVSFGRSVPEAPVGRSTLQEVVGIRSDLAVEAGAPYAERLAAGELRLGK
EGTDRYRMVVGGAQAGDAGTYHCTAAEWIQDPDGSWAQIAEKRAVLAHV
DVQTLSSQLAVTVGPGERRIGPGEPLELLCNVSGALPPAGRHAAYSVGW
EMAPAGAPGPGRLVAQLDTEGVGSLGPGYEGRHIAMEKVASRTYRLRLE
AARPGDAGTYRCLAKAYVRGSGTRLREAASARSRPLPVHVREEGVVLEA
VAWLAGGTVYRGETASLLCNISVRGGPPGLRLAASWWVERPEDGELSSV
PAQLVGGVGQDGVAELGVRPGGGPVSVELVGPRSHRLRLHSLGPEDEGV
YHCAPSAWVQHADYSWYQAGSARSGPVTVYPYMHALDTLFVPLLVGTGV
ALVTGATVLGTITCCFMKRLRKR.

An IGSF8 vesicle localization moiety fusion protein with three isopeptide domains is:

(SEQ ID NO: 204)
dykdhdgdykdhdidykddddkSSGLVPRGSHMASMTGGQQMGRGSSGL
SGETGQSGNTTIEEDSTTHVKFSKRDANGKELAGAMIELRNLSGQTIQS
WISDGTVKVFYLMPGTYQFVETAAPEGYELAAPITFTIDEKGQIWVDSA
MVDTLSGLSSEQGQSGDDSATHIKFSKRDEDGKELAGATMELRDSSGKT
ISTWISDGQVKDFYLYPGKYTFVETAAPDGYEVATAITFTVNEQGQVTV
NGGGGGSGGGGSGSHMKPLRGAVFSLQKQHPDYPDIYGAIDQNGTYQNV
RTGEDGKLTFKNLSDGKYRLFENSEPAGYKPVQNKPIVAFQIVNGEVRD
VTSIVPQDIPATYEFTNGKHYITNEPIPPKGSPANLKALEAQKQKEQRQ
AAEELANAKKLKEQLEKREVLVPEGPLYRVAGTAVSISCNVTGYEGPAQ
QNFEWFLYRPEAPDTALGIVSTKDTQFSYAVFKSRVVAGEVQVQRLQGD
AVVLKIARLQAQDAGIYECHTPSTDTRYLGSYSGKVELRVLPDVLQVSA
APPGPRGRQAPTSPPRMTVHEGQELALGCLARTSTQKHTHLAVSFGRSV
PEAPVGRSTLQEVVGIRSDLAVEAGAPYAERLAAGELRLGKEGTDRYRM
VVGGAQAGDAGTYHCTAAEWIQDPDGSWAQIAEKRAVLAHVDVQTLSSQ
LAVTVGPGERRIGPGEPLELLCNVSGALPPAGRHAAYSVGWEMAPAGAP
GPGRLVAQLDTEGVGSLGPGYEGRHIAMEKVASRTYRLRLEAARPGDAG
TYRCLAKAYVRGSGTRLREAASARSRPLPVHVREEGVVLEAVAWLAGGT
VYRGETASLLCNISVRGGPPGLRLAASWWVERPEDGELSSVPAQLVGGV
GQDGVAELGVRPGGGPVSVELVGPRSHRLRLHSLGPEDEGVYHCAPSAW
VQHADYSWYQAGSARSGPVTVYPYMHALDTLFVPLLVGTGVALVTGATV
LGTITCCFMKRLRKR.

The extracellular portion of IGSF8 may be fused with an isopeptide tag to make a vesicle localization moiety fusion protein:

Isopeptide-1 Tag-IGSF8
(SEQ ID NO: 216)
DykdhdgdykdhdidykddddkGSGAHIVMVDAYKPTKGSPANLKALEA
QKQKEQRQAAEELANAKKLKEQLEKREVLVPEGPLYRVAGTAVSISCNV
TGYEGPAQQNFEWFLYRPEAPDTALGIVSTKDTQFSYAVFKSRVVAGEV
QVQRLQGDAVVLKIARLQAQDAGIYECHTPSTDTRYLGSYSGKVELRVL
PDVLQVSAAPPGPRGRQAPTSPPRMTVHEGQELALGCLARTSTQKHTHL
AVSFGRSVPEAPVGRSTLQEVVGIRSDLAVEAGAPYAERLAAGELRLGK
EGTDRYRMVVGGAQAGDAGTYHCTAAEWIQDPDGSWAQIAEKRAVLAHV
DVQTLSSQLAVTVGPGERRIGPGEPLELLCNVSGALPPAGRHAAYSVGW
EMAPAGAPGPGRLVAQLDTEGVGSLGPGYEGRHIAMEKVASRTYRLRLE
AARPGDAGTYRCLAKAYVRGSGTRLREAASARSRPLPVHVREEGVVLEA
VAWLAGGTVYRGETASLLCNISVRGGPPGLRLAASWWVERPEDGELSSV
PAQLVGGVGQDGVAELGVRPGGGPVSVELVGPRSHRLRLHSLGPEDEGV
YHCAPSAWVQHADYSWYQAGSARSGPVTVYPYMHALDTLEVPLLVGTGV
ALVTGATVLGTITCCFMKRLRKR.

The sequence in lower case and italicized is an epitope tag. The sequence in bold is the isopeptide tag. The sequences in underline are linkers. The sequence in caps is IGSF8. An alternative IGSF8 vesicle localization moiety-isopeptide domain fusion protein is:

Isopeptide-2 Tag-IGSF8
(SEQ ID NO: 218)
DykdhdgdykdhdidykddddkGSGKLGDIEFIKVNKGSPANLKALEAQ
KQKEQRQAAEELANAKKLKEQLEKREVLVPEGPLYRVAGTAVSISCNVT
GYEGPAQQNFEWFLYRPEAPDTALGIVSTKDTQFSYAVFKSRVVAGEVQ
VQRLQGDAVVLKIARLQAQDAGIYECHTPSTDTRYLGSYSGKVELRVLP
DVLQVSAAPPGPRGRQAPTSPPRMTVHEGQELALGCLARTSTQKHTHLA
VSFGRSVPEAPVGRSTLQEVVGIRSDLAVEAGAPYAERLAAGELRLGKE
GTDRYRMVVGGAQAGDAGTYHCTAAEWIQDPDGSWAQIAEKRAVLAHVD
VQTLSSQLAVTVGPGERRIGPGEPLELLCNVSGALPPAGRHAAYSVGWE
MAPAGAPGPGRLVAQLDTEGVGSLGPGYEGRHIAMEKVASRTYRLRLEA
ARPGDAGTYRCLAKAYVRGSGTRLREAASARSRPLPVHVREEGVVLEAV
AWLAGGTVYRGETASLLCNISVRGGPPGLRLAASWWVERPEDGELSSVP
AQLVGGVGQDGVAELGVRPGGGPVSVELVGPRSHRLRLHSLGPEDEGVY
HCAPSAWVQHADYSWYQAGSARSGPVTVYPYMHALDTLFVPLLVGTGVA
LVTGATVLGTITCCFMKRLRKR.

An IGSF8 vesicle localization moiety fusion protein with two isopeptide tags is:

Isopeptide-1_Isopeptide-2 Tags-IGSF8
(SEQ ID NO: 220)
DykdhdgdykdhdidykddddkGSGAHIVMVDAYKPTKGGGGSGGGGSK
LGDIEFIKVNKGSPANLKALEAQKQKEQRQAAEELANAKKLKEQLEKRE
VLVPEGPLYRVAGTAVSISCNVTGYEGPAQQNFEWFLYRPEAPDTALGI
VSTKDTQFSYAVFKSRVVAGEVQVQRLQGDAVVLKIARLQAQDAGIYEC
HTPSTDTRYLGSYSGKVELRVLPDVLQVSAAPPGPRGRQAPTSPPRMTV
HEGQELALGCLARTSTQKHTHLAVSFGRSVPEAPVGRSTLQEVVGIRSD
LAVEAGAPYAERLAAGELRLQKEGTDRYRMVVGGAQAGDAGTYHCTAAE
WIQDPDGSWAQIAEKRAVLAHVDVQTLSSQLAVTVGPGERRIGPGEPLE
LLCNVSGALPPAGRHAAYSVGWEMAPAGAPGPGRLVAQLDTEGVGSLGP
GYEGRHIAMEKVASRTYRLRLEAARPGDAGTYRCLAKAYVRGSGTRLRE
AASARSRPLPVHVREEGVVLEAVAWLAGGTVYRGETASLLCNISVRGGP
PGLRLAASWWVERPEDGELSSVPAQLVGGVGQDGVAELGVRPGGGPVSV
ELVGPRSHRLRLHSLGPEDEGVYHCAPSAWVQHADYSWYQAGSARSGPV
TVYPYMHALDTLFVPLLVGTGVALVTGATVLGTITCCFMKRLRKR.

An IGSF8 vesicle localization moiety fusion protein with three isopeptide tags is:

Isopeptide-1_Isopeptide-2_Isopeptide-3 Tags-IGSF8
(SEQ ID NO: 222)
DykdhdgdykdhdidykddddkDPIVMIDNDKPITAMVDTLSGLSSEOGQSGDAHI
VMVDAYKPTKGGGGSGGGGSKLGDIEFIKVNKGSPANLKALEAQKQKEQRQAAEEL
ANAKKLKEQLEKREVLVPEGPLYRVAGTAVSISCNVTGYEGPAQQNFEWFLYRPEAPD
TALGIVSTKDTQFSYAVFKSRVVAGEVQVQRLQGDAVVLKIARLQAQDAGIYECHTPST
DTRYLGSYSGKVELRVLPDVLQVSAAPPGPRGRQAPTSPPRMTVHEGQELALGCLARTS
TQKHTHLAVSFGRSVPEAPVGRSTLQEVVGIRSDLAVEAGAPYAERLAAGELRLGKEGT
DRYRMVVGGAQAGDAGTYHCTAAEWIQDPDGSWAQIAEKRAVLAHVDVQTLSSQLA
VTVGPGERRIGPGEPLELLCNVSGALPPAGRHAAYSVGWEMAPAGAPGPGRLVAQLDT
EGVGSLGPGYEGRHIAMEKVASRTYRLRLEAARPGDAGTYRCLAKAYVRGSGTRLREA
ASARSRPLPVHVREEGVVLEAVAWLAGGTVYRGETASLLCNISVRGGPPGLRLAASWW
VERPEDGELSSVPAQLVGGVGQDGVAELGVRPGGGPVSVELVGPRSHRLRLHSLGPEDE
GVYHCAPSAWVQHADYSWYQAGSARSGPVTVYPYMHALDTLFVPLLVGTGVALVTG
ATVLGTITCCFMKRLRKR.

### Affinity Peptides as Targeting Moieties Fused to Isopeptide Tags or Isopeptide Domains:

In the following examples, the sequences in bold signify an affinity peptide THVSPNQGGLPS (SEQ ID NO: 196), also called PEPN, directed to glypican-3 (GPC3) cell surface protein. Other affinity peptides as targeting moieties may be used in place of the GPC3 affinity peptide; non-limiting examples of other affinity peptides include THRPPMWSPVWP (SEQ ID NO.: 194) and those in Table 6 (SEQ ID NO: 157-192). The sequences that are both bold and underlined signify an isopeptide tag or isopeptide domain. The sequences that are underlined signify a linker. Further, sequences that are in lowercase signify an epitope sequence.

Isopeptide(1) tag-GPC3 affinity peptide fusion
protein
(SEQ ID NO: 238)
AHIVMVDAYKPTKSGGGGSGGGGketaaakferqhmdsTHVSPNQGGL
PS.
(SEQ ID NO: 236)
THVSPNQGGLPSSGGGGSGGGGketaaakferqhmdsAHIVMYDAYKP
TK.
Isopeptide(2) tag-GPC3 affinity peptide fusion
protein
(SEQ ID NO: 240)
THVSPNQGGLPSSGGGGSGGGGeqkliseedlKLGDIEFIKVNK.
(SEQ ID NO: 242)
KLGDIEFIKVNKSGGGGSGGGGeqkliseedlTHVSPNQGGLPS.
Isopeptide(3) tag-GPC3 affinity peptide fusion
protein
(SEQ ID NO: 244)
THVSPNQGGLPSSGGGGSGGGGgkpipnpllgldstDPIVMIDNDKPI
T.
(SEQ ID NO: 246)
DPIVMIDNDKPITSGGGGSGGGGgkpipnpllgldstTHVSPNQGGLP
S.

### Affinity Peptides for GPC3 Cell Surface Receptor Fused to Isopeptide Domains:

(SEQ ID NO: 256)
DSATHIKFSKRDEDGKELAGATMELRDSSGKTISTWISDGQVKDFYLYPGKYTFVE
TAAPDGYEVATAITFTVNEQGQVTVNGSGGGGSGGGGketaaakferqhmdsTHVSPNQGG
LPS.
(SEQ ID NO: 254)
THVSPNQGGLPSSGGGGSGGGGketaaakferqhmdsDSATHIKFSKRDEDGKELAGATME
LRDSSGKTISTWISDGQVKDFYLYPGKYTFVETAAPDGYEVATAITFTYNEQGQVT
VNG.
(SEQ ID NO: 258)
THVSPNQGGLPSSGGGGSGGGGeqkliseedlGSHMKPLRGAVFSLQKQHPDYPDIYGAI
DQNGTYQNVRTGEDGKLTFKNLSDGKYRLFENSEPAGYKPVQNKPIVAFQIVNGE
VRDVTSIVPQDIPATYEFTNGKHYITNEPIPPK.
(SEQ ID NO: 260)
GSHMKPLRGAVESLQKQHPDYPDIYGAIDQNGTYQNVRTGEDGKLTFKNLSDGKY
RLFENSEPAGYKPVQNKPIVAFQIVNGEVRDVTSIVPQDIPATYEFTNGKHYITNEPI
PPKSGGGGSGGGGeqkliseedlTHVSPNQGGLPS.
(SEQ ID NO: 262)
THVSPNQGGLPSSGGGGSGGGGgkpipnpllgldstSSGLVPRGSHMASMTGGQQMGRGSS
GLSGETGQSGNTTIEEDSTTHVKFSKRDANGKELAGAMIELRNLSGQTIQSWISDG
TVKVFYLMPGTYQFVETAAPEGYELAAPITFTIDEKGQIWVDS.
(SEQ ID NO: 264)
SSGLVPRGSHMASMTGGQQMGRGSSGLSGETGQSGNTTIEEDSTTHVKFSKRDAN
GKELAGAMIELRNLSGQTIQSWISDGTVKVFYLMPGTYQFVETAAPEGYELAAPIT
FTIDEKGQIWVDSSGGGGSGGGGgkpipnpllgldstTHVSPNQGGLPS.

Single Chain Fv (scFv) as a Targeting Moiety Fused to Isopeptide Tags:

GC33, a single chain antibody that binds to GPC3 is also used as the molecule for the isopeptide tag. GPC3 is a cell surface proteoglycan that bears heparan sulfate that negatively regulates the hedgehog signaling pathway when attached via the GPI-anchor to the cell surface by competing with the hedgehog receptor PTC1 for binding to hedgehog proteins. GPC3 positively regulates the canonical Wnt signaling pathway by binding to the Wnt receptor Frizzled and stimulating the binding of the Frizzled receptor to Wnt ligands. Binds to CD81 which decreases the availability of free CD81 for binding to the transcriptional repressor HHEX, resulting in nuclear translocation of HHEX and transcriptional repression. Plays a role in limb patterning and skeletal development by controlling the cellular response to BMP4. Modulates the effects of growth factors BMP2, BMP7 and FGF7 on renal branching morphogenesis. Required for coronary vascular development. Non-limiting examples of fusion proteins of GC33 scFv as a targeting moiety and one or more isopeptide domain(s) or tag(s) follows.

GC33 scFv may be fused with an isopeptide tag to make a targeting moiety (to target GPC3 cell surface receptor on GPC3 expressing cells)-isopeptide tag fusion protein:

scFv-linker-epitope sequence-isopeptide(1) tag
(SEQ ID NO: 230)
QVQLVQSGAEVKKPGASVKVSCKASGYTFTDYEMHWVRQAPGQGLEWMGALDPKTG
DTAYSQKFKGRVTLTADKSTSTAYMELSSLTSEDTAVYYCTRFYSYTYWGQGTLVTVS
SSSGGSSRSSSSGGGGSGGGGDVVMTQSPLSLPVTPGEPASISCRSSQSLVHSNGNTYLH
WYLQKPGQSPQLLIYKVSNRFSGVPDRFSGSGSGTDFTLKISRVEAEDVGVYYCSQNTH
VPPTFGQGTKLEIKSGGGGGGGGketaaakferqhmdsAHIVMYDAYKPTK

The sequences in uppercase letters signify GC33 single chain Fv (scFv) as an antibody fragment and targeting moiety directed to glypican-3 (GPC3) cell surface protein. The sequences that are both bold and underlined signify an isopeptide tag with the amino acid participating in isopeptide bond formation in bold, underline and italics. The sequences that are underlined signify a linker. Further, sequences that are in lowercase signify an epitope sequence. Alternative targeting moiety (e.g., GC33 scFv)-isopeptide tag fusion proteins and their sequences are:

scFV-linker-epitope sequence-isopeptide(2) tag
(SEQ ID NO: 232)
QVQLVQSGAEVKKPGASVKVSCKASGYTFTDYEMHWVRQAPGQGLEWMGALDPKTG
DTAYSQKFKGRVTLTADKSTSTAYMELSSLTSEDTAVYYCTRFYSYTYWGQGTLVTVS
SSSGGSSRSSSSGGGGSGGGGDVVMTQSPLSLPVTPGEPASISCRSSQSLVHSNGNTYLH
WYLQKPGQSPQLLIYKVSNRFSGVPDRFSGSGSGTDFTLKISRVEAEDVGVYYCSQNTH
VPPTFGQGTKLEIKSGGGGSGGGGeqkliseedlKLGDIEFIKYNK
scFV-linker-epitope sequence-isopeptide(3) tag
(SEQ ID NO: 234)
QVQLVQSGAEVKKPGASYKVSCKASGYTFTDYEMHWVRQAPGQGLEWMGALDPKTGDTAY
SQKFKGRVTLTADKSTSTAYMELSSLTSEDTAVYYCTRFYSYTYWGQGTLVTVSSSSGGSSRSSS
SGGGGSGGGGDFFMTQSPLSLPVTPGEPASISCRSSQSLVHSNGNTYLHWYLQKPGQSPQLLI
YKYSNRFSGVPDRFSGSGSGTDFTLKISRVEAEDVGVYYCSQNTHVPPTFGQGTKLEIKSGGG
GSGGGGgkpipnpllgldstDPIVMIDNDKPIT.

### Additional Examples of Affinity Peptide Fused to Isopeptide Domains Either at N-Terminus or C-Terminus in Fusion Proteins:

Additional examples of an affinity peptide THVSPNQGGLPS (SEQ ID NO: 196) joined to isopeptide domains are as follows. The sequences that are in bold signify the affinity peptide. The sequences that are underlined signify a linker. The sequences that are in italics signify isopeptide-1, -2, -3 domains. The sequences in lowercase signify epitope sequences.

Affinity peptide + isopeptide domain (isopeptide-1)
N-terminal affinity peptide:
(SEQ ID NO: 254)
THVSPNQGGLPSSGGGGSGGGGketaaakferqhmdsDSATHIKFSKRDEDGKELAGATMELRD
SSGKTISTWISDGQVKDFYLYPGKYTFVETAAPDGYEVATAITFTVNEQGQVTVNG (S-tag)
C-terminal affinity peptide:
(SEQ ID NO: 256)
DSATHIKFSKRDEDGKELAGATMELRDSSGKTISTWISDGQVKDFYLYPGKYTFVETAAPDGY
EVATAITFTVNEQGQVTVNGSGGGGSGGGGketaaakferqhmdsTHVSPNQGGLPS (S-tag)
Affinity peptide + isopeptide domain (isopeptide-2)
N-terminal affinity peptide:
(SEQ ID NO: 258)
THVSPNQGGLPSSGGGGSGGGGeqkliseedIGSHMKPLRGAVESLQKQHPDYPDIYGAIDQ
NGTYQNVRTGEDGKLTFKNLSDGKYRLFENSEPAGYKPVQNKPIVAFQIVNGEVRDVTS
IVPQDIPATYEFTNGKHYITNEPIPPK
C-terminal affinity peptide:
(SEQ ID NO: 260)
GSHMKPLRGAVFSLQKQHPDYPDIYGAIDQNGTYQNVRTGEDGKLTFKNLSDGKYRLF
ENSEPAGYKPVQNKPIVAFQIVNGEVRDVTSIVPQDIPATYEFTNGKHYITNEPIPPKSGG
GGSGGGGeqkliseedlTHVSPNQGGLPS
Affinity peptide + isopeptide domain (isopeptide-3)
N-terminal affinity peptide:
(SEQ ID NO: 262)
THVSPNQGGLPSSGGGGSGGGGgkpipnpllgldstSSGLVPRGSHMASMTGGQQMGRGSSG
LSGETGQSGNTTIEEDSTTHVKFSKRDANGKELAGAMIELRNLSGQTIQSWISDGTVKVF
YLMPGTYQFVETAAPEGYELAAPITFTIDEKGQIWVDS
C-terminal affinity peptide:
(SEQ ID NO: 264)
SSGLVPRGSHMASMTGGQQMGRGSSGLSGETGQSGNTTIEEDSTTHVKFSKRDANGKE
LAGAMIELRNLSGQTIQSWISDGTVKVFYLMPGTYQFVETAAPEGYELAAPITFTIDEKG
QIWVDSSGGGGSGGGGgkpipnpllgldstTHVSPNQGGLPS

### Example 2. Making EVs with IGSF8 Vesicle Localization Moiety Fusion Protein

Constructs encoding IGSF8-isopeptide domain fusions of SEQ ID NO: 198, 200, 202 and 204 are made so as to express IGSF8-isopeptide domain fusion of SEQ ID NO: 197, 199, 201 and 203 corresponding to Isopeptide(1) domain-IGSF8 VLM, Isopeptide(2) domain-IGSF8 VLM, Isopeptide(1)+Isopeptide(2) domains-IGSF8 VLM (a DiCatcher-IGSF8) and Isopeptide(3)+Isopeptide(1)+Isopeptide(2) domains-IGSF8 VLM fusion proteins, respectively. The transgene encoding IGSF8-isopeptide domain fusion protein is translated by producer cell machinery into a single-pass transmembrane protein with a 13 amino acid cytosolic (lumenal) domain at the C-terminus. The surface domain contains 552 amino acids fused to a synthetic, recombinant N-terminal domain containing one or more isopeptide domains joined to IGSF8 and, in the case of a multi-isopeptide domain arrangement, to each other via a flexible linker sequence that encodes no secondary structural elements. The synthetic N-terminal domain also contains a 3× Flag epitope for detection in both western blot and flow cytometry applications.

The IGSF8-isopeptide domain fusion protein transgene is synthesized de novo and spliced into a plasmid containing the appropriate sequences for mammalian expression (promoter, terminator, origin of replication, etc.). The plasmid is then transfected into producer cells using a liposome-based transfection reagent to produce the IGSF8-isopeptide domain fusion protein. The nascent fusion protein can comprise a signal peptide at the amino-terminus of the IGSF8-isopeptide domain fusion protein, which is cleaved from the fusion protein following association with cellular membrane, so that the mature or processed fusion protein lacks an amino-terminal signal peptide present initially in the nascent fusion protein. Sequence elements encoded in the primary amino acid sequence of IGSF8 are recognized by producer cell membrane trafficking factors that sort the IGSF8 transgene protein product into exosome biogenesis sites. Exosomes budding from these membrane domains then incorporate the IGSF8 fusion protein.

After intracellular IGSF8 construct expression levels have peaked following transfection, EVs produced from these cells are separated from cells and cellular debris through differential centrifugation. The supernatant from these centrifugation steps is concentrated and buffer exchanged into PBS. The EVs contained in the PBS are then passed through size exclusion chromatography resin to remove unassociated proteins. The EVs are then sterile filtered through a 0.22 μm filter.

IGSF8-containing EVs are detected by vesicle flow cytometry. To ensure that EVs are displaying the construct encoded by the transfected plasmid, and that it is oriented in the correct transmembrane topology, isolated EVs are stained with fluorophore-conjugated anti-epitope tag (e.g., Flag) antibody and a membrane stain. The stained vesicles are evaluated using vesicle flow cytometry (Cytoflex—Beckman Coulter). EVs are identified as membrane stain-positive particles. The amount of recombinant protein on each EV is detected using an fluorophore-conjugated antibody that binds specifically to the epitope sequence included in the primary sequence of the protein, and would only be available on the EV surface if the protein were oriented in the intended topology (C-terminal domain in the lumen; N-terminal domain on the EV surface). The amount of recombinant protein on each evaluated EV is determined by the antibody signal/membrane-stained particle. The presence of coincident membrane stain and antibody-specific fluorescence indicates the presence of the IGSF8 fusion protein, oriented appropriately on the EV surface.

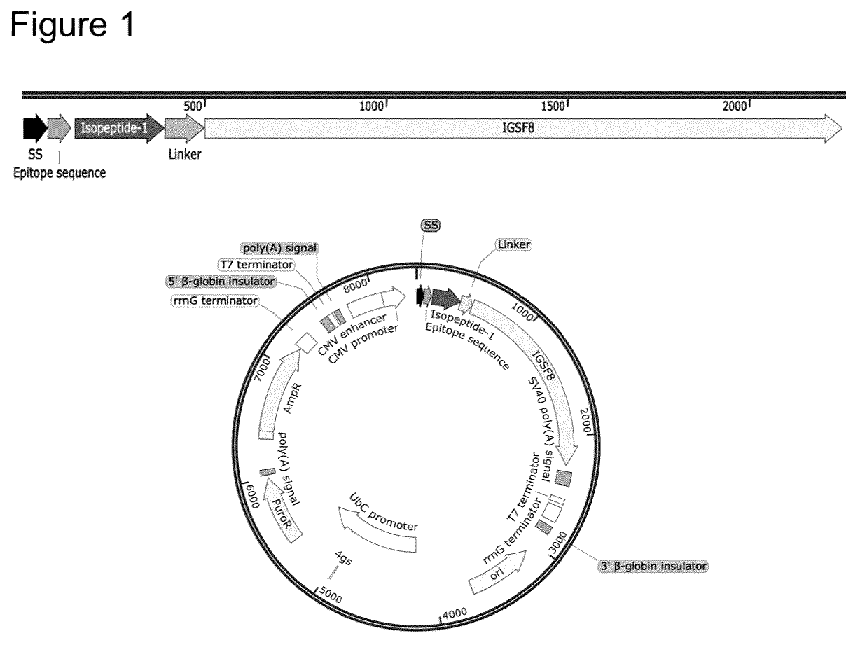

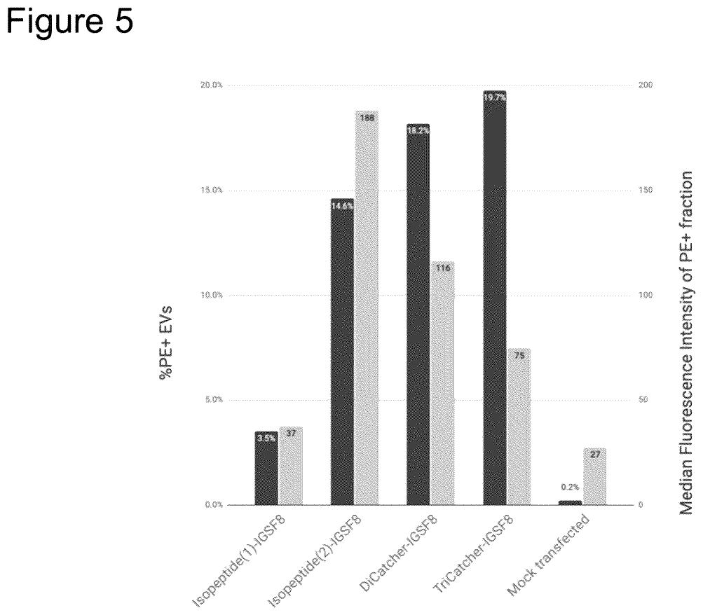

FIG. 5 shows expression characteristics of isopeptide domain fusion proteins in EVs. Plasmids encoding the indicated recombinant proteins (also see FIGS. 1-4 for expression plasmid and structure of the fusion proteins) were transiently transfected into producer cells using standard methods. 5 days post transfection, conditioned media was harvested from the producer cells. EVs were concentrated and purified using in-house methods. A fluorophore-conjugated Flag antibody was used to detect the presence of a Flag epitope tag—specifically encoded by the recombinant isopeptide fusion proteins expressed in this experiment—on purified, membrane-stained EVs, using flow cytometry. The dark gray columns correspond to the left vertical axis and reflect the percentage of analyzed EVs incorporating detectable levels of recombinant fusion protein. The light gray columns correspond to the right vertical axis and reflect the median fluorescent intensity—specific to the Flag antibody-conjugated fluorophore—of EVs incorporating detectable levels of recombinant protein. Comparison with bead-based standard curves suggests that these values (light gray bars) approximate the median number of recombinant proteins incorporated per EV.

### Example 3. Isopeptide Bond Formation Between Isopeptide Domain and Isopeptide Tag to Produce EVs Displaying Targeting Moiety(ies), Such as Affinity Peptide(s) or scFv(s), on their External Surface

EVs incorporating IGSF8-isopeptide domain fusion proteins are mixed with freely soluble targeting moieties fused to an isopeptide tag. Targeting moieties, GC33 scFv and affinity peptide THVSPNQGGLPS (SEQ ID NO: 196), target [symbol] hepatocellular carcinoma (HCC)-specific cell surface protein GPC3. Fusions of these proteins with an isopeptide tag are described in Example 1. The EVs with IGSF8 fusions (from Example I) are mixed with either GC33 scFv with an isopeptide tag or an affinity peptide with and isopeptide tag, for example Vesicle Localization Moiety Fusion Protein SEQ ID NO: 198 and Molecule Tag SEQ ID NO: 238; Vesicle Localization Moiety Fusion SEQ ID NO: 218 and Molecule Tag SEQ ID NO: 248; Chimeric VLM Fusion SEQ ID NO: 212 and Molecule Tag SEQ ID NO: 224; Chimeric VLM Fusion SEQ ID NO: 214 and Molecule Tag SEQ ID NO: 234; and Chimeric VLM Fusion SEQ ID NO: 214 and 2 different Molecule Tags SEQ ID NO: 234 and SEQ ID NO: 236.

In general, EV concentration is approximately 1E10 to 3E11 EVs/mL and isopeptide-targeting moiety fusion protein concentration is 0.02 to 100 μM in 1×PBS buffer. Following incubation at RT to permit binding of the isopeptide tag by the isopeptide domain and subsequent (spontaneous) formation of an isopeptide bond, the EVs along with unreacted isopeptide tag-targeting moiety fusion peptide or protein may be directly analyzed, or alternatively, are purified from unreacted isopeptide tag-targeting moiety fusion protein or peptide by Amicon® Centrifugal Filter Units (100 kDa cut-off), concentrated and buffer exchanged into PBS. EVs are then filtered using Capto™ Core700 (Cytiva) Size Exclusion Chromatography (SEC) resin to remove non-EV-associated protein. Finally, the EVs are sterile filtered, using a 0.22 μm centrifugal filter column unit.

The ability of the IGSF8 fusion protein to bind to GC33 scFv and an affinity peptide with isopeptide tags is assessed by vesicle flow cytometry (vFC) and western blot. In the case of bivalent and trivalent vesicle localization moiety constructs (e.g., vesicle localization moiety fusion proteins comprising 2 or 3 isopeptide domains) such as SEQ ID NO: 206 and 204, targeting moieties with appropriate isopeptide tags (e.g., SEQ ID NO: 236, 240, and 244) are combined and mixed with isolated EVs modified with IGSF8-multi-isopeptide domain vesicle localization moiety fusion proteins in 1×PBS or a buffer solution which supports isopeptide bond formation. Following removal of unligated targeting moieties (i.e., targeting moieties which are attached to isopeptide domains), multivalent modification of individual IGSF8-isopeptide domain vesicle localization moieties are verified by both vFC, immunoprecipitation and western blot. Individual targeting moiety and associated isopeptide-tag conjugate or fusion protein will incorporate unique epitope sequences (for convenience, in lieu of the epitope sequences V5, Myc and S-tag (S-peptide epitope tag) were sometimes used), which will facilitate verification of coincident covalent modification of individual IGSF8-isopeptide domain fusion proteins with up to three different targeting moieties.

### Example 4: Delivery to Target Cells by EVs with Targeting Moiety Fusion Protein Covalently Linked to an IGSF8 Vesicle Localization Moiety Fusion Protein Through an Isopeptide Bond

The affinity of EVs engineered with IGSF8 vesicle localization moiety fusions and a targeting moiety with affinity for GPC3 (the affinity peptide THVSPNQGGLPS (SEQ ID NO: 196; also called PEPN) or GC33 scFv) for the cell surface transmembrane protein GPC3 is measured. HepG2 cells expressing the target surface protein are mixed with PC3 cells that do not express the target protein to create a co-culture system. EVs with THVSPNQGGLPS-IGSF8 fusion or GC33 scFv-IGSF8 are made as describe in Example 3, and labeled with membrane stain. These engineered and labeled EVs are added to HepG2-PC3 cocultures. Binding and uptake of EVs into HepG2 cells versus PC3 cells is assessed by flow cytometry to discern the level of EV-associated fluorescence that becomes incorporated into each cell type in the co-culture.

EVs with the THVSPNQGGLPS-IGSF8 fusion or GC33 scFv-IGSF8 show preferential uptake into HepG2 cells.

### Example 5: In Vivo Biodistribution and Efficacy

EVs with THVSPNQGGLPS-IGSF8 fusion or GC33 scFv-IGSF8 fusion are made as described in Example 3, and labeled with DiR (DiIC18(7); 1,1′-dioctadecyl-3,3,3′,3′-tetramethylindotricarbocyanine iodide), fluorophores or radioactive isotopes. These engineered and labeled EVs are added to wild type (WT) mice. As a negative control, EVs with IGSF8-isopeptide domain, but conjugated to random peptide sequences are used. Delivery by the engineered EVs to the kidneys of the mice is assessed by differential detection of labeled EV enrichment in the target organ or tissue by assessing signal/gram of tissue.

### Example 6: Delivery of EVs to Tumor Cells In Vivo

EVs with affinity peptide-IGSF8 fusion or GC33 scFv-IGSF8 fusion are made as describe in Example 3, and a small molecule drug is loaded into the EVs as a payload. Drug is loaded into the EV by incubating concentrated EVs with drug. Sonication transiently permeabilizes the EV membrane allowing drug to equilibrate between the EV lumen and surrounding solution. Drug loading is validated by washing weakly associated drug from the EVs through several buffer exchange steps followed by solubilizing the EV membrane and assessing the released chemical entities by High Performance Liquid Chromatography (HPLC) to confirm the presence of drug.

The mouse-tumor xenograft model HepG2 is used. NOD.Cg-Prkdcscid Il2rgtm1Wjl/SzJ immune compromised mice are inoculated with 2 million HepG2 xenograft cells (liver cancer) and tumors are allowed to form. Mice are administered EVs carrying the small molecule payload doxorubicin. The tumors in mice receiving the Evs carrying the payload and the affinity peptide-IGSF8 fusion or GC33 scFv-IGSF8 fusion are assayed and examined to determine whether there is inhibition of the tumors in the mice.

### Example 7: Synthesis of Nucleic Acid-Isopeptide Tag Conjugate

Peptide-ASO conjugates, Nterm-AHIVMVDAYKPTK-Cterm-Cys-5′-[TGC GCT CCT GGA CGT AGC C /Cy3/]-3′ (molecular weight: 8747.1; and 1.8 mg yield with 81.9% purity as determined by HPLC; also called 5′-IsoPepTag1-ASO) and 5′-[/Cy3/-TGC GCT CCT GGA CGT AGC C]-3′-Cys-Nterm-AHIVMVDAYKPTK-Cterm (molecular weight: 8641.07; and 1.5 mg yield with 93.9% purity as determined by HPLC; also called 3′-IsoPepTag1-ASO), were synthesized via click chemistry. The purified peptide-ASO conjugates are lyophilized and stored at −70° C. until use.

### Example 8: Preparing EVs with Tricatcher-1 Fusion Protein and Control EVs

HEK293F cells are transfected with an expression plasmid for a fusion protein comprising three isopeptide domains and an IGSF8 VLM, which additionally comprises a signal peptide at the N-terminus so as to permit association of the newly synthesized fusion protein with cellular membrane and sorting to endosomes. 24 hours after transfection, the transfection media was exchanged for fresh media (exosome depleted media or chemically defined media), and the cells were grown for an additional 96 hours. 96 hours following media exchange, the cultures were transferred into 50-mL conical tubes and centrifuged at 3,220×g for 30 min. The supernatant from these cultures were transferred to Amicon® Centrifugal Filter Units (100 kDa cut-off), concentrated and buffer exchanged into PBS. EVs are then filtered using Capto™ Core700 (Cytiva) Size Exclusion Chromatography (SEC) resin to remove non-EV-associated protein. Finally, the EVs are sterile filtered, using a 0.22 μm centrifugal filter column unit.

Isolated EVs are analyzed for the presence of exosomes displaying the Isopeptide-1_Isopeptide-2_Isopeptide-3 Tags-IGSF8 fusion protein (also called “Tricatcher-1-IGSF8 fusion protein) lacking the N-terminal signal peptide sequence cleaved off during EV biogenesis.

Isopeptide-1_Isopeptide-2_Isopeptide-3-IGSFS (also called “Tricatcher-1-
IGSF8 fusion protein”)
(SEQ ID NO: 204)
dykdhdgdykdhdidykddddkSSGLVPRGSHMASMTGGQQMGRGSSGLSGET
GQSGNTTIEEDSTTHVKFSKRDANGKELAGAMIELRNLSGQTIQSWISDGTVKVFY
LMPGTYQFVETAAPEGYELAAPITFTIDEKGQIWVDSAMVDTLSGLSSEQGQSGDDS
ATHIKESKRDEDGKELAGATMELRDSSGKTISTWISDGQVKDFYLYPGKYTFVETA
APDGYEVATAITFTVNEQGQVTVNGGGGGSGGGGSGSHMKPLRGAVFSLQKQHPD
YPDIYGAIDONGTYQNVRTGEDGKLTFKNLSDGKYRLFENSEPAGYKPVQNKPIVA
FQIVNGEVRDVTSIVPQDIPATYEFTNGKHYITNEPIPPKGSPANLKALEAQKQKEQR
QAAEELANAKKLKEQLEKREVLVPEGPLYRVAGTAVSISCNVTGYEGPAQQNFEWFLY
RPEAPDTALGIVSTKDTQFSYAVFKSRVVAGEVQVQRLQGDAVVLKIARLQAQDAGIYE
CHTPSTDTRYLGSYSGKVELRVLPDVLQVSAAPPGPRGRQAPTSPPRMTVHEGQELALG
CLARTSTQKHTHLAVSFGRSVPEAPVGRSTLQEVVGIRSDLAVEAGAPYAERLAAGELR
LGKEGTDRYRMVVGGAQAGDAGTYHCTAAEWIQDPDGSWAQIAEKRAVLAHVDVQT
LSSQLAVTVGPGERRIGPGEPLELLCNVSGALPPAGRHAAYSVGWEMAPAGAPGPGRL
VAQLDTEGVGSLGPGYEGRHIAMEKVASRTYRLRLEAARPGDAGTYRCLAKAYVRGS
GTRLREAASARSRPLPVHVREEGVVLEAVAWLAGGTVYRGETASLLCNISVRGGPPGLR
LAASWWVERPEDGELSSVPAQLVGGVGQDGVAELGVRPGGGPVSVELVGPRSHRLRL
HSLGPEDEGVYHCAPSAWVQHADYSWYQAGSARSGPVTVYPYMHALDTLFVPLLVGT
GVALVTGATVLGTITCCFMKRLRKR.

To obtain control EVs lacking the isopeptide domain-IGSF8 fusion protein, unmodified EVs from untransfected or mock transfected HEK293F cells are produced separately and in parallel with EVs incorporating recombinant proteins: cells are seeded into a bioreactor at a density equivalent to cells being prepared for transfection. After a 24 hour growth period, the cells are exchanged into new media to mimic a post-transfection media exchange. 96 hours following media exchange, the cultures were transferred into 50-mL conical tubes and centrifuged at 3,220×g for 30 min. The supernatant from these cultures were transferred to Amicon® Centrifugal Filter Units (100 kDa cut-off), concentrated and buffer exchanged into PBS. EVs are then filtered using Capto™ Core700 (Cytiva) Size Exclusion Chromatography (SEC) resin to remove non-EV-associated protein. Finally, the EVs are sterile filtered, using a 0.22 μm centrifugal filter column unit.

### Example 9: Fusion Protein Detection on the EV Surface

To ensure that EVs displayed the fusion protein construct encoded by the transfected plasmid and the fusion protein is oriented with correct transmembrane topology, isolated EVs are stained with fluorophore-conjugated anti-FLAG tag antibody and a membrane stain. The stained vesicles are evaluated using vesicle flow cytometry (vFC) (Cytoflex—Beckman Coulter). EVs are identified as membrane stain-positive particles. The amount of recombinant protein on each EV is detected using a fluorophore-conjugated antibody that binds specifically to the epitope sequence included in the primary sequence of the protein, and would only be available on the EV surface if the fusion protein is oriented in the intended topology (C-terminal domain in the lumen; N-terminal domain on the EV surface). The amount of recombinant protein on each evaluated EV is determined by the antibody signal/membrane-stained particle.

### Example 10: Covalent Attachment of ASO-Isopeptide Tag Conjugate to Tricatcher-1-IGSF8 Fusion Protein on Surface of an EV

The ASO-isopeptide tag(1) conjugate of Example 7 is coupled to the IGSF8 fusion protein displayed on EVs of Example 8 through the isopeptide bond formation between isopeptide tag(1) of the former and isopeptide domain-1 of the latter. Briefly, the lyophilized peptide-ASO conjugate of Example 7 is resuspended in 100% DMSO to make a 2.5 mM stock solution. This 2.5 mM ASO-peptide stock solution is diluted in 1×PBS to obtain a working solution of 2.5 μM peptide-ASO conjugate. EVs modified with Tricatcher-1-IGSF8 fusion protein and control unmodified EVs are thawed to RT.

To a 1.5-ml Eppendorf tube, a reaction mixture is prepared containing:

| | | |
|---|---|---|
| 500 | μL | Tricatcher-1-IGSF8-modified EVs or unmodified EVs |
| | | (2.9E11 EVs/mL in 1x PBS) |
| 56 | μL | BSA (100 mg/mL) in 1x PBS or 1x PBS |
| 6 | μL | 2 μM 5′-IsoPepTag1-ASO or 3′-IsoPepTag1-ASO |
| | | conjugate. Following mixing |

by pipetting up and down several times, the reaction tube is placed flat on a plate shaker set at 350 rpm and the reaction leading to spontaneous formation of isopeptide bond formation following binding of isopeptide tag(1) by isopeptide domain-1 on the surface of the Tricatcher-1-IGSF8-modified EVs to proceed at RT for 3 hrs.

Formation of isopeptide bond leading to covalent attachment of the 5′-IsoPepTag1-ASO or 3′-IsoPepTag1-ASO conjugate to Tricatcher-1-IGSF8-modified EVs but not to the control unmodified EVs is analyzed by denaturing gel electrophoresis. Briefly, aliquots from different reaction mixtures are prepared in a microfuge tube as follow:
27 μL reaction sample
10 μL NuPAGE™ LDS sample buffer (4×) (Invitrogen™)

3 μL 500 mM DTT. Samples are mixed, vortexed briefly, spun and heated to 95° C. for 5 min before loading 15 μL per well of NuPAGE® Bis-Tris precast gel. Gel is run at 125V for 1 hr using 1×2-(N-morpholino)ethanesulfonic acid (MES) buffer, following gel manufacturer recommendation, with the loading dye front permitted to run off the gel in order to better separate high molecular weight bands. Seeblue Plus 2 Pre-stained Standard is used as molecular weight standard to follow the course of the electrophoresis and well as a reference for the determining the apparent molecular weight of the targeting moiety-VLM conjugate, in particular, the 5′-IsoPepTag1-ASO-Tricatcher-1-IGSF8 and 3′-IsoPepTag1-ASO-Tricatcher-1-IGSF8 conjugates.

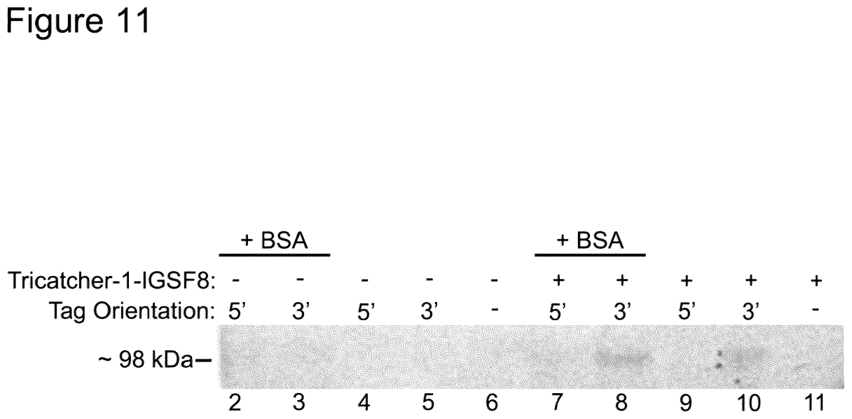

Following electrophoresis, the gel plates are disassembled. The gel is place on a backsupport and in a ChemiDoc™ MP Imaging System (Bio-Rad) to record the location of the pre-stained molecular weight markers based on absorbance and location of Cy3 dye based on fluorescence. Epi-illumination with 520-545 nm excitation (Epi-green) light was used to excite Cy3 dye and the resulting Cy3 fluorescence captured on a CCD camera through a 650±50 nm bandpass filter. FIG. 11 shows the detection of Cy3 fluorescence around the 98 kDa region (ranging from about 80-150 kDa). Samples present in each lane of the gel are:

### Gel Lanes

| Lane | Sample |
|---|---|
| 2 | Unmodified EVs + 0.02 μM 5′-IsoPep1Tag- |
| | ASO + 10 mg/mL BSA |
| 3 | Unmodified EVs + 0.02 μM 3′-IsoPep1Tag- |
| | ASO + 10 mg/mL BSA |
| 4 | Unmodified EVs + 0.02 μM 5′-IsoPep1Tag- |
| | ASO |
| 5 | Unmodified EVs + 0.02 μM 3′-IsoPep1Tag- |
| | ASO |
| 6 | Unmodified EVs |
| 7 | Tricatcher-1-IGSFS-modified EVs + 0.02 μM |
| | 5′-IsoPep1Tag-ASO + 10 mg/mL BSA |
| 8 | Tricatcher-1-IGSFS-modified EVs + 0.02 μM |
| | 3′-IsoPep1Tag-ASO + 10 mg/mL BSA |
| 9 | Tricatcher-1-IGSF8-modified EVs + 0.02 μM |
| | 5′-IsoPep1Tag-ASO |
| 10 | Tricatcher-1-IGSF8-modified EVs + 0.02 μM |
| | 3′-IsoPep1Tag-ASO |
| 11 | Tricatcher-1-IGSF8-modified EVs |

When Tricatcher-1-IGSF8-modified EVs are incubated with Cy3-labeled IsoPep1Tag-ASO conjugate, a protein band displaying Cy3 fluorescence may be detected around 98 kDa region corresponding to the approximately size of Tricatcher-1-IGSF8 fusion protein (see lanes 7-10). This band is not detected when unmodified EVs (lacking the Tricatcher-1-IGSF8 fusion protein) is incubated with the Cy3-labeled IsoPep1Tag-ASO conjugates (lanes 2-5) or when Cy3-labeled IsoPep1Tag-ASO conjugate is left out of the incubation mixture (lanes 6 and 11). Presence of a band most prominently for the sample where Tricatcher-1-IGSF8-modified EVs is incubated with 0.02 μM C-IsoPep1Tag-ASO in the presence of 10 mg/mL BSA (lane 8) is consistent with the formation of an isopeptide bond between the isopeptide-1 domain of the Tricatcher-1-IGSF8 fusion protein and the isopeptide tag-1 of the Cy3-labeled C-IsoPep1Tag-ASO conjugate. Formation of such an isopeptide bond would lead to a covalent attachment of the Cy3-labeled conjugate (m.w. ˜8.6 to 8.7 kDa) to the high molecular weight fusion protein (m.w. ˜106 kDa) and the appearance of a Cy3 fluorescence band at about 98 kDa.

FIG. 11 also shows the importance of the placement of the isopeptide-1 tag and Cy3 dye relative to the antisense oligonucleotide (ASO; 5′-TGCGCTCCTGGACGTAGCC-3′) in the IsoPep1Tag-ASO conjugate. In particular, isopeptide tag-1 at the 3′ end of the ASO with the Cy3 label at the ASO 5′end results in 3′-IsoPep1Tag-ASO conjugate (5′-[/Cy3/-TGC GCT CCT GGA CGT AGC C]-3′-Cys-Nterm-AHIVMVDAYKPTK-Cterm), which is more efficient at forming a covalent bond (i.e., an isopeptide bond) with Tricatcher-1-IGSF8 fusion protein (lane 8) than the same isopeptide tag-1 conjugated to the 5′ end of the ASO with a cy3 dye at its 3′end (5′-IsoPep1Tag-ASO: Nterm-AHIVMVDAYKPTK-Cterm-Cys-5′-[TGC GCT CCT GGA CGT AGC C /Cy3/]-3′; lane 7). Presence of BSA (10 mg/mL) as a carrier in the reaction increases the yield of the coupling reaction between isopeptide domain-1 and isopeptide tag-1, as evident from the fact that the barely visible band in lane 7 is no longer observed in lane 9. Lanes 7 and 9 differ in that the incubation condition contains BSA in the former (lane 7) and lacks BSA in the latter (lane 9).

In conclusion, modified EVs and/or exosomes can be prepared with an isopeptide domain attached to a VLM (or alternatively, chimeric VLM), these modified EVs and/or exosomes can react with a molecule (or targeting moiety or ASO) of interest conjugated to an isopeptide tag resulting in a covalent attachment of the molecule (or targeting moiety or ASO) of interest to the VLM (or chimeric VLM) fusion protein. Covalent attachment of a molecule (or targeting moiety or ASO) of interest-isopeptide tag conjugate (or fusion peptide) to an isopeptide domain-VLM (or chimeric VLM) fusion protein is consistent with the formation of an isopeptide bond between the complementary isopeptide domain and isopeptide tag. Unexpectedly, efficiency of isopeptide bond formation for ASO-isopeptide tag conjugate may be affected by location of the isopeptide tag in relation to the ASO. This dramatic difference in isopeptide bond formation efficiency of an isopeptide domain on the surface of an EV or exosome to an isopeptide tag placed 5′ (FIG. 11, lane 7) or 3′ (FIG. 11, lane 8) to an ASO illustrates an unpredictability in the system which may be unique or exaggerated by the presence of an isopeptide domain being displayed on the surface of an EV or exosome, as the isopeptide tag placed 3′ to the ASO results in a significantly greater coupling efficiency than one placed 5′ to the ASO. Presence of BSA in the reaction mixture presumably reduces loss to non-specific sticking increasing yield of covalent coupling reaction between an isopeptide domain fusion protein and an isopeptide tag conjugate (or fusion peptide).

### Example 11: Covalent Attachment of GPC3 Affinity Peptide-Isopeptide-2 Tag Fusion Peptide (as a Targeting Moiety-Isopeptide Fusion Peptide) to Modified EVs and/or Exosomes Having a Fusion Protein Comprising Isopeptide-2 and IGSF8

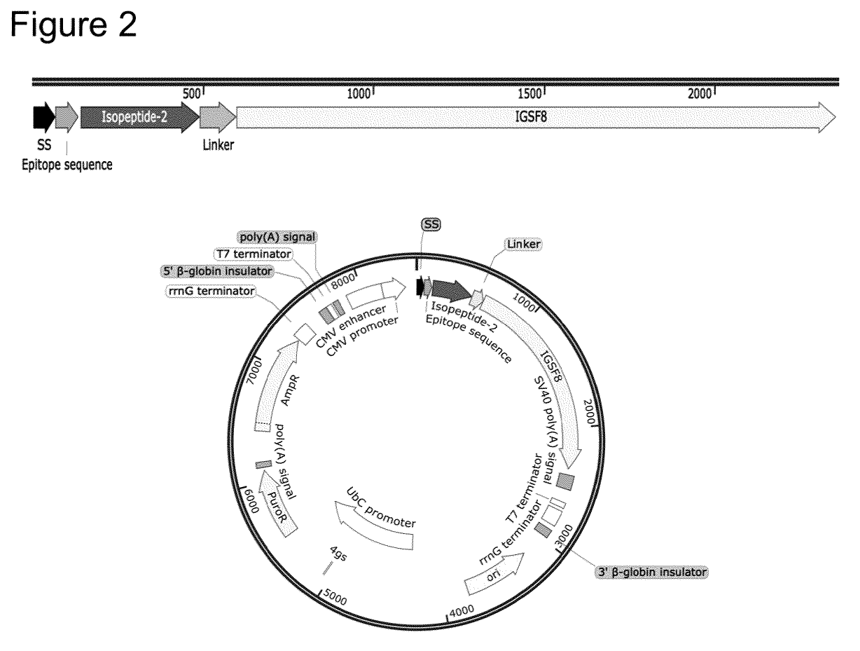

HEK293F cells are transfected with expression constructs for IGSF-isopeptide domain fusion proteins, and modified EVs are isolated from the culture media following transfection, as described in Examples 2 and 8. The expression plasmids used are provided in FIGS. 2, 3 and 4 to produce the proteins with SEQ ID NO: 200, 202, and 204 corresponding to Isopeptide(2) domain-IGSF8 VLM fusion protein (also referred to as “Iso-2” in FIGS. 12 and 13), Isopeptide(1) Isopeptide(2) domains-IGSF VLM fusion protein (also referred to as “Di” in FIGS. 12 and 13) and Isopeptide(3)_Isopeptide(1)_Isopeptide(2) domains-IGSF8 VLM fusion protein (also referred to as “Tricatcher-1-IGSF8” in FIG. 11 and “Tri” in FIGS. 12 and 13), respectively. To assess the presence of the isopeptide domains on the outside of the EV surface, standard vFC Flag staining assay with fluorescent dye-labelled anti-Flag antibody and fluorescent membrane dye is to detect recombinant protein on the EV surface. As negative control, unmodified HEK293F EVs are isolated from culture media of untreated or mock transfected HEK293F cells. Isolated EVs in 1×PBS flash frozen in liquid nitrogen and stored at −70° C. freezer.

Fusion peptide (or fusion protein or fusion polypeptide) comprising a GPC3 affinity peptide and isopeptide-2 tag is obtained from a custom peptide synthesis service at >98% purity (Thermo Fisher Scientific). The peptides are:

(N-IsoPep2Tag-PEPN; SEQ ID NO: 242)
KLGDIEFIKVNKSGGGGGGGGeqkliseedITHVSPNQGGLPS
and
(C-IsoPep2Tag-PEPN; SEQ ID NO: 240)
THVSPNQGGLPSSGGGGSGGGGeqkliseedlKLGDIEFIKVNK,

where the sequence in italics signifies isopeptide-2 tag, underline signifies a linker, lowercase letter signifies myc epitope tag, and bold signifies an affinity peptide or targeting moiety, THVSPNQGGLPS (SEQ ID NO: 196; “PEPN”), directed to glypican-3 (GPC3) cell surface protein. The lyophilized fusion peptide powder is stored at −70° C.

Before use, the frozen EV stock is thawed in a water bath and working EV solution is obtained by diluting in 1×PBS to 5E10 EV particles/mL. The lyophilized GPC3 affinity peptide-isopeptide tag-2 fusion protein is brought to room temperature and resuspended in 100% DMSO to make a 1 mM stock solution. This targeting moiety fusion protein stock solution is diluted in 1×PBS to obtain a working solution of 200 μM GPC3 affinity peptide-isopeptide-2 tag fusion protein.

Using a 96-well microtiter plate, a reaction mix is prepared for each sample:

| | | |
|---|---|---|
| 25 | μL | modified or unmodified EVs (5E10 EV particles/mL) |
| 12.5 | μL | 200 μM GPC3 affinity peptide-isopeptide-2 tag fusion protein |
| 12.5 | μL | 1x PBS (w/o calcium or magnesium) |

for a total volume of 50 μL. Final concentration of modified or unmodified EVs is 2.5E10 EV particles/mL and of the GPC3 affinity peptide-isopeptide tag-2 fusion protein is 50 μM in the reaction mixture, corresponding to about 0.3 mg/mL. Samples are mixed by repeated pipetting. The microtiter plate is covered, spun at 50×g for 1 min and incubated at RT with shaking on an orbital shaker for 3 hrs. Reaction is terminated by adding 12.5 μL of 5× termination buffer (5% SDS, 300 mM Tris-HCl (pH 6.8)), mixing and transferring to 1.5-mL microfuge tube, and heating the tube to 95° C., 5 min. Samples are analyzed on a combined automated capillary electrophoresis and Western blot system (Jess™ Simple Western system) according to the manufacturer ProteinSimple® (San Jose, CA), using anti-myc or anti-FLAG antibody as a primary antibody followed by a goat anti-mouse secondary antibody-conjugated to horseradish peroxidase for chemiluminescent detection.

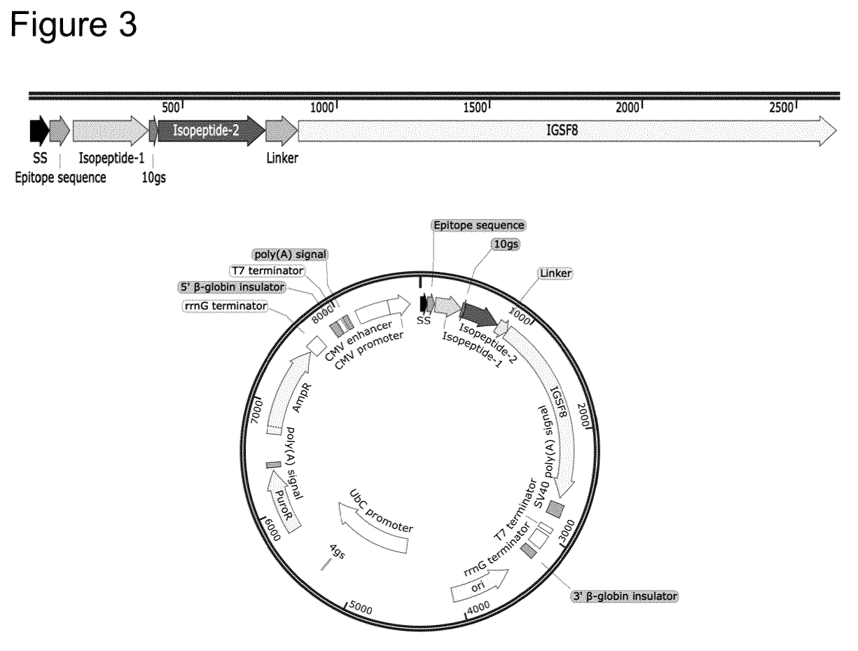

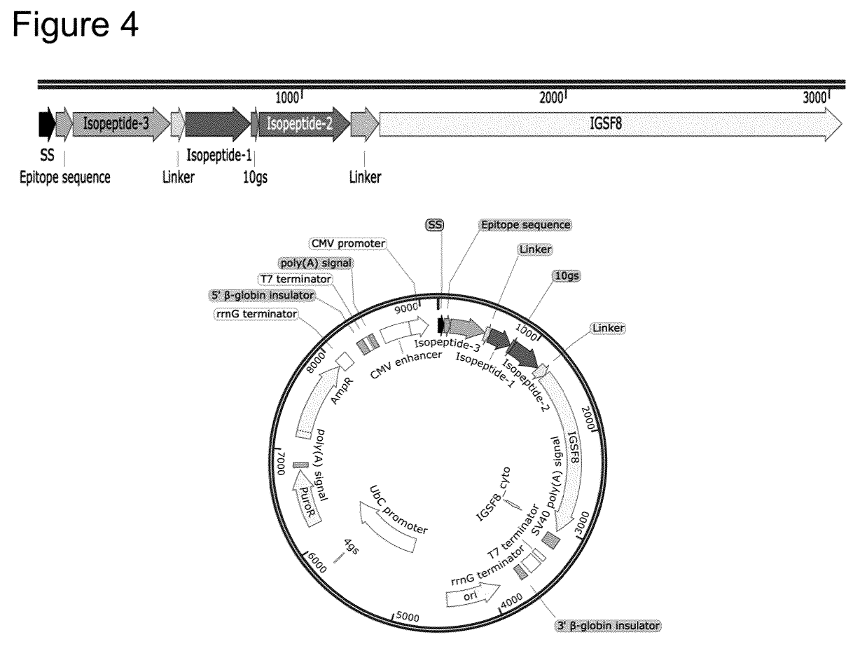

FIG. 12 shows a Western blot analysis obtained through the Jess™ Simple Western system of EVs modified with a fusion protein comprising an isopeptide-2 domain and IGSF8 VLM or unmodified EVs following with incubation with 50 μM Isopeptide-2 tag-PEPN fusion peptide having the amino sequence as provided in SEQ ID NO: 240 or 242. Anti-Flag antibody is directed to the Flag epitope tag present in IGSF8 fusion proteins linked to an Isopeptide-2 domain (SEQ ID NO: 200 from expression vector in FIG. 2), Isopeptide-1 and Isopeptide-2 domains (SEQ ID NO: 202 from expression vector in FIG. 3) or Isopeptide-1, Isopeptide-2 and Isopeptide-3 domains (SEQ ID NO: 204 from expression vector in FIG. 4; also called Tricatcher-1-IGSF8 VLM fusion protein), indicated as “Iso-2,” “Di” and “Tri,” respectively, for “Domain+VLM” status. As can be seen in the last four lanes, prominent band or group of bands corresponding to proteins with an apparent molecule weight of about 70 to 120 kDa are detected by the anti-Flag antibody, consistent with the anti-Flag antibody detecting the Flag epitope tag in the IGSF8 fusion proteins of the Iso-2-modified EVs or Di-modified EVs. The Iso-2 and Di IGSF8 fusion proteins have molecular weights of about 70.5 kDa and 72.6 kDa, respectively.

The “Iso-2,” “Di” and “Tri” modified EV or exosome all comprise a fusion protein comprising an isopeptide(2) domain and IGSF8 as a VLM. Binding of the Isopeptide-2 tag fusion peptide to the Isopeptide-2 domain results in formation of an isopeptide bond and presence of the Isopeptide-2 tag fusion peptide in the high molecular weight region at the location of the Isopeptide-2 domain-IGSF8 fusion protein (see left panel of FIG. 12) while unreacted Isopeptide-2 tag fusion peptide can be seen in the low molecular weight portion (around 12 kDa or less). Spreading of the anti-Flag signals from about 70 to 120 kDa (see right panel of FIG. 12) is consistent with covalent attachment of Isopeptide-2 tag-PEPN fusion peptide to the IGSF8 fusion protein upon binding of the isopeptide-2 domain with the isopeptide-2 tag and subsequent formation of an isopeptide bond. Such an attachment results in a IGSF fusion protein with a isopeptide-2 tag-PEPN branch, which not only increases the molecular weight of the resulting conjugate but may also retard gel mobility beyond what would be observed for a linear polymer with a similar molecular weight.

Use of the anti-myc antibody on an aliquot of the same sample showed that the myc-epitope-labelled Isopeptide-2 tag-PEPN fusion peptide can be detected at about the same region as that for the IGSF8 fusion proteins (see lanes corresponding to “Iso-2” and “Di” in the left panel and compare the bands observed for the anti-myc antibody with the bands seen in the respective lanes in the right panel observed for the anti-Flag antibody). Analysis of the control sample (unmodified EVs incubated with myc-epitope-labelled Isopeptide-2 tag-THV fusion peptide (last two lanes of the left panel labelled as “Un”)) with anti-myc antibody showed absence of any notable band in the high molecular weight region of the gel with signal only detected at the bottom of the gel corresponding to unreacted Isopeptide-2 tag-PEPN fusion peptide, indicating a requirement for a modified EV with IGSF8 fusion protein for the covalent attachment of the Isopeptide-2 tag-PEPN fusion peptide in order to detect anti-myc antibody signal in the high molecular weight region of the gel (e.g., around 70 to 120 kDa).

Presence of an isopeptide domain and isopeptide tag in the IGSF8 fusion protein and Isopeptide-2 tag-PEPN fusion peptide, respectively, can lead to covalent attachment of these two separate polypeptide chains as a result of formation of an isopeptide bond between the isopeptide domain and its complementary isopeptide tag. Unexpectedly, significant differences in coupling efficiency exists depending on placement of the interacting partners within a fusion polypeptide. As can be seen in FIG. 12, placement of the isopeptide tag at the N-terminus of the fusion peptide (lanes marked by “N” for “Tag orientation”) results in a significantly greater coupling efficiency than placement of the isopeptide at the C-terminus of the fusion peptide (lanes marked by “C”). While this difference may be due to differences in the binding affinity for such paired interaction in solution, alternatively, the differences may be a consequence of binding reaction or isopeptide bond formation occurring with a binding partner (e.g., an isopeptide domain) tethered to an EV or exosome through a transmembrane protein (e.g., a VLM or chimeric VLM). Such dramatic efficiencies illustrate unpredictability of isopeptide bond formation for a binding partner tethered to an EV or exosome through a transmembrane protein.

### Example 12: Covalent Attachment of GPC3 Affinity Peptide-Isopeptide-1 Tae Fusion Peptide or Isopeptide-3 Tag Fusion Peptide to IGSF8 Fusion Protein Modified EVs and/or Exosomes

EVs were harvested and purified from producer cells transfected with each of the indicated recombinant isopeptide domain-containing proteins as described above in Example 11. EVs denoted as “Un” were harvested and purified in parallel from untransfected cells. Two additional GPC3 affinity peptide-Isopeptide Tag fusion peptides, namely to either Isopeptide-1 tag or Isopeptide-3 tag, are tested for the ability form a covalent bond (i.e., isopeptide bond) with the IGSF8 fusion proteins comprising IGSF8 as a VLM and an Isopeptide-2 domain (SEQ ID NO: 200 from expression vector in FIG. 2), Isopeptide-1 and Isopeptide-2 domains (SEQ ID NO: 202 from expression vector in FIG. 3) or Isopeptide-1, Isopeptide-2 and Isopeptide-3 domains (SEQ ID NO: 204 from expression vector in FIG. 4; also called Tricatcher-1-IGSF8 fusion protein), indicated as “Iso-2,” “Di” and “Tri,” respectively, for the Isopeptide domain-VLM status (see FIG. 13). GPC3 affinity peptide (PEPN)-Isopeptide-1 Tag fusion peptide and GPC3 affinity peptide-Isopeptide-3 Tag fusion peptide additionally have an S-peptide epitope tag (S-tag) and V5 epitope tag, respectively; while the IGSF fusion proteins all have a Flag epitope tag.

Fusion peptides containing the indicated isopeptide tag displayed at either the N- or C-terminus were mixed with purified EVs and incubated 3 hours at room temperature. Samples from these mixtures were then removed and heated at 95° C. in 1% SDS, before being subjected to capillary electrophoresis and blotted to identify the approximate molecular weight of the reporter peptide. High molecular weight bands detected with the indicated reporter peptide-specific antibodies demonstrate the formation of a covalent bond between reactive isopeptide tag and isopeptide domain pairs. The band in lanes blotted with anti-Flag antibody indicates the approximate molecular weight of the isopeptide domain-IGSF fusion protein. Note that the “Di” construct contains the isopeptide-1 domain and the isopeptide-2 domain, while the “Tri” construct includes all 3 isopeptide domains. The pattern of high molecular weight bands formed between specific fusion peptides and their cognate isopeptide domains demonstrates the specificity of each fusion peptide for a specific isopeptide domain.

In conclusion, a stockpile of modified EVs displaying an isopeptide domain or isopeptide tag may be readily functionalized with a molecule of interest or targeting moiety of interest through the formation of a covalent bond (an isopeptide bond) between complementary isopeptide domain and isopeptide tag. A fusion peptide (or fusion protein or conjugate) comprising the molecule of interest or targeting moiety of interest and an isopeptide domain or isopeptide tag can be made and similarly stockpiled. When required, the modified EVs and/or exosomes comprising a fusion protein of an isopeptide domain or isopeptide tag and a VLM (or chimeric VLM) can be mixed with appropriate fusion peptide (or fusion protein or conjugate) comprising a molecule of interest or targeting moiety of interest and an isopeptide tag or isopeptide domain, and incubated to permit isopeptide bond formation resulting in an EV and/or exosome functionalized with a molecule of interest or targeting moiety of interest.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims. Those skilled in the art will recognize or be able to ascertain using no more than routine experimentation, many equivalents to the specific embodiments of the invention described herein. Such equivalents are intended to be encompassed by the following claims.

TABLE 1
Isopeptide Domains and Isopeptide Tags

SEQ ID NO: Sequence
1:
GGCGCCATGGTGGACACCCTGAGCGGCCTGAGCAGCGAGCAGGGCCAGAGCGGC
GACATGACCATCGAGGAGGACAGCGCCACCCACATCAAGTTCAGCAAGAGAGAC
GAGGACGGCAAGGAGCTGGCCGGCGCCACCATGGAGCTGAGAGACAGCAGCGGC
AAGACCATCAGCACCTGGATCAGCGACGGCCAGGTGAAGGACTTCTACCTGTACC
CCGGCAAGTACACCTTCGTGGAGACCGCCGCCCCCGACGGCTACGAGGTGGCCAC
CGCCATCACCTTCACCGIGAACGAGCAGGGCCAGGTGACCGTGAACGGCAAGGCC
ACCAAGGGCGACGCCCACATC
2:
GAMVDTLSGLSSEQGQSGDMTIEEDSATHIKFSKRDEDGKELAGATMELRDSSGKTIS
TWISDGQVKDFYLYPGKYTFVETAAPDGYEVATAITFTVNEQGQVTVNGKATKGDA
HI
3:
GCCATGGTGGACACCCTGAGCGGCCTGAGCAGCGAGCAGGGCCAGAGCGGCGAC
ATGACCATCGAGGAGGACAGCGCCACCCACATCAAGTTCAGCAAGAGAGACGAG
GACGGCAAGGAGCTGGCCGGCGCCACCATGGAGCTGAGAGACAGCAGCGGCAAG
ACCATCAGCACCTGGATCAGCGACGGCCAGGTGAAGGACTTCTACCTGTACCCCG
GCAAGTACACCTTCGTGGAGACCGCCGCCCCCGACGGCTACGAGGTGGCCACCGC
CATCACCTTCACCGTGAACGAGCAGGGCCAGGTGACCGTGAACGGCAAGGCCACC
AAGGGCGACGCCCACATC
4:
AMVDTLSGLSSEQGQSGDMTIEEDSATHIKFSKRDEDGKELAGATMELRDSSGKTIST
WISDGQVKDFYLYPGKYTFVETAAPDGYEVATAITFTVNEQGQVTVNGKATKGDAHI
5:
ATGGTGGACACCCTGAGCGGCCTGAGCAGCGAGCAGGGCCAGAGCGGCGACATG
ACCATCGAGGAGGACAGCGCCACCCACATCAAGTTCAGCAAGAGAGACGAGGAC
GGCAAGGAGCTGGCCGGCGCCACCATGGAGCTGAGAGACAGCAGCGGCAAGACC
ATCAGCACCTGGATCAGCGACGGCCAGGTGAAGGACTTCTACCTGTACCCCGGCA
AGTACACCTTCGTGGAGACCGCCGCCCCCGACGGCTACGAGGTGGCCACCGCCAT
CACCTTCACCGTGAACGAGCAGGGCCAGGTGACCGTGAACGGCAAGGCCACCAA
GGGCGACGCCCACATC
6:
MVDTLSGLSSEQGQSGDMTIEEDSATHIKFSKRDEDGKELAGATMELRDSSGKTISTW
ISDGQVKDFYLYPGKYTFVETAAPDGYEVATAITFTVNEQGQVTVNGKATKGDAHI
7:
GTGGACACCCTGAGCGGCCTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCAAGGCCACCAAGGG
CGACGCCCACATC
8:
VDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATKGDAHI
9:
GTGGACACCCTGAGCGGCCTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCAAGGCCACCAAGGG
CGACGCCCACATCGAC
10:
VDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATKGDAHID
11:
GTGGACACCCTGAGCGGCCTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCAAGGCCACCAAGGG
C
12:
VDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATKG
13:
GTGGACACCCTGAGCGGCCTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCAAGGCCACCAAG
14:
VDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATK
15:
GTGGACACCCTGAGCGGCCTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCAAGGCCACCAAGGG
CGACGCC
16:
VDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATKGDA
17:
GTGGACACCCTGAGCGGCCTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCAAGGCCACCAAGGG
CGACGCCCAC
18:
VDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATKGDAH
19:
GACACCCTGAGCGGCCTGAGCAGCGAGCAGGGCCAGAGCGGCGACATGACCATC
GAGGAGGACAGCGCCACCCACATCAAGTTCAGCAAGAGAGACGAGGACGGCAAG
GAGCTGGCCGGCGCCACCATGGAGCTGAGAGACAGCAGCGGCAAGACCATCAGC
ACCTGGATCAGCGACGGCCAGGTGAAGGACTTCTACCTGTACCCCGGCAAGTACA
CCTTCGTGGAGACCGCCGCCCCCGACGGCTACGAGGTGGCCACCGCCATCACCTT
CACCGTGAACGAGCAGGGCCAGGTGACCGTGAACGGCAAGGCCACCAAGGGCGA
CGCCCACATC
20:
DTLSGLSSEQGQSGDMTIEEDSATHIKFSKRDEDGKELAGATMELRDSSGKTISTWISD
GQVKDFYLYPGKYTFVETAAPDGYEVATAITFTVNEQGQVTVNGKATKGDAHI
21:
GACAGCGCCACCCACATCAAGTTCAGCAAGAGAGACGAGGACGGCAAGGAGCTG
GCCGGCGCCACCATGGAGCTGAGAGACAGCAGCGGCAAGACCATCAGCACCTGG
ATCAGCGACGGCCAGGTGAAGGACTTCTACCTGTACCCCGGCAAGTACACCTTCG
TGGAGACCGCCGCCCCCGACGGCTACGAGGTGGCCACCGCCATCACCTTCACCGT
GAACGAGCAGGGCCAGGTGACCGTGAACGGCAAGGCCACCAAGGGCGACGCCCA
CATC
22:
DSATHIKFSKRDEDGKELAGATMELRDSSGKTISTWISDGQVKDFYLYPGKYTFVETA
APDGYEVATAITFTVNEQGQVTVNGKATKGDAHI
23:
GTGGACACCCTGAGCGGCCTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCAAGGCCACCAAGGG
CGACGCCCACATCGAC
24:
VDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATKGDAHID
25:
GCCATGGTGGACACCCTGAGCGGCCTGAGCAGCGAGCAGGGCCAGAGCGGCGAC
ATGACCATCGAGGAGGACAGCGCCACCCACATCAAGTTCAGCAAGAGAGACGAG
GACGGCAAGGAGCTGGCCGGCGCCACCATGGAGCTGAGAGACAGCAGCGGCAAG
ACCATCAGCACCTGGATCAGCGACGGCCAGGTGAAGGACTTCTACCTGTACCCCG
GCAAGTACACCTTCGTGGAGACCGCCGCCCCCGACGGCTACGAGGTGGCCACCGC
CATCACCTTCACCGTGAACGAGCAGGGCCAGGTGACCGTGAACGGCAAGGCCACC
AAGGGCGACGCCCACATCGAC
26:
AMVDTLSGLSSEQGQSGDMTIEEDSATHIKFSKRDEDGKELAGATMELRDSSGKTIST
WISDGQVKDFYLYPGKYTFVETAAPDGYEVATAITFTVNEQGQVTVNGKATKGDAHI
D
27:
GTGGACACCCTGAGCAGACTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCTGCAGAAACAGAAGCACCAGAAGATACGGCGGCAGCACCGCCATCC
CCTACAGCATGGAGCAGGGCCAGGTGACCGTGATGGCCAGCAAC
28:
VDTLSRLSSEQGQSGDMTIEEDSATHIKFSKRDEDGKELAGATMELRDSSGKTISTWIS
DGQVKDFYLYPGKYTFCRNRSTRRYGGSTAIPYSMEQGQVTVMASN
29:
GACAGCGCCACCCACATCAAGTTCAGCAAGAGAGACGAGGACGGCAAGGAGCTG
GCCGGCGCCACCATGGAGCTGAGAGACAGCAGCGGCAAGACCATCAGCACCTGG
ATCAGCGACGGCCAGGTGAAGGACTICTACCTGTACCCCGGCAAGTACACCTTCG
TGGAGACCGCCGCCCCCGACGGCTACGAGGTGGCCACCGCCATCACCTTCACCGT
GAACGAGCAGGGCCAGGTGACCGTGAACGGCAAGGCCACCAAG
30:
DSATHIKFSKRDEDGKELAGATMELRDSSGKTISTWISDGQVKDFYLYPGKYTFVETA
APDGYEVATAITFTVNEQGQVTVNGKATK
31:
GACAGCGCCACCCACATCAAGTTCAGCAAGAGAGACGAGGACGGCAAGGAGCTG
GCCGGCGCCACCATGGAGCTGAGAGACAGCAGCGGCAAGACCATCAGCACCTGG
ATCAGCGACGGCCAGGTGAAGGACTICTACCTGTACCCCGGCAAGTACACCTTCG
TGGAGACCGCCGCCCCCGACGGCTACGAGGTGGCCACCGCCATCACCTTCACCGT
GAACGAGCAGGGCCAGGTGACCGTGAACGGC
32:
DSATHIKFSKRDEDGKELAGATMELRDSSGKTISTWISDGQVKDFYLYPGKYTFVETA
APDGYEVATAITFTVNEQGQVTVNG
33:
GACAGCGCCACCCACATCAAGTTCAGCAAGAGAGACGAGGACGGCAAGGAGCTG
GCCGGCGCCACCATGGAGCTGAGAGACAGCAGCGGCAAGACCATCAGCACCTGG
ATCAGCGACGGCCAGGTGAAGGACTTCTACCTGTACCCCGGCAAGTACACCTTCG
TGGAGACCGCCGCCCCCGACGGCTACGAGGTGGCCACCGCCATCACCTTCACCGT
GAACGAGCAGGGCCAGGTGACCGTGAACGGCAAGGCCACCAAGGGCGACGCCCA
CATC
34:
DSATHIKFSKRDEDGKELAGATMELRDSSGKTISTWISDGQVKDFYLYPGKYTFVETA
APDGYEVATAITFTVNEQGQVTVNGKATKGDAHI
35:
GACAGCGCCACCCACATCAAGTTCAGCAAGAGAGACATCGACGGCAAGGAGCTG
GCCGGCGCCACCATGGAGCTGAGAGACAGCAGCGGCAAGACCATCAGCACCTGG
ATCAGCGACGGCCAGGTGAAGGACTTCTACCTGATGCCCGGCAAGTACACCTTCG
TGGAGACCGCCGCCCCCGACGGCTACGAGGTGGCCACCGCCATCACCTTCACCGT
GAACGAGCAGGGCCAGGTGACCGTGAACGGCAAGGCCACCAAGGGCGACGCCCA
CGCCGTGATGGTGGCCGCC
36:
DSATHIKFSKRDIDGKELAGATMELRDSSGKTISTWISDGQVKDFYLMPGKYTFVETA
APDGYEVATAITFTVNEQGQVTVNGKATKGDAHAVMVAA
37:
GACAGCGCCACCCACATCAAGTTCAGCAAGAGAGACGAGGACGGCAAGGAGCTG
GCCGGCGCCACCATGGAGCTGAGAGACAGCAGCGGCAAGACCATCAGCACCTGG
ATCAGCGACGGCCAGGTGAAGGACTICTACCTGTACCCCGGCAAGTACACCTTCG
TGGAGACCGCCGCCCCCGACGGCTACGAGGTGGCCACCGCCATCACCTTCACCGT
GAACGAGCAGGGCCAGGTGACCGTGAACGGCAAGGCCACCAAGGGC
38:
DSATHIKFSKRDEDGKELAGATMELRDSSGKTISTWISDGQVKDFYLYPGKYTFVETA
APDGYEVATAITFTVNEQGQVTVNGKATKG
39:
GTGGACACCCTGAGCGGCCTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCCTGGAG
40:
VDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGLE
41:
ATGGTGGACACCCTGAGCGGCCTGAGCAGCGAGCAGGGCCAGAGCGGCGACATG
ACCATCGAGGAGGACAGCGCCACCCACATCAAGTTCAGCAAGAGAGACGAGGAC
GGCAAGGAGCTGGCCGGCGCCACCATGGAGCTGAGAGACAGCAGCGGCAAGACC
ATCAGCACCTGGATCAGCGACGGCCAGGTGAAGGACTTCTACCTGTACCCCGGCA
AGTACACCTTCGTGGAGACCGCCGCCCCCGACGGCTACGAGGTGGCCACCGCCAT
CACCTTCACCGTGAACGAGCAGGGCCAGGTGACCGTGAACGGCAAGGCCACCAA
G
42:
MVDTLSGLSSEQGQSGDMTIEEDSATHIKFSKRDEDGKELAGATMELRDSSGKTISTW
ISDGQVKDFYLYPGKYTFVETAAPDGYEVATAITFTVNEQGQVTVNGKATK
43:
GTGGACACCCTGAGCGGCCTGAGCAGCGAGCAGGGCCAGAGCGGCGACATGACC
ATCGAGGAGGACAGCGCCACCCACATCAAGTTCAGCAAGAGAGACGAGGACGGC
AAGGAGCTGGCCGGCGCCACCATGGAGCTGAGAGACAGCAGCGGCAAGACCATC
AGCACCTGGATCAGCGACGGCCAGGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCAAGGCCACCAAGGG
C
44:
VDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATKG
45:
GTGACCACCCTGAGCGGCCTGAGCGGCGAGCAGGGCCCCAGCGGCGACATGACC
ACCGAGGAGGACAGCGCCACCCACATCAAGTTCAGCAAGAGAGACGAGGACGGC
AGAGAGCTGGCCGGCGCCACCATGGAGCTGAGAGACAGCAGCGGCAAGACCATC
AGCACCTGGATCAGCGACGGCCACGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCGAGGCCACCAAGGG
CGACGCCCACACC
46:
VTTLSGLSGEQGPSGDMTTEEDSATHIKFSKRDEDGRELAGATMELRDSSGKTISTWIS
DGHVKDFYLYPGKYTFVETAAPDGYEVATAITFTVNEQGQVTVNGEATKGDAHT
47:
ATGACCATCGAGGAGGACAGCGCCACCCACATCAAGTTCAGCAAGAGAGACGAG
GACGGCAAGGAGCTGGCCGGCGCCACCATGGAGCTGAGAGACAGCAGCGGCAAG
ACCATCAGCACCTGGATCAGCGACGGCCAGGTGAAGGACTTCTACCTGTACCCCG
GCAAGTACACCTTCGTGGAGACCGCCGCCCCCGACGGCTACGAGGTGGCCACCGC
CATCACCTTCACCGTGAACGAGCAGGGCCAGGTGACC
48:
MTIEEDSATHIKFSKRDEDGKELAGATMELRDSSGKTISTWISDGQVKDFYLYPGKYT
FVETAAPDGYEVATAITFTVNEQGQVT
49:
ATCGAGACCGAGCAGAACCTGCCCAACGAGGACGGCCAGAGCGGCAACATCATC
GAGCAGGAGGACAGCAAGACCCTGGTGAAGTTCAGCAAGAGAGACATCAAGGGC
AACGAGCTGGCCGGCGCCACCATCGAGCTGAGAGACCTGAGCGGCAAGAGCATC
CAGAGCTGGGTGAGCGACGGCAAGGCCAAGGACTTCTACCTGCTGCCCGGCAGCT
ACGAGTTCGTGGAGACCGCCGCCCCCGAGGGCTACCAGATCGCCACCAAGATCAT
GTTCACCATCAGCACCGACGGCAGAATCACCGTGGACGGCCAGCTGGTG
50:
IETEQNLPNEDGQSGNIIEQEDSKTLVKFSKRDIKGNELAGATIELRDLSGKSIQSWVSD
GKAKDFYLLPGSYEFVETAAPEGYQIATKIMFTISTDGRITVDGQLV
51:
GAGGAGGACAGCGCCACCCACATCAAGTTCAGCAAGAGAGACGAGGACGGCAAG
GAGCTGGCCGGCGCCACCATGGAGCTGAGAGACAGCAGCGGCAAGACCATCAGC
ACCTGGATCAGCGACGGCCAGGTGAAGGACTTCTACCTGTACCCCGGCAAGTACA
CCTTCGTGGAGACCGCCGCCCCCGACGGCTACGAGGTGGCCACCGCCATCACCTT
CACCGTGAACGAGCAGGGCCAGGTGACCGTGAACGGCAAGGCCACCAAGGGC
52:
EEDSATHIKFSKRDEDGKELAGATMELRDSSGKTISTWISDGQVKDFYLYPGKYTFVE
TAAPDGYEVATAITFTVNEQGQVTVNGKATKG
53:
GTGACCACCCTGAGCGGCCTGAGCGGCGAGCAGGGCCCCAGCGGCGACATGACC
ACCGAGGAGGACAGCGCCACCCACATCAAGTTCAGCAAGAGAGACGAGGACGGC
AGAGAGCTGGCCGGCGCCACCATGGAGCTGAGAGACAGCAGCGGCAAGACCATC
AGCACCTGGATCAGCGACGGCCACGTGAAGGACTTCTACCTGTACCCCGGCAAGT
ACACCTTCGTGGAGACCGCCGCCCCCGACGGCTACGAGGTGGCCACCGCCATCAC
CTTCACCGTGAACGAGCAGGGCCAGGTGACCGTGAACGGCGAGGCCACCAAGGG
CGACGCCCACACC
54:
VTTLSGLSGEQGPSGDMTTEEDSATHIKFSKRDEDGRELAGATMELRDSSGKTISTWIS
DGHVKDFYLYPGKYTFVETAAPDGYEVATAITFTVNEQGQVTVNGEATKGDAHT
55:
AAGCCCCTGAGAGGCGCCGTGTTCAGCCTGCAGAAGCAGCACCCCGACTACCCCG
ACATCTACGGCGCCATCGACCAGAACGGCACCTACCAGAACGTGAGAACCGGCG
AGGACGGCAAGCTGACCTTCAAGAACCTGAGCGACGGCAAGTACAGACTGTTCG
AGAACAGCGAGCCCGCCGGCTACAAGCCCGTGCAGAACAAGCCCATCGTGGCCTT
CCAGATCGTGAACGGCGAGGTGAGAGACGTGACCAGCATCGTGCCCCAGGACAT
CCCCGCCACCTACGAGTTCACCAACGGCAAGCACTACATCACCAACGAGCCCATC
CCCCCCAAG
56:
KPLRGAVFSLQKQHPDYPDIYGAIDQNGTYQNVRTGEDGKLTFKNLSDGKYRLFENS
EPAGYKPVONKPIVAFQIVNGEVRDVTSIVPQDIPATYEFTNGKHYITNEPIPPK
57:
GGCAGCCACATGAAGCCCCTGAGAGGCGCCGTGTTCAGCCTGCAGAAGCAGCACC
CCGACTACCCCGACATCTACGGCGCCATCGACCAGAACGGCACCTACCAGAACGT
GAGAACCGGCGAGGACGGCAAGCTGACCTTCAAGAACCTGAGCGACGGCAAGTA
CAGACTGTTCGAGAACAGCGAGCCCGCCGGCTACAAGCCCGTGCAGAACAAGCC
CATCGTGGCCTTCCAGATCGTGAACGGCGAGGTGAGAGACGTGACCAGCATCGTG
CCCCAGGACATCCCCGCCACCTACGAGTTCACCAACGGCAAGCACTACATCACCA
ACGAGCCCATCCCCCCCAAG
58:
GSHMKPLRGAVFSLQKQHPDYPDIYGAIDQNGTYQNVRTGEDGKLTFKNLSDGKYR
LFENSEPAGYKPVQNKPIVAFQIVNGEVRDVTSIVPQDIPATYEFTNGKHYITNEPIPPK
59:
AGCAGCGGCCTGGTGCCCAGAGGCAGCCACATGGCCAGCATGACCGGCGGCCAG
CAGATGGGCAGAGGCAGCAGCGGCCTGAGCGGCGAGACCGGCCAGAGCGGCAAC
ACCACCATCGAGGAGGACAGCACCACCCACGTGAAGTTCAGCAAGAGAGACGCC
AACGGCAAGGAGCTGGCCGGCGCCATGATCGAGCTGAGAAACCTGAGCGGCCAG
ACCATCCAGAGCTGGATCAGCGACGGCACCGTGAAGGTGTTCTACCTGATGCCCG
GCACCTACCAGTTCGTGGAGACCGCCGCCCCCGAGGGCTACGAGCTGGCCGCCCC
CATCACCTTCACCATCGACGAGAAGGGCCAGATCTGGGTGGACAGC
60:
SSGLVPRGSHMASMTGGQQMGRGSSGLSGETGQSGNTTIBEDSTTHVKFSKRDANGK
ELAGAMIELRNLSGQTIQSWISDGTVKVFYLMPGTYQFVETAAPEGYELAAPITFTIDE
KGQIWVDS
61: GCCCACATCGTGATGGTGGACGCCTACAAGCCCACCAAG
62: AHIVMVDAYKPTK
63: AAGCTGGGCGACATCGAGTTCATCAAGGTGAACAAG
64: KLGDIEFIKVNK
65: GACCCCATCGTGATGATCGACAACGACAAGCCCATCACC
66: DPIVMIDNDKPIT

TABLE 2
Preferred VLM which may be used as a VLM or used for the production of
chimeric VLM*

SEQ ID NO: Sequence; Source
67:
ATGGTGTTGCTGAGAGTGTTAATTCTGCTCCTCTCCTGGGCGGGGGGATGGGAG
GTCAGTATGGGAATCCTTTAAATAAATATATCAGACATTATGAAGGATTATCTTAC
AATGTGGATTCATTACACCAAAAACACCAGCGTGCCAAAAGAGCAGTCTCACATG
AAGACCAATTTTTACGTCTAGATTTCCATGCCCATGGAAGACATTTCAACCTACGA
ATGAAGAGGGACACTTCCCTTTTCAGTGATGAATTTAAAGTAGAAACATCAAATA
AAGTACTTGATTATGATACCTCTCATATTTACACTGGACATATTTATGGTGAAGAA
GGAAGTTTTAGCCATGGGTCTGTTATTGATGGAAGATTTGAAGGATTCATCCAGA
CTCGTGGTGGCACATTTTATGTTGAGCCAGCAGAGAGATATATTAAAGACCGAAC
TCTGCCATTTCACTCTGTCATTTATCATGAAGATGATATTAACTATCCCCATAAAT
ACGGTCCTCAGGGGGGCTGTGCAGATCATTCAGTATTTGAAAGAATGAGGAAATA
CCAGATGACTGGTGTAGAGGAAGTAACACAGATACCTCAAGAAGAACATGCTGCT
AATGGTCCAGAACTTCTGAGGAAAAAACGTACAACTTCAGCTGAAAAAAATACTT
GTCAGCTTTATATTCAGACTGATCATTTGTTCTTTAAATATTACGGAACACGAGAA
GCTGTGATTGCCCAGATATCCAGTCATGTTAAAGCGATTGATACAATTTACCAGAC
CACAGACTTCTCCGGAATCCGTAACATCAGTTTCATGGTGAAACGCATAAGAATC
AATACAACTGCTGATGAGAAGGACCCTACAAATCCTTTCCGTTTCCCAAATATTGG
TGTGGAGAAGTTTCTGGAATTGAATTCTGAGCAGAATCATGATGACTACTGTTTGG
CCTATGTCTTCACAGACCGAGATTTTGATGATGGCGTACTTGGTCTGGCTTGGGTT
GGAGCACCTTCAGGAAGCTCTGGAGGAATATGTGAAAAAAGTAAACTCTATTCAG
ATGGTAAGAAGAAGTCCTTAAACACTGGAATTATTACTGTTCAGAACTATGGGTC
TCATGTACCTCCCAAAGTCTCTCACATTACTTTTGCTCACGAAGTTGGACATAACT
TTGGATCCCCACATGATTCTGGAACAGAGTGCACACCAGGAGAATCTAAGAATTT
GGGTCAAAAAGAAAATGGCAATTACATCATGTATGCAAGAGCAACATCTOGOGA
CAAACTTAACAACAATAAATTCTCACTCTGTAGTATTAGAAATATAAGCCAAGTT
CTTGAGAAGAAGAGAAACAACTGTTTTGTTGAATCTGGCCAACCTATTTGTGGAA
ATGGAATGGTAGAACAAGGTGAAGAATGTGATTGTGGCTATAGTGACCAGTGTAA
AGATGAATGCTGCTTCGATGCAAATCAACCAGAGGGAAGAAAATGCAAACTGAA
ACCTGGGAAACAGTGCAGTCCAAGTCAAGGTCCTTGTTGTACAGCACAGTGTGCA
TTCAAGTCAAAGTCTGAGAAGTGTCGGGATGATTCAGACTGTGCAAGGGAAGGAA
TATGTAATGGCTTCACAGCTCTCTGCCCAGCATCTGACCCTAAACCAAACTTCACA
GACTGTAATAGGCATACACAAGTGTGCATTAATGGGCAATGTGCAGGTTCTATCT
GTGAGAAATATGGCTTAGAGGAGTGTACGTGTGCCAGTTCTGATGGCAAAGATGA
TAAAGAATTATGCCATGTATGCTGTATGAAGAAAATGGACCCATCAACTTGTGCC
AGTACAGGGTCTGTGCAGTGGAGTAGGCACTTCAGTGGTCGAACCATCACCCTGC
AACCTGGATCCCCTTGCAACGATTTTAGAGGTTACTGTGATGTTTTCATGCGGTGC
AGATTAGTAGATGCTGATGGTCCTCTAGCTAGGCTTAAAAAAGCAATTTTTAGTCC
AGAGCTCTATGAAAACATTGCTGAATGGATTGTGGCTCATTGGTGGGCAGTATTA
CTTATGGGAATTGCTCTGATCATGCTAATGGCTGGATTTATTAAGATATGCAGTGT
TCATACTCCAAGTAGTAATCCAAAGTTGCCTCCTCCTAAACCACTTCCAGGCACTT
TAAAGAGGAGGAGACCTCCACAGCCCATTCAGCAACCCCAGCGTCAGCGGCCCCG
AGAGAGTTATCAAATGGGACACATGAGACGCTAA; Transcript ID
ENST00000260408; Homo sapiens
68:
MVLLRVLILLLSWAAGMGGQYGNPLNKYIRHYEGLSYNVDSLHQKHQRAKRAVSHE
DQFLRLDFHAHGRHFNLRMKRDTSLFSDEFKVETSNKVLDYDTSHIYTGHIYGEEGSF
SHGSVIDGRFEGFIQTRGGTFYVEPAERYIKDRTLPFHSVIYHEDDINYPHKYGPQGGC
ADHSVFERMRKYQMTGVEEVTQIPQEEHAANGPELLRKKRTTSAEKNTCQLYIQTDH
LFFKYYGTREAVIAQISSHVKAIDTIYQTTDFSGIRNISFMVKRIRINTTADEKDPTNPFR
FPNIGVEKFLELNSEQNHDDYCLAYVFTDRDFDDGVLGLAWVGAPSGSSGGICEKSK
LYSDGKKKSLNTGIITVQNYGSHVPPKVSHITFAHEVGHNFGSPHDSGTECTPGESKNL
GQKENGNYIMYARATSGDKLNNNKFSLCSIRNISQVLEKKENNCFVESGQPICGNGM
VEQGEECDCGYSDQCKDECCFDANQPEGRKCKLKPGKQCSPSQGPCCTAQCAFKSKS
EKCRDDSDCAREGICNGFTALCPASDPKPNFTDCNRHTQVCINGQCAGSICEKYGLEE
CTCASSDGKDDKELCHVCCMKKMDPSTCASTGSVQWSRHFSGRTITLQPGSPCNDFR
GYCDVFMRCRLVDADGPLARLKKAIFSPELYENIAEWIVAHWWAVLLMGIALIMLM
AGFIKICSVHTPSSNPKLPPPKPLPGTLKRRRPPQPIQQPQRQRPRESYQMGHMRR;
ADAM10 protein (ENSP00000260408) encoded by Transcript ID ENST00000260408
from Gene ID ENSG00000137845; Homo sapiens
69:
ATGGAATCCAAGGGGGCCAGTTCCTGCCGTCTGCTCTTCTGCCTCTTGATCTCCGC
CACCGTCTTCAGGCCAGGCCTTGGATGGTATACTGTAAATTCAGCATATGGAGAT
ACCATTATCATACCTTGCCGACTTGACGTACCTCAGAATCTCATGTTTGGCAAATG
GAAATATGAAAAGCCCGATGGCTCCCCAGTATTTATTGCCTTCAGATCCTCTACAA
AGAAAAGTGTGCAGTACGACGATGTACCAGAATACAAAGACAGATTGAACCTCTC
AGAAAACTACACTTTGTCTATCAGTAATGCAAGGATCAGTGATGAAAAGAGATTT
GTGTGCATGCTAGTAACTGAGGACAACGTGTTTGAGGCACCTACAATAGTCAAGG
TGTTCAAGCAACCATCTAAACCTGAAATTGTAAGCAAAGCACTGTTTCTCGAAAC
AGAGCAGCTAAAAAAGTTGGGTGACTGCATTTCAGAAGACAGTTATCCAGATGGC
AATATCACATGGTACAGGAATGGAAAAGTGCTACATCCCCTTGAAGGAGCGGTGG
TCATAATTTTTAAAAAGGAAATGGACCCAGTGACTCAGCTCTATACCATGACTTCC
ACCCTGGAGTACAAGACAACCAAGGCTGACATACAAATGCCATTCACCTGCTCGG
TGACATATTATGGACCATCTGGCCAGAAAACAATTCATTCTGAACAGGCAGTATT
TGATATTTACTATCCTACAGAGCAGGTGACAATACAAGTGCTGCCACCAAAAAAT
GCCATCAAAGAAGGGGATAACATCACTCTTAAATGCTTAGGGAATGGCAACCCTC
CCCCAGAGGAATTTTTGTTTTACTTACCAGGACAGCCCGAAGGAATAAGAAGCTC
AAATACTTACACACTGACGGATGTGAGGCGCAATGCAACAGGAGACTACAAGTGT
TCCCTGATAGACAAAAAAAGCATGATTGCTTCAACAGCTATCACAGTTCACTATTT
GGATTTGTCCTTAAACCCAAGTGGAGAAGTGACTAGACAGATTGGTGATGCCCTA
CCCGTGTCATGCACAATATCTGCTAGCAGGAATGCAACTGTGGTATGGATGAAAG
ATAACATCAGGCTTCGATCTAGCCCGTCATTTTCTAGTCTTCATTATCAGGATGCT
GGAAACTATGTCTGCGAAACTGCTCTGCAGGAGGTTGAAGGACTAAAGAAAAGA
GAGTCATTGACTCTCATTGTAGAAGGCAAACCTCAAATAAAAATGACAAAGAAAA
CTGATCCCAGTGGACTATCTAAAACAATAATCTGCCATGTGGAAGGTTTTOCAAA
GCCAGCCATTCAATGGACAATTACTGGCAGTGGAAGCGTCATAAACCAAACAGAG
GAATCTCCTTATATTAATGGCAGGTATTATAGTAAAATTATCATTTCCCCTGAAGA
GAATGTTACATTAACTTGCACAGCAGAAAACCAACTGGAGAGAACAGTAAACTCC
TTGAATGTCTCTGCTATAAGTATTCCAGAACACGATGAGGCAGACGAGATAAGTG
ATGAAAACAGAGAAAAGGTGAATGACCAGGCAAAACTAATTGTGGGAATOGTTG
TTGGTCTCCTCCTTGCTGCCCTTGTTGCTGGTGTCGTCTACTGGCTGTACATGAAGA
AGTCAAAGACTGCATCAAAACATGTAAACAAGGACCTCGGTAATATGGAAGAAA
ACAAAAAGTTAGAAGAAAACAATCACAAAACTGAAGCCTAA; Transcript ID
ENST00000306107; Homo sapiens
70;
MESKGASSCRLLFCLLISATVFRPGLGWYTVNSAYGDTIIIPCRLDVPQNLMFGKWKY
EKPDGSPVFIAFRSSTKKSVQYDDVPEYKDRLNLSENYTLSISNARISDEKRFVCMLVT
EDNVFEAPTIVKVFKQPSKPEIVSKALFLETEQLKKLGDCISEDSYPDGNITWYRNGKV
LHPLEGAVVIIFKKEMDPVTQLYTMTSTLEYKTTKADIQMPFTCSVTYYGPSGQKTTH
SEQAVFDIYYPTEQVTIQVLPPKNAIKEGDNITLKCLGNGNPPPEEFLFYLPGQPEGIRS
SNTYTLTDVRRNATGDYKCSLIDKKSMIASTAITVHYLDLSLNPSGEVTRQIGDALPVS
CTISASRNATVVWMKDNIRLRSSPSFSSLHYQDAGNYVCETALQEVEGLKKRESLTLI
VEGKPQIKMTKKTDPSGLSKTIICHVEGFPKPAIQWTITGSGSVINQTEESPYINGRYYS
KIIISPEENVTLTCTAENQLERTVNSLNVSAISIPEHDEADEISDENREKVNDQAKLIVGI
VVGLLLAALVAGVVYWLYMKKSKTASKHVNKDLGNMEENKKLEENNHKTEA;
ALCAM protein (ENSP00000305988) encoded by Transcript ID ENST00000306107
from Gene ID ENSG00000170017; Homo sapiens
71:
ATGGAATCCAAGGGGGCCAGTTCCTGCCGTCTGCTCTTCTGCCTCTTGATCTCCGC
CACCGTCTTCAGGCCAGGCCTTGGATGGTATACTGTAAATTCAGCATATGGAGAT
ACCATTATCATACCTTGCCGACTTGACGTACCTCAGAATCTCATGTTTGGCAAATG
GAAATATGAAAAGCCCGATGGCTCCCCAGTATTTATTGCCTTCAGATCCTCTACAA
AGAAAAGTGTGCAGTACGACGATGTACCAGAATACAAAGACAGATTGAACCTCTC
AGAAAACTACACTTTGTCTATCAGTAATGCAAGGATCAGTGATGAAAAGAGATTT
GTGTGCATGCTAGTAACTGAGGACAACGTGTTTGAGGCACCTACAATAGTCAAGG
TGTTCAAGCAACCATCTAAACCTGAAATTGTAAGCAAAGCACTGTTTCTCGAAAC
AGAGCAGCTAAAAAAGTTGGGTGACTGCATTTCAGAAGACAGTTATCCAGATGGC
AATATCACATGGTACAGGAATGGAAAAGTGCTACATCCCCTTGAAGGAGCGGTGG
TCATAATTTTTAAAAAGGAAATGGACCCAGTGACTCAGCTCTATACCATGACTTCC
ACCCTGGAGTACAAGACAACCAAGGCTGACATACAAATGCCATTCACCTGCTCGG
TGACATATTATGGACCATCTGGCCAGAAAACAATTCATTCTGAACAGGCAGTATT
TGATATTTACTATCCTACAGAGCAGGTGACAATACAAGTGCTGCCACCAAAAAAT
GCCATCAAAGAAGGGGATAACATCACTCTTAAATGCTTAGGGAATGGCAACCCTC
CCCCAGAGGAATTTTTGTTTTACTTACCAGGACAGCCCGAAGGAATAAGAAGCTC
AAATACTTACACACTGACGGATGTGAGGCGCAATGCAACAGGAGACTACAAGTGT
TCCCTGATAGACAAAAAAAGCATGATTGCTTCAACAGCTATCACAGTTCACTATTT
GGATTTGTCCTTAAACCCAAGTGGAGAAGTGACTAGACAGATTGGTGATGCCCTA
CCCGTGTCATGCACAATATCTGCTAGCAGGAATGCAACTGTGGTATGGATGAAAG
ATAACATCAGGCTTCGATCTAGCCCGTCATTTTCTAGTCTTCATTATCAGGATGCT
GGAAACTATGTCTGCGAAACTGCTCTGCAGGAGGTTGAAGGACTAAAGAAAAGA
GAGTCATTGACTCTCATTGTAGAAGGCAAACCTCAAATAAAAATGACAAAGAAAA
CTGATCCCAGTGGACTATCTAAAACAATAATCTGCCATGTGGAAGGTTTTCCAAA
GCCAGCCATTCAATGGACAATTACTGGCAGTGGAAGCGTCATAAACCAAACAGAG
GAATCTCCTTATATTAATGGCAGGTATTATAGTAAAATTATCATTTCCCCTGAAGA
GAATGTTACATTAACTTGCACAGCAGAAAACCAACTGGAGAGAACAGTAAACTCC
TTGAATGTCTCTGCTAATGAAAACAGAGAAAAGGTGAATGACCAGGCAAAACTA
ATTGTGGGAATCGTTGTTGGTCTCCTCCTTGCTGCCCTTGTTGCTGGTGTCGTCTAC
TGGCTGTACATGAAGAAGTCAAAGACTGCATCAAAACATGTAAACAAGGACCTCG
GTAATATGGAAGAAAACAAAAAGTTAGAAGAAAACAATCACAAAACTGAAGCCT
AA; Transcript ID ENST00000472644; Homo sapiens
72:
MESKGASSCRLLFCLLISATVFRPGLGWYTVNSAYGDTIIIPCRLDVPQNLMFGKWKY
EKPDGSPVFIAFRSSTKKSVQYDDVPEYKDRLNLSENYTLSISNARISDEKRFVCMLVT
EDNVFEAPTIVKVFKQPSKPEIVSKALFLETEQLKKLGDCISEDSYPDGNITWYRNGKV
LHPLEGAVVIIFKKEMDPVTQLYTMTSTLEYKTTKADIQMPFTCSVTYYGPSGQKTIH
SEQAVFDIYYPTEQVTIQVLPPKNAIKEGDNITLKCLGNGNPPPEEFLFYLPGQPEGIRS
SNTYTLTDVRRNATGDYKCSLIDKKSMIASTAITVHYLDLSLNPSGEVTRQIGDALPVS
CTISASRNATVVWMKDNIRLRSSPSFSSLHYQDAGNYVCETALQEVEGLKKRESLTLI
VEGKPQIKMTKKTDPSGLSKTIICHVEGFPKPAIQWTITGSGSVINQTEESPYINGRYYS
KIIISPEENVTLTCTAENQLERTVNSLNVSANENREKVNDQAKLIVGIVVGLLLAALVA
GVVYWLYMKKSKTASKHVNKDLGNMEENKKLEENNHKTEA; ALCAM protein
(ENSP00000419236) encoded by Transcript ID ENST00000472644 from Gene ID
ENSG00000170017; Homo sapiens
73:
ATGCTGCGCCGCCCCGCTCCCGCGCTGGCCCCGGCCGCCCGGCTGCTGCTGGCCG
GGCTGCTGTGCGGCGGCGGGGTCTGGGCCGCGCGAGTTAACAAGCACAAGCCCTG
GCTGGAGCCCACCTACCACGGCATAGTCACAGAGAACGACAACACCGTGCTCCTC
GACCCCCCACTGATCGCGCTGGATAAAGATGCGCCTCTGCGATTTGCAGGTGAGA
TTTGTGGATTTAAAATTCACGGGCAGAATGTCCCCTTTGATGCAGTGGTAGTGGAT
AAATCCACTGGTGAGGGAGTCATTCGCTCCAAAGAGAAACTGGACTGTGAGCTGC
AGAAAGACTATTCATTCACCATCCAGGCCTATGATTGTGGGAAGGGACCTGATGG
CACCAACGTGAAAAAGTCTCATAAAGCAACTGTTCATATTCAGGTGAACGACGTG
AATGAGTACGCGCCCGTGTTCAAGGAGAAGTCCTACAAAGCCACGGTCATCGAGG
GGAAGCAGTACGACAGCATTTTGAGGGTGGAGGCCGTGGATGCCGACTGCTCCCC
TCAGTTCAGCCAGATTTGCAGCTACGAAATCATCACTCCAGACGTGCCCTTTACTG
TTGACAAAGATGGTTATATAAAAAACACAGAGAAATTAAACTACGGGAAAGAAC
ATCAATATAAGCTGACCGTCACTGCCTATGACTGTGGGAAGAAAAGAGCCACAGA
AGATGTTTTGGTGAAGATCAGCATTAAGCCCACCTGCACCCCTGGGTGGCAAGGA
TGGAACAACAGGATTGAGTATGAGCCGGGCACCGGCGCGTTGGCCGTCTTTCCAA
ATATCCACCTGGAGACATGTGACGAGCCAGTCGCCTCAGTACAGGCCACAGTGGA
GCTAGAAACCAGCCACATAGGGAAAGGCTGCGACCGAGACACCTACTCAGAGAA
GTCCCTCCACCGGCTCTGTGGTGCGGCCGCGGGCACTGCCGAGCTGCTGCCATCCC
CGAGTGGATCCCTCAACTGGACCATGGGCCTGCCCACCGACAATGGCCACGACAG
CGACCAGGTGTTTGAGTTCAACGGCACCCAGGCAGTGAGGATCCCGGATGGCGTC
GTGTCGGTCAGCCCCAAAGAGCCGTTCACCATCTCGGTGTGGATGAGACATGGGC
CATTCGGCAGGAAGAAGGAGACAATTCTTTGCAGTTCTGATAAAACAGATATGAA
TCGGCACCACTACTCCCTCTATGTCCACGGGTGCCGGCTGATCTTCCTCTTCCGTC
AGGATCCTTCTGAGGAGAAGAAATACAGACCTGCAGAGTTCCACTGGAAGTTGAA
TCAGGTCTGTGATGAGGAATGGCACCACTACGTCCTCAATGTAGAATTCCCGAGT
GTGACTCTCTATGTGGATGGCACGTCCCACGAGCCCTTCTCTGTGACTGAGGATTA
CCCGCTCCATCCATCCAAGATAGAAACTCAGCTCGTGGTGGGGGCTTGCTGGCAA
GAGTTTTCAGGAGTTGAAAATGACAATGAAACTGAGCCTGTGACTGTGGCCTCTG
CAGGTGGCGACCTGCACATGACCCAGTTTTTCCGAGGCAATCTGGCTGGCTTAACT
CTCCGTTCCGGGAAACTCGCGGATAAGAAGGTGATCGACTGTCTGTATACCTGCA
AGGAGGGGCTGGACCTGCAGGTCCTCGAAGACAGTGGCAGAGGCGTGCAGATCC
AAGCACACCCCAGCCAGITGGTATTGACCTTGGAGGGAGAAGACCTCGGGGAATT
GGATAAGGCCATGCAGCACATCTCGTACCTGAACTCCCGGCAGTTCCCCACGCCC
GGAATTCGCAGACTCAAAATCACCAGCACAATCAAGTGTTTTAACGAGGCCACCT
GCATTTCGGTCCCCCCGGTAGATGGCTACGTGATGGTITTACAGCCCGAGGAGCC
CAAGATCAGCCTGAGTGGCGTCCACCATTTTGCCCGAGCAGCTTCTGAATTTGAA
AGCTCAGAAGGGGTGTTCCTTTTCCCTGAGCTTCGCATCATCAGCACCATCACGAG
AGAAGTGGAGCCTGAAGGGGACGGGGCTGAGGACCCCACAGTTCAAGAATCACT
GGTGTCCGAGGAGATCGTGCACGACCTGGATACCTGTGAGGTCACGGTGGAGGGA
GAGGAGCTGAACCACGAGCAGGAGAGCCTGGAGGTGGACATGGCCCGCCTGCAG
CAGAAGGGCATTGAAGTGAGCAGCTCTGAACTGGGCATGACCTTCACAGGCGTGG
ACACCATGGCCAGCTACGAGGAGGTTTTGCACCTGCTGCGCTATCGGAACTGGCA
TGCCAGGTCCTTGCTTGACCGGAAGTTTAAGCTCATCTGCTCAGAGCTGAATGGCC
GCTACATCAGCAACGAATTTAAGGTGGAGGTGAATGTAATCCACACGGCCAACCC
CATGGAACACGCCAACCACATGGCTGCCCAGCCACAGTTCGTGCACCCGGAACAC
CGCTCCTTTGTTGACCTGTCAGGCCACAACCTGGCCAACCCCCACCCGTTCGCAGT
CGTCCCCAGCACTGCGACAGTIGTGATCGTGGTGTGCGTCAGCTTCCTGGTGTTCA
TGATTATCCTGGGGGTATTTCGGATCCGGGCCGCACATCGGCGGACCATGCGGGA
TCAGGACACCGGGAAGGAGAACGAGATGGACTGGGACGACTCTGCCCTGACCAT
CACCGTCAACCCCATGGAGACCTATGAGGACCAGCACAGCAGTGAGGAGGAGGA
GGAAGAGGAAGAGGAAGAGGAAAGCGAGGACGGCGAAGAAGAGGATGACATCA
CCAGCGCCGAGTCGGAGAGCAGCGAGGAGGAGGAGGGGGAGCAGGGCGACCCC
CAGAACGCAACCCGGCAGCAGCAGCTGGAGTGGGATGACTCCACCCTCAGCTACT
GA; Transcript ID ENST00000361311; Homo sapiens
74:
MLRRPAPALAPAARLLLAGLLCGGGVWAARVNKHKPWLEPTYHGIVTENDNTVLLD
PPLIALDKDAPLRFAGEICGFKIHGQNVPFDAVVVDKSTGEGVIRSKEKLDCELQKDY
SFTIQAYDCGKGPDGTNVKKSHKATVHIQVNDVNEYAPVFKEKSYKATVIEGKQYDS
ILRVEAVDADCSPQFSQICSYELITPDVPFTVDKDGYIKNTEKLNYGKEHQYKLTVTAY
DCGKKRATEDVLVKISIKPTCTPGWQGWNNRIEYEPGTGALAVFPNIHLETCDEPVAS
VQATVELETSHIGKGCDRDTYSEKSLHRLCGAAAGTAELLPSPSGSLNWTMGLPTDN
GHDSDQVFEFNGTQAVRIPDGVVSVSPKEPFTISVWMRHGPFGRKKETILCSSDKTDM
NRHHYSLYVHGCRLIFLFRQDPSEEKKYRPAEFHWKLNQVCDEEWHHYVLNVEFPSV
TLYVDGTSHEPFSVTEDYPLHPSKIETQLVVGACWQEFSGVENDNETEPVTVASAGG
DLHMTQFFRGNLAGLTLRSGKLADKKVIDCLYTCKEGLDLQVLEDSGRGVQIQAHPS
QLVLTLEGEDLGELDKAMQHISYLNSRQFPTPGIRRLKITSTIKCFNEATCISVPPVDGY
VMVLQPEEPKISLSGVHHFARAASEFESSEGVFLFPELRIISTITREVEPEGDGAEDPTV
QESLVSEEIVHDLDTCEVTVEGEELNHEQESLEVDMARLQQKGIEVSSSELGMTFTGV
DTMASYEEVLHLLRYRNWHARSLLDRKFKLICSELNGRYISNEFKVEVNVIHTANPM
EHANHMAAQPQFVHPEHRSFVDLSGHNLANPHPFAVVPSTATVVIVVCVSFLVFMIIL
GVFRIRAAHRRTMRDQDTGKENEMDWDDSALTITVNPMETYEDQHSSEEEEEEEEEE
ESEDGEEEDDITSAESESSEEEEGEQGDPQNATRQQQLEWDDSTLSY; CLSTN1 protein
(ENSP00000354997) encoded by Transcript ID ENST00000361311 from Gene ID
ENSG00000171603; Homo sapiens
75:
ATGCTGCGCCGCCCCGCTCCCGCGCTGGCCCCGGCCGCCCGGCTGCTGCTGGCCG
GGCTGCTGTGCGGCGGGGGGGTCTGGGCCGCGCGAGTTAACAAGCACAAGCCCTG
GCTGGAGCCCACCTACCACGGCATAGTCACAGAGAACGACAACACCGTGCTCCTC
GACCCCCCACTGATCGCGCTGGATAAAGATGCGCCTCTGCGATTTGCAGAGAGTT
TTGAGGTGACAGTCACCAAAGAAGGTGAGATTTGTGGATTTAAAATTCACGGGCA
GAATGTCCCCTTTGATGCAGTGGTAGTGGATAAATCCACTGGTGAGGGAGTCATT
CGCTCCAAAGAGAAACTGGACTGTGAGCTGCAGAAAGACTATTCATTCACCATCC
AGGCCTATGATTGTGGGAAGGGACCTGATGGCACCAACGTGAAAAAGTCTCATAA
AGCAACTGTTCATATTCAGGTGAACGACGTGAATGAGTACGCGCCCGTGTTCAAG
GAGAAGTCCTACAAAGCCACGGTCATCGAGGGGAAGCAGTACGACAGCATTTTG
AGGGTGGAGGCCGTGGATGCCGACTGCTCCCCTCAGTTCAGCCAGATTTGCAGCT
ACGAAATCATCACTCCAGACGTGCCCTTTACTGTTGACAAAGATGGTTATATAAA
AAACACAGAGAAATTAAACTACGGGAAAGAACATCAATATAAGCTGACCGTCAC
TGCCTATGACTGTGGGAAGAAAAGAGCCACAGAAGATGTTTTGGTGAAGATCAGC
ATTAAGCCCACCTGCACCCCTGGGTGGCAAGGATGGAACAACAGGATTGAGTATG
AGCCGGGCACCGGCGCGTTGGCCGTCTTTCCAAATATCCACCTGGAGACATGTGA
CGAGCCAGTCGCCTCAGTACAGGCCACAGTGGAGCTAGAAACCAGCCACATAGG
GAAAGGCTGCGACCGAGACACCTACTCAGAGAAGTCCCTCCACCGGCTCTGTGGT
GCGGCCGCGGGCACTGCCGAGCTGCTGCCATCCCCGAGTGGATCCCTCAACTGGA
CCATGGGCCTGCCCACCGACAATGGCCACGACAGCGACCAGGTGTTTGAGTTCAA
CGGCACCCAGGCAGTGAGGATCCCGGATGGCGTCGTGTCGGTCAGCCCCAAAGAG
CCGTTCACCATCTCGGTGTGGATGAGACATGGGCCATTCGGCAGGAAGAAGGAGA
CAATTCTTTGCAGTTCTGATAAAACAGATATGAATCGGCACCACTACTCCCTCTAT
GTCCACGGGTGCCGGCTGATCTTCCTCTTCCGTCAGGATCCTTCTGAGGAGAAGAA
ATACAGACCTGCAGAGTTCCACTGGAAGTTGAATCAGGTCTGTGATGAGGAATGG
CACCACTACGTCCTCAATGTAGAATTCCCGAGTGTGACTCTCTATGTGGATGGCAC
GTCCCACGAGCCCTTCTCTGTGACTGAGGATTACCCGCTCCATCCATCCAAGATAG
AAACTCAGCTCGTGGTGGGGGCTTGCTGGCAAGAGTTTTCAGGAGTTGAAAATGA
CAATGAAACTGAGCCTGTGACTGTGGCCTCTGCAGGTGGCGACCTGCACATGACC
CAGTTTTTCCGAGGCAATCTGGCTGGCTTAACTCTCCGTTCCGGGAAACTCGCGGA
TAAGAAGGTGATCGACTGTCTGTATACCTGCAAGGAGGGGCTGGACCTGCAGGTC
CTCGAAGACAGTGGCAGAGGCGTGCAGATCCAAGCACACCCCAGCCAGTTGGTAT
TGACCTTGGAGGGAGAAGACCTCGGGGAATTGGATAAGGCCATGCAGCACATCTC
GTACCTGAACTCCCGGCAGTTCCCCACGCCCGGAATTCGCAGACTCAAAATCACC
AGCACAATCAAGTGTTTTAACGAGGCCACCTGCATTTCGGTCCCCCCGGTAGATG
GCTACGTGATGGTTTTACAGCCCGAGGAGCCCAAGATCAGCCTGAGTGGCGTCCA
CCATTTTGCCCGAGCAGCTTCTGAATTTGAAAGCTCAGAAGGGGTGTTCCTTTTCC
CTGAGCTTCGCATCATCAGCACCATCACGAGAGAAGTGGAGCCTGAAGGGGACG
GGGCTGAGGACCCCACAGTTCAAGAATCACTGGTGTCCGAGGAGATCGTGCACGA
CCTGGATACCTGTGAGGTCACGGTGGAGGGAGAGGAGCTGAACCACGAGCAGGA
GAGCCTGGAGGTGGACATGGCCCGCCTGCAGCAGAAGGGCATTGAAGTGAGCAG
CTCTGAACTGGGCATGACCTTCACAGGCGTGGACACCATGGCCAGCTACGAGGAG
GTTTTGCACCTGCTGCGCTATCGGAACTGGCATGCCAGGTCCTTGCTTGACCGGAA
GTTTAAGCTCATCTGCTCAGAGCTGAATGGCCGCTACATCAGCAACGAATTTAAG
GTGGAGGTGAATGTAATCCACACGGCCAACCCCATGGAACACGCCAACCACATGG
CTGCCCAGCCACAGTTCGTGCACCCGGAACACCGCTCCTTTGTTGACCTGTCAGGC
CACAACCTGGCCAACCCCCACCCGTTCGCAGTCGTCCCCAGCACTGCGACAGTTG
TGATCGTGGTGTGCGTCAGCTTCCTGGTGTTCATGATTATCCTGGGGGTATTTCGG
ATCCGGGCCGCACATCGGCGGACCATGCGGGATCAGGACACCGGGAAGGAGAAC
GAGATGGACTGGGACGACTCTGCCCTGACCATCACCGTCAACCCCATGGAGACCT
ATGAGGACCAGCACAGCAGTGAGGAGGAGGAGGAAGAGGAAGAGGAAGAGGAA
AGCGAGGACGGCGAAGAAGAGGATGACATCACCAGCGCCGAGTCGGAGAGCAGC
GAGGAGGAGGAGGGGGAGCAGGGCGACCCCCAGAACGCAACCCGGCAGCAGCA
GCTGGAGTGGGATGACTCCACCCTCAGCTACTGA; Transcript ID ENST00000377298;
Homo sapiens
76:
MLRRPAPALAPAARLLLAGLLCGGGVWAARVNKHKPWLEPTYHGIVTENDNTVLLD
PPLIALDKDAPLRFAESFEVTVTKEGEICGFKIHGQNVPFDAVVVDKSTGEGVIRSKEK
LDCELQKDYSFTIQAYDCGKGPDGTNVKKSHKATVHIQVNDVNEYAPVFKEKSYKA
TVIEGKQYDSILRVEAVDADCSPQFSQICSYEIITPDVPFTVDKDGYIKNTEKLNYGKE
HQYKLTVTAYDCGKKRATEDVLVKISIKPTCTPGWQGWNNRIEYEPGTGALAVFPNI
HLETCDEPVASVQATVELETSHIGKGCDRDTYSEKSLHRLCGAAAGTAELLPSPSGSL
NWTMGLPTDNGHDSDQVFEFNGTQAVRIPDGVVSVSPKEPFTISVWMRHGPFGRKKE
TILCSSDKTDMNRHHYSLYVHGCRLIFLFRQDPSEEKKYRPAEFHWKLNQVCDEEWH
HYVLNVEFPSVTLYVDGTSHEPFSVTEDYPLHPSKIETQLVVGACWQEFSGVENDNET
EPVTVASAGGDLHMTQFFRGNLAGLTLRSGKLADKKVIDCLYTCKEGLDLQVLEDSG
RGVQIQAHPSQLVLTLEGEDLGELDKAMQHISYLNSRQFPTPGIRRLKITSTIKCFNEAT
CISVPPVDGYVMVLQPEEPKISLSGVHHFARAASEFESSEGVFLFPELRIISTITREVEPE
GDGAEDPTVQESLVSEEIVHDLDTCEVTVEGEELNHEQESLEVDMARLQQKGIEVSSS
ELGMTFTGVDTMASYEEVLHLLRYRNWHARSLLDRKFKLICSELNGRYISNEFKVEV
NVIHTANPMEHANHMAAQPQFVHPEHRSFVDLSGHNLANPHPFAVVPSTATVVIVVC
VSFLVFMIILGVFRIRAAHRRTMRDQDTGKENEMDWDDSALTITVNPMETYEDQHSS
EEEEEEEEEEESEDGEEEDDITSAESESSEEEEGEQGDPQNATRQQQLEWDDSTLSY;
CLSTN1 protein (ENSP00000366513) encoded by Transcript ID ENST00000377298
from Gene ID ENSG00000171603; Homo sapiens
77:
ATGGGCGCCCTCAGGCCCACGCTGCTGCCGCCTTCGCTGCCGCTGCTGCTGCTGCT
AATGCTAGGAATGGGATGCTGGGCCCGGGAGGTGCTGGTCCCCGAGGGGCCCTTG
TACCGCGTGGCTGGCACAGCTGTCTCCATCTCCTGCAATGTGACCGGCTATGAGG
GCCCTGCCCAGCAGAACTTCGAGTGGTTCCTGTATAGGCCCGAGGCCCCAGATAC
TGCACTGGGCATTGTCAGTACCAAGGATACCCAGTTCTCCTATGCTGTCTTCAAGT
CCCGAGTGGTGGCGGGTGAGGTGCAGGTGCAGCGCCTACAAGGTGATGCCGTGGT
GCTCAAGATTGCCCGCCTGCAGGCCCAGGATGCCGGCATTTATGAGTGCCACACC
CCCTCCACTGATACCCGCTACCTGGGCAGCTACAGCGGCAAGGTGGAGCTGAGAG
TTCTTCCAGATGTCCTCCAGGTGTCTGCTGCCCCCCCAGGGCCCCGAGGCCGCCAG
GCCCCAACCTCACCCCCACGCATGACGGTGCATGAGGGGCAGGAGCTGGCACTGG
GCTGCCTGGCGAGGACAAGCACACAGAAGCACACACACCTGGCAGTGTCCTTTGG
GCGATCTGTGCCCGAGGCACCAGTTGGGCGGTCAACTCTGCAGGAAGTGGTGGGA
ATCCGGTCAGACTTGGCCGTGGAGGCTGGAGCTCCCTATGCTGAGCGATTGGCTG
CAGGGGAGCTTCGTCTGGGCAAGGAAGGGACCGATCGGTACCGCATGGTAGTAG
GGGGTGCCCAGGCAGGGGACGCAGGCACCTACCACTGCACTGCCGCTGAGTGGAT
TCAGGATCCTGATGGCAGCTGGGCCCAGATTGCAGAGAAAAGGGCCGTCCTGGCC
CACGTGGATGTGCAGACGCTGTCCAGCCAGCTGGCAGTGACAGTGGGGCCTGGTG
AACGTCGGATCGGCCCAGGGGAGCCCTTGGAACTGCTGTGCAATGTGTCAGGGGC
ACTTCCCCCAGCAGGCCGTCATGCTGCATACTCTGTAGGTTGGGAGATGGCACCT
GCGGGGGCACCTGGGCCCGGCCGCCTGGTAGCCCAGCTGGACACAGAGGGTGTG
GGCAGCCTGGGCCCTGGCTATGAGGGCCGACACATTGCCATGGAGAAGGTGGCAT
CCAGAACATACCGGCTACGGCTAGAGGCTGCCAGGCCTGGTGATGCGGGCACCTA
CCGCTGCCTCGCCAAAGCCTATGTTCGAGGGTCTGGGACCCGGCTTCGTGAAGCA
GCCAGTGCCCGTTCCCGGCCTCTCCCTGTACATGTGCGGGAGGAAGGTGTGGTGC
TGGAGGCTGTGGCATGGCTAGCAGGAGGCACAGTGTACCGCGGGGAGACTGCCTC
CCTGCTGTGCAACATCTCTGTGCGGGGTGGCCCCCCAGGACTGCGGCTGGCCGCC
AGCTGGTGGGTGGAGCGACCAGAGGACGGAGAGCTCAGCTCTGTCCCTGCCCAGC
TGGTGGGTGGCGTAGGCCAGGATGGTGTGGCAGAGCTGGGAGTCCGGCCTGGAG
GAGGCCCTGTCAGCGTAGAGCTGGTGGGGCCCCGAAGCCATCGGCTGAGACTACA
CAGCTTGGGGCCCGAGGATGAAGGCGTGTACCACTGTGCCCCCAGCGCCTGGGTG
CAGCATGCCGACTACAGCTGGTACCAGGCGGGCAGTGCCCGCTCAGGGCCTOTTA
CAGTCTACCCCTACATGCATGCCCTGGACACCCTATTTGTGCCTCTGCTGGTGGGT
ACAGGGGGGCCCTAGTCACTGGTGCCACTGTCCTTGGTACCATCACTTGCTGCTT
CATGAAGAGGCTTCGAAAACGGTGA; Transcript ID ENST00000314485; Homo
sapiens, Transcript ID ENST00000368086; Homo sapiens, Transcript ID
ENST00000614243; Homo sapiens
78:
MGALRPTLLPPSLPLLLLLMLGMGCWAREVLVPEGPLYRVAGTAVSISCNVTGYEGP
AQQNFEWFLYRPEAPDTALGIVSTKDTQFSYAVFKSRVVAGEVQVQRLOGDAVVLKI
ARLQAQDAGIYECHTPSTDTRYLGSYSGKVELRVLPDVLQVSAAPPGPRGRQAPTSPP
RMTVHEGQELALGCLARTSTQKHTHLAVSFGRSVPEAPVGRSTLQEVVGIRSDLAVE
AGAPYAERLAAGELRLGKEGTDRYRMVVGGAQAGDAGTYHCTAAEWIQDPDGSW
AQIAEKRAVLAHVDVQTLSSQLAVTVGPGERRIGPGEPLELLCNVSGALPPAGRHAA
YSVGWEMAPAGAPGPGRLVAQLDTEGVGSLGPGYEGRHIAMEKVASRTYRLRLEAA
RPGDAGTYRCLAKAYVRGSGTRLREAASARSRPLPVHVREEGVVLEAVAWLAGGTV
YRGETASLLCNISVRGGPPGLRLAASWWVERPEDGELSSVPAQLVGGVGQDGVAELG
VRPGGGPVSVELVGPRSHRLRLHSLGPEDEGVYHCAPSAWVQHADYSWYQAGSARS
GPVTVYPYMHALDTLFVPLLVGTGVALVTGATVLGTITCCFMKRLRKR; IGSF8
protein (ENSP00000316664) encoded by Transcript ID ENST00000314485 from
Gene ID ENSG00000162729; Homo sapiens, IGSF8 protein (ENSP00000357065)
encoded by Transcript ID ENST00000368086 from Gene ID ENSG00000162729;
Homo sapiens, IGSF8 protein (ENSP00000477565) encoded by Transcript ID
ENST00000614243 from Gene ID ENSG00000162729; Homo sapiens
79:
ATGGTCCTCCTTTGGCTCACGCTGCTCCTGATCGCCCTGCCCTGTCTCCTGCAAAC
GAAGGAAGATCCAAACCCACCAATCACGAACCTAAGGATGAAAGCAAAGGCTCA
GCAGTTGACCTGGGACCTTAACAGAAATGTGACCGATATCGAGTGTGTTAAAGAC
GCCGACTATTCTATGCCGGCAGTGAACAATAGCTATTGCCAGTTTGGAGCAATTTC
CTTATGTGAAGTGACCAACTACACCGTCCGAGTGGCCAACCCACCATTCTCCACGT
GGATCCTCTTCCCTGAGAACAGTGGGAAGCCTTGGGCAGGTGCGGAGAATCTGAC
CTGCTGGATTCATGACGTGGATTTCTTGAGCTGCAGCTGGGCGGTAGGCCCGGGG
GCCCCCGCGGACGTCCAGTACGACCTGTACTTGAACGTTGCCAACAGGCGTCAAC
AGTACGAGTGTCTTCACTACAAAACGGATGCTCAGGGAACACGTATCGGGTGTCG
TTTCGATGACATCTCTCGACTCTCCAGCGGTTCTCAAAGTTOCCACATCCTGOTGC
GGGGCAGGAGCGCAGCCTTCGGTATCCCCTGCACAGATAAGTTTGTCGTCTTTTCA
CAGATTGAGATATTAACTCCACCCAACATGACTGCAAAGTGTAATAAGACACATT
CCTTTATGCACTGGAAAATGAGAAGTCATTTCAATCGCAAATTTCGCTATGAGCTT
CAGATACAAAAGAGAATGCAGCCTGTAATCACAGAACAGGTCAGAGACAGAACC
TCCTTCCAGCTACTCAATCCTGGAACGTACACAGTACAAATAAGAGCCCGGGAAA
GAGTGTATGAATTCTTGAGCGCCTGGAGCACCCCCCAGCGCTTCGAGTGCGACCA
GGAGGAGGGCGCAAACACACGTGCCTGGCGGACGTCGCTGCTGATCGCGCTGGG
GACGCTGCTGGCCCTGGTCTGTGTCTTCGTGATCTGCAGAAGGTATCTGGTGATGC
AGAGACTCTTTCCCCGCATCCCTCACATGAAAGACCCCATCGGTGACAGCTTCCA
AAACGACAAGCTGGTGGTCTGGGAGGCGGGCAAAGCCGGCCTGGAGGAGTGTCT
GGTGACTGAAGTACAGGTCGTGCAGAAAACTTGA; Transcript ID ENST00000331035;
Homo sapiens
80:
MVLLWLTLLLIALPCLLQTKEDPNPPITNLRMKAKAQQLTWDLNRNVTDIECVKDAD
YSMPAVNNSYCQFGAISLCEVTNYTVRVANPPFSTWILFPENSGKPWAGAENLTCWI
HDVDFLSCSWAVGPGAPADVQYDLYLNVANRRQQYECLHYKTDAQGTRIGCREDDI
SRLSSGSQSSHILVRGRSAAFGIPCTDKFVVFSQIEILTPPNMTAKCNKTHSFMHWKMR
SHENRKFRYELQIQKRMQPVITEQVRDRTSFQLLNPGTYTVQIRARERVYEFLSAWST
PQRFECDQEEGANTRAWRTSLLIALGTLLALVCVFVICRRYLVMQRLFPRIPHMKDPI
GDSFQNDKLVVWEAGKAGLEECLVTEVQVVQKT; IL3RA protein (ENSP00000327890)
encoded by Transcript ID ENST00000331035 from Gene ID ENSG00000185291;
Homo sapiens
81:
ATGGTCCTCCTTTGGCTCACGCTGCTCCTGATCGCCCTGCCCTGTCTOCTGCAAAC
GAAGGAAGGTGGGAAGCCTTGGGCAGGTGCGGAGAATCTGACCTGCTGGATTCAT
GACGTGGATTTCTTGAGCTGCAGCTGGGCGGTAGGCCCGGGGGCCCCCGCGGACG
TCCAGTACGACCTGTACTTGAACGTTGCCAACAGGCGTCAACAGTACGAGTGTCT
TCACTACAAAACGGATGCTCAGGGAACACGTATCGGGTGTCGTTTCGATGACATC
TCTCGACTCTCCAGCGGTTCTCAAAGTTCCCACATCCTGGTGCGGGGCAGGAGCG
CAGCCTTCGGTATCCCCTGCACAGATAAGTTTGTCGTCTTTTCACAGATTGAGATA
TTAACTCCACCCAACATGACTGCAAAGTGTAATAAGACACATTCCTTTATGCACTG
GAAAATGAGAAGTCATTTCAATCGCAAATTTCGCTATGAGCTTCAGATACAAAAG
AGAATGCAGCCTGTAATCACAGAACAGGTCAGAGACAGAACCTCCTTCCAGCTAC
TCAATCCTGGAACGTACACAGTACAAATAAGAGCCCGGGAAAGAGTGTATGAATT
CTTGAGCGCCTGGAGCACCCCCCAGCGCTTCGAGTGCGACCAGGAGGAGGGCGCA
AACACACGTGCCTGGCGGACGTCGCTGCTGATCGCGCTGGGGACGCTGCTGGCCC
TGGTCTGTGTCTTCGTGATCTGCAGAAGGTATCTGGTGATGCAGAGACTCTTTCCC
CGCATCCCTCACATGAAAGACCCCATCGGTGACAGCTTCCAAAACGACAAGCTGG
TGGTCTGGGAGGCGGGCAAAGCCGGCCTGGAGGAGTGTCTGGTGACTGAAGTAC
AGGTCGTGCAGAAAACTTGA; Transcript ID ENST00000381469; Homo sapiens
82:
MVLLWLTLLLIALPCLLQTKEGGKPWAGAENLTCWIHDVDFLSCSWAVGPGAPADV
QYDLYLNVANRRQQYECLHYKTDAQGTRIGCRFDDISRLSSGSQSSHILVRGRSAAFG
IPCTDKFVVFSQIEILTPPNMTAKCNKTHSFMHWKMRSHFNRKFRYELQIQKRMQPVI
TEQVRDRTSFQLLNPGTYTVQIRARERVYEFLSAWSTPQRFECDQEEGANTRAWRTS
LLIALGTLLALVCVFVICRRYLVMQRLFPRIPHMKDPIGDSFQNDKLVVWEAGKAGLE
ECLVTEVQVVQKT; IL3RA protein (ENSP00000370878) encoded by Transcript ID
ENST00000381469 from Gene ID ENSG00000185291; Homo sapiens
83:
ATGGGCCCCGGCCCCAGCCGCGCGCCCCGCGCCCCACGCCTGATGCTCTGTGCGC
TCGCCTTGATGGTGGCGGCCGGCGGCTGCGTCGTCTCCGCCTTCAACCTGGATACC
CGATTCCTGGTAGTGAAGGAGGCCGGGAACCCGGGCAGCCTCTTCGGCTACTCGG
TCGCCCTCCATCGGCAGACAGAGCGGCAGCAGCGCTACCTGCTCCTGGCTGGTGC
CCCCCGGGAGCTCGCTGTGCCCGATGGCTACACCAACCGGACTGGTGCTGTGTAC
CTGTGCCCACTCACTGCCCACAAGGATGACTGTGAGCGGATGAACATCACAGTGA
AAAATGACCCTGGCCATCACATTATTGAGGACATGTGGCTTGGAGTGACTGTGGC
CAGCCAGGGCCCTGCAGGCAGAGTTCTGGTCTGTGCCCACCGCTACACCCAGGTG
CTGTGGTCAGGGTCAGAAGACCAGCGGCGCATGGTGGGCAAGTGCTACGTGCGA
GGCAATGACCTAGAGCTGGACTCCAGTGATGACTGGCAGACCTACCACAACGAGA
TGTGCAATAGCAACACAGACTACCTGGAGACGGGCATGTGCCAGCTGGGCACCAG
CGGTGGCTTCACCCAGAACACTGTGTACTTCGGCGCCCCCGGTGCCTACAACTGG
AAAGGAAACAGCTACATGATTCAGCGCAAGGAGTGGGACTTATCTGAGTATAGTT
ACAAGGACCCAGAGGACCAAGGAAACCTCTATATTGGGTACACGATGCAGGTAG
GCAGCTTCATCCTGCACCCCAAAAACATCACCATTGTGACAGGTGCCCCACGGCA
CCGACATATGGGCGCGGTGTTCTTGCTGAGCCAGGAGGCAGGCGGAGACCTGCGG
AGGAGGCAGGTGCTGGAGGGCTCGCAGGTGGGCGCCTATTTTGGCAGCGCCATTG
CCCTGGCAGACCTGAACAATGATGGGTGGCAGGACCTCCTGGTGGGCGCCCCCTA
CTACTTCGAGAGGAAAGAGGAAGTAGGGGGTGCCATCTATGTCTTCATGAACCAG
GCGGGAACCTCCTTCCCTGCTCACCCCTCACTCCTTCTTCATGGCCCCAGTGGCTC
TGCCTTTGGTTTATCTGTGGCCAGCATTGGTGACATCAACCAGGATGGATTTCAGG
ATATTGCTGTGGGAGCTCCGTTTGAAGGCTTGGGCAAAGTGTACATCTATCACAGT
AGCTCTAAGGGGCTCCTTAGACAGCCCCAGCAGGTAATCCATGGAGAGAAGCTGG
GACTGCCTGGGTTGGCCACCTTCGGCTATTCCCTCAGTGGGCAGATGGATGTGGAT
GAGAACTTCTACCCAGACCTTCTAGTGGGAAGCCTGTCAGACCACATTGTGCTGCT
GCGGGCCCGGCCCGTCATCAACATCGTCCACAAGACCTTGGTGCCCAGGCCAGCT
GTGCTGGACCCTGCACTTTGCACGGCCACCTCTTGTGTGCAAGTGGAGCTGTGCTT
TGCTTACAACCAGAGTGCCGGGAACCCCAACTACAGGCGAAACATCACCCTGGCC
TACACTCTGGAGGCTGACAGGGACCGCCGGCCGCCCCGGCTCCGCTTTGCCGGCA
GTGAGTCCGCTGTCTTCCACGGCTTCTTCTCCATGCCCGAGATGCGCTGCCAGAAG
CTGGAGCTGCTCCTGATGGACAACCTCCGTGACAAACTCCGCCCCATCATCATCTC
CATGAACTACTCTTTACCTTTGCGGATGCCCGATCGCCCCCGGCTGGGGCTGCGGT
CCCTGGACGCCTACCCGATCCTCAACCAGGCACAGGCTCTGGAGAACCACACTGA
GGTCCAGTTCCAGAAGGAGTGCGGGCCTGACAACAAGTGTGAGAGCAACTTGCA
GATGCGGGCAGCCTTCGTGTCAGAGCAGCAGCAGAAGCTGAGCAGGCTCCAGTAC
AGCAGAGACGTCCGGAAATTGCTCCTGAGCATCAACGTGACGAACACCCGGACCT
CGGAGCGCTCCGGGGAGGACGCCCACGAGGCGCTGCTCACCCTGGTGGTGCCTCC
CGCCCTGCTGCTGTCCTCAGTGCGCCCCCCCGGGGCCTGCCAAGCTAATGAGACC
ATCTTTTGCGAGCTGGGGAACCCCTTCAAACGGAACCAGAGGATGGAGCTGCTCA
TCGCCTTTGAGGTCATCGGGGTGACCCTGCACACAAGGGACCTTCAGGTGCAGCT
GCAGCTCTCCACGTCGAGTCACCAGGACAACCTGTGGCCCATGATCCTCACTCTGC
TGGTGGACTATACACTCCAGACCTCGCTTAGCATGGTAAATCACCGGCTACAAAG
CTTCTTTGGGGGGACAGTGATGGGTGAGTCTGGCATGAAAACTGTGGAGGATGTA
GGAAGCCCCCTCAAGTATGAATTCCAGGTGGGCCCAATGGGGGAGGGGCTGGTG
GGCCTGGGGACCCTGGTCCTAGGTCTGGAGTGGCCCTACGAAGTCAGCAATGGCA
AGTGGCTGCTGTATCCCACGGAGATCACCGTCCATGGCAATGGGTCCTGGCCCTG
CCGACCACCTGGAGACCTTATCAACCCTCTCAACCTCACTCTTTCTGACCCTGGGG
ACAGGCCATCATCCCCACAGCGCAGGOGGCGACAGCTGGATCCAGGGGGAGGCC
AGGGCCCCCCACCTGTCACTCTGGCTGCTGCCAAAAAAGCCAAGTCTGAGACTGT
GCTGACCTGTGCCACAGGGCGTGCCCACTGTGTGTGGCTAGAGTGCCCCATCCCT
GATGCCCCCGTTGTCACCAACGTGACTGTGAAGGCACGAGTGTGGAACAGCACCT
TCATCGAGGATTACAGAGACTTTGACCGAGTCCGGGTAAATGGCTGGGCTACCCT
ATTCCTCCGAACCAGCATCCCCACCATCAACATGGAGAACAAGACCACGTGGTTC
TCTGTGGACATTGACTCGGAGCTGGTGGAGGAGCTGCCGGCCGAAATCGAGCTGT
GGCTGGTGCTGGTGGCCGTGGGTGCAGGGCTGCTGCTGCTGGGGCTGATCATCCT
CCTGCTGTGGAAGTGTGACTTCTTTAAGCGGACCCGCTATTATCAGATCATGCCCA
AGTACCACGCAGTGOGGATCCGGGAGGAGGAGCGCTACCCACCTCCAGGGAGCA
CCCTGCCCACCAAGAAGCACTGGGTGACCAGCTGGCAGACTCGGGACCAATACTA
CTGA; Transcript ID ENST00000007722; Homo sapiens
84:
MGPGPSRAPRAPRLMLCALALMVAAGGCVVSAFNLDTRFLVVKEAGNPGSLFGYSV
ALHRQTERQQRYLLLAGAPRELAVPDGYTNRTGAVYLCPLTAHKDDCERMNITVKN
DPGHHIIEDMWLGVTVASQGPAGRVLVCAHRYTQVLWSGSEDQRRMVGKCYVRGN
DLELDSSDDWQTYHNEMCNSNTDYLETGMCQLGTSGGFTQNTVYFGAPGAYNWKG
NSYMIQRKEWDLSEYSYKDPEDQGNLYIGYTMQVGSFILHPKNITIVTGAPRHRHMG
AVFLLSQEAGGDLRRRQVLEGSQVGAYFGSAIALADLNNDGWQDLLVGAPYYFERK
EEVGGAIYVFMNQAGTSFPAHPSLLLHGPSGSAFGLSVASIGDINQDGFQDIAVGAPFE
GLGKVYIYHSSSKGLLRQPQQVIHGEKLGLPGLATFGYSLSGQMDVDENFYPDLLVG
SLSDHIVLLRARPVINIVHKTLVPRPAVLDPALCTATSCVQVELCFAYNQSAGNPNYR
RNITLAYTLEADRDRRPPRLRFAGSESAVFHGFFSMPEMRCQKLELLLMDNLRDKLRP
IIISMNYSLPLRMPDRPRLGLRSLDAYPILNQAQALENHTEVQFQKECGPDNKCESNLQ
MRAAFVSEQQQKLSRLQYSRDVRKLLLSINVINTRTSERSGEDAHEALLTLVVPPALL
LSSVRPPGACQANETIFCELGNPFKRNQRMELLIAFEVIGVTLHTRDLQVQLQLSTSSH
QDNLWPMILTLLVDYTLQTSLSMVNHRLQSFFGGTVMGESGMKTVEDVGSPLKYEF
QVGPMGEGLVGLGTLVLGLEWPYEVSNGKWLLYPTEITVHGNGSWPCRPPGDLINPL
NLTLSDPGDRPSSPQRRRRQLDPGGGQGPPPVTLAAAKKAKSETVLTCATGRAHCVW
LECPIPDAPVVTNVTVKARVWNSTFIEDYRDFDRVRVNGWATLFLRTSIPTINMENKT
TWFSVDIDSELVEELPAEIELWLVLVAVGAGLLLLGLIILLLWKCDFFKRTRYYQIMPK
YHAVRIREEERYPPPGSTLPTKKHWVTSWQTRDQYY; ITGA3 protein
(ENSP00000007722) encoded by Transcript ID ENST00000007722 from Gene ID
ENSG00000005884; Homo sapiens
85:
ATGGGCCCCGGCCCCAGCCGCGCGCCCCGCGCCCCACGCCTGATGCTCTGTGCGC
TCGCCTTGATGGTGGCGGCCGGCGGCTGCGTCGTCTCCGCCTTCAACCTGGATACC
CGATTCCTGGTAGTGAAGGAGGCCGGGAACCCGGGCAGCCTCTTCGGCTACTCGG
TCGCCCTCCATCGGCAGACAGAGCGGCAGCAGCGCTACCTGCTCCTGGCTGGTGC
CCCCCGGGAGCTCGCTGTGCCCGATGGCTACACCAACCGGACTGGTGCTGTGTAC
CTGTGCCCACTCACTGCCCACAAGGATGACTGTGAGCGGATGAACATCACAGTGA
AAAATGACCCTGGCCATCACATTATTGAGGACATGTGGCTTGGAGTGACTGTGGC
CAGCCAGGGCCCTGCAGGCAGAGTTCTGGTCTGTGCCCACCGCTACACCCAGGTG
CTGTGGTCAGGGTCAGAAGACCAGCGGCGCATGGTGGGCAAGTGCTACGTGCGA
GGCAATGACCTAGAGCTGGACTCCAGTGATGACTGGCAGACCTACCACAACGAGA
TGTGCAATAGCAACACAGACTACCTGGAGACGGGCATGTGCCAGCTGGGCACCAG
CGGTGGCTTCACCCAGAACACTGTGTACTTCGGCGCCCCCGGTGCCTACAACTGG
AAAGGAAACAGCTACATGATTCAGCGCAAGGAGTGGGACTTATCTGAGTATAGTT
ACAAGGACCCAGAGGACCAAGGAAACCTCTATATTGGGTACACGATGCAGGTAG
GCAGCTTCATCCTGCACCCCAAAAACATCACCATTGTGACAGGTGCCCCACGGCA
CCGACATATGGGCGCGGTGTTCTTGCTGAGCCAGGAGGCAGGCGGAGACCTGCGG
AGGAGGCAGGTGCTGGAGGGCTCGCAGGTGGGCGCCTATTTTGGCAGOGCCATTG
CCCTGGCAGACCTGAACAATGATGGGTGGCAGGACCTCCTGGTGGGCGCCCCCTA
CTACTTCGAGAGGAAAGAGGAAGTAGGGGGTGCCATCTATGTCTTCATGAACCAG
GCGGGAACCTCCTTCCCTGCTCACCCCTCACTCCTTCTTCATGGCCCCAGTGGCTC
TGCCTTTGGTTTATCTGTGGCCAGCATTGGTGACATCAACCAGGATGGATTTCAGG
ATATTGCTGTGGGAGCTCCGTTTGAAGGCTTGGGCAAAGTGTACATCTATCACAGT
AGCTCTAAGGGGCTCCTTAGACAGCCCCAGCAGGTAATCCATGGAGAGAAGCTGG
GACTGCCTGGGTTGGCCACCTTCGGCTATTCCCTCAGTGGGCAGATGGATGTGGAT
GAGAACTTCTACCCAGACCTTCTAGTGGGAAGCCTGTCAGACCACATTGTGCTGCT
GCGGGCCCGGCCCGTCATCAACATCGTCCACAAGACCTTGGTGCCCAGGCCAGCT
GTGCTGGACCCTGCACTTTGCACGGCCACCTCTTGTGTGCAAGTGGAGCTGTGCTT
TGCTTACAACCAGAGTGCCGGGAACCCCAACTACAGGCGAAACATCACCCTGGCC
TACACTCTGGAGGCTGACAGGGACCGCCGGCCGCCCCGGCTCCGCTTTGCCGGCA
GTGAGTCCGCTGTCTTCCACGGCTTCTTCTCCATGCCCGAGATGCGCTGCCAGAAG
CTGGAGCTGCTCCTGATGGACAACCTCCGTGACAAACTCCGCCCCATCATCATCTC
CATGAACTACTCTTTACCTTTGOGGATGCCCGATCGCCCCCGGCTGGGGCTGCGGT
CCCTGGACGCCTACCCGATCCTCAACCAGGCACAGGCTCTGGAGAACCACACTGA
GGTCCAGTTCCAGAAGGAGTGCGGGCCTGACAACAAGTGTGAGAGCAACTTGCA
GATGCGGGCAGCCTTCGTGTCAGAGCAGCAGCAGAAGCTGAGCAGGCTCCAGTAC
AGCAGAGACGTCCGGAAATTGCTCCTGAGCATCAACGTGACGAACACCCGGACCT
CGGAGCGCTCCGGGGAGGACGCCCACGAGGCGCTGCTCACCCTGGTGGTGCCTCC
CGCCCTGCTGCTGTCCTCAGTGCGCCCCCCCGGGGCCTGCCAAGCTAATGAGACC
ATCTTTTGCGAGCTGGGGAACCCCTTCAAACGGAACCAGAGGATGGAGCTGCTCA
TCGCCTTTGAGGTCATCGGGGTGACCCTGCACACAAGGGACCTTCAGGTGCAGCT
GCAGCTCTCCACGTCGAGTCACCAGGACAACCTGTGGCCCATGATCCTCACTCTGC
TGGTGGACTATACACTCCAGACCTCGCTTAGCATGGTAAATCACCGGCTACAAAG
CTTCTTTGGGGGGACAGTGATGGGTGAGTCTGGCATGAAAACTGTGGAGGATGTA
GGAAGCCCCCTCAAGTATGAATTCCAGGTGGGCCCAATGGGGGAGGGGCTGGTG
GGCCTGGGGACCCTGGTCCTAGGTCTGGAGTGGCCCTACGAAGTCAGCAATGGCA
AGTGGCTGCTGTATCCCACGGAGATCACCGTCCATGGCAATGGGTCCTGGCCCTG
CCGACCACCTGGAGACCTTATCAACCCTCTCAACCTCACTCTTTCTGACCCTGGGG
ACAGGCCATCATCCCCACAGCGCAGGCGGCGACAGCTGGATCCAGGGGGAGGCC
AGGGCCCCCCACCTGTCACTCTGGCTGCTGCCAAAAAAGCCAAGTCTGAGACTGT
GCTGACCTGTGCCACAGGGCGTGCCCACTGTGTGTGGCTAGAGTGCCCCATCCCT
GATGCCCCCGTTGTCACCAACGTGACTGTGAAGGCACGAGTGTGGAACAGCACCT
TCATCGAGGATTACAGAGACTTTGACCGAGTCCGGGTAAATGGCTGGGCTACCCT
ATTCCTCCGAACCAGCATCCCCACCATCAACATGGAGAACAAGACCACGTGGTTC
TCTGTGGACATTGACTCGGAGCTGGTGGAGGAGCTGCCGGCCGAAATCGAGCTGT
GGCTGGTGCTGGTGGCCGTGGGTGCAGGGCTGCTGCTGCTGGGGCTGATCATCCT
CCTGCTGTGGAAGTGCGGCTTCTTCAAGCGAGCCCGCACTCGCGCCCTGTATGAA
GCTAAGAGGCAGAAGGCGGAGATGAAGAGCCAGCCGTCAGAGACAGAGAGGCTG
ACCGACGACTACTGA; Transcript ID ENST00000320031; Homo sapiens
86:
MGPGPSRAPRAPRLMLCALALMVAAGGCVVSAFNLDTRFLVVKEAGNPGSLFGYSV
ALHRQTERQQRYLLLAGAPRELAVPDGYTNRTGAVYLCPLTAHKDDCERMNITVKN
DPGHHIIEDMWLGVTVASQGPAGRVLVCAHRYTQVLWSGSEDQRRMVGKCYVRGN
DLELDSSDDWQTYHNEMCNSNTDYLETGMCQLGTSGGFTQNTVYFGAPGAYNWKG
NSYMIQRKEWDLSEYSYKDPEDQGNLYIGYTMQVGSFILHPKNITIVTGAPRHRHMG
AVFLLSQEAGGDLRRRQVLEGSQVGAYFGSAIALADLNNDGWQDLLVGAPYYFERK
EEVGGAIYVFMNQAGTSFPAHPSLLLHGPSGSAFGLSVASIGDINQDGFQDIAVGAPFE
GLGKVYTYHSSSKGLLRQPQQVIHGEKLGLPGLATFGYSLSGQMDVDENFYPDLLVG
SLSDHIVLLRARPVINIVHKTLVPRPAVLDPALCTATSCVQVELCFAYNQSAGNPNYR
RNITLAYTLEADRDRRPPRLRFAGSESAVFHGFFSMPEMRCQKLELLLMDNLRDKLRP
IIISMNYSLPLRMPDRPRLGLRSLDAYPILNQAQALENHTEVQFQKECGPDNKCESNLQ
MRAAFVSEQQQKLSRLQYSRDVRKLLLSINVINTRTSERSGEDAHEALLTLVVPPALL
LSSVRPPGACQANETIFCELGNPFKRNQRMELLIAFEVIGVTLHTRDLQVQLQLSTSSH
QDNLWPMILTLLVDYTLQTSLSMVNHRLQSFFGGTVMGESGMKTVEDVGSPLKYEF
QVGPMGEGLVGLGTLVLGLEWPYEVSNGKWLLYPTEITVHGNGSWPCRPPGDLINPL
NLTLSDPGDRPSSPQRRRRQLDPGGGQGPPPVTLAAAKKAKSETVLTCATGRAHCVW
LECPIPDAPVVTNVTVKARVWNSTFIEDYRDFDRVRVNGWATLFLRTSIPTINMENKT
TWFSVDIDSELVEELPAEIELWLVLVAVGAGLLLLGLIILLLWKCGFFKRARTRALYE
AKRQKAEMKSQPSETERLTDDY; ITGA3 protein (ENSP00000315190) encoded by
Transcript ID ENST00000320031 from Gene ID ENSG00000005884; Homo sapiens
87:
ATGAATTTACAACCAATTTTCTGGATTGGACTGATCAGTTCAGTTTGCTGTGTGTT
TGCTCAAACAGATGAAAATAGATGTTTAAAAGCAAATGCCAAATCATGTGGAGAA
TGTATACAAGCAGGGCCAAATTGTGGGTGGTGCACAAATTCAACATTTTTACAGG
AAGGAATGCCTACTTCTGCACGATGTGATGATTTAGAAGCCTTAAAAAAGAAGGG
TTGCCCTCCAGATGACATAGAAAATCCCAGAGGCTCCAAAGATATAAAGAAAAAT
AAAAATGTAACCAACCGTAGCAAAGGAACAGCAGAGAAGCTCAAGCCAGAGGAT
ATTACTCAGATCCAACCACAGCAGTTGGTTTTGCGATTAAGATCAGGGGAGCCAC
AGACATTTACATTAAAATTCAAGAGAGCTGAAGACTATOCCATTGACCTCTACTA
CCTTATGGACCTGTCTTACTCAATGAAAGACGATTTGGAGAATGTAAAAAGTCTT
GGAACAGATCTGATGAATGAAATGAGGAGGATTACTTCGGACTTCAGAATTGGAT
TTGGCTCATTTGTGGAAAAGACTGTGATGCCTTACATTAGCACAACACCAGCTAA
GCTCAGGAACCCTTGCACAAGTGAACAGAACTGCACCAGCCCATTTAGCTACAAA
AATGTGCTCAGTCTTACTAATAAAGGAGAAGTATTTAATGAACTTGTTGGAAAAC
AGCGCATATCTGGAAATTTGGATTCTCCAGAAGGTGGTTTCGATGCCATCATGCA
AGTTGCAGTTTGTGGATCACTGATTGGCTGGAGGAATGTTACACGGCTGCTGGTGT
TTTCCACAGATGCCGGGTTTCACTTTGCTGGAGATGGGAAACTTGGTGGCATTGTT
TTACCAAATGATGGACAATGTCACCTGGAAAATAATATGTACACAATGAGCCATT
ATTATGATTATCCTTCTATTGCTCACCTTGTCCAGAAACTGAGTGAAAATAATATT
CAGACAATTTTTGCAGTTACTGAAGAATTTCAGCCTGTTTACAAGGAGCTGAAAA
ACTTGATCCCTAAGTCAGCAGTAGGAACATTATCTGCAAATTCTAGCAATGTAATT
CAGTTGATCATTGATGCATACAATTCCCTTTCCTCAGAAGTCATTTTGGAAAACGG
CAAATTGTCAGAAGGCGTAACAATAAGTTACAAATCTTACTGCAAGAACGGGGTG
AATGGAACAGGGGAAAATGGAAGAAAATGTTCCAATATTTCCATTGGAGATGAG
GTTCAATTTGAAATTAGCATAACTTCAAATAAGTGTCCAAAAAAGGATTCTGACA
GCTTTAAAATTAGGCCTCTGGGCTTTACGGAGGAAGTAGAGGTTATTCTTCAGTAC
ATCTGTGAATGTGAATGCCAAAGCGAAGGCATCCCTGAAAGTCCCAAGTGTCATG
AAGGAAATGGGACATTTGAGTGTGGCGCGTGCAGGTGCAATGAAGGGCGTGTTG
GTAGACATTGTGAATGCAGCACAGATGAAGTTAACAGTGAAGACATGGATGCTTA
CTGCAGGAAAGAAAACAGTTCAGAAATCTGCAGTAACAATGGAGAGTGCGTCTG
CGGACAGTGTGTTTGTAGGAAGAGGGATAATACAAATGAAATTTATTCTGGCAAA
TTCTGCGAGTGTGATAATTTCAACTGTGATAGATCCAATGGCTTAATTTGTGGAGG
AAATGGTGTTTGCAAGTGTCGTGTGTGTGAGTGCAACCCCAACTACACTGGCAGT
GCATGTGACTGTTCTTTGGATACTAGTACTTGTGAAGCCAGCAACGGACAGATCT
GCAATGGCCGGGGCATCTGCGAGTGTGGTGTCTGTAAGTGTACAGATCCGAAGTT
TCAAGGGCAAACGTGTGAGATGTGTCAGACCTGCCTTGGTGTCTGTGCTGAGCAT
AAAGAATGTGTTCAGTGCAGAGCCTTCAATAAAGGAGAAAAGAAAGACACATGC
ACACAGGAATGTTCCTATTTTAACATTACCAAGGTAGAAAGTCGGGACAAATTAC
CCCAGCCGGTCCAACCTGATCCTGTGTCCCATTGTAAGGAGAAGGATGTTGACGA
CTGTTGGTTCTATTTTACGTATTCAGTGAATGGGAACAACGAGGTCATGGTTCATG
TTGTGGAGAATCCAGAGTGTCCCACTGGTCCAGACATCATTCCAATTGTAGCTGGT
GTGGTTGCTGGAATTGTTCTTATTGGCCTTGCATTACTGCTGATATGGAAGCTTTT
AATGATAATTCATGACAGAAGGGAGTTTGCTAAATTTGAAAAGGAGAAAATGAAT
GCCAAATGGGACACGGGTGAAAATCCTATTTATAAGAGTGCCGTAACAACTGTGG
TCAATCCGAAGTATGAGGGAAAATGA; Transcript ID ENST00000302278; Homo
sapiens, Transcript ID ENST00000396033; Homo sapiens
88:
MNLQPIFWIGLISSVCCVFAQTDENRCLKANAKSCGECIQAGPNCGWCINSTFLQEG
MPTSARCDDLEALKKKGCPPDDIENPRGSKDIKKNKNVTNRSKGTAEKLKPEDITQIQ
PQQLVLRLRSGEPQTFTLKFKRAEDYPIDLYYLMDLSYSMKDDLENVKSLGTDLMNE
MRRITSDFRIGFGSFVEKTVMPYISTTPAKLRNPCTSEQNCTSPFSYKNVLSLINKGEV
FNELVGKORISGNLDSPEGGFDAIMQVAVCGSLIGWRNVTRLLVFSTDAGFHFAGDG
KLGGIVLPNDGOCHLENNMYTMSHYYDYPSIAHLVQKLSENNIQTIFAVTEEFQPVYK
ELKNLIPKSAVGTLSANSSNVIQLIIDAYNSLSSEVILENGKLSEGVTISYKSYCKNGVN
GTGENGRKCSNISIGDEVOFEISITSNKCPKKDSDSFKIRPLGFTEEVEVILQYICECECQ
SEGIPESPKCHEGNGTFECGACRCNEGRVGRHCECSTDEVNSEDMDAYCRKENSSEIC
SNNGECVCGQCVCRKRDNTNEIYSGKFCECDNFNCDRSNGLICGGNGVCKCRVCECN
PNYTGSACDCSLDTSTCEASNGQICNGRGICECGVCKCTDPKFQGQTCEMCQTCLGV
CAEHKECVQCRAFNKGEKKDTCTQECSYFNITKVESRDKLPQPVQPDPVSHCKEKDV
DDCWFYFTYSVNGNNEVMVHVVENPECPTGPDIIPIVAGVVAGIVLIGLALLLIWKLL
MIIHDRREFAKFEKEKMNAKWDTGENPIYKSAVTTVVNPKYEGK; ITGB1 protein
(ENSP00000303351) encoded by Transcript ID ENST00000302278 from Gene ID
ENSG00000150093; Homo sapiens, ITGBI protein (ENSP00000379350) encoded by
Transcript ID ENST00000396033 from Gene ID ENSG00000150093; Homo sapiens
89:
ATGAATTTACAACCAATTTTCTGGATTGGACTGATCAGTTCAGTTTGCTGTGTGTT
TGCTCAAACAGATGAAAATAGATGTTTAAAAGCAAATGCCAAATCATGTGGAGAA
TGTATACAAGCAGGGCCAAATTGTGGGTGGTGCACAAATTCAACATTTTTACAGG
AAGGAATGCCTACTTCTGCACGATGTGATGATTTAGAAGCCTTAAAAAAGAAGGG
TTGCCCTCCAGATGACATAGAAAATCCCAGAGGCTCCAAAGATATAAAGAAAAAT
AAAAATGTAACCAACCGTAGCAAAGGAACAGCAGAGAAGCTCAAGCCAGAGGAT
ATTACTCAGATCCAACCACAGCAGTTGGTTTTGCGATTAAGATCAGGGGAGCCAC
AGACATTTACATTAAAATTCAAGAGAGCTGAAGACTATCCCATTGACCTCTACTA
CCTTATGGACCTGTCTTACTCAATGAAAGACGATTTGGAGAATGTAAAAAGTCTT
GGAACAGATCTGATGAATGAAATGAGGAGGATTACTTCGGACTTCAGAATTGGAT
TTGGCTCATTTGTGGAAAAGACTGTGATGCCTTACATTAGCACAACACCAGCTAA
GCTCAGGAACCCTTGCACAAGTGAACAGAACTGCACCAGCCCATTTAGCTACAAA
AATGTGCTCAGTCTTACTAATAAAGGAGAAGTATTTAATGAACTTGTTGGAAAAC
AGCGCATATCTGGAAATTTGGATTCTCCAGAAGGTGGTTTCGATGCCATCATGCA
AGTTGCAGTTTGTGGATCACTGATTGGCTGGAGGAATGTTACACGGCTGCTGGTGT
TTTCCACAGATGCCGGGTTTCACTTTGCTGGAGATGGGAAACTTGGTGGCATTGTT
TTACCAAATGATGGACAATGTCACCTGGAAAATAATATGTACACAATGAGCCATT
ATTATGATTATCCTTCTATTGCTCACCTTGTCCAGAAACTGAGTGAAAATAATATT
CAGACAATTTTTGCAGTTACTGAAGAATTTCAGCCTGTTTACAAGGAGCTGAAAA
ACTTGATCCCTAAGTCAGCAGTAGGAACATTATCTGCAAATTCTAGCAATGTAATT
CAGTTGATCATTGATGCATACAATTCCCTTTCCTCAGAAGTCATTTTGGAAAACGG
CAAATTGTCAGAAGGCGTAACAATAAGTTACAAATCTTACTGCAAGAACGGGGTG
AATGGAACAGGGGAAAATGGAAGAAAATGTTCCAATATTTCCATTGGAGATGAG
GTTCAATTTGAAATTACCATAACTTCAAATAAGTGTCCAAAAAAGGATTCTGACA
GCTTTAAAATTAGGCCTCTGGGCTTTACGGAGGAAGTAGAGGTTATTCTTCAGTAC
ATGTGTGAATGTGAATGCCAAAGCGAAGGCATCCCTGAAAGTCCCAAGTGTCATG
AAGGAAATGGGACATTTGAGTGTGGCGCGTGCAGGTGCAATGAAGGGCGTGTTG
GTAGACATTGTGAATGCAGCACAGATGAAGTTAACAGTGAAGACATGGATGCTTA
CTGCAGGAAAGAAAACAGTTCAGAAATCTGCAGTAACAATGGAGAGTGCGTCTG
CGGACAGTGTGTTTGTAGGAAGAGGGATAATACAAATGAAATTTATTCTGGCAAA
TTCTGCGAGTGTGATAATTTCAACTGTGATAGATCCAATGGCTTAATTTGTGGAGG
AAATGGTGTTTGCAAGTGTCGTGTGTGTGAGTGCAACCCCAACTACACTGGCAGT
GCATGTGACTGTTCTTTGGATACTAGTACTTGTGAAGCCAGCAACGGACAGATCT
GCAATGGCCGGGGCATCTGCGAGTGTGGTGTCTGTAAGTGTACAGATCCGAAGTT
TCAAGGGCAAACGTGTGAGATGTGTCAGACCTGCCTTGGTGTCTGTGCTGAGCAT
AAAGAATGTGTTCAGTGCAGAGCCTTCAATAAAGGAGAAAAGAAAGACACATGC
ACACAGGAATGTTCCTATTTTAACATTACCAAGGTAGAAAGTCGGGACAAATTAC
CCCAGCCGGTCCAACCTGATCCTGTGTCCCATTGTAAGGAGAAGGATGTTGACGA
CTGTTGGTTCTATTTTACGTATTCAGTGAATGGGAACAACGAGGTCATGGTTCATG
TTGTGGAGAATCCAGAGTGTCCCACTGGTCCAGACATCATTCCAATTGTAGCTGGT
GTGGTTGCTGGAATTGTTCTTATTGGCCTTGCATTACTGCTGATATGGAAGCTTTT
AATGATAATTCATGACAGAAGGGAGTTTGCTAAATTTGAAAAGGAGAAAATGAAT
GCCAAATGGGACACGCAAGAAAATCCGATTTACAAGAGTCCTATTAATAATTTCA
AGAATCCAAACTACGGACGTAAAGCTGGTCTCTAA; Transcript ID
ENST00000423113; Homo sapiens
90:
MNLQPIFWIGLISSVCCVFAQTDENRCLKANAKSCGECIQAGPNCGWCTNSTFLQEG
MPTSARCDDLEALKKKGCPPDDIENPRGSKDIKKNKNVTNRSKGTAEKLKPEDITQIQ
PQQLVLRLRSGEPQTFTLKFKRAEDYPIDLYYLMDLSYSMKDDLENVKSLGTDLMNE
MRRITSDFRIGFGSFVEKTVMPYISTTPAKLRNPCTSEQNCTSPFSYKNVLSLINKGEV
FNELVGKQRISGNLDSPEGGFDAIMQVAVCGSLIGWRNVTRLLVFSTDAGFHFAGDG
KLGGIVLPNDGQCHLENNMYTMSHYYDYPSIAHLVQKLSENNIQTIFAVTEEFQPVYK
ELKNLIPKSAVGTLSANSSNVIQLIIDAYNSLSSEVILENGKLSEGVTISYKSYCKNGVN
GTGENGRKCSNISIGDEVQFEISITSNKCPKKDSDSFKIRPLGFTEEVEVILQYICECECQ
SEGIPESPKCHEGNGTFECGACRCNEGRVGRHCECSTDEVNSEDMDAYCRKENSSEIC
SNNGECVCGQCVCRKRDNTNEIYSGKFCECDNFNCDRSNGLICGGNGVCKCRVCECN
PNYTGSACDCSLDTSTCEASNGQICNGRGICECGVCKCTDPKFQGQTCEMCQTCLGV
CAEHKECVQCRAFNKGEKKDTCTQECSYFNITKVESRDKLPQPVQPDPVSHCKEKDV
DDCWFYFTYSVNGNNEVMVHVVENPECPTGPDIIPIVAGVVAGIVLIGLALLLIWKLL
MIIHDRREFAKFEKEKMNAKWDTQENPIYKSPINNFKNPNYGRKAGL; ITGB1 protein
(ENSP00000388694) encoded by Transcript ID ENST00000423113 from Gene ID
ENSG00000150093; Homo sapiens
91:
ATGGTGTGCTTCCGCCTCTTCCCGGTTCCGGGCTCAGGGCTCGTTCTGGTCTGCCT
AGTCCTGGGAGCTGTGCGGTCTTATGCATTGGAACTTAATTTGACAGATTCAGAA
AATGCCACTTGCCTTTATGCAAAATGGCAGATGAATTTCACAGTACGCTATGAAA
CTACAAATAAAACTTATAAAACTGTAACCATTTCAGACCATGGCACTGTGACATA
TAATGGAAGCATTTGTGGGGATGATCAGAATGGTCCCAAAATAGCAGTGCAGTTC
GGACCTGGCTTTTCCTGGATTGCGAATTTTACCAAGGCAGCATCTACTTATTCAAT
TGACAGCGTCTCATTTTCCTACAACACTGGTGATAACACAACATTTCCTGATGCTG
AAGATAAAGGAATTCTTACTGTTGATGAACTTTTGGCCATCAGAATTCCATTGAAT
GACCTTTTTAGATGCAATAGTTTATCAACTTTGGAAAAGAATGATGTTGTCCAACA
CTACTGGGATGTTCTTGTACAAGCTTTTGTCCAAAATGGCACAGTGAGCACAAAT
GAGTTCCTGTGTGATAAAGACAAAACTTCAACAGTGGCACCCACCATACACACCA
CTGTGCCATCTCCTACTACAACACCTACTCCAAAGGAAAAACCAGAAGCTGGAAC
CTATTCAGTTAATAATGGCAATGATACTTGTCTGCTGGCTACCATGGGGCTGCAGC
TGAACATCACTCAGGATAAGGTTGCTTCAGTTATTAACATCAACCCCAATACAACT
CACTCCACAGGCAGCTGCCGTTCTCACACTGCTCTACTTAGACTCAATAGCAGCAC
CATTAAGTATCTAGACTTTGTCTTTGCTGTGAAAAATGAAAACCGATTTTATCTGA
AGGAAGTGAACATCAGCATGTATTTGGTTAATGGCTCCGTTTTCAGCATTGCAAAT
AACAATCTCAGCTACTGGGATGCCCCCCTGGGAAGTTCTTATATGTGCAACAAAG
AGCAGACTGTTTCAGTGTCTGGAGCATTTCAGATAAATACCTTTGATCTAAGGGTT
CAGCCTTTCAATGTGACACAAGGAAAGTATTCTACAGCTCAAGACTGCAGTGCAG
ATGACGACAACTTCCTTGTGCCCATAGCGGTGGGAGCTGCCTTGGCAGGAGTACT
TATTCTAGTGTTGCTGGCTTATTTTATTGGTCTCAAGCACCATCATGCTGGATATG
AGCAATTTTAG; Transcript ID ENST00000200639; Homo sapiens
92:
MVCFRLFPVPGSGLVLVCLVLGAVRSYALELNLTDSENATCLYAKWQMNFTVRYET
TNKTYKTVTISDHGTVTYNGSICGDDQNGPKIAVQFGPGFSWIANFTKAASTYSIDSVS
FSYNTGDNTTFPDAEDKGILTVDELLAIRIPLNDLFRCNSLSTLEKNDVVQHYWDVLV
QAFVQNGTVSTNEFLCDKDKTSTVAPTIHTTVPSPTTTPTPKEKPEAGTYSVNNGNDT
CLLATMGLQLNITQDKVASVININPNTTHSTGSCRSHTALLRLNSSTIKYLDFVFAVKN
ENRFYLKEVNISMYLVNGSVFSIANNNLSYWDAPLGSSYMCNKEQTVSVSGAFQINTF
DLRVQPFNVTQGKYSTAQDCSADDDNFLVPIAVGAALAGVLILVLLAYFIGLKHHHA
GYEQF; LAMP2 protein (ENSP00000200639) encoded by Transcript ID
ENST00000200639 from Gene ID ENSG00000005893; Homo sapiens
93:
ATGGTGTGCTTCCGCCTCTTCCCGGTTCCGGGCTCAGGGCTCGTTCTGGTCTGCCT
AGTCCTGGGAGCTGTGCGGTCTTATGCATTGGAACTTAATTTGACAGATTCAGAA
AATGCCACTTGCCTTTATGCAAAATGGCAGATGAATTTCACAGTACGCTATGAAA
CTACAAATAAAACTTATAAAACTGTAACCATTTCAGACCATGGCACTGTGACATA
TAATGGAAGCATTTGTGGGGATGATCAGAATGGTCCCAAAATAGCAGTGCAGTTC
GGACCTGGCTTTTCCTGGATTGCGAATTTTACCAAGGCAGCATCTACTTATTCAAT
TGACAGCGTCTCATTTTCCTACAACACTGGTGATAACACAACATTTCCTGATGCTG
AAGATAAAGGAATTCTTACTGTTGATGAACTTTTGGCCATCAGAATTCCATTGAAT
GACCTTTTTAGATGCAATAGTTTATCAACTTTGGAAAAGAATGATGTTGTCCAACA
CTACTGGGATGTTCTTGTACAAGCTTTTGTCCAAAATGGCACAGTGAGCACAAAT
GAGTTCCTGTGTGATAAAGACAAAACTTCAACAGTGGCACCCACCATACACACCA
CTGTGCCATCTCCTACTACAACACCTACTCCAAAGGAAAAACCAGAAGCTGGAAC
CTATTCAGTTAATAATGGCAATGATACTTGTCTGCTGGCTACCATGGGGCTGCAGC
TGAACATCACTCAGGATAAGGTTGCTTCAGTTATTAACATCAACCCCAATACAACT
CACTCCACAGGCAGCTGCCGTTCTCACACTGCTCTACTTAGACTCAATAGCAGCAC
CATTAAGTATCTAGACTTTGTCTTTGCTGTGAAAAATGAAAACCGATTTTATCTGA
AGGAAGTGAACATCAGCATGTATTTGGTTAATGGCTCCGTTTTCAGCATTGCAAAT
AACAATCTCAGCTACTGGGATGCCCCCCTGGGAAGTTCTTATATGTGCAACAAAG
AGCAGACTGTTTCAGTGTCTGGAGCATTTCAGATAAATACCTTTGATCTAAGGGTT
CAGCCTTTCAATGTGACACAAGGAAAGTATTCTACAGCCCAAGAGTGTTCGCTGG
ATGATGACACCATTCTAATCCCAATTATAGTTGGTGCTGGTCTTTCAGGCTTGATT
ATCGTTATAGTGATTGCTTACGTAATTGGCAGAAGAAAAAGTTATGCTGGATATC
AGACTCTGTAA; Transcript ID ENST00000371335; Homo sapiens
94:
MVCFRLFPVPGSGLVLVCLVLGAVRSYALELNLTDSENATCLYAKWQMNFTVRYET
TNKTYKTVTISDHGTVTYNGSICGDDQNGPKIAVQFGPGFSWIANFTKAASTYSIDSVS
FSYNTGDNTTFPDAEDKGILTVDELLAIRIPLNDLFRCNSLSTLEKNDVVQHYWDVLV
QAFVQNGTVSTNEFLCDKDKTSTVAPTIHTTVPSPTTTPTPKEKPEAGTYSVNNGNDT
CLLATMGLQLNITQDKVASVININPNTTHSTGSCRSHTALLRLNSSTIKYLDFVFAVKN
ENRFYLKEVNISMYLVNGSVFSIANNNLSYWDAPLGSSYMCNKEQTVSVSGAFQINTF
DLRVQPFNVTQGKYSTAQECSLDDDTILIPIIVGAGLSGLIIVIVIAYVIGRRKSYAGYQ
TL; LAMP2 protein (ENSP00000360386) encoded by Transcript ID
ENST00000371335 from Gene ID ENSG00000005893; Homo sapiens
95:
ATGGTGTGCTTCCGCCTCTTCCCGGTTCCGGGCTCAGGGCTCGTTCTGGTCTGCCT
AGTCCTGGGAGCTGTGCGGTCTTATGCATTGGAACTTAATTTGACAGATTCAGAA
AATGCCACTTGCCTTTATGCAAAATGGCAGATGAATTTCACAGTACGCTATGAAA
CTACAAATAAAACTTATAAAACTGTAACCATTTCAGACCATGGCACTGTGACATA
TAATGGAAGCATTTGTGGGGATGATCAGAATGGTCCCAAAATAGCAGTGCAGTTC
GGACCTGGCTTTTCCTGGATTGCGAATTTTACCAAGGCAGCATCTACTTATTCAAT
TGACAGCGTCTCATTTTCCTACAACACTGGTGATAACACAACATTTCCTGATGCTG
AAGATAAAGGAATTCTTACTGTTGATGAACTTTTGGCCATCAGAATTCCATTGAAT
GACCTTTTTAGATGCAATAGTTTATCAACTTTGGAAAAGAATGATGTTGTCCAACA
CTACTGGGATGTTCTTGTACAAGCTTTTGTCCAAAATGGCACAGTGAGCACAAAT
GAGTTCCTGTGTGATAAAGACAAAACTTCAACAGTGGCACCCACCATACACACCA
CTGTGCCATCTCCTACTACAACACCTACTCCAAAGGAAAAACCAGAAGCTGGAAC
CTATTCAGTTAATAATGGCAATGATACTTGTCTGCTGGCTACCATGGGGCTGCAGC
TGAACATCACTCAGGATAAGGTTGCTTCAGTTATTAACATCAACCCCAATACAACT
CACTCCACAGGCAGCTGCCGTTCTCACACTGCTCTACTTAGACTCAATAGCAGCAC
CATTAAGTATCTAGACTTTGTCTTTGCTGTGAAAAATGAAAACCGATTTTATCTGA
AGGAAGTGAACATCAGCATGTATTTGGTTAATGGCTCCGTTTTCAGCATTGCAAAT
AACAATCTCAGCTACTGGGATGCCCCCCTGGGAAGTTCTTATATGTGCAACAAAG
AGCAGACTGTTTCAGTGTCTGGAGCATTTCAGATAAATACCTTTGATCTAAGGGTT
CAGCCTTTCAATGTGACACAAGGAAAGTATTCTACAGCTGAAGAATGTTCTGCTG
ACTCTGACCTCAACTTTCTTATTCCTGTTGCAGTGGGTGTGGCCTTGGGCTTCCTTA
TAATTGTTGTCTTTATCTCTTATATGATTGGAAGAAGGAAAAGTCGTACTGGTTAT
CAGTCTGTGTAA; Transcript ID ENST00000434600; Homo sapiens
96:
MVCFRLFPVPGSGLVLVCLVLGAVRSYALELNLTDSENATCLYAKWQMNFTVRYET
TNKTYKTVTISDHGTVTYNGSICGDDQNGPKIAVOFGPGFSWIANFTKAASTYSIDSVS
FSYNTGDNTTFPDAEDKGILTVDELLAIRIPLNDLFRCNSLSTLEKNDVVQHYWDVLV
QAFVQNGTVSTNEFLCDKDKTSTVAPTIHTTVPSPTTTPTPKEKPEAGTYSVNNGNDT
CLLATMGLQLNITQDKVASVININPNTTHSTGSCRSHTALLRLNSSTIKYLDFVFAVKN
ENRFYLKEVNISMYLVNGSVFSIANNNLSYWDAPLGSSYMCNKEQTVSVSGAFQINTF
DLRVQPFNVTQGKYSTAEECSADSDLNFLIPVAVGVALGFLIIVVFISYMIGRRKSRTG
YQSV; LAMP2 protein (ENSP00000408411) encoded by Transcript ID
ENST00000434600 from Gene ID ENSG00000005893; Homo sapiens
97:
ATGATCCCCACCTTCACGGCTCTGCTCTGCCTCGGGCTGAGTCTGGGCCCCAGGAC
CCACATGCAGGCAGGGCCCCTCCCCAAACCCACCCTCTGGGCTGAGCCAGGCTCT
GTGATCAGCTGGGGGAACTCTGTGACCATCTGGTGTCAGGGGACCCTGGAGGCTC
GGGAGTACCGTCTGGATAAAGAGGAAAGCCCAGCACCCTGGGACAGACAGAACC
CACTGGAGCCCAAGAACAAGGCCAGATTCTCCATCCCATCCATGACAGAGGACTA
TGCAGGGAGATACCGCTGTTACTATCGCAGCCCTGTAGGCTGGTCACAGCCCAGT
GACCCCCTGGAGCTGGTGATGACAGGAGCCTACAGTAAACCCACCCTTTCAGCCC
TGCCGAGTCCTCTTGTGACCTCAGGAAAGAGCGTGACCCTGCTGTGTCAGTCACG
GAGCCCAATGGACACTTTTCTTCTGATCAAGGAGCGGGCAGCCCATCCCCTACTG
CATCTGAGATCAGAGCACGGAGCTCAGCAGCACCAGGCTGAATTCCCCATGAGTC
CTGTGACCTCAGTGCACGGGGGGACCTACAGGTGCTTCAGCTCACACGGCTTCTC
CCACTACCTGCTGTCACACCCCAGTGACCCCCTGGAGCTCATAGTCTCAGGATCCT
TGGAGGGTCCCAGGCCCTCACCCACAAGGTCCGTCTCAACAGCTGCAGGCCCTGA
GGACCAGCCCCTCATGCCTACAGGGTCAGTCCCCCACAGTGGTCTGAGAAGGCAC
TGGGAGGTACTGATCGGGGTCTTGGTGGTCTCCATCCTGCTTCTCTCCCTCCTCCTC
TTCCTCCTCCTCCAACACTGGCGTCAGGGAAAACACAGGACATTGGCCCAGAGAC
AGGCTGATTTCCAACGTCCTCCAGGGGCTGCCGAGCCAGAGCCCAAGGACGGGGG
CCTACAGAGGAGGTCCAGCCCAGCTGCTGACGTCCAGGGAGAAAACTTCTGTGCT
GCCGTGAAGAACACACAGCCTGAGGACGGGGTGGAAATGGACACTCGGCAGAGC
CCACACGATGAAGACCCCCAGGCAGTGACGTATGCCAAGGTGAAACACTCCAGA
CCTAGGAGAGAAATGGCCTCTCCTCCCTCCCCACTGTCTGGGGAATTCCTGGACAC
AAAGGACAGACAGGCAGAAGAGGACAGACAGATGGACACTGAGGCTGCTGCATC
TGAAGCCCCCCAGGATGTGACCTACGCCCGGCTGCACAGCTTTACCCTCAGACAG
AAGGCAACTGAGCCTCCTCCATCCCAGGAAGGGGCCTCTCCAGCTGAGCCCAGTG
TCTATGCCACTCTGGCCATCCACTAA; Transcript ID ENST00000391736; Homo sapiens
98:
MIPTFTALLCLGLSLGPRTHMQAGPLPKPTLWAEPGSVISWGNSVTIWCQGTLEAREY
RLDKEESPAPWDRQNPLEPKNKARFSIPSMTEDYAGRYRCYYRSPVGWSQPSDPLEL
VMTGAYSKPTLSALPSPLVTSGKSVTLLCQSRSPMDTFLLIKERAAHPLLHLRSEHGA
QQHQAEFPMSPVTSVHGGTYRCFSSHGFSHYLLSHPSDPLELIVSGSLEGPRPSPTRSVS
TAAGPEDQPLMPTGSVPHSGLRRHWEVLIGVLVVSILLLSLLLFLLLQHWRQGKHRTL
AQROADFORPPGAAEPEPKDGGLQRRSSPAADVQGENFCAAVKNTQPEDGVEMDTR
QSPHDEDPQAVTYAKVKHSRPRREMASPPSPLSGEFLDTKDRQAEEDROMDTEAAAS
EAPQDVTYARLHSFTLRQKATEPPPSQEGASPAEPSVYATLAIH; LILRB4 protein
(ENSP00000375616) encoded by Transcript ID ENST00000391736 from Gene ID
ENSG00000186818; Homo sapiens
99:
ATGATCCCCACCTTCACGGCTCTGCTCTGCCTCGGGCTGAGTCTGGGCCCCAGGAC
CCACATGCAGGCAGGGCCCCTCCCCAAACCCACCCTCTGGGCTGAGCCAGGCTCT
GTGATCAGCTGGGGGAACTCTGTGACCATCTGGTGTCAGGGGACCCTGGAGGCTC
GGGAGTACCGTCTGGATAAAGAGGAAAGCCCAGCACCCTGGGACAGACAGAACC
CACTGGAGCCCAAGAACAAGGCCAGATTCTCCATCCCATCCATGACAGAGGACTA
TGCAGGGAGATACCGCTGTTACTATCGCAGCCCTGTAGGCTGGTCACAGCCCAGT
GACCCCCTGGAGCTGGTGATGACAGGAGCCTACAGTAAACCCACCCTTTCAGCCC
TGCCGAGTCCTCTTGTGACCTCAGGAAAGAGCGTGACCCTGCTGTGTCAGTCACG
GAGCCCAATGGACACTTTCCTTCTGATCAAGGAGCGGGCAGCCCATCCCCTACTG
CATCTGAGATCAGAGCACGGAGCTCAGCAGCACCAGGCTGAATTCCCCATGAGTC
CTGTGACCTCAGTGCACGGGGGGACCTACAGGTGCTTCAGCTCACACGGCTTCTC
CCACTACCTGCTGTCACACCCCAGTGACCCCCTGGAGCTCATAGTCTCAGGATCCT
TGGAGGATCCCAGGCCCTCACCCACAAGGTCCGTCTCAACAGCTGCAGGCCCTGA
GGACCAGCCCCTCATGCCTACAGGGTCAGTCCCCCACAGTGGTCTGAGAAGGCAC
TGGGAGGTACTGATCGGGGTCTTGGTGGTCTCCATCCTGCTTCTCTCCCTCCTCCTC
TTCCTCCTCCTCCAACACTGGCGTCAGGGAAAACACAGGACATTGGCCCAGAGAC
AGGCTGATTTCCAACGTCCTCCAGGGGCTGCCGAGCCAGAGCCCAAGGACGGGGG
CCTACAGAGGAGGTCCAGCCCAGCTGCTGACGTCCAGGGAGAAAACTTCTGTGCT
GCCGTGAAGAACACACAGCCTGAGGACGGGGTGGAAATGGACACTCGGCAGAGC
CCACACGATGAAGACCCCCAGGCAGTGACGTATGCCAAGGTGAAACACTCCAGA
CCTAGGAGAGAAATGGCCTCTCCTCCCTCCCCACTGTCTGGGGAATTCCTGGACAC
AAAGGACAGACAGGCAGAAGAGGACAGACAGATGGACACTGAGGCTGCTGCATC
TGAAGCCCCCCAGGATGTGACCTACGCCCAGCTGCACAGCTTTACCCTCAGACAG
AAGGCAACTGAGCCTCCTCCATCCCAGGAAGGGGCCTCTCCAGCTGAGOCCAGTG
TCTATGCCACTCTGGCCATCCACTAA; Transcript ID ENST00000612454; Homo sapiens
100:
MIPTFTALLCLGLSLGPRTHMQAGPLPKPTLWAEPGSVISWGNSVTIWCQGTLEAREY
RLDKEESPAPWDRQNPLEPKNKARFSIPSMTEDYAGRYRCYYRSPVGWSQPSDPLEL
VMTGAYSKPTLSALPSPLVTSGKSVTLLCQSRSPMDTFLLIKERAAHPLLHLRSEHGA
QQHQAEFPMSPVTSVHGGTYRCFSSHGFSHYLLSHPSDPLELIVSGSLEDPRPSPTRSVS
TAAGPEDQPLMPTGSVPHSGLRRHWEVLIGVLVVSILLLSLLLFLLLQHWRQGKHRTL
AQRQADFORPPGAAEPEPKDGGLQRRSSPAADVQGENFCAAVKNTQPEDGVEMDTR
QSPHDEDPQAVTYAKVKHSRPRREMASPPSPLSGEFLDTKDRQABEDRQMDTEAAAS
EAPQDVTYAQLHSFTLRQKATEPPPSQEGASPAEPSVYATLAIH; LILRB4 protein
(ENSP00000479829) encoded by Transcript ID ENST00000612454 from Gene ID
ENSG00000275730; Homo sapiens
101:
ATGATCCCCACCTTCACGGCTCTGCTCTGCCTCGGGCTGAGTCTGGGCCCCAGGAC
CCACATGCAGGCAGGGCCCCTCCCCAAACCCACCCTCTGGGCTGAGCCAGGCTCT
GTGATCAGCTGGGGGAACTCTGTGACCATCTGGTGTCAGGGGACCCTGGAGGCTC
GGGAGTACCGTCTGGATAAAGAGGAAAGCCCAGCACCCTGGGACAGACAGAACC
CACTGGAGCCCAAGAACAAGGCCAGATTCTCCATCCCATCCATGACAGAGGACTA
TGCAGGGAGATACCGCTGTTACTATCGCAGCCCTGTAGGCTGGTCACAGCCCAGT
GACCCCCTGGAGCTGGTGATGACAGGAGCCTACAGTAAACCCACCCTTTCAGCCC
TGCCGAGTCCTCTTGTGACCTCAGGAAAGAGCGTGACCCTGCTGTGTCAGTCACG
GAGCCCAATGGACACTTTCCTTCTGATCAAGGAGCGGGCAGCCCATCCCCTACTG
CATCTGAGATCAGAGCACGGAGCTCAGCAGCACCAGGCTGAATTCCCCATGAGTC
CTGTGACCTCAGTGCACGGGGGGACCTACAGGTGCTTCAGCTCACACGGCTTCTC
CCACTACCTGCTGTCACACCCCAGTGACCCCCTGGAGCTCATAGTCTCAGGATCCT
TGGAGGATCCCAGGCCCTCACCCACAAGGTCCGTCTCAACAGCTGCAGGCCCTGA
GGACCAGCCCCTCATGCCTACAGGGTCAGTCCCCCACAGTGGTCTGAGAAGGCAC
TGGGAGGTACTGATCGGGGTCTTGGTGGTCTCCATCCTGCTTCTCTCCCTCCTCCTC
TTCCTCCTCCTCCAACACTGGCGTCAGGGAAAACACAGGACATTGGCCCAGAGAC
AGGCTGATTTCCAACGTCCTCCAGGGGCTGCCGAGCCAGAGCCCAAGGACGGGGG
CCTACAGAGGAGGTCCAGCCCAGCTGCTGACGTCCAGGGAGAAAACTTCTCAGGT
GCTGCCGTGAAGAACACACAGCCTGAGGACGGGGTGGAAATGGACACTCGGCAG
AGCCCACACGATGAAGACCCCCAGGCAGTGACGTATGCCAAGGTGAAACACTCC
AGACCTAGGAGAGAAATGGCCTCTCCTCCCTCCCCACTGTCTGGGGAATTCCTGG
ACACAAAGGACAGACAGGCAGAAGAGGACAGACAGATGGACACTGAGGCTGCTG
CATCTGAAGCCCCCCAGGATGTGACCTACGCCCAGCTGCACAGCTTTACCCTCAG
ACAGAAGGCAACTGAGCCTCCTCCATCCCAGGAAGGGGCCTCTCCAGCTGAGCCC
AGTGTCTATGCCACTCTGGCCATCCACTAA; Transcript ID ENST00000614699; Homo
sapiens
102:
MIPTFTALLCLGLSLGPRTHMQAGPLPKPTLWAEPGSVISWGNSVTIWCQGTLEAREY
RLDKEESPAPWDRQNPLEPKNKARFSIPSMTEDYAGRYRCYYRSPVGWSQPSDPLEL
VMTGAYSKPTLSALPSPLVTSGKSVTLLCQSRSPMDTFLLIKERAAHPLLHLRSEHGA
QQHQAEFPMSPVTSVHGGTYRCFSSHGFSHYLLSHPSDPLELIVSGSLEDPRPSPTRSVS
TAAGPEDQPLMPTGSVPHSGLRRHWEVLIGVLVVSILLLSLLLFLLLQHWRQGKHRTL
AQRQADFORPPGAAEPEPKDGGLQRRSSPAADVQGENFSGAAVKNTQPEDGVEMDT
RQSPHDEDPQAVTYAKVKHSRPRREMASPPSPLSGEFLDTKDRQAEEDRQMDTEAAA
SEAPQDVTYAQLHSFTLRQKATEPPPSQEGASPAEPSVYATLAIH; LILRB4 protein
(ENSP00000478542) encoded by Transcript ID ENST00000614699 from Gene ID
ENSG00000275730; Homo sapiens
103:
ATGATCCCCACCTTCACGGCTCTGCTCTGCCTCGGGCTGAGTCTGGGCCCCAGGAC
CCACATGCAGGCAGGGCCCCTCCCCAAACCCACCCTCTGGGCTGAGCCAGGCTCT
GTGATCAGCTGGGGGAACTCTGTGACCATCTGGTGTCAGGGGACCCTGGAGGCTC
GGGAGTACCGTCTGGATAAAGAGGAAAGCCCAGCACCCTGGGACAGACAGAACC
CACTGGAGCCCAAGAACAAGGCCAGATTCTCCATCCCATCCATGACAGAGGACTA
TGCAGGGAGATACCGCTGTTACTATCGCAGCCCTGTAGGCTGGTCACAGCCCAGT
GACCCCCTGGAGCTGGTGATGACAGGAGCCTACAGTAAACCCACCCTTTCAGCCC
TGCCGAGTCCTCTTGTGACCTCAGGAAAGAGCGTGACCCTGCTGTGTCAGTCACG
GAGCCCAATGGACACTTTCCTTCTGATCAAGGAGCGGGCAGCCCATCCCCTACTG
CATCTGAGATCAGAGCACGGAGCTCAGCAGCACCAGGCTGAATTCCCCATGAGTC
CTGTGACCTCAGTGCACGGGGGGACCTACAGGTGCTTCAGCTCACACGGCTTCTC
CCACTACCTGCTGTCACACCCCAGTGACCCCCTGGAGCTCATAGTCTCAGGATCCT
TGGAGGATCCCAGGCCCTCACCCACAAGGTCCGTCTCAACAGCTGCAGGCCCTGA
GGACCAGCCCCTCATGCCTACAGGGTCAGTCCCCCACAGTGGTCTGAGAAGGCAC
TGGGAGGTACTGATCGGGGTCTTGGTGGTCTCCATCCTGCTTCTCTCCCTCCTCCTC
TTCCTCCTCCTCCAACACTGGCGTCAGGGAAAACACAGGACATTGGCCCAGAGAC
AGGCTGATTTCCAACGTCCTCCAGGGGCTGCCGAGCCAGAGCCCAAGGACGGGGG
CCTACAGAGGAGGTCCAGCCCAGCTGCTGACGTCCAGGGAGAAAACTTCTGTGCT
GCCGTGAAGAACACACAGCCTGAGGACGGGGTGGAAATGGACACTCGGAGCCCA
CACGATGAAGACCCCCAGGCAGTGACGTATGCCAAGGTGAAACACTCCAGACCTA
GGAGAGAAATGGCCTCTCCTCCCTCCCCACTGTCTGGGGAATTCCTGGACACAAA
GGACAGACAGGCAGAAGAGGACAGACAGATGGACACTGAGGCTGCTGCATCTGA
AGCCCCCCAGGATGTGACCTACGCCCAGCTGCACAGCTTTACCCTCAGACAGAAG
GCAACTGAGCCTCCTCCATCCCAGGAAGGGGCCTCTCCAGCTGAGCCCAGTGTCT
ATGCCACTCTGGCCATCCACTAA; Transcript ID ENST00000621693; Homo sapiens
104:
MIPTFTALLCLGLSLGPRTHMQAGPLPKPTLWAEPGSVISWGNSVTIWCQGTLEAREY
RLDKEESPAPWDRONPLEPKNKARFSIPSMTEDYAGRYRCYYRSPVGWSQPSDPLEL
VMTGAYSKPTLSALPSPLVTSGKSVTLLCQSRSPMDTFLLIKERAAHPLLHLRSEHGA
QQHQAEFPMSPVTSVHGGTYRCFSSHGFSHYLLSHPSDPLELIVSGSLEDPRPSPTRSVS
TAAGPEDQPLMPTGSVPHSGLRRHWEVLIGVLVVSILLLSLLLFLLLQHWRQGKHRTL
AQRQADFQRPPGAAEPEPKDGGLQRRSSPAADVQGENFCAAVKNTQPEDGVEMDTR
SPHDEDPQAVTYAKVKHSRPRREMASPPSPLSGEFLDTKDRQAEEDRQMDTEAAASE
APQDVTYAQLHSFTLRQKATEPPPSQEGASPAEPSVYATLAIH; LILRB4 protein
(ENSP00000482234) encoded by Transcript ID ENST00000621693 from Gene ID
ENSG00000275730; Homo sapiens
105:
ATGGGGCGCCTGGCCTCGAGGCCGCTGCTGCTGGCGCTCCTGTCGTTGGCTCTTTG
CCGAGGGCGTGTGGTGAGAGTCCCCACAGCGACCCTGGTTCGAGTGGTGGGCACT
GAGCTGGTCATCCCCTGCAACGTCAGTGACTATGATGGCCCCAGCGAGCAAAACT
TTGACTGGAGCTTCTCATCTTTGGGGAGCAGCTTTGTGGAGCTTGCAAGCACCTGG
GAGGTGGGGTTCCCAGCCCAGCTGTACCAGGAGCGGCTGCAGAGGGGCGAGATC
CTGTTAAGGCGGACTGCCAACGACGCCGTGGAGCTCCACATAAAGAACGTCCAGC
CTTCAGACCAAGGCCACTACAAATGTTCAACCCCCAGCACAGATGCCACTGTCCA
GGGAAACTATGAGGACACAGTGCAGGTTAAAGTGCTGGCCGACTCCCTGCACGTG
GGCCCCAGCGCGCGGCCCCCGCCGAGCCTGAGCCTGCGGGGGGGGAGCCCTTCG
AGCTGCGCTGCACCGCCGCCTCCGCCTCGCCGCTGCACACGCACCTGGCGCTGCT
GTGGGAGGTGCACCGCGGCCCGGCCAGGCGGAGCGTCCTCGCCCTGACCCACGAG
GGCAGGTTCCACCCGGGOCTGGGGTACGAGCAGCGCTACCACAGTGGGGACGTGC
GCCTCGACACCGTGGGCAGCGACGCCTACCGCCTCTCAGTGTCCCGGGCTCTGTCT
GCCGACCAGGGCTCCTACAGGTGTATCGTCAGCGAGTGGATCGCCGAGCAGGGCA
ACTGGCAGGAAATCCAAGAAAAGGCCGTGGAAGTTGCCACCGTGGTGATCCAGC
CATCAGTTCTGCGAGCAGCTGTGCCCAAGAATGTGTCTGTGGCTGAAGGAAAGGA
ACTGGACCTGACCTGTAACATCACAACAGACCGAGCCGATGACGTCCGGCCCGAG
GTGACGTGGTCCTTCAGCAGGATGCCTGACAGCACCCTACCTGGCTCCCGCGTGTT
GGCGCGGCTTGACCGTGATTCCCTGGTGCACAGCTCGCCTCATGTTGCTTTGAGTC
ATGTGGATGCACGCTCCTACCATTTACTGGTTCGGGATGTTAGCAAAGAAAACTCT
GGCTACTATTACTGCCACGTGTCCCTGTGGGCACCCGGACACAACAGGAGCTGGC
ACAAAGTGGCAGAGGCCGTGTCTTCCCCAGCTGGTGTGGGTGTGACCTGGCTAGA
ACCAGACTACCAGGTGTACCTGAATGCTTCCAAGGTCCCCGGGTTTGCGGATGAC
CCCACAGAGCTGGCATGCCGGGTGGTGGACACGAAGAGTGGGGAGGCGAATGTC
CGATTCACGGTTTCGTGGTACTACAGGATGAACCGGCGCAGCGACAATGTGGTGA
CCAGCGAGCTGCTTGCAGTCATGGACGGGGACTGGACGCTAAAATATGGAGAGA
GGAGCAAGCAGCGGGCCCAGGATGGAGACTTTATTTTTTCTAAGGAACATACAGA
CACGTTCAATTTCCGGATCCAAAGGACTACAGAGGAAGACAGAGGCAATTATTAC
TGTGTTGTGTCTGCCTGGACCAAACAGCGGAACAACAGCTGGGTGAAAAGCAAGG
ATGTCTTCTCCAAGCCTGTTAACATATTTTGGGCATTAGAAGATTCCGTGCTTGTG
GTGAAGGCGAGGCAGCCAAAGCCTTTCTTTGCTGCCGGAAATACATTTGAGATGA
CTTGCAAAGTATCTTCCAAGAATATTAAGTCGCCACGCTACTCTGTTCTCATCATG
GCTGAGAAGCCTGTCGGCGACCTCTCCAGTCCCAATGAAACGAAGTACATCATCT
CTCTGGACCAGGATTCTGTGGTGAAGCTGGAGAATTGGACAGATGCATCACGGGT
GGATGGCGTTGTTTTAGAAAAAGTGCAGGAGGATGAGTTCCGCTATCGAATGTAC
CAGACTCAGGTCTCAGACGCAGGGCTGTACCGCTGCATGGTGACAGCCTGGTCTC
CTGTCAGGGGCAGCCTTTGGCGAGAAGCAGCAACCAGTCTCTCCAATCCTATTGA
GATAGACTTCCAAACCTCAGGTCCTATATTTAATGCTTCTGTGCATTCAGACACAC
CATCAGTAATTCGGGGAGATCTGATCAAATTGTTCTGTATCATCACTGTCGAGGGA
GCAGCACTGGATCCAGATGACATGGCCTTTGATGTGTCCTGGTTTGCGGTGCACTC
TTTTGGCCTGGACAAGGCTCCTGTGCTCCTGTCTTCCCTGGATCGGAAGGGCATCG
TGACCACCTCCCGGAGGGACTGGAAGAGCGACCTCAGCCTGGAGCGCGTGAGTGT
GCTGGAATTCTTGCTGCAAGTGCATGGCTCCGAGGACCAGGACTTTGGCAACTAC
TACTGTTCCGTGACTCCATGGGTGAAGTCACCAACAGGTTCCTGGCAGAAGGAGG
CAGAGATCCACTCCAAGCCCGTTTTTATAACTGTGAAGATGGATGTGCTGAACGC
CTTCAAGTATCCCTTGCTGATCGGCGTCGGTCTGTCCACGGTCATCGGGCTCCTGT
CCTGTCTCATCGGGTACTGCAGCTCCCACTGGTGTTGTAAGAAGGAGGTTCAGGA
GACACGGCGCGAGCGCCGCAGGCTCATGTCGATGGAGATGGACTAG; Transcript ID
ENST00000393203; Homo sapiens
106:
MGRLASRPLLLALLSLALCRGRVVRVPTATLVRVVGTELVIPCNVSDYDGPSEQNFD
WSFSSLGSSFVELASTWEVGFPAQLYQERLQRGEILLRRTANDAVELHIKNVQPSDQG
HYKCSTPSTDATVQGNYEDTVQVKVLADSLHVGPSARPPPSLSLREGEPFELRCTAAS
ASPLHTHLALLWEVHRGPARRSVLALTHEGRFHPGLGYEQRYHSGDVRLDTVGSDA
YRLSVSRALSADQGSYRCIVSEWIAEQGNWQEIQEKAVEVATVVIQPSVLRAAVPKN
VSVAEGKELDLTCNITTDRADDVRPEVTWSFSRMPDSTLPGSRVLARLDRDSLVHSSP
HVALSHVDARSYHLLVRDVSKENSGYYYCHVSLWAPGHNRSWHKVAEAVSSPAGV
GVTWLEPDYQVYLNASKVPGFADDPTELACRVVDTKSGEANVRFTVSWYYRMNRR
SDNVVTSELLAVMDGDWTLKYGERSKQRAQDGDFIFSKEHTDTFNFRIQRTTEEDRG
NYYCVVSAWTKQRNNSWVKSKDVFSKPVNIFWALEDSVLVVKARQPKPFFAAGNTF
EMTCKVSSKNIKSPRYSVLIMAEKPVGDLSSPNETKYHISLDQDSVVKLENWTDASRV
DGVVLEKVQEDEFRYRMYQTQVSDAGLYRCMVTAWSPVRGSLWREAATSLSNPIEI
DFQTSGPIFNASVHSDTPSVIRGDLIKLFCIITVEGAALDPDDMAFDVSWFAVHSFGLD
KAPVLLSSLDRKGIVTTSRRDWKSDLSLERVSVLEFLLQVHGSEDQDFGNYYCSVTP
WVKSPTGSWQKEAEIHSKPVFITVKMDVLNAFKYPLLIGVGLSTVIGLLSCLIGYCSSH
WCCKKEVQETRRERRRLMSMEMD; PTGFRN protein (ENSP00000376899) encoded by
Transcript ID ENST00000393203 from Gene ID ENSG00000134247; Homo sapiens
107:
ATGGCAGTGGGGGCCAGTGGTCTAGAAGGAGATAAGATGGCTGGTGCCATGCCTC
TGCAACTCCTCCTGTTGCTGATCCTACTGGGCCCTGGCAACAGCTTGCAGCTGTGG
GACACCTGGGCAGATGAAGCCGAGAAAGCCTTGGGTCCCCTGCTTGCCCGGGACC
GGAGACAGGCCACCGAATATGAGTACCTAGATTATGATTTCCTGCCAGAAACGGA
GCCTCCAGAAATGCTGAGGAACAGCACTGACACCACTCCTCTGACTGGGCCTGGA
ACCCCTGAGTCTACCACTGTGGAGCCTGCTGCAAGGCGTTCTACTGGCCTGGATGC
AGGAGGGGCAGTCACAGAGCTGACCACGGAGCTGGCCAACATGGGGAACCTGTC
CACGGATTCAGCAGCTATGGAGATACAGACCACTCAACCAGCAGCCACGGAGGC
ACAGACCACTCAACCAGTGCCCACGGAGGCACAGACCACTCCACTGGCAGCCACA
GAGGCACAGACAACTCGACTGACGGCCACGGAGGCACAGACCACTCCACTGGCA
GCCACAGAGGCACAGACCACTCCACCAGCAGCCACGGAAGCACAGACCACTCAA
CCCACAGGCCTGGAGGCACAGACCACTGCACCAGCAGCCATGGAGGCACAGACC
ACTGCACCAGCAGCCATGGAAGCACAGACCACTCCACCAGCAGCCATGGAGGCA
CAGACCACTCAAACCACAGCCATGGAGGCACAGACCACTGCACCAGAAGCCACG
GAGGCACAGACCACTCAACCCACAGCCACGGAGGCACAGACCACTCCACTGGCA
GCCATGGAGGCCCTGTCCACAGAACCCAGTGCCACAGAGGCCCTGTCCATGGAAC
CTACTACCAAAAGAGGTCTGTTCATACCCTTTTCTGTGTCCTCTGTTACTCACAAG
GGCATTCCCATGGCAGCCAGCAATTTGTCCGTCAACTACCCAGTGGGGGCCCCAG
ACCACATCTCTGTGAAGCAGTGCCTGCTGGCCATCCTAATCTTGGCGCTGGTGGCC
ACTATCTTCTTCGTGTGCACTGTGGTGCTGGCGGTCCGCCTCTCCCGCAAGGGCCA
CATGTACCCCGTGCGTAATTACTCCCCCACCGAGATGGTCTGCATCTCATCCCTGT
TGCCTGATGGGGGTGAGGGGCCCTCTGCCACAGCCAATGGGGGCCTGTCCAAGGC
CAAGAGCCCGGGCCTGACGCCAGAGCCCAGGGAGGACCGTGAGGGGGATGACCT
CACCCTGCACAGCTTCCTCCCTTAG; Transcript ID ENST00000228463; Homo sapiens
108:
MAVGASGLEGDKMAGAMPLQLLLLLILLGPGNSLQLWDTWADEAEKALGPLLARD
RRQATEYEYLDYDFLPETEPPEMLRNSTDTTPLTGPGTPESTTVEPAARRSTGLDAGG
AVTELTTELANMGNLSTDSAAMEIQTTQPAATEAQTTQPVPTEAQTTPLAATEAQTTR
LTATEAQTTPLAATEAQTTPPAATEAQTTQPTGLEAQTTAPAAMEAQTTAPAAMEAQ
TTPPAAMEAQTTOTTAMEAQTTAPEATEAQTTOPTATEAQTTPLAAMEALSTEPSAT
EALSMEPTTKRGLFIPFSVSSVTHKGIPMAASNLSVNYPVGAPDHISVKQCLLAILILAL
VATIFFVCTVVLAVRLSRKGHMYPVRNYSPTEMVCISSLLPDGGEGPSATANGGLSKA
KSPGLTPEPREDREGDDLTLHSFLP; SELPLG protein (ENSP00000228463) encoded by
Transcript ID ENST00000228463 from Gene ID ENSG00000110876; Homo sapiens
109:
ATGCCTCTGCAACTCCTCCTGTTGCTGATCCTACTGGGCCCTGGCAACAGCTTGCA
GCTGTGGGACACCTGGGCAGATGAAGCCGAGAAAGCCTTGGGTCCCCTGCTTGCC
CGGGACCGGAGACAGGCCACCGAATATGAGTACCTAGATTATGATTTCCTGCCAG
AAACGGAGCCTCCAGAAATGCTGAGGAACAGCACTGACACCACTCCTCTGACTGG
GCCTGGAACCCCTGAGTCTACCACTGTGGAGCCTGCTGCAAGGCGTTCTACTGGG
CTGGATGCAGGAGGGGCAGTCACAGAGCTGACCACGGAGCTGGCCAACATGGGG
AACCTGTCCACGGATTCAGCAGCTATGGAGATACAGACCACTCAACCAGCAGCCA
CGGAGGCACAGACCACTCAACCAGTGCCCACGGAGGCACAGACCACTCCACTGG
CAGCCACAGAGGCACAGACAACTCGACTGACGGCCACGGAGGCACAGACCACTC
CACTGGCAGCCACAGAGGCACAGACCACTCCACCAGCAGCCACGGAAGCACAGA
CCACTCAACCCACAGGCCTGGAGGCACAGACCACTGCACCAGCAGCCATGGAGG
CACAGACCACTGCACCAGCAGCCATGGAAGCACAGACCACTCCACCAGCAGCCAT
GGAGGCACAGACCACTCAAACCACAGCCATGGAGGCACAGACCACTGCACCAGA
AGCCACGGAGGCACAGACCACTCAACCCACAGCCACGGAGGCACAGACCACTCC
ACTGGCAGCCATGGAGGCCCTGTCCACAGAACCCAGTGCCACAGAGGCCCTGTCC
ATGGAACCTACTACCAAAAGAGGTCTGTTCATACCCTTTTCTGTGTCCTCTGTTAC
TCACAAGGGCATTCCCATGGCAGCCAGCAATTTGTCCGTCAACTACCCAGTGGGG
GCCCCAGACCACATCTCTGTGAAGCAGTGCCTGCTGGCCATCCTAATCTTGGCGCT
GGTGGCCACTATCTTCTTCGTGTGCACTGTGGTGCTGGGGGTCCGCCTCTCCCGCA
AGGGCCACATGTACCCCGTGCGTAATTACTCCCCCACCGAGATGGTCTGCATCTCA
TCCCTGTTGCCTGATGGGGGTGAGGGGCCCTCTGCCACAGCCAATGGGGGCCTGT
CCAAGGCCAAGAGCCCGGGCCTGACGCCAGAGCCCAGGGAGGACCGTGAGGGGG
ATGACCTCACCCTGCACAGCTTCCTCCCTTAG; Transcript ID ENST00000550948;
Homo sapiens
110:
MPLQLLLLLILLGPGNSLQLWDTWADEAEKALGPLLARDRRQATEYEYLDYDFLPET
EPPEMLRNSTDTTPLTGPGTPESTTVEPAARRSTGLDAGGAVTELTTELANMGNLSTD
SAAMEIQTTQPAATEAQTTQPVPTEAQTTPLAATEAQTTRLTATEAQTTPLAATEAQT
TPPAATEAQTTQPTGLEAQTTAPAAMEAQTTAPAAMEAQTTPPAAMEAQTTQTTAM
EAQTTAPEATEAQTTQPTATEAQTTPLAAMEALSTEPSATEALSMEPTTKRGLFIPFSV
SSVTHKGIPMAASNLSVNYPVGAPDHISVKQCLLAILILALVATIFFVCTVVLAVRLSR
KGHMYPVRNYSPTEMVCISSLLPDGGEGPSATANGGLSKAKSPGLTPEPREDREGDDL
TLHSFLP; SELPLG protein (ENSP00000447752) encoded by Transcript ID
ENST00000550948 from Gene ID ENSG00000110876; Homo sapiens
*based on assembled sequence in Genome Reference Consortium Human Build 38 patch release 13 (GRCh38.p13; GenBank assembly accession GCA_000001405.28 and RefSeq assembly accession GCF_000001405.39); note multiple listings for the same vesicle localization moiety reflect different transcripts (different ENST numbers) resulting potentially in multiple isoforms of a vesicle localization moiety when transcripts differ outside the 5′ and 3′ untranslated region (UTR) (i.e., differ in the coding sequences).

TABLE 3
Additional VLM which may be used as a VLM or used
to produce a chimeric VLM^{#, @}

Gene Symbol; Protein (PROT ID NO); Sequence Identifiers
ACE; P12821 (1); ENST00000290866, ENST00000290863, ENST00000413513
ADAM15; Q13444 (2); ENST00000529473, ENST00000526491, ENST00000356955,
ENST00000449910, ENST00000359280, ENST00000360674, ENST00000368412,
ENST00000355956, ENST00000271836, ENST00000368413, ENST00000531455,
ENST00000447332
ADAM9; Q13443 (3); ENST00000487273, ENST00000379917
AGRN; O00468 (4); ENST00000379370
ANPEP; P15144 (5); ENST00000300060
ANTXR2; P58335 (6); ENST00000403729, ENST00000346652, ENST00000307333
ATP1A1; P05023 (7); ENST00000295598, ENST00000369496, ENST00000537345
ATP1B3; P54709 (8); ENST00000286371
BSG; P35613 (9); ENST00000545507, ENST00000346916, ENST00000333511,
ENST00000353555
BTN2A1; Q7KYR7 (10); ENST00000312541, ENST00000429381, ENST00000469185,
ENST00000541522
CALM1; P0DP23 (11); ENST00000356978
CANX; P27824 (12); ENST00000504734, ENST00000247461, ENST00000452673,
ENST00000638425, ENST00000639938, ENST00000638706
CD151; P48509 (13); ENST00000322008, ENST00000397420, ENST00000530726,
ENST00000397421
CD19; P15391 (14); ENST00000538922, ENST00000324662
CD1A; P06126 (15); ENST00000289429
CD1B; P29016 (16); ENST00000368168
CD1C; P29017 (17); ENST00000368170
CD2; P06729 (18); ENST00000369478
CD200; P41217 (19); ENST00000473539, ENST00000315711
CD200R1; Q8TD46 (20); ENST00000471858, ENST00000308611, ENST00000440122,
ENST00000490004
CD226; Q15762 (21); ENST00000280200, ENST00000582621
CD247; P20963 (22); ENST00000362089, ENST00000392122
CD274; Q9NZQ7 (23); ENST00000381577, ENST00000381573
CD276; Q5ZPR3 (24); ENST00000318443, ENST00000561213, ENST00000564751,
ENST00000318424
CD33; P20138 (25); ENST00000421133, ENST00000391796, ENST00000262262
CD34; P28906 (26); ENST00000310833, ENST00000356522
CD36; P16671 (27); ENST00000435819, ENST00000309881, ENST00000394788,
ENST00000447544, ENST00000433696, ENST00000432207, ENST00000538969,
ENST00000544133
CD37; P11049 (28); ENST00000598095, ENST00000426897, ENST00000323906
CD3E; P07766 (29); ENST00000361763
CD40; P25942 (30); ENST00000372285, ENST00000372276
CD40LG; P29965 (31); ENST00000370629
CD44; P16070 (32); ENST00000263398, ENST00000428726, ENST00000415148,
ENST00000433892, ENST00000278386, ENST00000434472, ENST00000352818
CD47; Q08722 (33); ENST00000355354, ENST00000361309
CD53; P19397 (34); ENST00000648608, ENST00000271324
CD58; P19256 (35); ENST00000369489, ENST00000464088, ENST00000457047
CD63; P08962 (36); ENST00000546939, ENST00000552692, ENST00000549117,
ENST00000257857, ENST00000552754, ENST00000550776, ENST00000420846
CD81; P60033 (37); ENST00000263645
CD82; P27701 (38); ENST00000227155, ENST00000342935
CD84; Q9UIB8 (39); ENST00000368054, ENST00000368048, ENST00000311224,
ENST00000368051, ENST00000534968
CD86; P42081 (40); ENST00000469710, ENST00000493101, ENST00000330540
ENST00000393627, ENST00000264468
CD9; P21926 (41); ENST00000382518, ENST00000538834, ENST00000009180
CHMP1A; Q9HD42 (42); ENST00000397901
CHMP1B; Q7LBR1 (43); ENST00000526991
CHMP2A; O43633 (44); ENST00000600118, ENST00000601220, ENST00000312547
CHMP3; Q9Y3E7 (45); ENST00000263856, ENST00000409727, ENST00000409225
CHMP4A; Q9BY43 (46); ENST00000347519, ENST00000609024, ENST00000645308,
ENST00000645179
CHMP4B; Q9H444 (47); ENST00000217402
CHMP5; Q9NZZ3 (48); ENST00000223500, ENST00000419016
CHMP6; Q96FZ7 (49); ENST00000325167
COL6A1; P12109 (50); ENST00000361866
CR1; P17927 (51); ENST00000400960, ENST00000367051, ENST00000367053
CSF1R; P07333 (52); ENST00000286301, ENST00000543093
CXCR4; P61073 (53); ENST00000409817, ENST00000241393
DDOST; P39656 (54); ENST00000375048, ENST00000415136
DLL1; O00548 (55); ENST00000616526, ENST00000366756
DLL4; Q9NR61 (56); ENST00000249749
DSG1; Q02413 (57); ENST00000257192
EMB; Q6PCB8 (58); ENST00000303221, ENST00000514111
ENG; P17813 (59); ENST00000373203, ENST00000344849
EVI2B; P34910 (60); ENST00000330927, ENST00000577894
F11R; Q9Y624 (61); ENST00000368026, ENST00000537746
FASN; P49327 (62); ENST00000306749
FCER1G; P30273 (63); ENST00000289902
FCGR2C; P31995 (64); * P31995-1, P31995-2, P31995-3, P31995-4
FLOT1; O75955 (65); ENST00000436822, ENST00000383562, ENST00000376389,
ENST00000444632, ENST00000383382
FLOT2; Q14254 (66); ENST00000394908
FLT3; P36888 (67); ENST00000241453
FN1; P02751 (68); ENST00000421182, ENST00000323926, ENST00000336916,
ENST00000357867, ENST00000354785, ENST00000446046, ENST00000443816,
ENST00000432072, ENST00000356005, ENST00000426059, ENST00000359671
GAPDH; P04406 (69); ENST00000229239, ENST00000396861, ENST00000396859,
ENST00000396858, ENST00000619601
GLG1; Q92896 (70); ENST00000205061, ENST00000422840, ENST00000447066
GRIA2; P42262 (71); ENST00000507898, ENST00000393815, ENST00000645636,
ENST00000296526, ENST00000264426
GRIA3; P42263 (72); ENST00000541091, ENST00000620443, ENST00000622768
GYPA; P02724 (73); ENST00000324022, ENST00000646447, ENST00000642713
HSPG2; P98160 (74); ENST00000374695
ICAM1; P05362 (75); ENST00000264832
ICAM2; P13598 (76); ENST00000449662, ENST00000579788, ENST00000579687,
ENST00000412356, ENST00000418105
ICAM3; P32942 (77); ENST00000160262
IL1RAP; Q9NPH3 (78); ENST00000072516, ENST00000439062, ENST00000447382,
ENST00000422485, ENST00000422940, ENST00000413869, ENST00000342550,
ENST00000317757, ENST00000443369, ENST00000412504
IL5RA; Q01344 (79); ENST00000446632, ENST00000438560, ENST00000256452,
ENST00000383846, ENST00000311981, ENST00000430514, ENST00000456302
IST1; P53990 (80); ENST00000544564, ENST00000541571, ENST00000378799,
ENST00000329908, ENST00000538850, ENST00000378798, ENST00000606369,
ENST00000535424
ITGA2; P17301 (81); ENST00000296585
ITGA2B; P08514 (82); ENST00000262407
ITGA4; P13612 (83); ENST00000339307, ENST00000397033
ITGA5; P08648 (84); ENST00000293379
ITGA6; P23229 (85); ENST00000409532, ENST00000264107, ENST00000409080,
ENST00000442250, ENST00000458358
ITGAL; P20701 (86); ENST00000356798, ENST00000358164
ITGAM; P11215 (87); ENST00000648685, ENST00000544665
ITGAV; P06756 (88); ENST00000261023, ENST00000374907, ENST00000433736
ITGAX; P20702 (89); ENST00000268296
ITGB2; P05107 (90); ENST00000397852, ENST00000397857, ENST00000355153,
ENST00000397850, ENST00000302347
ITGB3; P05106 (91); ENST00000559488
ITGB4; P16144 (92); ENST00000579662, ENST00000200181, ENST00000450894,
ENST00000449880
ITGB5; P18084 (93); ENST00000296181
ITGB6; P18564 (94), ENST00000283249, ENST00000409967, ENST00000409872
ITGB7; P26010 (95); ENST00000267082, ENST00000422257, ENST00000550743
JAG1; P78504 (96); ENST00000254958
JAG2; Q9Y219 (97); ENST00000331782, ENST00000347004
KIT; P10721 (98); ENST00000412167, ENST00000288135
LGALS3BP; Q08380 (99); ENST00000262776
LILRA6; Q6PI73 (100); ENST00000613333, ENST00000621570, ENST00000616720,
ENST00000430421, ENST00000396365, ENST00000614434
LILRB1; Q8NHL6 (101); ENST00000616408, ENST00000618055, ENST00000618681,
ENST00000617686, ENST00000612636
LILRB2; Q8N423 (102); ENST00000619122, ENST00000621020, ENST00000614225,
ENST00000618705, ENST00000391748, ENST00000314446, ENST00000391746,
ENST00000391749, ENST00000434421, ENST00000617886, ENST00000617341,
ENST00000610886, ENST00000618392
LILRB3; O75022 (103); ENST00000611086, ENST00000391750, ENST00000245620,
ENST00000613698
LMAN2; Q12907 (104); ENST00000303127
LRRC25; Q8N386 (105); ENST00000339007, ENST00000595840
LY75; O60449 (106); ENST00000263636
M6PR; P20645 (107); ENST00000000412
MFGE8; Q08431 (108); ENST00000268151, ENST00000268150, ENST00000566497,
ENST00000542878
MMP14; P50281 (109); ENST00000311852
MPL; P40238 (110); ENST00000372470
MRC1; P22897 (111); ENST00000569591
MVB12B; Q9H7P6 (112); ENST00000361171, ENST00000489637
NECTIN1; Q15223 (113); ENST00000341398, ENST00000264025, ENST00000340882
NOMO1; Q15155 (114); ENST00000619292, ENST00000287667
NOTCH1; P46531 (115); ENST00000651671
NOTCH2; Q04721 (116); ENST00000256646
NOTCH3; Q9UM47 (117); ENST00000263388
NOTCH4; Q99466 (118); ENST00000457094, ENST00000375023, ENST00000425600,
ENST00000439349
NPTN; Q9Y639 (119); ENST00000345330, ENST00000351217, ENST00000562924,
ENST00000563691
NRP1; O14786 (120); ENST00000265371, ENST00000374821, ENST00000374822,
ENST00000374867
PDCD1; Q15116 (121); ENST00000618185, ENST00000334409
PDCD1LG2; Q9BQ51 (122); ENST00000397747
PDCD6IP; Q8WUM4 (123); ENST00000307296, ENST00000457054
PDGFRB; P09619 (124); ENST00000261799
PECAM1; P16284 (125); ENST00000563924
PLXNB2; O15031 (126); ENST00000449103, ENST00000359337
PLXND1; Q9Y4D7 (127); ENST00000324093
PROM1; O43490 (128); ENST00000505450, ENST00000508167, ENST00000510224,
ENST00000447510, ENST00000540805, ENST00000539194
PTGES2; Q9H7Z7 (129); ENST00000338961
PTPRA; P18433 (130); ENST00000380393, BNST00000216877, ENST00000318266,
ENST00000356147, ENST00000399903
PTPRC; P08575 (131); ENST00000573679, ENST00000573477, ENST00000348564,
ENST00000442510
PTPRJ; Q12913 (132); ENST00000418331, ENST00000440289
PTPRO; Q16827 (133); ENST00000281171, ENST00000543886, ENST00000348962,
ENST00000442921, ENST00000542557, ENST00000445537, ENST00000544244
RPN1; P04843 (134); ENST00000296255
SDC1; P18827 (135); ENST00000254351, ENST00000381150
SDC2; P34741 (136); ENST00000302190
SDC3; Q75056 (137); ENST00000339394
SDC4; P31431 (138); ENST00000372733
SDCBP; O00560 (139); ENST00000260130, ENST00000447182, ENST00000413219,
ENST00000424270
SDCBP2; Q9H190 (140); ENST00000381812, ENST00000381808, ENST00000339987,
ENST00000360779
SIGLEC7; Q9Y286 (141); ENST00000317643, ENST00000305628, ENST00000536156,
ENST00000600577
SIGLEC9; Q9Y336 (142); ENST00000250360, ENST00000440804
SIRPA; P78324 (143); ENST00000622179, ENST00000356025, ENST00000358771,
ENST00000400068
SLIT2; O94813 (144); ENST00000504154
SNF8; Q96H20 (145); ENST00000502492, ENST00000290330
SPN; P16150 (146); ENST00000395389, ENST00000563039, ENST00000652691,
ENST00000360121
STX3; Q13277 (147); ENST00000337979, ENST00000529177
TACSTD2; P09758 (148); ENST00000371225
TFRC; P02786 (149); ENST00000360110, ENST00000392396
TLR2; O60603 (150); ENST00000642580, ENST00000642700, ENST00000260010
TMED10; P49755 (151); ENST00000303575
TNFRSF8; P28908 (152); ENST00000263932, ENST00000413146, ENST00000417814
TRAC; P01848 (153); * P01848-1
TSG101; Q99816 (154); ENST00000251968
TSPAN14; Q8NG11 (155); ENST00000429989, ENST00000481124, ENST00000372164,
ENST00000372158, ENST00000372156, ENST00000616406
TSPAN7; P41732 (156); ENST00000378482
TSPAN8; P19075 (157); ENST00000393330, ENST00000247829, ENST00000546561
TYROBP; O43914 (158); ENST00000544690, ENST00000262629, ENST00000589517
VPS25; Q9BRG1 (159); ENST00000253794
VPS28; Q9UK41 (160); ENST00000529182, ENST00000526054, ENST00000292510,
ENST00000377348, ENST00000646588, ENST00000642202, ENST00000642867,
ENST00000643186
VPS36; Q86VN1 (161); ENST00000378060, ENST00000611132
VPS37A; Q8NEZ2 (162); ENST00000324849, ENST00000425020, ENST00000521829
VPS37B; Q9H9H4 (163); ENST00000267202
VPS37C; ASD8V6 (164); ENST00000301765
VPS37D; Q86XT2 (165); ENST00000324941
VPS4A; Q9UN37 (166); ENST00000254950
VPS4B; O75351 (167); ENST00000238497
VTI1A; Q96AJ9 (168); ENST00000393077
VTI1B; Q9UEU0 (169); ENST00000554659
^{#} and * UniProt Release 2019_11 (11 Dec. 2019); note amino acid sequence as well as functional and domain structure of vesicle localization moieties may be found under each accession number.
^{@}based on assembled sequence in Genome Reference Consortium Human Build 38 patch release 13 (GRCh38.p13; GenBank assembly accession GCA_000001405.28 and RefSeq assembly accession GCF_000001405.39); nucleic acid sequence coding a vesicle localization moiety may be found within sequence associated with an ENST number; note multiple ENST numbers associated with each vesicle localization moiety referred through its Gene Symbol or UniProtKB accession number potentially indicate multiple isoforms of a vesicle localization moiety.

TABLE 4
Chimeric VLM and LAMP2, CLSTN1 and IGSF VLM (without signal sequence)

SEQ ID NO: Sequence; Source
111:
CTCGAACTTAATTTGACCGATTCAGAGAATGCCACATGCCTTTATGCGAAATGGC
AGATGAATTTCACTGTTCGGTATGAAACCACAAATAAAACTTATAAAACCGTTAC
CATAAGCGACCATGGAACTGTGACCTATAATGGAAGCATATGTGGAGATGATCAG
AATGGTCCCAAAATTGCTGTTCAGTTCGGACCTGGTTTCTCCTGGATTGCTAATTT
TACTAAGGCAGCCTCTACCTATTCCATAGACTCAGTTTCTTTTAGTTACAACACAG
GGGATAACACAACGTTTCCTGATGCCGAAGATAAAGGCATACTCACCGTTGATGA
ACTCTTGGCCATCAGAATACCTCTTAATGACCTGTTTAGATGCAATAGCCTCTCCA
CCCTGGAGAAGAATGATGTGGTACAACACTACTGGGATGTGTTGGTTCAAGCTTT
TGTACAAAATGGGACCGTCTCTACAAATGAGTTCCTCTGTGATAAAGACAAAACC
AGTACTGTGGCACCAACCATACACACAACAGTGCCATCTCCAACGACCACCCCTA
CACCCAAGGAGAAACCTGAAGCCGGTACATATTCAGTGAATAATGGAAATGATAC
ATGCCTTCTGGCCACCATGGGCCTTCAGCTCAACATCACTCAGGATAAGGTCGCTT
CAGTCATTAACATTAACCCCAATACTACTCACTCTACAGGCTCTTGCAGGAGTCAC
ACGGCGCTCCTGCGGTTGAATAGCAGCACCATTAAGTATCTTGACTTTGTCTTTGC
TGTCAAGAATGAGAACAGATTTTATCTGAAAGAGGTCAACATCTCTATGTATTTG
GICAATGGGAGTGTGTTCTCCATTGCTAATAACAATCTCAGCTACTGGGATGCCCC
TCTGGGTTCTTCCTATATGTGCAACAAAGAGCAGACTGTTTCAGTGTCCGGCGCAT
TTCAGATTAATACTTTTGATCTTCGGGTGCAGCCTTTCAATGTGACACAAGGAAAG
TATTCCACCGCCCAAGAGTGTTCTTTGGATGATGACACCATACTGATCCCCATCAT
TGTAGGTGCCGGCCTGAGCGGCCTTATTATCGTTATCGTCATTGCATACGTGATTG
GACGGCGGAAATCTTATGCCGGTTATCAGACGCTT; Construct coding sequence from
vector 91 (for Lamp2 VLM); Artificial Sequence
112:
LELNLTDSENATCLYAKWQMNFTVRYETTNKTYKTVTISDHGTVTYNGSICGDDQN
GPKIAVQFGPGFSWIANFTKAASTYSIDSVSFSYNTGDNTTFPDAEDKGILTVDELLAIR
IPLNDLFRCNSLSTLEKNDVVQHYWDVLVQAFVQNGTVSTNEFLCDKDKTSTVAPTI
HTTVPSPTTTPTPKEKPEAGTYSVNNGNDTCLLATMGLQLNITQDKVASVININPNTT
HSTGSCRSHTALLRLNSSTIKYLDFVFAVKNENRFYLKEVNISMYLVNGSVFSIANNN
LSYWDAPLGSSYMCNKEQTVSVSGAFQINTFDLRVQPFNVTQGKYSTAQECSLDDDT,
ILIPIIVGAGLSGLIIVIVIA, YVIGRRKSYAGYQTL; Construct peptide sequence from
vector 91 (for Lamp2 VLM); Artificial Sequence
113:
GCACGCGTGAATAAACATAAACCGTGGTTGGAACCAACATATCATGGGATCGTTA
CCGAAAATGATAATACAGTACTTCTGGATCCACCTCTCATTGCTTTGGACAAGGAC
GCACCCCTCAGGTTCGCTGAATCATTCGAAGTTACCGTTACGAAGGAAGGGGAAA
TATGCGGTTTCAAGATCCATGGTCAAAACGTTCCTTTCGACGCCGTCGTGGTTGAC
AAGAGCACCGGCGAAGGGGTTATAAGATCTAAGGAAAAGCTCGATTGCGAACTT
CAAAAGGATTACAGCTTTACTATACAAGCGTACGACTGCGGCAAAGGGCCCGACG
GGACAAATGTTAAGAAATCCCACAAGGCCACGGTCCACATCCAAGTCAATGATGT
TAACGAATATGCACCTGTTTTCAAAGAGAAAAGCTATAAGGCTACTGTGATAGAA
GGAAAACAATATGATAGTATCCTGAGAGTCGAAGCTGTCGACGCAGATTGTAGCC
CACAATTTTCCCAAATATGTTCCTATGAGATTATAACACCTGATGTCCCTTTCACC
GTAGATAAGGACGGATACATCAAGAATACTGAAAAGCTGAATTATGGTAAAGAG
CACCAGTACAAACTCACGGTGACGGCGTACGATTGCGGAAAGAAGCGTGCAACT
GAGGACGTACTTGTTAAAATTAGTATCAAACCGACGTGTACACCAGGCTGGCAGG
GCTGGAATAATCGGATCGAATACGAACCCGGAACAGGAGCACTGGCTGTGTTCCC
TAACATTCATCTCGAAACTTGCGATGAACCTGTGGCAAGCGTCCAAGCTACGGTA
GAACTGGAGACATCTCATATTGGTAAGGGATGTGATAGAGATACTTATAGCGAGA
AAAGCCTTCATCGCTTGTGCGGCGCCGCAGCCGGAACAGCAGAACTCTTGCCTTC
TCCCTCTGGCAGCCTTAATTGGACTATGGGATTGCCTACTGATAACGGTCATGATT
CCGATCAAGTCTTCGAATTTAATGGAACACAAGCTGTACGCATTCCTGACGGAGT
GGTAAGTGTTTCTCCGAAGGAACCCTTTACAATTAGCGTATGGATGCGCCACGGC
CCCTTTGGACGGAAGAAAGAAACTATCCTGTGTAGCTCAGACAAGACTGACATGA
ACCGCCATCATTATTCTTTGTACGTACATGGTTGTCGTCTTATTTTCCTGTTTCGCC
AAGACCCATCCGAAGAAAAGAAGTATAGGCCCGCCGAATTTCATTGGAAACTCAA
CCAAGTGTGCGACGAAGAGTGGCATCATTATGTTCTGAACGTTGAGTTTCCATCCG
TCACACTGTACGTCGACGGTACCAGCCATGAACCATTTAGTGTCACAGAAGACTA
TCCCCTGCACCCGAGTAAAATCGAGACGCAACTGGTTGTCGGCGCATGTTGGCAG
GAATTTAGTGGCGTCGAGAACGATAACGAGACCGAACCCGTCACCGTAGCGTCCG
CCGGCGGGGATCTCCATATGACGCAATTCTTTCGGGGTAACTTGGCCGGGCTGAC
ACTGCGCTCTGGCAAGCTGGCTGACAAGAAAGTTATTGATTGCTTGTACACGTGT
AAAGAAGGCCTTGATCTCCAAGTTCTGGAAGATTCAGGACGAGGGGTCCAAATTC
AGGCTCATCCATCCCAACTGGTGCTTACACTGGAAGGCGAGGATCTGGGAGAGCT
GGACAAAGCTATGCAACATATTTCCTATCTCAATAGTCGCCAATTTCCAACACCTG
GCATCCGACGACTGAAGATTACGTCAACCATTAAATGCTTCAATGAAGCAACATG
TATCAGCGTGCCACCTGTGGACGGATATGTTATGGTACTGCAACCTGAAGAACCA
AAGATTTCCCTCTCTGGGGTTCATCACTTCGCAAGGGCCGCAAGTGAGTTCGAGTC
CTCTGAGGGAGTCTTTCTCTTTCCCGAACTGCGGATAATAAGTACTATTACAAGGG
AAGTCGAACCAGAGGGAGATGGAGCCGAAGATCCAACCGTGCAGGAGTCTCTCG
TATCAGAAGAAATTGTCCATGATCTTGACACGTGCGAAGTGACAGTAGAAGGGGA
AGAACTCAATCATGAACAAGAATCATTGGAAGTAGATATGGCACGATTGCAACAA
AAGGGAATCGAGGTCTCCTCATCCGAGCTTGGTATGACTTTTACTGGAGTAGATA
CGATGGCTTCCTATGAAGAAGTGCTGCATCTTCTCAGATACCGCAATTGGCACGC
GCGTTCTCTGCTGGACAGAAAATTCAAACTGATTTGTAGCGAACTTAACGGACGG
TACATATCTAATGAGTTCAAAGTAGAAGTTAACGTGATTCATACTGCAAATCCTAT
GGAGCATGCGGCCGCTGCCGCCGCTCAACCTCAATTTGTCCATCCCGAGCATAGG
TCATTCGTGGATCTCTCTGGTCATAATTTGGCAAATCCACATCCCTTTGCTGTGGTT
CCATCTACAGCAACTGTAGTTATTGTAGTATGTGTGTCCTTTCTCGTCTTTATGATC
ATATTGGGCGTCTTCCGCATAAGAGCGGCCCACAGGAGAACAATGAGGGACCAA
GATACAGGAAAAGAAAATGAAATGGATTGGGATGATAGCGCACTCACAATAACG
GTGAATCCAATGGAAACGTACGAAGATCAACATTCTAGCGAAGAAGAAGAAGAG
GAAGAGGAAGAGGAAGAGTCAGAAGATGGAGAAGAGGAAGACGATATTACATC
AGCTGAAAGCGAATCTTCAGAAGAAGAAGAAGGTGAACAAGGTGATCCTCAAAA
TGCCACACGCCAACAACAACTCGAATGGGACGATTCTACATTGTCCTAT; Construct
coding sequence from vector 112 (for CLSTN1 VLM); Artificial Sequence
114:
ARVNKHKPWLEPTYHGIVTENDNTVLLDPPLIALDKDAPLRFAESFEVTVTKEGEICG
FKIHGQNVPFDAVVVDKSTGEGVIRSKEKLDCELQKDYSFTIQAYDCGKGPDGTNVK
KSHKATVHIQVNDVNEYAPVFKEKSYKATVIEGKQYDSILRVEAVDADCSPQFSQICS
YEIITPDVPFTVDKDGYIKNTEKLNYGKEHQYKLTVTAYDCGKKRATEDVLVKISIKP
TCTPGWQGWNNRIEYEPGTGALAVFPNIHLETCDEPVASVQATVELETSHIGKGCDR
DTYSEKSLHRLCGAAAGTAELLPSPSGSLNWTMGLPTDNGHDSDQVFEFNGTQAVRI
PDGVVSVSPKEPFTISVWMRHGPFGRKKETILCSSDKTDMNRHHYSLYVHGCRLIFLF
RQDPSEEKKYRPAEFHWKLNQVCDEEWHHYVLNVEFPSVTLYVDGTSHEPFSVTED
YPLHPSKIETQLVVGACWQEFSGVENDNETEPVTVASAGGDLHMTQFFRGNLAGLTL
RSGKLADKKVIDCLYTCKEGLDLQVLEDSGRGVQIQAHPSQLVLTLEGEDLGELDKA
MQHISYLNSRQFPTPGIRRLKITSTIKCFNEATCISVPPVDGYVMVLQPEEPKISLSGVH
HFARAASEFESSEGVFLFPELRIISTITREVEPEGDGAEDPTVQESLVSEEIVHDLDTCEV
TVEGEELNHEQESLEVDMARLQQKGIEVSSSELGMTFTGVDTMASYEEVLHLLRYRN
WHARSLLDRKFKLICSELNGRYISNEFKVEVNVIHTANPMEHAAAAAAQPQFVHPEH
RSFVDLSGHNLANPHPFAVVPST, ATVVIVVCVSFLVFMIILGVF,
RIRAAHRRTMRDQDTGKENEMDWDDSALTITVNPMETYEDQHSSEEEEEEEEEEESE
DGEEEDDITSAESESSEEEEGEQGDPQNATRQQQLEWDDSTLSY; Construct peptide
sequence from vector 112 (for CLSTN1 VLM); Artificial Sequence
115:
TTGGAACTTAATTTGACAGATTCAGAAAATGCCACTTGCCTTTATGCAAAATGGCA
GATGAATTTCACAGTACGCTATGAAACTACAAATAAAACTTATAAAACTGTAACC
ATTTCAGACCATGGCACTGTGACATATAATGGAAGCATTTGTGGGGATGATCAGA
ATGGTCCCAAAATAGCAGTGCAGTTCGGACCTGGCTTTTCCTGGATTGCGAATTTT
ACCAAGGCAGCATCTACTTATTCAATTGACAGCGTCTCATTTTCCTACAACACTGG
TGATAACACAACATTTCCTGATGCTGAAGATAAAGGAATTCTTACTGTTGATGAA
CTTTTGGCCATCAGAATTCCATTGAATGACCTTTTTAGATGCAATAGTTTATCAAC
TTTGGAAAAGAATGATGTTGTCCAACACTACTGGGATGTTCTTGTACAAGCTTTTG
TCCAAAATGGCACAGTGAGCACAAATGAGTTCCTGTGTGATAAAGACAAAACTTC
AACAGTGGCACCCACCATACACACCACTGTGCCATCTCCTACTACAACACCTACTC
CAAAGGAAAAACCAGAAGCTGGAACCTATTCAGTTAATAATGGCAATGATACTTG
TCTGCTGGCTACCATGGGGCTGCAGCTGAACATCACTCAGGATAAGGTTGCTTCA
GTTATTAACATCAACCCCAATACAACTCACTCCACAGGCAGCTGCCGTTCTCACAC
TGCTCTACTTAGACTCAATAGCAGCACCATTAAGTATCTAGACTTTGTCTTTGCTG
TGAAAAATGAAAACCGATTTTATCTGAAGGAAGTGAACATCAGCATGTATTTGGT
TAATGGCTCCGTTTTCAGCATTGCAAATAACAATCTCAGCTACTGGGATGCCCCCC
TGGGAAGTTCTTATATGTGCAACAAAGAGCAGACTGTTTCAGTGTCTGGAGCATTT
CAGATAAATACCTTTGATCTAAGGGTTCAGCCTTTCAATGTGACACAAGGAAAGT
ATTCTACAGCCCAAGAGTGTTCGCTGGATGATGACACCATTCTAATCCCAATTATA
GTTGGTGCTGGTCTTTCAGGCTTGATTATCGTTATAGTGATTGCTAGCTCCCACTG
GTGTTGTAAGAAGGAGGTTCAGGAGACACGGCGCGAGCGCCGCAGGCTCATGTC
GATGGAGATGGAC; Construct coding sequence from vector 135 (for Lamp2 surface-and-
transmembrane domains-PTGERN cytosolic domain chimeric VLM); Artificial Sequence
116:
LELNLTDSENATCLYAKWQMNFTVRYETTNKTYKTVTISDHGTVTYNGSICGDDQN
GPKIAVQFGPGFSWIANFTKAASTYSIDSVSFSYNTGDNTTFPDAEDKGILTVDELLAIR
IPLNDLERCNSLSTLEKNDVVQHYWDVLVQAFVQNGTVSTNEFLCDKDKTSTVAPTI
HTTVPSPTTTPTPKEKPEAGTYSVNNGNDTCLLATMGLQLNITQDKVASVININPNTT
HSTGSCRSHTALLRLNSSTIKYLDFVFAVKNENRFYLKEVNISMYLVNGSVFSIANNN
LSYWDAPLGSSYMCNKEQTVSVSGAFQINTFDLRVQPFNVTQGKYSTAQECSLDDDT,
ILIPIIVGAGLSGLIIVIVIA, SSHWCCKKEVQETRRERRRLMSMEMD; Construct peptide
sequence from vector 135 (for Lamp2 surface-and-transmembrane domains-PTGFRN
cytosolic domain chimeric VLM); Artificial Sequence
117:
TTGGAACTTAATTTGACAGATTCAGAAAATGCCACTTGCCTTTATGCAAAATGGCA
GATGAATTTCACAGTACGCTATGAAACTACAAATAAAACTTATAAAACTGTAACC
ATTTCAGACCATGGCACTGTGACATATAATGGAAGCATTTGTGGGGATGATCAGA
ATGGTCCCAAAATAGCAGTGCAGTTCGGACCTGGCTTTTCCTGGATTGCGAATTTT
ACCAAGGCAGCATCTACTTATTCAATTGACAGCGTCTCATTTTCCTACAACACTGG
TGATAACACAACATTTCCTGATGCTGAAGATAAAGGAATTCTTACTGTTGATGAA
CTTTTGGCCATCAGAATTCCATTGAATGACCTTTTTAGATGCAATAGTTTATCAAC
TTTGGAAAAGAATGATGTTGTCCAACACTACTGGGATGTTCTTGTACAAGCTTTTG
TCCAAAATGGCACAGTGAGCACAAATGAGTTCCTGTGTGATAAAGACAAAACTTC
AACAGTGGCACCCACCATACACACCACTGTGCCATCTCCTACTACAACACCTACTC
CAAAGGAAAAACCAGAAGCTGGAACCTATTCAGTTAATAATGGCAATGATACTTG
TCTGCTGGCTACCATGGGGCTGCAGCTGAACATCACTCAGGATAAGGTTGCTTCA
GTTATTAACATCAACCCCAATACAACTCACTCCACAGGCAGCTGCCGTTCTCACAC
TGCTCTACTTAGACTCAATAGCAGCACCATTAAGTATCTAGACTTTGTCTTTGCTG
TGAAAAATGAAAACCGATTTTATCTGAAGGAAGTGAACATCAGCATGTATTTGGT
TAATGGCTCCGTTTTCAGCATTGCAAATAACAATCTCAGCTACTGGGATGCCCCCC
TGGGAAGTTCTTATATGTGCAACAAAGAGCAGACTGTTTCAGTGTCTGGAGCATTT
CAGATAAATACCTTTGATCTAAGGGTTCAGCCTTTCAATGTGACACAAGGAAAGT
ATTCTACAGCCCAAGAGTGTTCGCTGGATGATGACACCATTCTAATCCCAATTATA
GTTGGTGCTGGTCTTTCAGGCTTGATTATCGTTATAGTGATTGCTAAGTGCGGCTT
CTTCAAGCGAGCCCGCACTCGCGCCCTGTATGAAGCTAAGAGGCAGAAGGCGGA
GATGAAGAGCCAGCCGTCAGAGACAGAGAGGCTGACCGACGACTAC; Construct
coding sequence from vector 140 (for Lamp2 surface-and-transmembrane domains-ITGA3
cytosolic domain chimeric VLM); Artificial Sequence
118:
LELNLTDSENATCLYAKWQMNFTVRYETTNKTYKTVTISDHGTVTYNGSICGDDQN
GPKIAVQFGPGFSWIANFTKAASTYSIDSVSFSYNTGDNTTFPDAEDKGILTVDELLAIR
IPLNDLERCNSLSTLEKNDVVQHYWDVLVQAFVQNGTVSTNEFLCDKDKTSTVAPTI
HTTVPSPTTTPTPKEKPEAGTYSVNNGNDTCLLATMGLQLNITQDKVASVININPNTT
HSTGSCRSHTALLRLNSSTIKYLDFVFAVKNENRFYLKEVNISMYLVNGSVFSIANNN
LSYWDAPLGSSYMCNKEQTVSVSGAFQINTFDLRVQPFNVTQGKYSTAQECSLDDDT,
ILIPIIVGAGLSGLIIVIVIA, KCGFFKRARTRALYEAKRQKAEMKSQPSETERLTDDY;
Construct peptide sequence from vector 140 (for Lamp2 surface-and-transmembrane
domains-ITGA3 cytosolic domain chimeric VLM); Artificial Sequence
119:
TTGGAACTTAATTTGACAGATTCAGAAAATGCCACTTGCCTTTATGCAAAATGGCA
GATGAATTTCACAGTACGCTATGAAACTACAAATAAAACTTATAAAACTGTAACC
ATTTCAGACCATGGCACTGTGACATATAATGGAAGCATTTGTGGGGATGATCAGA
ATGGTCCCAAAATAGCAGTGCAGTTCGGACCTGGCTTTTCCTGGATTGCGAATTTT
ACCAAGGCAGCATCTACTTATTCAATTGACAGCGTCTCATTTTCCTACAACACTGG
TGATAACACAACATTTCCTGATGCTGAAGATAAAGGAATTCTTACTGTTGATGAA
CTTTTGGCCATCAGAATTCCATTGAATGACCTTTTTAGATGCAATAGTTTATCAAC
TTTGGAAAAGAATGATGTTGTCCAACACTACTGGGATGTTCTTGTACAAGCTTTTG
TCCAAAATGGCACAGTGAGCACAAATGAGTTCCTGTGTGATAAAGACAAAACTTC
AACAGTGGCACCCACCATACACACCACTGTGCCATCTCCTACTACAACACCTACTC
CAAAGGAAAAACCAGAAGCTGGAACCTATTCAGTTAATAATGGCAATGATACTTG
TCTGCTGGCTACCATGGGGCTGCAGCTGAACATCACTCAGGATAAGGTTGCTTCA
GTTATTAACATCAACCCCAATACAACTCACTCCACAGGCAGCTGCCGTTCTCACAC
TGCTCTACTTAGACTCAATAGCAGCACCATTAAGTATCTAGACTTTGTCTTTGCTG
TGAAAAATGAAAACCGATTTTATCTGAAGGAAGTGAACATCAGCATGTATTTGGT
TAATGGCTCCGTTTTCAGCATTGCAAATAACAATCTCAGCTACTGGGATGCCCCCC
TGGGAAGTTCTTATATGTGCAACAAAGAGCAGACTGTTTCAGTGTCTGGAGCATTT
CAGATAAATACCTTTGATCTAAGGGTTCAGCCTTTCAATGTGACACAAGGAAAGT
ATTCTACAGCCCAAGAGTGTTCGCTGGATGATGACACCATTCTAATCCCAATTATA
GTTGGTGCTGGTCTTTCAGGCTTGATTATCGTTATAGTGATTGCTGTGATGCAGAG
ACTCTTTCCCCGCATCCCTCACATGAAAGACCCCATCGGTGACAGCTTCCAAAACG
ACAAGCTGGTGGTCTGGGAGGCGGGCAAAGCCGGCCTGGAGGAGTGTCTGGTGA
CTGAAGTACAGGTCGTGCAGAAAACT; Construct coding sequence from vector 141 (for
Lamp2 surface-and-transmembrane domains-IL3RA cytosolic domain chimeric VLM);
Artificial Sequence
120:
LELNLTDSENATCLYAKWQMNFTVRYETTNKTYKTVTISDHGTVTYNGSICGDDQN
GPKIAVQFGPGFSWIANFTKAASTYSIDSVSFSYNTGDNTTFPDAEDKGILTVDELLAIR
IPLNDLFRCNSLSTLEKNDVVQHYWDVLVQAFVQNGTVSTNEFLCDKDKTSTVAPTI
HTTVPSPTTTPTPKEKPEAGTYSVNNGNDTCLLATMGLQLNITQDKVASVININPNTT
HSTGSCRSHTALLRLNSSTIKYLDFVFAVKNENRFYLKEVNISMYLVNGSVFSIANNN
LSYWDAPLGSSYMCNKEQTVSVSGAFQINTFDLRVQPFNVTQGKYSTAQECSLDDDT,
ILIPIIVGAGLSGLIIVIVIA,
VMQRLFPRIPHMKDPIGDSFQNDKLVVWEAGKAGLEECLVTEVQVVQKT; Construct
peptide sequence from vector 141 (for Lamp2 surface-and-transmembrane domains-IL3RA
cytosolic domain chimeric VLM); Artificial Sequence
121:
TTGGAACTTAATTTGACAGATTCAGAAAATGCCACTTGCCTTTATGCAAAATGGCA
GATGAATTTCACAGTACGCTATGAAACTACAAATAAAACTTATAAAACTGTAACC
ATTTCAGACCATGGCACTGTGACATATAATGGAAGCATTTGTGGGGATGATCAGA
ATGGTCCCAAAATAGCAGTGCAGTTCGGACCTGGCTTTTCCTGGATTGCGAATTTT
ACCAAGGCAGCATCTACTTATTCAATTGACAGCGTCTCATTTTCCTACAACACTGG
TGATAACACAACATTTCCTGATGCTGAAGATAAAGGAATTCTTACTGTTGATGAA
CTTTTGGCCATCAGAATTCCATTGAATGACCTTTTTAGATGCAATAGTTTATCAAC
TTTGGAAAAGAATGATGTTGTCCAACACTACTGGGATGTTCTTGTACAAGCTTTTG
TCCAAAATGGCACAGTGAGCACAAATGAGTTCCTGTGTGATAAAGACAAAACTTC
AACAGTGGCACCCACCATACACACCACTGTGCCATCTCCTACTACAACACCTACTC
CAAAGGAAAAACCAGAAGCTGGAACCTATTCAGTTAATAATGGCAATGATACTTG
TCTGCTGGCTACCATGGGGCTGCAGCTGAACATCACTCAGGATAAGGTTGCTTCA
GTTATTAACATCAACCCCAATACAACTCACTCCACAGGCAGCTGCCGTTCTCACAC
TGCTCTACTTAGACTCAATAGCAGCACCATTAAGTATCTAGACTTTGTCTTTGCTG
TGAAAAATGAAAACCGATTTTATCTGAAGGAAGTGAACATCAGCATGTATTTGGT
TAATGGCTCCGTTTTCAGCATTGCAAATAACAATCTCAGCTACTGGGATGCCCCCC
TGGGAAGTTCTTATATGTGCAACAAAGAGCAGACTGTTTCAGTGTCTGGAGCATTT
CAGATAAATACCTTTGATCTAAGGGTTCAGCCTTTCAATGTGACACAAGGAAAGT
ATTCTACAGCCCAAGAGTGTTCGCTGGATGATGACACCATTCTAATCCCAATTATA
GTTGGTGCTGGTCTTTCAGGCTTGATTATCGTTATAGTGATTGCTCGCCTCTCCCGC
AAGGGCCACATGTACCCCGTGCGTAATTACTCCCCCACCGAGATGGTCTGCATCTC
ATCCCTGTTGCCTGATGGGGGTGAGGGGCCCTCTGCCACAGCCAATGGGGGCCTG
TCCAAGGCCAAGAGCCCGGGCCTGACGCCAGAGCCCAGGGAGGACCGTGAGGGG
GATGACCTCACCCTGCACAGCTTCCTCCCT; Construct coding sequence from vector
142 (for Lamp2 surface-and-transmembrane domains-SELPLG cytosolic domain chimeric
VLM); Artificial Sequence
122:
LELNLTDSENATCLYAKWQMNFTVRYETTNKTYKTVTISDHGTVTYNGSICGDDQN
GPKIAVQFGPGFSWIANFTKAASTYSIDSVSFSYNTGDNTTFPDAEDKGILTVDELLAIR
IPLNDLFRCNSLSTLEKNDVVQHYWDVLVQAFVQNGTVSTNEFLCDKDKTSTVAPTI
HTTVPSPTTTPTPKEKPEAGTYSVNNGNDTCLLATMGLQLNITQDKVASVININPNTT
HSTGSCRSHTALLRLNSSTIKYLDFVFAVKNENRFYLKEVNISMYLVNGSVFSIANNN
LSYWDAPLGSSYMCNKEQTVSVSGAFQINTFDLRVQPFNVTQGKYSTAQECSLDDDT,
ILIPIIVGAGLSGLIIVIVIA,
RLSRKGHMYPVRNYSPTEMVCISSLLPDGGEGPSATANGGLSKAKSPGLTPEPREDRE
GDDLTLHSFLP; Construct peptide sequence from vector 142 (for Lamp2 surface-and-
transmembrane domains-SELPLG cytosolic domain chimeric VLM); Artificial Sequence
123:
TTGGAACTTAATTTGACAGATTCAGAAAATGCCACTTGCCTTTATGCAAAATGGCA
GATGAATTTCACAGTACGCTATGAAACTACAAATAAAACTTATAAAACTGTAACC
ATTTCAGACCATGGCACTGTGACATATAATGGAAGCATTTGTGGGGATGATCAGA
ATGGTCCCAAAATAGCAGTGCAGTTCGGACCTGGCTTTTCCTGGATTGCGAATTTT
ACCAAGGCAGCATCTACTTATTCAATTGACAGCGTCTCATTTTCCTACAACACTGG
TGATAACACAACATTTCCTGATGCTGAAGATAAAGGAATTCTTACTGTTGATGAA
CTTTTGGCCATCAGAATTCCATTGAATGACCTTTTTAGATGCAATAGTTTATCAAC
TTTGGAAAAGAATGATGTTGTCCAACACTACTGGGATGTTCTTGTACAAGCTTTTG
TCCAAAATGGCACAGTGAGCACAAATGAGTTCCTGTGTGATAAAGACAAAACTTC
AACAGTGGCACCCACCATACACACCACTGTGCCATCTCCTACTACAACACCTACTC
CAAAGGAAAAACCAGAAGCTGGAACCTATTCAGTTAATAATGGCAATGATACTTG
TCTGCTGGCTACCATGGGGCTGCAGCTGAACATCACTCAGGATAAGGTTGCTTCA
GTTATTAACATCAACCCCAATACAACTCACTCCACAGGCAGCTGCCGTTCTCACAC
TGCTCTACTTAGACTCAATAGCAGCACCATTAAGTATCTAGACTTTGTCTTTGCTG
TGAAAAATGAAAACCGATTTTATCTGAAGGAAGTGAACATCAGCATGTATTTGGT
TAATGGCTCCGTTTTCAGCATTCCAAATAACAATCTCAGCTACTGGGATGCCCCCC
TGGGAAGTTCTTATATGTGCAACAAAGAGCAGACTGTTTCAGTGTCTGGAGCATTT
CAGATAAATACCTTTGATCTAAGGGTTCAGCCTTTCAATGTGACACAAGGAAAGT
ATTCTACAGCCCAAGAGTGTTCGCTGGATGATGACACCATTCTAATCCCAATTATA
GTTGGTGCTGGTCTTTCAGGCTTGATTATCGTTATAGTGATTGCTCTTTTAATGATA
ATTCATGACAGAAGGGAGTTTGCTAAATTTGAAAAGGAGAAAATGAATGCCAAAT
GGGACACGGGTGAAAATCCTATTTATAAGAGTGCCGTAACAACTGTGGTCAATCC
GAAGTATGAGGGAAAA; Construct coding sequence from vector 143 (for Lamp2 surface-
and-transmembrane domains-ITGB1 cytosolic domain chimeric VLM); Artificial Sequence
124:
LELNLTDSENATCLYAKWQMNFTVRYETTNKTYKTVTISDHGTVTYNGSICGDDQN
GPKIAVQFGPGFSWIANFTKAASTYSIDSVSFSYNTGDNTTFPDAEDKGILTVDELLAIR
IPLNDLFRCNSLSTLEKNDVVQHYWDVLVQAFVQNGTVSTNEFLCDKDKTSTVAPTI
HTTVPSPTTTPTPKEKPEAGTYSVNNGNDTCLLATMGLQLNITQDKVASVININPNTT
HSTGSCRSHTALLRLNSSTIKYLDFVFAVKNENRFYLKEVNISMYLVNGSVFSIANNN
LSYWDAPLGSSYMCNKEQTVSVSGAFQINTFDLRVQPFNVTQGKYSTAQECSLDDDT,
ILIPIIVGAGLSGLIIVIVIA,
LLMIIHDRREFAKFEKEKMNAKWDTGENPIYKSAVTTVVNPKYEGK; Construct
peptide sequence from vector 143 (for Lamp2 surface-and-transmembrane domains-ITGB]
cytosolic domain chimeric VLM); Artificial Sequence
125:
TTGGAACTTAATTTGACAGATTCAGAAAATGCCACTTGCCTTTATGCAAAATGGCA
GATGAATTTCACAGTACGCTATGAAACTACAAATAAAACTTATAAAACTGTAACC
ATTTCAGACCATGGCACTGTGACATATAATGGAAGCATTTGTGGGGATGATCAGA
ATGGTCCCAAAATAGCAGTGCAGTTCGGACCTGGCTTTTCCTGGATTGCGAATTTT
ACCAAGGCAGCATCTACTTATTCAATTGACAGCGTCTCATTTTCCTACAACACTGG
TGATAACACAACATTTCCTGATGCTGAAGATAAAGGAATTCTTACTGTTGATGAA
CTTTTGGCCATCAGAATTCCATTGAATGACCTTTTTAGATGCAATAGTTTATCAAC
TTTGGAAAAGAATGATGTTGTCCAACACTACTGGGATGTTCTTGTACAAGCTTTTG
TCCAAAATGGCACAGTGAGCACAAATGAGTTCCTGTGTGATAAAGACAAAACTTC
AACAGTGGCACCCACCATACACACCACTGTGCCATCTCCTACTACAACACCTACTC
CAAAGGAAAAACCAGAAGCTGGAACCTATTCAGTTAATAATGGCAATGATACTTG
TCTGCTGGCTACCATGGGGCTGCAGCTGAACATCACTCAGGATAAGGTTGCTTCA
GTTATTAACATCAACCCCAATACAACTCACTCCACAGGCAGCTGCCGTTCTCACAC
TGCTCTACTTAGACTCAATAGCAGCACCATTAAGTATCTAGACTTTGTCTTTGCTG
TGAAAAATGAAAACCGATTTTATCTGAAGGAAGTGAACATCAGCATGTATTTGGT
TAATGGCTCCGTTTTCAGCATTGCAAATAACAATCTCAGCTACTGGGATGCCCCCC
TGGGAAGTTCTTATATGTGCAACAAAGAGCAGACTGTTTCAGTGTCTGGAGCATTT
CAGATAAATACCTTTGATCTAAGGGTTCAGCCTTTCAATGTGACACAAGGAAAGT
ATTCTACAGCCCAAGAGTGTTCGCTGGATGATGACACCATTCTAATCCCAATTATA
GTTGGTGCTGGTCTTTCAGGCTTGATTATCGTTATAGTGATTGCTCGGATCCGGGC
CGCACATCGGCGGACCATGCGGGATCAGGACACCGGGAAGGAGAACGAGATGGA
CTGGGACGACTCTGCCCTGACCATCACCGTCAACCCCATGGAGACCTATGAGGAC
CAGCACAGCAGTGAGGAGGAGGAGGAAGAGGAAGAGGAAGAGGAAAGCGAGGA
CGGCGAAGAAGAGGATGACATCACCAGCGCCGAGTCGGAGAGCAGCGAGGAGGA
GGAGGGGGAGCAGGGCGACCCCCAGAACGCAACCCGGCAGCAGCAGCTGGAGTG
GGATGACTCCACCCTCAGCTAC; Construct coding sequence from vector 144 (for Lamp2
surface-and-transmembrane domains-CLSTN1 cytosolic domain chimeric VLM); Artificial
Sequence
126:
LELNLTDSENATCLYAKWQMNFTVRYETTNKTYKTVTISDHGTVTYNGSICGDDQN
GPKIAVQFGPGFSWIANFTKAASTYSIDSVSFSYNTGDNTTFPDAEDKGILTVDELLAIR
IPLNDLERCNSLSTLEKNDVVQHYWDVLVQAFVQNGTVSTNEFLCDKDKTSTVAPTI
HTTVPSPTTTPTPKEKPEAGTYSVNNGNDTCLLATMGLQLNITQDKVASVININPNTT
HSTGSCRSHTALLRLNSSTIKYLDFVFAVKNENRFYLKEVNISMYLYNGSVESIANNN
LSYWDAPLGSSYMCNKEQTVSVSGAFQINTFDLRVQPFNVTQGKYSTAQECSLDDDT,
ILIPIIVGAGLSGLIIVIVIA,
RIRAAHRRTMRDQDTGKENEMDWDDSALTITVNPMETYEDQHSSEEEEEEEEEEESE
DGEEEDDITSAESESSEEEEGEQGDPQNATRQQQLEWDDSTLSY; Construct peptide
sequence from vector 144 (for Lamp2 surface-and-transmembrane domains-CLSTNI
cytosolic domain chimeric VLM); Artificial Sequence
127:
CGGGAGGTGCTGGTCCCCGAGGGGCCCTTGTACCGCGTGGCTGGCACAGCTGTCT
CCATCTCCTGCAATGTGACCGGCTATGAGGGCCCTGCCCAGCAGAACTTCGAGTG
GTTCCTGTATAGGCCCGAGGCCCCAGATACTGCACTGGGCATTGTCAGTACCAAG
GATACCCAGTTCTCCTATGCTGTCTTCAAGTCCCGAGTGGTGGCGGGTGAGGTGCA
GGTGCAGCGCCTACAAGGTGATGCCGTGGTGCTCAAGATTGCCCGCCTGCAGGCC
CAGGATGCCGGCATTTATGAGTGCCACACCCCCTCCACTGATACCCGCTACCTGG
GCAGCTACAGCGGCAAGGTGGAGCTGAGAGTTCTTCCAGATGTCCTCCAGGTGTC
TGCTGCCCCCCCAGGGCCCCGAGGCCGCCAGGCCCCAACCTCACCCCCACGCATG
ACGGTGCATGAGGGGCAGGAGCTGGCACTGGGCTGCCTGGCGAGGACAAGCACA
CAGAAGCACACACACCTGGCAGTGTCCTTTGGGCGATCTGTGCCCGAGGCACCAG
TTGGGCGGTCAACTCTGCAGGAAGTGGTGGGAATCCGGTCAGACTTGGCCGTGGA
GGCTGGAGCTCCCTATGCTGAGCGATTGGCTGCAGGGGAGCTTCGTCTGGGCAAG
GAAGGGACCGATCGGTACCGCATGGTAGTAGGGGGTGCCCAGGCAGGGGACGCA
GGCACCTACCACTGCACTGCCGCTGAGTGGATTCAGGATCCTGATGGCAGCTGGG
CCCAGATTGCAGAGAAAAGGGCCGTCCTGGCCCACGTGGATGTGCAGACGCTGTC
CAGCCAGCTGGCAGTGACAGTGGGGCCTGGTGAACGTCGGATCGGCCCAGGGGA
GCCCTTGGAACTGCTGTGCAATGTGTCAGGGGCACTTCCCCCAGCAGGCCGTCAT
GCTGCATACTCTGTAGGTTGGGAGATGGCACCTGCGGGGGCACCTGGGCCCGGCC
GCCTGGTAGCCCAGCTGGACACAGAGGGTGTGGGCAGCCTGGGCCCTGGCTATGA
GGGCCGACACATTGCCATGGAGAAGGTGGCATCCAGAACATACCGGCTACGGCTA
GAGGCTGCCAGGCCTGGTGATGCGGGCACCTACCGCTGCCTCGCCAAAGCCTATG
TTCGAGGGTCTGGGACCCGGCTTCGTGAAGCAGCCAGTGCCCGTTCCCGGCCTCTC
CCTGTACATGTGCGGGAGGAAGGTGTGGTGCTGGAGGCTGTGGCATGGCTAGCAG
GAGGCACAGTGTACCGCGGGGAGACTGCCTCCCTGCTGTGCAACATCTCTGTGCG
GGGTGGCCCCCCAGGACTGCGGCTGGCCGCCAGCTGGTGGGTGGAGCGACCAGA
GGACGGAGAGCTCAGCTCTGTCCCTGCCCAGCTGGTGGGTGGCGTAGGCCAGGAT
GGTGTGGCAGAGCTGGGAGTCCGGCCTGGAGGAGGCCCTGTCAGCGTAGAGCTG
GTGGGGCCCCGAAGCCATCGGCTGAGACTACACAGCTTGGGGCCCGAGGATGAA
GGCGTGTACCACTGTGCCCCCAGCGCCTGGGTGCAGCATGCCGACTACAGCTGGT
ACCAGGCGGGCAGTGCCCGCTCAGGGCCTGTTACAGTCTACCCCTACATGCATGC
CCTGGACACCCTATTTGTGCCTCTGCTGGTGGGTACAGGGGTGGCCCTAGTCACTG
GTGCCACTGTCCTTGGTACCATCACTTGCTGCTTCATGAAGAGGCTTCGAAAACGG;
Construct coding sequence from vector 157 (for IGSF8 VLM); Artificial Sequence
128:
REVLVPEGPLYRVAGTAVSISCNVTGYEGPAQQNFEWFLYRPEAPDTALGIVSTKDTQ
FSYAVFKSRVVAGEVQVQRLQGDAVVLKIARLQAQDAGIYECHTPSTDTRYLGSYSG
KVELRVLPDVLQVSAAPPGPRGRQAPTSPPRMTVHEGQELALGCLARTSTQKHTHLA
VSFGRSVPEAPVGRSTLQEVVGIRSDLAVEAGAPYAERLAAGELRLGKEGTDRYRMV
VGGAQAGDAGTYHCTAAEWIQDPDGSWAQIAEKRAVLAHVDVQTLSSQLAVTVGP
GERRIGPGEPLELLCNVSGALPPAGRHAAYSVGWEMAPAGAPGPGRLVAQLDTEGV
GSLGPGYEGRHIAMEKVASRTYRLRLEAARPGDAGTYRCLAKAYVRGSGTRLREAA
SARSRPLPVHVREEGVVLEAVAWLAGGTVYRGETASLLCNISVRGGPPGLRLAASW
WVERPEDGELSSVPAQLVGGVGQDGVAELGVRPGGGPVSVELVGPRSHRLRLHSLGP
EDEGVYHCAPSAWVQHADYSWYQAGSARSGPVTVYPYMHALDTLFVPLLVGTGVA
LVTGATVLGTITCCFMKRLRKR; Construct peptide sequence from vector 157 (for IGSF8
VLM); Artificial Sequence

TABLE 5
VLM Domains

SEQ ID NO: Sequence; Source
129:
CTGGAGCTGAACCTGACCGACAGCGAGAACGCCACCTGCCTGTACGCCAAGTGGC
AGATGAACTTCACCGTGAGATACGAGACCACCAACAAGACCTACAAGACCGTGA
CCATCAGCGACCACGGCACCGTGACCTACAACGGCAGCATCTGCGGCGACGACCA
GAACGGCCCCAAGATCGCCGTGCAGTTCGGCCCCGGCTTCAGCTGGATCGCCAAC
TTCACCAAGGCCGCCAGCACCTACAGCATCGACAGCGTGAGCTTCAGCTACAACA
CCGGCGACAACACCACCTTCCCCGACGCCGAGGACAAGGGCATCCTGACCGTGGA
CGAGCTGCTGGCCATCAGAATCCCCCTGAACGACCTGTTCAGATGCAACAGCCTG
AGCACCCTGGAGAAGAACGACGTGGTGCAGCACTACTGGGACGTGCTGGTGCAG
GCCTTCGTGCAGAACGGCACCGTGAGCACCAACGAGTTCCTGTGCGACAAGGACA
AGACCAGCACCGTGGCCCCCACCATCCACACCACCGTGCCCAGCCCCACCACCAC
CCCCACCCCCAAGGAGAAGCCCGAGGCCGGCACCTACAGCGTGAACAACGGCAA
CGACACCTGCCTGCTGGCCACCATGGGCCTGCAGCTGAACATCACCCAGGACAAG
GTGGCCAGCGTGATCAACATCAACCCCAACACCACCCACAGCACCGGCAGCTGCA
GAAGCCACACCGCCCTGCTGAGACTGAACAGCAGCACCATCAAGTACCTGGACTT
CGTGTTCGCCGTGAAGAACGAGAACAGATTCTACCTGAAGGAGGTGAACATCAGC
ATGTACCTGGTGAACGGCAGCGTGTTCAGCATCGCCAACAACAACCTGAGCTACT
GGGACGCCCCCCTGGGCAGCAGCTACATGTGCAACAAGGAGCAGACCGTGAGCG
TGAGCGGCGCCTTCCAGATCAACACCTTCGACCTGAGAGTGCAGCCCTTCAACGT
GACCCAGGGCAAGTACAGCACCGCCCAGGAGTGCAGCCTGGACGACGACACC;
Coding sequence of surface domain for fusion proteins produced from LAMP2; Artificial
Sequence
130:
LELNLTDSENATCLYAKWQMNFTVRYETTNKTYKTVTISDHGTVTYNGSICGDDQN
GPKIAVQFGPGFSWIANFTKAASTYSIDSVSFSYNTGDNTTFPDAEDKGILTVDELLAIR
IPLNDLFRCNSLSTLEKNDVVQHYWDVLVQAFVQNGTVSTNEFLCDKDKTSTVAPTI
HTTVPSPTTTPTPKEKPEAGTYSVNNGNDTCLLATMGLQLNITQDKVASVININPNTT
HSTGSCRSHTALLRLNSSTIKYLDFVFAVKNENRFYLKEVNISMYLVNGSVFSIANNN
LSYWDAPLGSSYMCNKEQTVSVSGAFQINTFDLRVQPFNVTQGKYSTAQECSLDDDT;
Peptide sequence of surface domain for fusion proteins produced from LAMP2; Artificial
Sequence
131:
ATCCTGATCCCCATCATCGTGGGCGCCGGCCTGAGCGGCCTGATCATCGTGATCGT
GATCCCC; Coding sequence of transmembrane domain for fusion proteins produced from
LAMP2; Artificial Sequence
132: ILIPIIVGAGLSGLIIVIVIA; Peptide sequence of transmembrane domain for fusion
proteins produced from LAMP2; Artificial Sequence
133: TACGTGATCGGCAGAAGAAAGAGCTACGCCGGCTACCAGACCCTG; Coding
sequence of cytosolic domain for fusion proteins produced from LAMP2; Artificial
Sequence
134: YVIGRRKSYAGYQTL; Peptide sequence of cytosolic domain for fusion proteins
produced from LAMP2; Artificial Sequence
135:
GCCAGAGTGAACAAGCACAAGCCCTGGCTGGAGCCCACCTACCACGGCATCGTGA
CCGAGAACGACAACACCGTGCTGCTGGACCCCCCCCTGATCGCCCTGGACAAGGA
CGCCCCCCTGAGATTCGCCGAGAGCTTCGAGGTGACCGTGACCAAGGAGGGCGAG
ATCTGCGGCTTCAAGATCCACGGCCAGAACGTGCCCTTCGACGCCGTGGTGGTGG
ACAAGAGCACCGGCGAGGGCGTGATCAGAAGCAAGGAGAAGCTGGACTGCGAGC
TGCAGAAGGACTACAGCTTCACCATCCAGGCCTACGACTGCGGCAAGGGCCCCGA
CGGCACCAACGTGAAGAAGAGCCACAAGGCCACCGTGCACATCCAGGTGAACGA
CGTGAACGAGTACGCCCCCGTGTTCAAGGAGAAGAGCTACAAGGCCACCGTGATC
GAGGGCAAGCAGTACGACAGCATCCTGAGAGTGGAGGCCGTGGACGCCGACTGC
AGCCCCCAGTTCAGCCAGATCTGCAGCTACGAGATCATCACCCCCGACGTGCCCT
TCACCGTGGACAAGGACGGCTACATCAAGAACACCGAGAAGCTGAACTACGGCA
AGGAGCACCAGTACAAGCTGACCGTGACCGCCTACGACTGCGGCAAGAAGAGAG
CCACCGAGGACGTGCTGGTGAAGATCAGCATCAAGCCCACCTGCACCCCCGGCTG
GCAGGGCTGGAACAACAGAATCGAGTACGAGCCCGGCACCGGCGCCCTGGCCGT
GTTCCCCAACATCCACCTGGAGACCTGCGACGAGCCCGTGGCCAGCGTGCAGGCC
ACCGTGGAGCTGGAGACCAGCCACATCGGCAAGGGCTGCGACAGAGACACCTAC
AGCGAGAAGAGCCTGCACAGACTGTGCGGCGCCGCCGCCGGCACCGCCGAGCTG
CTGCCCAGCCCCAGCGGCAGCCTGAACTGGACCATGGGCCTGCCCACCGACAACG
GCCACGACAGCGACCAGGTGTTCGAGTTCAACGGCACCCAGGCCGTGAGAATCCC
CGACGGCGTGGTGAGCGTGAGCCCCAAGGAGCCCTTCACCATCAGCGTGTGGATG
AGACACGGCCCCTTCGGCAGAAAGAAGGAGACCATCCTGTGCAGCAGCGACAAG
ACCGACATGAACAGACACCACTACAGCCTGTACGTGCACGGCTGCAGACTGATCT
TCCTGTTCAGACAGGACCCCAGCGAGGAGAAGAAGTACAGACCCGCCGAGTTCCA
CTGGAAGCTGAACCAGGTGTGCGACGAGGAGTGGCACCACTACGTGCTGAACGTG
GAGTTCCCCAGCGTGACCCTGTACGTGGACGGCACCAGCCACGAGCCCTTCAGCG
TGACCGAGGACTACCCCCTGCACCCCAGCAAGATCGAGACCCAGCTGGTGGTGGG
CGCCTGCTGGCAGGAGTTCAGCGGCGTGGAGAACGACAACGAGACCGAGCCCGT
GACCGTGGCCAGCGCCGGCGGCGACCTGCACATGACCCAGTTCTTCAGAGGCAAC
CTGGCCGGCCTGACCCTGAGAAGCGGCAAGCTGGCCGACAAGAAGGTGATCGAC
TGCCTGTACACCTGCAAGGAGGGCCTGGACCTGCAGGTGCTGGAGGACAGCGGCA
GAGGCGTGCAGATCCAGGCCCACCCCAGCCAGCTGGTGCTGACCCTGGAGGGCGA
GGACCTGGGCGAGCTGGACAAGGCCATGCAGCACATCAGCTACCTGAACAGCAG
ACAGTTCCCCACCCCCGGCATCAGAAGACTGAAGATCACCAGCACCATCAAGTGC
TTCAACGAGGCCACCTGCATCAGCGTGCCCCCCGTGGACGGCTACGTGATGGTGC
TGCAGCCCGAGGAGCCCAAGATCAGCCTGAGCGGCGTGCACCACTTCGCCAGAGC
CGCCAGCGAGTTCGAGAGCAGCGAGGGCGTGTTCCTGTTCCCCGAGCTGAGAATC
ATCAGCACCATCACCAGAGAGGTGGAGCCCGAGGGCGACGGCGCCGAGGACCCC
ACCGTGCAGGAGAGCCTGGTGAGCGAGGAGATCGTGCACGACCTGGACACCTGC
GAGGTGACCGTGGAGGGCGAGGAGCTGAACCACGAGCAGGAGAGCCTGGAGGTG
GACATGGCCAGACTGCAGCAGAAGGGCATCGAGGTGAGCAGCAGCGAGCTGGGC
ATGACCTTCACCGGCGTGGACACCATGGCCAGCTACGAGGAGGTGCTGCACCTGC
TGAGATACAGAAACTGGCACGCCAGAAGCCTGCTGGACAGAAAGTTCAAGCTGA
TCTGCAGCGAGCTGAACGGCAGATACATCAGCAACGAGTTCAAGGTGGAGGTGA
ACGTGATCCACACCGCCAACCCCATGGAGCACGCCGCCGCCGCCGCCGCCCAGCC
CCAGTTCGTGCACCCCGAGCACAGAAGCTTCGTGGACCTGAGCGGCCACAACCTG
GCCAACCCCCACCCCTTCGCCGTGGTGCCCAGCACC; Coding sequence of surface
domain for fusion proteins produced from CSTN1; Artificial Sequence
136:
ARVNKHKPWLEPTYHGIVTENDNTVLLDPPLIALDKDAPLRFAESFEVTVTKEGEICG
FKIHGQNVPFDAVVVDKSTGEGVIRSKEKLDCELQKDYSFTIQAYDCGKGPDGTNVK
KSHKATVHIQVNDVNEYAPVFKEKSYKATVIEGKQYDSILRVEAVDADCSPQFSQICS
YEIITPDVPFTVDKDGYIKNTEKLNYGKEHQYKLTVTAYDCGKKRATEDVLVKISIKP
TCTPGWQGWNNRIEYEPGTGALAVFPNIHLETCDEPVASVQATVELETSHIGKGCDR
DTYSEKSLHRLCGAAAGTAELLPSPSGSLNWTMGLPTDNGHDSDQVFEFNGTQAVRI
PDGVVSVSPKEPFTISVWMRHGPFGRKKETILCSSDKTDMNRHHYSLYVHGCRLIFLF
RQDPSEEKKYRPAEFHWKLNQVCDEEWHHYVLNVEFPSVTLYVDGTSHEPFSVTED
YPLHPSKIETQLVVGACWQEFSGVENDNETEPVTVASAGGDLHMTQFFRGNLAGLTL
RSGKLADKKVIDCLYTCKEGLDLQVLEDSGRGVQIQAHPSQLVLTLEGEDLGELDKA
MQHISYLNSRQFPTPGIRRLKITSTIKCFNEATCISVPPVDGYVMVLQPEEPKISLSGVH
HFARAASEFESSEGVFLFPELRIISTITREVEPEGDGAEDPTVQESLVSEEIVHDLDTCEV
TVEGEELNHEQESLEVDMARLQQKGIEVSSSELGMTFTGVDTMASYEEVLHLLRYRN
WHARSLLDRKFKLICSELNGRYISNEFKVEVNVIHTANPMEHAAAAAAQPQFVHPEH
RSFVDLSGHNLANPHPFAVVPST; Peptide sequence of surface domain for fusion proteins
produced from CSTN1; Artificial Sequence
137:
GCCACCGTGGTGATCGTGGTGTGCGTGAGCTTCCTGGTGTTCATGATCATCCTGGG
CGTGTTC; Coding sequence of transmembrane domain for fusion proteins produced from
CSTN1; Artificial Sequence
138: ATVVIVVCVSFLVFMIILGVF; Peptide sequence of transmembrane domain for fusion
proteins produced from CSTN1; Artificial Sequence
139:
AGAATCAGAGCCGCCCACAGAAGAACCATGAGAGACCAGGACACCGGCAAGGAG
AACGAGATGGACTGGGACGACAGCGCCCTGACCATCACCGTGAACCCCATGGAG
ACCTACGAGGACCAGCACAGCAGCGAGGAGGAGGAGGAGGAGGAGGAGGAGGA
GGAGAGCGAGGACGGCGAGGAGGAGGACGACATCACCAGCGCCGAGAGCGAGA
GCAGCGAGGAGGAGGAGGGCGAGCAGGGCGACCCCCAGAACGCCACCAGACAG
CAGCAGCTGGAGTGGGACGACAGCACCCTGAGCTAC; Coding sequence of cytosolie
domain for fusion proteins produced from CSTN1; Artificial Sequence
140:
RIRAAHRRTMRDQDTGKENEMDWDDSALTITVNPMETYEDQHSSEEEEEEEEEEESE
DGEEEDDITSAESESSEEEEGEQGDPQNATRQQQLEWDDSTLSY; Peptide sequence of
cytosolic domain for fusion proteins produced from CSTN1; Artificial Sequence
141
AGCAGCCACTGGTGCTGCAAGAAGGAGGTGCAGGAGACCAGAAGAGAGAGAAG
AAGACTGATGAGCATGGAGATGGAC; Coding sequence of cytosolic domain for fusion
proteins produced from PTGRN; Artificial Sequence
142: SSHWCCKKEVQETRRERRRLMSMEMD; Peptide sequence of cytosolic domain for
fusion proteins produced from PTGRN; Artificial Sequence
143:
AAGTGCGGCTTCTTCAAGAGAGCCAGAACCAGAGCCCTGTACGAGGCCAAGAGA
CAGAAGGCCGAGATGAAGAGCCAGCCCAGCGAGACCGAGAGACTGACCGACGAC
TAC; Coding sequence of cytosolic domain for fusion proteins produced from ITGA3;
Artificial Sequence
144: KCGFFKRARTRALYEAKRQKAEMKSQPSETERLTDDY; Peptide sequence of
cytosolic domain for fusion proteins produced from ITGA3; Artificial Sequence
145:
GTGATGCAGAGACTGTTCCCCAGAATCCCCCACATGAAGGACCCCATCGGCGACA
GCTTCCAGAACGACAAGCTGGTGGTGTGGGAGGCCGGCAAGGCCGGCCTGGAGG
AGTGCCTGGTGACCGAGGTGCAGGTGGTGCAGAAGACC; Coding sequence of
cytosolic domain for fusion proteins produced from IL3RA; Artificial Sequence
146: VMQRLFPRIPHMKDPIGDSFQNDKLVVWEAGKAGLEECLVTEVQVVQKT;
Peptide sequence of cytosolic domain for fusion proteins produced from IL3RA; Artificial
Sequence
147:
AGACTGAGCAGAAAGGGCCACATGTACCCCGTGAGAAACTACAGCCCCACCGAG
ATGGTGTGCATCAGCAGCCTGCTGCCCGACGGCGGCGAGGGCCCCAGCGCCACCG
CCAACGGCGGCCTGAGCAAGGCCAAGAGCCCCGGCCTGACCCCCGAGCCCAGAG
AGGACAGAGAGGGCGACGACCTGACCCTGCACAGCTTCCTGCCC; Coding sequence
of cytosolic domain for fusion proteins produced from SELPL; Artificial Sequence
148:
RLSRKGHMYPVRNYSPTEMVCISSLLPDGGEGPSATANGGLSKAKSPGLTPEPREDRE
GDDLTLHSFLP; Peptide sequence of cytosolic domain for fusion proteins produced from
SELPL; Artificial Sequence
149;
CTGCTGATGATCATCCACGACAGAAGAGAGTTCGCCAAGTTCGAGAAGGAGAAG
ATGAACGCCAAGTGGGACACCGGCGAGAACCCCATCTACAAGAGCGCCGTGACC
ACCGTGGTGAACCCCAAGTACGAGGGCAAG; Coding sequence of cytosolic domain for
fusion proteins produced from ITGB1; Artificial Sequence
150: LLMIIHDRREFAKFEKEKMNAKWDTGENPIYKSAVTTVVNPKYEGK; Peptide
sequence of cytosolic domain for fusion proteins produced from ITGB1; Artificial Sequence

TABLE 6
Targeting Moieties

SEQ ID NO: Sequence; Source
151:
CAAGTGCAGCTCGTCCAATCCGGGGCCGAGGTCAAGAAACCAGGTGCATCCGTCA
AGGTGTCTTGCAAGGCCAGCGGGTATACGTTCACTGATTACGAAATGCATTGGGT
CCGTCAGGCCCCCGGGCAAGGCCTTGAGTGGATGGGCGCTCTTGATCCTAAAACA
GGGGATACTGCTTACAGCCAGAAATTCAAAGGAAGAGTGACACTTACAGCAGAC
AAAAGTACTTCCACCGCATACATGGAACTGAGTTCACTCACATCCGAAGACACTG
CTGTATACTACTGTACAAGATTTTATTCTTATACCTATTGGGGCCAGGGGACTCTC
GTCACAGTGTCTAGCTCCTCAGGTGGAAGCTCCAGATCTTCTAGCTCCGGTGGTGG
CGGCTCCGGCGGGGGGGGCGATGTAGTAATGACTCAATCCCCTCTGTCATTGCCT
GTCACCCCTGGCGAGCCAGCCAGCATCTCTTGCAGATCTTCTCAAAGCCTCGTGCA
TTCCAATGGAAACACGTACCTGCATTGGTACCTGCAGAAACCTGGACAGTCACCA
CAACTGCTGATCTATAAGGTGAGCAACCGGTTCAGTGGAGTGCCGGATAGGTTTA
GCGGATCTGGCAGCGGCACGGACTTCACACTGAAGATAAGCCGTGTCGAAGCTGA
GGATGTTGGAGTCTATTATTGCTCACAGAACACTCATGTGCCACCAACCTTTGGGC
AAGGAACTAAACTTGAGATTAAG; coding sequence of GC33 scFv
152:
QVQLVQSGAEVKKPGASVKVSCKASGYTFTDYEMHWVRQAPGQGLEWMGALDPK
TGDTAYSQKFKGRVTLTADKSTSTAYMELSSLTSEDTAVYYCTRFYSYTYWGQGTLV
TVSSSSGGSSRSSSSGGGGSGGGGDVVMTQSPLSLPVTPGEPASISCRSSQSLVHSNGN
TYLHWYLQKPGQSPQLLIYKVSNRFSGVPDRESGSGSGTDFTLKISRVEAEDVGVYYC
SQNTHVPPTFGQGTKLEIK; peptide sequence of GC33 scFv
153:
CAAATGCAGCTGGTTCAAAGTGGTGCTGAGGTCAAAAAACCAGGCGCGAGCGTA
AAACTGTCCTGTAAAGCCAGCGGATACACCTTCTCCAGCTACTGGATGCACTGGG
TCCGACAGGCCCCAGGGCAGAGGCTCGAATGGATGGGCGAGATCAACCCCGGCA
ATGGTCACACCAATTACAATGAAAAGTTCAAGAGCCGCGTGACCATTACTGTCGA
TAAATCTGCATCTACAGCATACATGGAACTTTCCAGCCTTAGATCAGAGGACACA
GCCGTATATTATTGTGCCAAGATCTGGGGACCGTCCCTTACAAGTCCTTTCGATTA
CTGGGGTCAGGGGACGCTTGTAACGGTATCCGGGGGGGGAGGTTCCGGCGGAGG
CGGTTCAGGAGGGGGCGGTTCCGGGGGCGGTGGATCTAATTTTATGCTTACGCAA
CCCCCGTCTGTAAGTGTTTCCCCAGGGAAAACCGCTCGAATAACCTGTCGAGGAG
ACAACCTCGGCGATGTTAATGTCCATTGGTATCAACAGCGACCTGGGCAAGCCCC
GGTTTTGGTCATGTACTATGATGCGGACCGCCCCAGTGGCATACCGGAACGGTTC
AGTGGAAGTAACTCTGGCAATACTGCAACGCTGACCATCAGTGGCGTTGAGGCGG
GTGACGAGGCAGATTATTACTGTCAGGTCTGGGACCGGACCAGTGAGTATGTTTT
CGGAACCGGCACAAAAGTAACTGTACTCGGG; coding sequence of 6A6 scFv
154:
QMQLVQSGAEVKKPGASVKLSCKASGYTFSSYWMHWVRQAPGQRLEWMGEINPGN
GHTNYNEKFKSRVTITVDKSASTAYMELSSLRSEDTAVYYCAKIWGPSLTSPFDYWG
QGTLVTVSGGGGSGGGGSGGGGSGGGGSNFMLTQPPSVSVSPGKTARITCRGDNLGD
VNVHWYQQRPGQAPVLVMYYDADRPSGIPERFSGSNSGNTATLTISGVEAGDEADY
YCQVWDRTSEYVFGTGTKVTVLG; peptide sequence of 6A6 scFv
155:
GAAGTGCAGCTTGTAGAAAGTGGGGGGGGACTGGTACAGCCGGGCGGGAGCCTC
AGATTGTCATGCGCCGCTTCTGGTTTCACTTTTTCTTCCTACGGTATGTCCTGGGTT
AGACAAGCTCCTGGGAAGGGTCTTGAGTGGGTGGCTACAATTACTAGTGGTGGTT
CATACACGTACTATGTTGACAGTGTTAAGGGGCGATTTACTATAAGTAGAGATAA
TGCCAAGAACACACTCTACCTTCAGATGAATAGCTTGCGGGCGGAAGATACAGCA
GTTTATTATTGCGTTCGGATTGGCGAGGACGCACTCGACTATTGGGGACAAGGGA
CTCTTGTTACGGTGTCTAGTGGGGGGGGAGGTTCCGGCGGAGGCGGTTCAGGAGG
GGGCGGTTCCGGGGGCGGTGGATCTGACATCCAGATGACGCAATCCCCAAGTTCA
CTTAGCGCTTCAGTCGGCGACCGCGTTACCATAACATGCAGAGCAAGTCAAGACA
TTGCAGGGAGTCTTAATTGGTTGCAGAAGCCAGGTAAAGCTATAAAGCGCCTTAT
ATATGCCACCAGCAGTCTGGATTCTGGTGTACCGAAGAGATTCAGCGGTTCCAGA
AGTGGCAGTGACTATACTCTGACCATTTCTTCTCTCCAGCCTGAAGATTTCGCCAC
TTACTATTGTCTGCAATATGGTTCTTTCCCACCAACATTCGGACAAGGTACTAAGG
TCGAGATTAAG; coding sequence of ALAC scFv
156
EVQLVESGGGLVQPGGSLRLSCAASGFTFSSYGMSWVRQAPGKGLEWVATITSGGSY
TYYVDSVKGRFTISRDNAKNTLYLQMNSLRAEDTAVYYCVRIGEDALDYWGQGTLV
TVSSGGGGSGGGGSGGGGSGGGGSDIQMTQSPSSLSASVGDRVTITCRASQDIAGSLN
WLQKPGKAIKRLIYATSSLDSGVPKRFSGSRSGSDYTLTISSLQPEDFATYYCLQYGSF
PPTFGQGTKVEIK; peptide sequence of ALAC scFv
157: TGCCTGGTGAGCGGCGGCATGGCCTGC; coding sequence of peptide selected from
a random peptide library; Artificial Sequence
158: CLVSGGMAC; peptide sequence of peptide selected from a random peptide library;
Artificial Sequence
159: TGCCTGGTGAGCGGCTGCAACACCTGC; coding sequence of peptide selected from
a random peptide library; Artificial Sequence
160: CLVSGCNTC; peptide sequence of peptide selected from a random peptide library;
Artificial Sequence
161: TGCGACCTGGTGAGCGGCTACGGCTGC; coding sequence of peptide selected from
a random peptide library; Artificial Sequence
162: CDLVSGYGC, peptide sequence of peptide selected from a random peptide library;
Artificial Sequence
163: TGCCTGGTGAGCACCAGCGCCACCTGC; coding sequence of peptide selected from
a random peptide library; Artificial Sequence
164: CLVSTSATC; peptide sequence of peptide selected from a random peptide library;
Artificial Sequence
165: TGCACCGCCCTGGTGAGCCAGACCTGC; coding sequence of peptide selected from
a random peptide library; Artificial Sequence
166: CTALVSQTC; peptide sequence of peptide selected from a random peptide library;
Artificial Sequence
167: TGCTGGCTGGTGAGCGGCATCGGCTGC; coding sequence of peptide selected from
a random peptide library; Artificial Sequence
168: CWLVSGIGC; peptide sequence of peptide selected from a random peptide library;
Artificial Sequence
169: TGCCTGGTGAGCAGCGTGTTCCCCTGC; coding sequence of peptide selected from
a random peptide library; Artificial Sequence
170: CLVSSVFPC; peptide sequence of peptide selected from a random peptide library;
Artificial Sequence
171: TGCCCCAGCCTGGTGAGCAGCGTGTGC; coding sequence of peptide selected from
a random peptide library; Artificial Sequence
172: CPSLVSSVC; peptide sequence of peptide selected from a random peptide library;
Artificial Sequence
173: TGCGGCGTGAGCCTGGTGAGCACCTGC; coding sequence of peptide selected from
a random peptide library; Artificial Sequence
174: CGVSLVSTC; peptide sequence of peptide selected from a random peptide library;
Artificial Sequence
175: TGCCAGCTGGTGAGCGGCGAGCCCTGC; coding sequence of peptide selected from
a random peptide library; Artificial Sequence
176: CQLVSGEPC; peptide sequence of peptide selected from a random peptide library;
Artificial Sequence
177: TGCAACCTGGTGAGCAGAAGACTGTGC; coding sequence of peptide selected from
a random peptide library; Artificial Sequence
178: CNLVSRRLC; peptide sequence of peptide selected from a random peptide library;
Artificial Sequence
179: TGCCTGGTGAGCTGGAGAGGCAGCTGC; coding sequence of peptide selected from
a random peptide library; Artificial Sequence
180: CLVSWRGSC; peptide sequence of peptide selected from a random peptide library;
Artificial Sequence
181: TGCGACCACTTCCTGGTGAGCCCCTGC; coding sequence of peptide selected from
a random peptide library; Artificial Sequence
182: CDHFLVSPC; peptide sequence of peptide selected from a random peptide library;
Artificial Sequence
183: TGCGGCAGAGGCCTGGTGAGCCTGTGC; coding sequence of peptide selected from
a random peptide library: Artificial Sequence
184: CGRGLVSLC; peptide sequence of peptide selected from a random peptide library;
Artificial Sequence
185: TGCTTCCCCGTGGCCCTGGTGAGCTGC; coding sequence of peptide selected from a
random peptide library; Artificial Sequence
186: CFPVALVSC; peptide sequence of peptide selected from a random peptide library;
Artificial Sequence
187: TGCAGATGGAGCAGCCTGGTGAGCTGC, coding sequence of peptide selected from
a random peptide library; Artificial Sequence
188: CRWSSLVSC; peptide sequence of peptide selected from a random peptide library;
Artificial Sequence
189: TGCTGGAGCAAGAGCCTGGTGAGCTGC; coding sequence of peptide selected from
a random peptide library, Artificial Sequence
190: CWSKSLVSC; peptide sequence of peptide selected from a random peptide library;
Artificial Sequence
191: TGCCCCGGCAGAAGCCTGGTGAGCTGC; coding sequence of peptide selected from
a random peptide library; Artificial Sequence
192: CPGRSLVSC; peptide sequence of peptide selected from a random peptide library;
Artificial Sequence
193: ACCCACAGACCCCCCATGTGGAGCCCCGTGTGGCCC; coding sequence of
peptide selected from a random peptide library; Artificial Sequence
194: THRPPMWSPVWP; peptide sequence of peptide selected from a random peptide library;
Artificial Sequence
195: ACCCACGTGAGCCCCAACCAGGGCGGCCTGCCCAGC; coding sequence of PEPN
196: THVSPNQGGLPS; peptide sequence of PEPN

TABLE 7
Fusion Protein Comprising Isopeptide Domain and VLM or Chimeric VLM

SEQ ID NO: Sequence; Source
197:
GACTACAAAGACCACGACGGGGATTATAAAGATCATGACATCGATTACAAGGAT
GACGATGATAAGGGCTCTGGCGACAGCGCAACACACATCAAGTTCTCAAAACGG
GATGAGGATGGAAAAGAACTGGCCGGAGCGACAATGGAACTGAGAGATTCTTCC
GGCAAGACTATCTCCACATGGATTAGTGACGGGCAAGTCAAAGACTTCTACTTGT
ACCCCGGTAAGTACACCTTCGTTGAGACTGCCGCTCCTGACGGGTATGAAGTCGC
CACGGCGATCACATTCACTGTGAATGAACAGGGACAGGTGACGGTCAATGGAGG
ATCCCCCGCCAACCTGAAGGCCCTGGAGGCCCAGAAGCAGAAGGAGCAGAGACA
GGCCGCCGAGGAGCTGGCCAACGCCAAGAAGCTGAAGGAGCAGCTGGAGAAGCG
GGAGGTGCTGGTCCCCGAGGGGCCCTTGTACCGCGTGGCTGGCACAGCTGTCTCC
ATCTCCTGCAATGTGACCGGCTATGAGGGCCCTGCCCAGCAGAACTTCGAGTGGT
TCCTGTATAGGCCCGAGGCCCCAGATACTGCACTGGGCATTGTCAGTACCAAGGA
TACCCAGTTCTCCTATGCTGTCTTCAAGTCCCGAGTGGTGGCGGGTGAGGTGCAGG
TGCAGCGCCTACAAGGTGATGCCGTGGTGCTCAAGATTGCCCGCCTGCAGGCCCA
GGATGCCGGCATTTATGAGTGCCACACCCCCTCCACTGATACCCGCTACCTGGGC
AGCTACAGCGGCAAGGTGGAGCTGAGAGTTCTTCCAGATGTCCTCCAGGTGTCTG
CTGCCCCCCCAGGGCCCCGAGGCCGCCAGGCCCCAACCTCACCCCCACGCATGAC
GGTGCATGAGGGGCAGGAGCTGGCACTGGGCTGCCTGGCGAGGACAAGCACACA
GAAGCACACACACCTGGCAGTGTCCTTTGGGCGATCTGTGCCCGAGGCACCAGTT
GGGCGGTCAACTCTGCAGGAAGTGGTGGGAATCCGGTCAGACTTGGCCGTGGAGG
CTGGAGCTCCCTATGCTGAGCGATTGGCTGCAGGGGAGCTTCGTCTGGGCAAGGA
AGGGACCGATCGGTACCGCATGGTAGTAGGGGGTGCCCAGGCAGGGGACGCAGG
CACCTACCACTGCACTGCCGCTGAGTGGATTCAGGATCCTGATGGCAGCTGGGCC
CAGATTGCAGAGAAAAGGGCCGTCCTGGCCCACGTGGATGTGCAGACGCTGTCCA
GCCAGCTGGCAGTGACAGTGGGGCCTGGTGAACGTCGGATCGGCCCAGGGGAGC
CCTTGGAACTGCTGTGCAATGTGTCAGGGGCACTTCCCCCAGCAGGCCGTCATGCT
GCATACTCTGTAGGTTGGGAGATGGCACCTGCGGGGGCACCTGGGCCCGGCCGCC
TGGTAGCCCAGCTGGACACAGAGGGTGTGGGCAGCCTGGGCCCTGGCTATGAGGG
CCGACACATTGCCATGGAGAAGGTGGCATCCAGAACATACCGGCTACGGCTAGAG
GCTGCCAGGCCTGGTGATGCGGGCACCTACCGCTGCCTCGCCAAAGCCTATGTTC
GAGGGTCTGGGACCCGGCTTCGTGAAGCAGCCAGTGCCCGTTCCCGGCCTCTCCC
TGTACATGTGCGGGAGGAAGGTGTGGTGCTGGAGGCTGTGGCATGGCTAGCAGGA
GGCACAGTGTACCGCGGGGAGACTGCCTCCCTGCTGTGCAACATCTCTGTGCGGG
GTGGCCCCCCAGGACTGCGGCTGGCCGCCAGCTGGTGGGTGGAGCGACCAGAGG
ACGGAGAGCTCAGCTCTGTCCCTGCCCAGCTGGTGGGTGGCGTAGGCCAGGATGG
TGTGGCAGAGCTGGGAGTCCGGCCTGGAGGAGGCCCTGTCAGCGTAGAGCTGGTG
GGGCCCCGAAGCCATCGGCTGAGACTACACAGCTTGGGGCCCGAGGATGAAGGC
GTGTACCACTGTGCCCCCAGCGCCTGGGTGCAGCATGCCGACTACAGCTGGTACC
AGGCGGGCAGTGCCCGCTCAGGGCCTGTTACAGTCTACCCCTACATGCATGCCCT
GGACACCCTATTTGTGCCTCTGCTGGTGGGTACAGGGGTGGCCCTAGTCACTGGTG
CCACTGTCCTTGGTACCATCACTTGCTGCTTCATGAAGAGGCTTCGAAAACGG;
Coding sequence of Isopeptide(1) domain-IGSF8 VLM.
198:
DYKDHDGDYKDHDIDYKDDDDKGSGDSATHIKFSKRDEDGKELAGATMELRDSSGK
TISTWISDGQVKDFYLYPGKYTFVETAAPDGYEVATAITFTVNEQGQVTVNGGSPANL
KALEAQKQKEQRQAAEELANAKKLKEQLEKREVLVPEGPLYRVAGTAVSISCNVTG
YEGPAQQNFEWFLYRPEAPDTALGIVSTKDTQFSYAVFKSRVVAGEVQVQRLQGDA
VVLKIARLQAQDAGIYECHTPSTDTRYLGSYSGKVELRVLPDVLQVSAAPPGPRGRQ
APTSPPRMTVHEGQELALGCLARTSTQKHTHLAVSFGRSVPEAPVGRSTLQEVVGIRS
DLAVEAGAPYAERLAAGELRLGKEGTDRYRMVVGGAQAGDAGTYHCTAAEWIQDP
DGSWAQIAEKRAVLAHVDVQTLSSQLAVTVGPGERRIGPGEPLELLCNVSGALPPAG
RHAAYSVGWEMAPAGAPGPGRLVAQLDTEGVGSLGPGYEGRHIAMEKVASRTYRLR
LEAARPGDAGTYRCLAKAYVRGSGTRLREAASARSRPLPVHVREEGVVLEAVAWLA
GGTVYRGETASLLCNISVRGGPPGLRLAASWWVERPEDGELSSVPAQLVGGVGQDG
VAELGVRPGGGPVSVELVGPRSHRLRLHSLGPEDEGVYHCAPSAWVQHADYSWYQA
GSARSGPVTVYPYMHALDTLFVPLLVGTGVALVTGATVLGTITCCEMKRLRKR;
Peptide sequence of Isopeptide(1) domain-IGSF8 VLM
199:
GACTACAAAGACCACGACGGGGATTATAAAGATCATGACATCGATTACAAGGAT
GACGATGATAAGGGCTCTGGCGGATCCCATATGAAGCCGCTGCGTGGTGCCGTGT
TTAGCCTGCAGAAACAGCATCCCGACTATCCCGATATCTATGGCGCGATTGATCA
GAATGGGACCTATCAAAATGTGCGTACCGGCGAAGATGGTAAACTGACCTTTAAG
AATCTGAGCGATGGCAAATATCGCCTGTTTGAAAATAGCGAACCCGCTGGCTATA
AACCGGTGCAGAATAAGCCGATTGTGGCGTTTCAGATTGTGAATGGCGAAGTGCG
TGATGTGACCAGCATTGTGCCGCAGGATATTCCGGCTACATATGAATTTACCAAC
GGTAAACATTATATCACCAATGAACCGATACCGCCGAAAGGATCCCCCGCCAACC
TGAAGGCCCTGGAGGCCCAGAAGCAGAAGGAGCAGAGACAGGCCGCCGAGGAGC
TGGCCAACGCCAAGAAGCTGAAGGAGCAGCTGGAGAAGCGGGAGGTGCTGGTCC
CCGAGGGGCCCTTGTACCGCGTGGCTGGCACAGCTGTCTCCATCTCCTGCAATGTG
ACCGGCTATGAGGGCCCTGCCCAGCAGAACTTCGAGTGGTTCCTGTATAGGCCCG
AGGCCCCAGATACTGCACTGGGCATTGTCAGTACCAAGGATACCCAGTTCTCCTA
TGCTGTCTTCAAGTCCCGAGTGGTGGCGGGTGAGGTGCAGGTGCAGCGCCTACAA
GGTGATGCCGTGGTGCTCAAGATTGCCCGCCTGCAGGCCCAGGATGCCGGCATTT
ATGAGTGCCACACCCCCTCCACTGATACCCGCTACCTGGGCAGCTACAGCGGCAA
GGTGGAGCTGAGAGTTCTTCCAGATGTCCTCCAGGTGTCTGCTGCCCCCCCAGGGC
CCCGAGGCCGCCAGGCCCCAACCTCACCCCCACGCATGACGGTGCATGAGGGGCA
GGAGCTGGCACTGGGCTGCCTGGCGAGGACAAGCACACAGAAGCACACACACCT
GGCAGTGTCCTTTGGGCGATCTGTGCCCGAGGCACCAGTTGGGGGGTCAACTCTG
CAGGAAGTGGTGGGAATCCGGTCAGACTTGGCCGTGGAGGCTGGAGCTCCCTATG
CTGAGCGATTGGCTGCAGGGGAGCTTCGTCTGGGCAAGGAAGGGACCGATCGGTA
CCGCATGGTAGTAGGGGGTGCCCAGGCAGGGGACGCAGGCACCTACCACTGCACT
GCCGCTGAGTGGATTCAGGATCCTGATGGCAGCTGGGCCCAGATTGCAGAGAAAA
GGGCCGTCCTGGCCCACGTGGATGTGCAGACGCTGTCCAGCCAGCTGGCAGTGAC
AGTGGGGCCTGGTGAACGTCGGATCGGCCCAGGGGAGCCCTTGGAACTGCTGTGC
AATGTGTCAGGGGCACTTCCCCCAGCAGGCCGTCATGCTGCATACTCTGTAGGTTG
GGAGATGGCACCTGCGGGGGCACCTGGGCCCGGCCGCCTGGTAGCCCAGCTGGAC
ACAGAGGGTGTGGGCAGCCTGGGCCCTGGCTATGAGGGCCGACACATTGCCATGG
AGAAGGTGGCATCCAGAACATACCGGCTACGGCTAGAGGCTGCCAGGCCTGGTG
ATGCGGGCACCTACCGCTGCCTCGCCAAAGCCTATGTTCGAGGGTCTGGGACCCG
GCTTCGTGAAGCAGCCAGTGCCCGTTCCCGGCCTCTCCCTGTACATGTGCGGGAG
GAAGGTGTGGTGCTGGAGGCTGTGGCATGGCTAGCAGGAGGCACAGTGTACCGC
GGGGAGACTGCCTCCCTGCTGTGCAACATCTCTGTGCGGGGTGGCCCCCCAGGAC
TGCGGCTGGCCGCCAGCTGGTGGGTGGAGCGACCAGAGGACGGAGAGCTCAGCT
CTGTCCCTGCCCAGCTGGTGGGTGGCGTAGGCCAGGATGGTGTGGCAGAGCTGGG
AGTCCGGCCTGGAGGAGGCCCTGTCAGCGTAGAGCTGGTGGGGCCCCGAAGCCAT
CGGCTGAGACTACACAGCTTGGGGCCCGAGGATGAAGGCGTGTACCACTGTGCCC
CCAGCGCCTGGGTGCAGCATGCCGACTACAGCTGGTACCAGGCGGGCAGTGCCCG
CTCAGGGCCTGTTACAGTCTACCCCTACATGCATGCCCTGGACACCCTATTTGTGC
CTCTGCTGGTGGGTACAGGGGTGGCCCTAGTCACTGGTGCCACTGTCCTTGGTACC
ATCACTTGCTGCTTCATGAAGAGGCTTCGAAAACGG; Coding sequence of
Isopeptide(2) domain-IGSF8 VLM
200:
DYKDHDGDYKDHDIDYKDDDDKGSGGSHMKPLRGAVFSLQKQHPDYPDIYGAIDQN
GTYQNVRTGEDGKLTFKNLSDGKYRLFENSEPAGYKPVQNKPIVAFQIVNGEVRDVT
SIVPQDIPATYEFTNGKHYITNEPIPPKGSPANLKALEAQKQKEQRQAAEELANAKKL
KEQLEKREVLVPEGPLYRVAGTAVSISCNVTGYEGPAQQNFEWFLYRPEAPDTALGIV
STKDTQFSYAVFKSRVVAGEVQVQRLQGDAVVLKIARLQAQDAGIYECHTPSTDTRY
LGSYSGKVELRVLPDVLQVSAAPPGPRGRQAPTSPPRMTVHEGQELALGCLARTSTQ
KHTHLAVSFGRSVPEAPVGRSTLQEVVGIRSDLAVEAGAPYAERLAAGELRLGKEGT
DRYRMVVGGAQAGDAGTYHCTAAEWIQDPDGSWAQIAEKRAVLAHVDVQTLSSQL
AVTVGPGERRIGPGEPLELLCNVSGALPPAGRHAAYSVGWEMAPAGAPGPGRLVAQL
DTEGVGSLGPGYEGRHIAMEKVASRTYRLRLEAARPGDAGTYRCLAKAYVRGSGTR
LREAASARSRPLPVHVREEGVVLEAVAWLAGGTVYRGETASLLCNISVRGGPPGLRL
AASWWVERPEDGELSSVPAQLVGGVGQDGVAELGVRPGGGPVSVELVGPRSHRLRL
HSLGPEDEGVYHCAPSAWVQHADYSWYQAGSARSGPVTVYPYMHALDTLFVPLLV
GTGVALVTGATVLGTITCCFMKRLRKR; Peptide sequence of Isopeptide(2) domain-
IGSF8 VLM
201:
GACTACAAAGACCACGACGGGGATTATAAAGATCATGACATCGATTACAAGGAT
GACGATGATAAGGGCTCTGGCGACAGCGCAACACACATCAAGTTCTCAAAACGG
GATGAGGATGGAAAAGAACTGGCCGGAGCGACAATGGAACTGAGAGATTCTTCC
GGCAAGACTATCTCCACATGGATTAGTGACGGGCAAGTCAAAGACTTCTACTTGT
ACCCCGGTAAGTACACCTTCGTTGAGACTGCCGCTCCTGACGGGTATGAAGTCGC
CACGGCGATCACATTCACTGTGAATGAACAGGGACAGGTGACGGTCAATGGAGG
CGGTGGCGGCTCTGGCGGAGGAGGCTCAGGATCCCATATGAAGCCGCTGCGTGGT
GCCGTGTTTAGCCTGCAGAAACAGCATCCCGACTATCCCGATATCTATGGCGCGA
TTGATCAGAATGGGACCTATCAAAATGTGCGTACCGGCGAAGATGGTAAACTGAC
CTTTAAGAATCTGAGCGATGGCAAATATCGCCTGTTTGAAAATAGCGAACCCGCT
GGCTATAAACCGGTGCAGAATAAGCCGATTGTGGCGTTTCAGATTGTGAATGGCG
AAGTGCGTGATGTGACCAGCATTGTGCCGCAGGATATTCCGGCTACATATGAATT
TACCAACGGTAAACATTATATCACCAATGAACCGATACCGCCGAAAGGATCCCCC
GCCAACCTGAAGGCCCTGGAGGCCCAGAAGCAGAAGGAGCAGAGACAGGCCGCC
GAGGAGCTGGCCAACGCCAAGAAGCTGAAGGAGCAGCTGGAGAAGCGGGAGGTG
CTGGTCCCCGAGGGGCCCTTGTACCGCGTGGCTGGCACAGCTGTCTCCATCTCCTG
CAATGTGACCGGCTATGAGGGCCCTGCCCAGCAGAACTTCGAGTGGTTCCTGTAT
AGGCCCGAGGCCCCAGATACTGCACTGGGCATTGTCAGTACCAAGGATACCCAGT
TCTCCTATGCTGTCTTCAAGTCCCGAGTGGTGGCGGGTGAGGTGCAGGTGCAGCG
CCTACAAGGTGATGCCGTGGTGCTCAAGATTGCCCGCCTGCAGGCCCAGGATGCC
GGCATTTATGAGTGCCACACCCCCTCCACTGATACCCGCTACCTGGGCAGCTACA
GCGGCAAGGTGGAGCTGAGAGTTCTTCCAGATGTCCTCCAGGTGTCTGCTGCCCC
CCCAGGGCCCCGAGGCCGCCAGGCCCCAACCTCACCCCCACGCATGACGGTGCAT
GAGGGGCAGGAGCTGGCACTGGGCTGCCTGGCGAGGACAAGCACACAGAAGCAC
ACACACCTGGCAGTGTCCTTTGGGCGATCTGTGCCCGAGGCACCAGTTGGGCGGT
CAACTCTGCAGGAAGTGGTGGGAATCCGGTCAGACTTGGCCGTGGAGGCTGGAGC
TCCCTATGCTGAGCGATTGGCTGCAGGGGAGCTTCGTCTGGGCAAGGAAGGGACC
GATCGGTACCGCATGGTAGTAGGGGGTGCCCAGGCAGGGGACGCAGGCACCTAC
CACTGCACTGCCGCTGAGTGGATTCAGGATCCTGATGGCAGCTGGGCCCAGATTG
CAGAGAAAAGGGCCGTCCTGGCCCACGTGGATGTGCAGACGCTGTCCAGCCAGCT
GGCAGTGACAGTGGGGCCTGGTGAACGTCGGATCGGCCCAGGGGAGCCCTTGGA
ACTGCTGTGCAATGTGTCAGGGGCACTTCCCCCAGCAGGCCGTCATGCTGCATACT
CTGTAGGTTGGGAGATGGCACCTGCGGGGGCACCTGGGCCCGGCCGCCTGGTAGC
CCAGCTGGACACAGAGGGTGTGGGCAGCCTGGGCCCTGGCTATGAGGGCCGACA
CATTGCCATGGAGAAGGTGGCATCCAGAACATACCGGCTACGGCTAGAGGCTGCC
AGGCCTGGTGATGCGGGCACCTACCGCTGCCTCGCCAAAGCCTATGTTCGAGGGT
CTGGGACCCGGCTTCGTGAAGCAGCCAGTGCCCGTTCCCGGCCTCTCCCTGTACAT
GTGCGGGAGGAAGGTGTGGTGCTGGAGGCTGTGGCATGGCTAGCAGGAGGCACA
GTGTACCGCGGGGAGACTGCCTCCCTGCTGTGCAACATCTCTGTGCGGGGTGGCC
CCCCAGGACTGCGGCTGGCCGCCAGCTGGTGGGTGGAGCGACCAGAGGACGGAG
AGCTCAGCTCTGTCCCTGCCCAGCTGGTGGGTGGCGTAGGCCAGGATGGTGTGGC
AGAGCTGGGAGTCCGGCCTGGAGGAGGCCCTGTCAGCGTAGAGCTGGTGGGGCC
CCGAAGCCATCGGCTGAGACTACACAGCTTGGGGCCCGAGGATGAAGGCGTGTAC
CACTGTGCCCCCAGCGCCTGGGTGCAGCATGCCGACTACAGCTGGTACCAGGCGG
GCAGTGCCCGCTCAGGGCCTGTTACAGTCTACCCCTACATGCATGCCCTGGACACC
CTATTTGTGCCTCTGCTGGTGGGTACAGGGGTGGCCCTAGTCACTGGTGCCACTGT
CCTTGGTACCATCACTTGCTGCTTCATGAAGAGGCTTCGAAAACGG; Coding
sequence of Isopeptide(1) + Isopeptide(2) domains-IGSF8 VLM
202:
DYKDHDGDYKDHDIDYKDDDDKGSGDSATHIKFSKRDEDGKELAGATMELRDSSGK
TISTWISDGQVKDFYLYPGKYTFVETAAPDGYEVATAITFTVNEQGQVTVNGGGGGS
GGGGSGSHMKPLRGAVFSLQKQHPDYPDIYGAIDQNGTYQNVRTGEDGKLTFKNLS
DGKYRLFENSEPAGYKPVQNKPIVAFQIVNGEVRDVTSIVPQDIPATYEFTNGKHYITN
EPIPPKGSPANLKALEAQKQKEQRQAAEELANAKKLKEQLEKREVLVPEGPLYRVAG
TAVSISCNVTGYEGPAQQNFEWFLYRPEAPDTALGIVSTKDTQFSYAVFKSRVVAGEV
QVQRLQGDAVVLKIARLQAQDAGIYECHTPSTDTRYLGSYSGKVELRVLPDVLQVSA
APPGPRGRQAPTSPPRMTVHEGQELALGCLARTSTQKHTHLAVSFGRSVPEAPVGRST
LQBVVGIRSDLAVEAGAPYAERLAAGELRLGKEGTDRYRMVVGGAQAGDAGTYHC
TAAEWIQDPDGSWAQIAEKRAVLAHVDVQTLSSQLAVTVGPGERRIGPGEPLELLCN
VSGALPPAGRHAAYSVGWEMAPAGAPGPGRLVAQLDTEGVGSLGPGYEGRHIAMEK
VASRTYRLRLEAARPGDAGTYRCLAKAYVRGSGTRLREAASARSRPLPVHVREEGVV
LEAVAWLAGGTVYRGETASLLCNISVRGGPPGLRLAASWWVERPEDGELSSVPAQLV
GGVGQDGVAELGVRPGGGPVSVELVGPRSHRLRLHSLGPEDEGVYHCAPSAWVQHA
DYSWYQAGSARSGPVTVYPYMHALDTLEVPLLVGTGVALVTGATVIGTITCCFMKR.
LRKR; Peptide sequence of Isopeptide(1) + Isopeptide(2) domains-IGSF8 VLM
203:
GACTACAAAGACCACGACGGGGATTATAAAGATCATGACATCGATTACAAGGAT
GACGATGATAAGTCTTCTGGGTTGGTCCCCAGAGGCAGCCACATGGCTTCTATGA
CCGGGGGACAACAAATGGGCAGAGGCTCAAGCGGCCTTAGCGGTGAAACTGGAC
AGAGCGGTAATACCACGATTGAGGAGGACTCCACTACCCATGTCAAATTTTCTAA
AAGAGACGCGAACGGAAAGGAATTGGCTGGCGCGATGATTGAACTGAGGAACCT
CTCTGGACAGACCATACAAAGCTGGATTTCAGACGGGACCGTTAAGGTTTTCTAT
CTGATGCCTGGCACCTACCAGTTTGTTGAAACTGCGGCGCCAGAAGGATATGAGT
TGGCGGCTCCCATCACATTCACCATTGATGAAAAGGGTCAAATTTGGGTCGACTC
AGCCATGGTTGATACCTTATCAGGTTTATCAAGTGAGCAAGGTCAGTCCGGTGAT
GACAGCGCAACACACATCAAGTTCTCAAAACGGGATGAGGATGGAAAAGAACTG
GCCGGAGCGACAATGGAACTGAGAGATTCTTCCGGCAAGACTATCTCCACATGGA
TTAGTGACGGGCAAGTCAAAGACTTCTACTTGTACCCCGGTAAGTACACCTTCGTT
GAGACTGCCGCTCCTGACGGGTATGAAGTCGCCACGGCGATCACATTCACTGTGA
ATGAACAGGGACAGGTGACGGTCAATGGAGGCGGTGGCGGCTCTGGCGGAGGAG
GCTCAGGATCCCATATGAAGCCGCTGCGTGGTGCCGTGTTTAGCCTGCAGAAACA
GCATCCCGACTATCCCGATATCTATGGCGCGATTGATCAGAATGGGACCTATCAA
AATGTGCGTACCGGCGAAGATGGTAAACTGACCTTTAAGAATCTGAGCGATGGCA
AATATCGCCTGTTTGAAAATAGCGAACCCGCTGGCTATAAACCGGTGCAGAATAA
GCCGATTGTGGCGTTTCAGATTGTGAATGGCGAAGTGCGTGATGTGACCAGCATT
GTGCCGCAGGATATTCCGGCTACATATGAATTTACCAACGGTAAACATTATATCA
CCAATGAACCGATACCGCCGAAAGGATCCCCCGCCAACCTGAAGGCCCTGGAGGC
CCAGAAGCAGAAGGAGCAGAGACAGGCCGCCGAGGAGCTGGCCAACGCCAAGA
AGCTGAAGGAGCAGCTGGAGAAGCGGGAGGTGCTGGTCCCCGAGGGGCCCTTGT
ACCGCGTGGCTGGCACAGCTGTCTCCATCTCCTGCAATGTGACCGGCTATGAGGG
CCCTGCCCAGCAGAACTTCGAGTGGTTCCTGTATAGGCCCGAGGCCCCAGATACT
GCACTGGGCATTGTCAGTACCAAGGATACCCAGTTCTCCTATGCTGTCTTCAAGTC
CCGAGTGGTGGCGGGTGAGGTGCAGGTGCAGCGCCTACAAGGTGATGCCGTGGTG
CTCAAGATTGCCCGCCTGCAGGCCCAGGATGCCGGCATTTATGAGTGCCACACCC
CCTCCACTGATACCCGCTACCTGGGCAGCTACAGCGGCAAGGTGGAGCTGAGAGT
TCTTCCAGATGTCCTCCAGGTGTCTGCTGCCCCCCCAGGGCCCCGAGGCCGCCAGG
CCCCAACCTCACCCCCACGCATGACGGTGCATGAGGGGCAGGAGCTGGCACTGGG
CTGCCTGGCGAGGACAAGCACACAGAAGCACACACACCTGGCAGTGTCCTTTGGG
CGATCTGTGCCCGAGGCACCAGTTGGGCGGTCAACTCTGCAGGAAGTGGTGGGAA
TCCGGTCAGACTTGGCCGTGGAGGCTGGAGCTCCCTATGCTGAGCGATTGGCTGC
AGGGGAGCTTCGTCTGGGCAAGGAAGGGACCGATCGGTACCGCATGGTAGTAGG
GGGTGCCCAGGCAGGGGACGCAGGCACCTACCACTGCACTGCCGCTGAGTGGATT
CAGGATCCTGATGGCAGCTGGGCCCAGATTGCAGAGAAAAGGGCCGTCCTGGCCC
ACGTGGATGTGCAGACGCTGTCCAGCCAGCTGGCAGTGACAGTGGGGCCTGGTGA
ACGTCGGATCGGCCCAGGGGAGCCCTTGGAACTGCTGTGCAATGTGTCAGGGGCA
CTTCCCCCAGCAGGCCGTCATGCTGCATACTCTGTAGGTTGGGAGATGGCACCTGC
GGGGGCACCTGGGCCCGGCCGCCTGGTAGCCCAGCTGGACACAGAGGGTGTGGG
CAGCCTGGGCCCTGGCTATGAGGGCCGACACATTGCCATGGAGAAGGTGGCATCC
AGAACATACCGGCTACGGCTAGAGGCTGCCAGGCCTGGTGATGCGGGCACCTACC
GCTGCCTCGCCAAAGCCTATGTTCGAGGGTCTGGGACCCGGCTTCGTGAAGCAGC
CAGTGCCCGTTCCCGGCCTCTCCCTGTACATGTGCGGGAGGAAGGTGTGGTGCTG
GAGGCTGTGGCATGGCTAGCAGGAGGCACAGTGTACCGCGGGGAGACTGCCTCCC
TGCTGTGCAACATCTCTGTGCGGGGTGGCCCCCCAGGACTGCGGCTGGCCGCCAG
CTGGTGGGTGGAGCGACCAGAGGACGGAGAGCTCAGCTCTGTCCCTGCCCAGCTG
GTGGGTGGCGTAGGCCAGGATGGTGTGGCAGAGCTGGGAGTCCGGCCTGGAGGA
GGCCCTGTCAGCGTAGAGCTGGTGGGGCCCCGAAGCCATCGGCTGAGACTACACA
GCTTGGGGCCCGAGGATGAAGGCGTGTACCACTGTGCCCCCAGCGCCTGGGTGCA
GCATGCCGACTACAGCTGGTACCAGGGGGGCAGTGCCCGCTCAGGGCCTGTTACA
GTCTACCCCTACATGCATGCCCTGGACACCCTATTTGTGCCTCTGCTGGTGGGTAC
AGGGGTGGCCCTAGTCACTGGTGCCACTGTCCTTGGTACCATCACTTGCTGCTTCA
TGAAGAGGCTTCGAAAACGG; Coding sequence of Isopeptide(3) + Isopeptide(1) +
Isopeptide(2) domains-IGSF8 VLM
204:
DYKDHDGDYKDHDIDYKDDDDKSSGLVPRGSHMASMTGGQQMGRGSSGLSGETGQ
SGNTTIEEDSTTHVKFSKRDANGKELAGAMIELRNLSGQTIQSWISDGTVKVFYLMPG
TYQFVETAAPEGYELAAPITFTIDEKGQIWVDSAMVDTLSGLSSEQGQSGDDSATHIK
FSKRDEDGKELAGATMELRDSSGKTISTWISDGQVKDFYLYPGKYTFVETAAPDGYE
VATAITFTVNEQGQVTVNGGGGGSGGGGSGSHMKPLRGAVFSLQKQHPDYPDIYGAI
DQNGTYQNVRTGEDGKLTFKNLSDGKYRLFENSEPAGYKPVQNKPIVAFQIVNGEVR
DVTSIVPQDIPATYEFTNGKHYITNEPIPPKGSPANLKALEAQKQKEQRQAAEELANA
KKLKEQLEKREVLVPEGPLYRVAGTAVSISCNVTGYEGPAQQNFEWFLYRPEAPDTA
LGIVSTKDTQFSYAVFKSRVVAGEVQVQRLQGDAVVLKIARLQAQDAGIYECHTPST
DTRYLGSYSGKVELRVLPDVLQVSAAPPGPRGRQAPTSPPRMTVHEGQELALGCLAR
TSTQKHTHLAVSFGRSVPEAPVGRSTLQEVVGIRSDLAVEAGAPYAERLAAGELRLG
KEGTDRYRMVVGGAQAGDAGTYHCTAAEWIQDPDGSWAQIAEKRAVLAHVDVQTL
SSQLAVTVGPGERRIGPGEPLELLCNVSGALPPAGRHAAYSVGWEMAPAGAPGPGRL
VAQLDTEGVGSLGPGYEGRHIAMEKVASRTYRLRLEAARPGDAGTYRCLAKAYVRG
SGTRLREAASARSRPLPVHVREEGVVLEAVAWLAGGTVYRGETASLLCNISVRGGPP
GLRLAASWWVERPEDGELSSVPAQLVGGVGQDGVAELGVRPGGGPVSVELVGPRSH
RLRLHSLGPEDEGVYHCAPSAWVQHADYSWYQAGSARSGPVTVYPYMHALDTLFVP
LLVGTGVALVTGATVLGTITCCFMKRLRKR; Peptide sequence of Isopeptide(3) +
Isopeptide(1) + Isopeptide(2) domains-IGSF8 VLM
205:
GGTTCCGATTACAAGGATGACGATGACAAGGGTTCCGACAGCGCAACACACATCA
AGTTCTCAAAACGGGATGAGGATGGAAAAGAACTGGCCGGAGCGACAATGGAAC
TGAGAGATTCTTCCGGCAAGACTATCTCCACATGGATTAGTGACGGGCAAGTCAA
AGACTTCTACTTGTACCCCGGTAAGTACACCTTCGTTGAGACTGCCGCTCCTGACG
GGTATGAAGTCGCCACGGCGATCACATTCACTGTGAATGAACAGGGACAGGTGAC
GGTCAATGGAGGCGGTGGCGGCTCTGGCGGAGGAGGCTCATCTTCTGGGTTGGTC
CCCAGAGGCAGCCACATGGCTTCTATGACCGGGGGACAACAAATGGGCAGAGGC
TCAAGCGGCCTTAGCGGTGAAACTGGACAGAGCGGTAATACCACGATTGAGGAG
GACTCCACTACCCATGTCAAATTTTCTAAAAGAGACGCGAACGGAAAGGAATTGG
CTGGCGCGATGATTGAACTGAGGAACCTCTCTGGACAGACCATACAAAGCTGGAT
TTCAGACGGGACCGTTAAGGTTTTCTATCTGATGCCTGGCACCTACCAGTTTGTTG
AAACTGCGGCGCCAGAAGGATATGAGTTGGCGGCTCCCATCACATTCACCATTGA
TGAAAAGGGTCAAATTTGGGTCGACTCAGGCGGTGGCGGTTCAGGTGGGGGCGGT
AGTGGCGGAGGCGGAAGCCGGGAGGTGCTGGTCCCCGAGGGGCCCTTGTACCGC
GTGGCTGGCACAGCTGTCTCCATCTCCTGCAATGTGACCGGCTATGAGGGCCCTGC
CCAGCAGAACTTCGAGTGGTTCCTGTATAGGCCCGAGGCCCCAGATACTGCACTG
GGCATTGTCAGTACCAAGGATACCCAGTTCTCCTATGCTGTCTTCAAGTCCCGAGT
GGTGGCGGGTGAGGTGCAGGTGCAGCGCCTACAAGGTGATGCCGTGGTGCTCAAG
ATTGCCCGCCTGCAGGCCCAGGATGCCGGCATTTATGAGTGCCACACCCCCTCCA
CTGATACCCGCTACCTGGGCAGCTACAGCGGCAAGGTGGAGCTGAGAGTTCTTCC
AGATGTCCTCCAGGTGTCTGCTGCCCCCCCAGGGCCCCGAGGCCGCCAGGCCCCA
ACCTCACCCCCACGCATGACGGTGCATGAGGGGCAGGAGCTGGCACTGGGCTGCC
TGGCGAGGACAAGCACACAGAAGCACACACACCTGGCAGTGTCCTTTGGGCGATC
TGTGCCCGAGGCACCAGTTGGGCGGTCAACTCTGCAGGAAGTGGTGGGAATCCGG
TCAGACTTGGCCGTGGAGGCTGGAGCTCCCTATGCTGAGCGATTGGCTGCAGGGG
AGCTTCGTCTGGGCAAGGAAGGGACCGATCGGTACCGCATGGTAGTAGGGGGTGC
CCAGGCAGGGGACGCAGGCACCTACCACTGCACTGCCGCTGAGTGGATTCAGGAT
CCTGATGGCAGCTGGGCCCAGATTGCAGAGAAAAGGGCCGTCCTGGCCCACGTGG
ATGTGCAGACGCTGTCCAGCCAGCTGGCAGTGACAGTGGGGCCTGGTGAACGTCG
GATCGGCCCAGGGGAGCCCTTGGAACTGCTGTGCAATGTGTCAGGGGCACTTCCC
CCAGCAGGCCGTCATGCTGCATACTCTGTAGGTTGGGAGATGGCACCTGCGGGGG
CACCTGGGCCCGGCCGCCTGGTAGCCCAGCTGGACACAGAGGGTGTGGGCAGCCT
GGGCCCTGGCTATGAGGGCCGACACATTGCCATGGAGAAGGTGGCATCCAGAAC
ATACCGGCTACGGCTAGAGGCTGCCAGGCCTGGTGATGCGGGCACCTACCGCTGC
CTCGCCAAAGCCTATGTTCGAGGGTCTGGGACCCGGCTTCGTGAAGCAGCCAGTG
CCCGTTCCCGGCCTCTCCCTGTACATGTGCGGGAGGAAGGTGTGGTGCTGGAGGC
TGTGGCATGGCTAGCAGGAGGCACAGTGTACCGCGGGGAGACTGCCTCCCTGCTG
TGCAACATCTCTGTGCGGGGTGGCCCCCCAGGACTGCGGCTGGCCGCCAGCTGGT
GGGTGGAGCGACCAGAGGACGGAGAGCTCAGCTCTGTCCCTGCCCAGCTGGTGGG
TGGCGTAGGCCAGGATGGTGTGGCAGAGCTGGGAGTCCGGCCTGGAGGAGGCCC
TGTCAGCGTAGAGCTGGTGGGGCCCCGAAGCCATCGGCTGAGACTACACAGCTTG
GGGCCCGAGGATGAAGGCGTGTACCACTGTGCCCCCAGCGCCTGGGTGCAGCATG
CCGACTACAGCTGGTACCAGGGGGGCAGTGCCCGCTCAGGGCCTGTTACAGTCTA
CCCCTACATGCATGCCCTGGACACCCTATTTGTGCCTCTGCTGGTGGGTACAGGGG
TGGCCCTAGTCACTGGTGCCACTGTCCTTGGTACCATCACTTGCTGCTTCATGAAG
AGGCTTCGAAAACGG; Coding sequence of Isopeptide(1) + Isopeptide(3) IGSF8 VLM
206:
GSDYKDDDDKGSDSATHIKFSKRDEDGKELAGATMELRDSSGKTISTWISDGQVKDF
YLYPGKYTFVETAAPDGYEVATAITFTVNEQGQVTVNGGGGGSGGGGSSSGLVPRGS
HMASMTGGQQMGRGSSGLSGETGQSGNTTIEEDSTTHVKFSKRDANGKELAGAMIE
LRNLSGQTIQSWISDGTVKVFYLMPGTYQFVETAAPEGYELAAPITFTIDEKGQIWVDS
GGGGSGGGGSGGGGSREVLVPEGPLYRVAGTAVSISCNVTGYEGPAQQNFEWFLYRP
EAPDTALGIVSTKDTQFSYAVFKSRVVAGEVQVQRLQGDAVVLKIARLQAQDAGIYE
CHTPSTDTRYLGSYSGKVELRVLPDVLQVSAAPPGPRGRQAPTSPPRMTVHEGQELAL
GCLARTSTQKHTHLAVSFGRSVPEAPVGRSTLQEVVGIRSDLAVEAGAPYAERLAAG
ELRLGKEGTDRYRMVVGGAQAGDAGTYHCTAAEWIQDPDGSWAQIAEKRAVLAHV
DVQTLSSQLAVTVGPGERRIGPGEPLELLCNVSGALPPAGRHAAYSVGWEMAPAGAP
GPGRLVAQLDTEGVGSLGPGYEGRHIAMEKVASRTYRLRLEAARPGDAGTYRCLAK
AYVRGSGTRLREAASARSRPLPVHVREEGVVLEAVAWLAGGTVYRGETASLLCNISV
RGGPPGLRLAASWWVERPEDGELSSVPAQLVGGVGQDGVAELGVRPGGGPVSVELV
GPRSHRLRLHSLGPEDEGVYHCAPSAWVQHADYSWYQAGSARSGPVTVYPYMHAL
DTLFVPLLVGTGVALVTGATVLGTITCCFMKRLRKR; Peptide sequence of Isopeptide(1)
+ Isopeptide(3) IGSF8 VLM
207:
GGTTCCGATTACAAGGATGACGATGACAAGGGTTCCTCTTCTGGGTTGGTCCCCA
GAGGCAGCCACATGGCTTCTATGACCGGGGGACAACAAATGGGCAGAGGCTCAA
GCGGCCTTAGCGGTGAAACTGGACAGAGCGGTAATACCACGATTGAGGAGGACT
CCACTACCCATGTCAAATTTTCTAAAAGAGACGCGAACGGAAAGGAATTGGCTGG
CGCGATGATTGAACTGAGGAACCTCTCTGGACAGACCATACAAAGCTGGATTTCA
GACGGGACCGTTAAGGTTTTCTATCTGATGCCTGGCACCTACCAGTTTGTTGAAAC
TGCGGCGCCAGAAGGATATGAGTTGGCGGCTCCCATCACATTCACCATTGATGAA
AAGGGTCAAATTTGGGTCGACTCAGGCGGTGGCGGCTCTGGCGGAGGAGGCTCAG
ACAGCGCAACACACATCAAGTTCTCAAAACGGGATGAGGATGGAAAAGAACTGG
CCGGAGCGACAATGGAACTGAGAGATTCTTCCGGCAAGACTATCTCCACATGGAT
TAGTGACGGGCAAGTCAAAGACTTCTACTTGTACCCCGGTAAGTACACCTTCGTTG
AGACTGCCGCTCCTGACGGGTATGAAGTCGCCACGGCGATCACATTCACTGTGAA
TGAACAGGGACAGGTGACGGTCAATGGAGGCGGTGGCGGTTCAGGTGGGGGGGG
TAGTGGCGGAGGCGGAAGCCGGGAGGTGCTGGTCCCCGAGGGGCCCTTGTACCGC
GTGGCTGGCACAGCTGTCTCCATCTCCTGCAATGTGACCGGCTATGAGGGCCCTGC
CCAGCAGAACTTCGAGTGGTTCCTGTATAGGCCCGAGGCCCCAGATACTGCACTG
GGCATTGTCAGTACCAAGGATACCCAGTTCTCCTATGCTGTCTTCAAGTCCCGAGT
GGTGGCGGGTGAGGTGCAGGTGCAGCGCCTACAAGGTGATGCCGTGGTGCTCAAG
ATTGCCCGCCTGCAGGCCCAGGATGCCGGCATTTATGAGTGCCACACCCCCTCCA
CTGATACCCGCTACCTGGGCAGCTACAGCGGCAAGGTGGAGCTGAGAGTTCTTCC
AGATGTCCTCCAGGTGTCTGCTGCCCCCCCAGGGCCCCGAGGCCGCCAGGCCCCA
ACCTCACCCCCACGCATGACGGTGCATGAGGGGCAGGAGCTGGCACTGGGCTGCC
TGGCGAGGACAAGCACACAGAAGCACACACACCTGGCAGTGTCCTTTGGGCGATC
TGTGCCCGAGGCACCAGTTGGGCGGTCAACTCTGCAGGAAGTGGTGGGAATCCGG
TCAGACTTGGCCGTGGAGGCTGGAGCTCCCTATGCTGAGCGATTGGCTGCAGGGG
AGCTTCGTCTGGGCAAGGAAGGGACCGATCGGTACCGCATGGTAGTAGGGGGTGC
CCAGGCAGGGGACGCAGGCACCTACCACTGCACTGCCGCTGAGTGGATTCAGGAT
CCTGATGGCAGCTGGGCCCAGATTGCAGAGAAAAGGGCCGTCCTGGCCCACGTGG
ATGTGCAGACGCTGTCCAGCCAGCTGGCAGTGACAGTGGGGCCTGGTGAACGTCG
GATCGGCCCAGGGGAGCCCTTGGAACTGCTGTGCAATGTGTCAGGGGCACTTCCC
CCAGCAGGCCGTCATGCTGCATACTCTGTAGGTTGGGAGATGGCACCTGCGGGGG
CACCTGGGCCCGGCCGCCTGGTAGCCCAGCTGGACACAGAGGGTGTGGGCAGCCT
GGGCCCTGGCTATGAGGGCCGACACATTGCCATGGAGAAGGTGGCATCCAGAAC
ATACCGGCTACGGCTAGAGGCTGCCAGGCCTGGTGATGCGGGCACCTACCGCTGC
CTCGCCAAAGCCTATGTTCGAGGGTCTGGGACCCGGCTTCGTGAAGCAGCCAGTG
CCCGTTCCCGGCCTCTCCCTGTACATGTGCGGGAGGAAGGTGTGGTGCTGGAGGC
TGTGGCATGGCTAGCAGGAGGCACAGTGTACCGCGGGGAGACTGCCTCCCTGCTG
TGCAACATCTCTGTGCGGGGTGGCCCCCCAGGACTGCGGCTGGCCGCCAGCTGGT
GGGTGGAGCGACCAGAGGACGGAGAGCTCAGCTCTGTCCCTGCCCAGCTGGTGGG
TGGCGTAGGCCAGGATGGTGTGGCAGAGCTGGGAGTCCGGCCTGGAGGAGGCCC
TGTCAGCGTAGAGCTGGTGGGGCCCCGAAGCCATCGGCTGAGACTACACAGCTTG
GGGCCCGAGGATGAAGGCGTGTACCACTGTGCCCCCAGCGCCTGGGTGCAGCATG
CCGACTACAGCTGGTACCAGGCGGGCAGTGCCCGCTCAGGGCCTGTTACAGTCTA
CCCCTACATGCATGCCCTGGACACCCTATTTGTGCCTCTGCTGGTGGGTACAGGGG
TGGCCCTAGTCACTGGTGCCACTGTCCTTGGTACCATCACTTGCTGCTTCATGAAG
AGGCTTCGAAAACGG; Coding sequence of Isopeptide(3) + Isopeptide(1) IGSF8 VLM
208:
GSDYKDDDDKGSSSGLVPRGSHMASMTGGQQMGRGSSGLSGETGQSGNTTIEEDSTT
HVKFSKRDANGKELAGAMIELRNLSGQTIQSWISDGTVKVFYLMPGTYQFVETAAPE
GYELAAPITFTIDEKGQIWVDSGGGGSGGGGSDSATHIKFSKRDEDGKELAGATMELR
DSSGKTISTWISDGQVKDFYLYPGKYTFVETAAPDGYEVATAITFTVNEQGQVTVNG
GGGGSGGGGSGGGGSREVLVPEGPLYRVAGTAVSISCNVTGYEGPAQQNFEWFLYRP
EAPDTALGIVSTKDTQFSYAVFKSRVVAGEVQVQRLQGDAVVLKIARLQAQDAGIYE
CHTPSTDTRYLGSYSGKVELRVLPDVLQVSAAPPGPRGRQAPTSPPRMTVHEGQELAL
GCLARTSTQKHTHLAVSFGRSVPEAPVGRSTLQEVVGIRSDLAVEAGAPYAERLAAG
ELRLGKEGTDRYRMVVGGAQAGDAGTYHCTAAEWIQDPDGSWAQIAEKRAVLAHV
DVQTLSSQLAVTVGPGERRIGPGEPLELLCNVSGALPPAGRHAAYSVGWEMAPAGAP
GPGRLVAQLDTEGVGSLGPGYEGRHIAMEKVASRTYRLRLEAARPGDAGTYRCLAK
AYVRGSGTRLREAASARSRPLPVHVREEGVVLEAVAWLAGGTVYRGETASLLCNISV
RGGPPGLRLAASWWVERPEDGELSSVPAQLVGGVGQDGVAELGVRPGGGPVSVELV
GPRSHRLRLHSLGPEDEGVYHCAPSAWVQHADYSWYQAGSARSGPVTVYPYMHAL
DTLFVPLLVGTGVALVTGATVLGTITCCFMKRLRKR; Peptide sequence of Isopeptide(3)
+ Isopeptide(1) IGSF8 VLM
209:
GGTTCCGATTACAAGGATGACGATGACAAGGGTTCCTCTTCTGGGTTGGTCCCCA
GAGGCAGCCACATGGCTTCTATGACCGGGGGACAACAAATGGGCAGAGGCTCAA
GCGGCCTTAGCGGTGAAACTGGACAGAGCGGTAATACCACGATTGAGGAGGACT
CCACTACCCATGTCAAATTTTCTAAAAGAGACGCGAACGGAAAGGAATTGGCTGG
CGCGATGATTGAACTGAGGAACCTCTCTGGACAGACCATACAAAGCTGGATTTCA
GACGGGACCGTTAAGGTTTTCTATCTGATGCCTGGCACCTACCAGTTTGTTGAAAC
TGCGGCGCCAGAAGGATATGAGTTGGCGGCTCCCATCACATTCACCATTGATGAA
AAGGGTCAAATTTGGGTCGACTCAGGCGGTGGCGGCTCTGGCGGAGGAGGCTCAG
ACAGCGCAACACACATCAAGTTCTCAAAACGGGATGAGGATGGAAAAGAACTGG
CCGGAGCGACAATGGAACTGAGAGATTCTTCCGGCAAGACTATCTCCACATGGAT
TAGTGACGGGCAAGTCAAAGACTTCTACTTGTACCCCGGTAAGTACACCTTCGTTG
AGACTGCCGCTCCTGACGGGTATGAAGTCGCCACGGCGATCACATTCACTGTGAA
TGAACAGGGACAGGTGACGGTCAATGGAGGCGGTGGCGGTTCAGGTGGGGGGGG
TAGTGGCGGAGGCGGAAGCTTGGAACTTAATTTGACAGATTCAGAAAATGCCACT
TGCCTTTATGCAAAATGGCAGATGAATTTCACAGTACGCTATGAAACTACAAATA
AAACTTATAAAACTGTAACCATTTCAGACCATGGCACTGTGACATATAATGGAAG
CATTTGTGGGGATGATCAGAATGGTCCCAAAATAGCAGTGCAGTTCGGACCTGGC
TTTTCCTGGATTGCGAATTTTACCAAGGCAGCATCTACTTATTCAATTGACAGCGT
CTCATTTTCCTACAACACTGGTGATAACACAACATTTCCTGATGCTGAAGATAAAG
GAATTCTTACTGTTGATGAACTTTTGGCCATCAGAATTCCATTGAATGACCTTTTT
AGATGCAATAGTTTATCAACTTTGGAAAAGAATGATGTTGTCCAACACTACTGGG
ATGTTCTTGTACAAGCTTTTGTCCAAAATGGCACAGTGAGCACAAATGAGTTCCTG
TGTGATAAAGACAAAACTTCAACAGTGGCACCCACCATACACACCACTGTGCCAT
CTCCTACTACAACACCTACTCCAAAGGAAAAACCAGAAGCTGGAACCTATTCAGT
TAATAATGGCAATGATACTTGTCTGCTGGCTACCATGGGGCTGCAGCTGAACATC
ACTCAGGATAAGGTTGCTTCAGTTATTAACATCAACCCCAATACAACTCACTCCAC
AGGCAGCTGCCGTTCTCACACTGCTCTACTTAGACTCAATAGCAGCACCATTAAGT
ATCTAGACTTTGTCTTTGCTGTGAAAAATGAAAACCGATTTTATCTGAAGGAAGTG
AACATCAGCATGTATTTGGTTAATGGCTCCGTTTTCAGCATTGCAAATAACAATCT
CAGCTACTGGGATGCCCCCCTGGGAAGTTCTTATATGTGCAACAAAGAGCAGACT
GTTTCAGTGTCTGGAGCATTTCAGATAAATACCTTTGATCTAAGGGTTCAGCCTTT
CAATGTGACACAAGGAAAGTATTCTACAGCCCAAGAGTGTTCGCTGGATGATGAC
ACCATTCTAATCCCAATTATAGTTGGTGCTGGTCTTTCAGGCTTGATTATCGTTATA
GTGATTGCTGTGATGCAGAGACTCTTTCCCCGCATCCCTCACATGAAAGACCCCAT
CGGTGACAGCTTCCAAAACGACAAGCTGGTGGTCTGGGAGGCGGGCAAAGCCGG
CCTGGAGGAGTGTCTGGTGACTGAAGTACAGGTCGTGCAGAAAACTTGA; Coding
sequence of Isopeptide(3) + Isopeptide(1) Lamp2-IL3RA VLM (chimeric)
210:
GSDYKDDDDKGSSSGLVPRGSHMASMTGGQQMGRGSSGLSGETGQSGNTTIEEDSTT
HVKFSKRDANGKELAGAMIELRNLSGQTIQSWISDGTVKVFYLMPGTYQFVETAAPE
GYELAAPITFTIDEKGQIWVDSGGGGSGGGGSDSATHIKFSKRDEDGKELAGATMELR
DSSGKTISTWISDGQVKDFYLYPGKYTFVETAAPDGYEVATAITFTVNEQGQVTVNG
GGGGSGGGGSGGGGSLELNLTDSENATCLYAKWQMNFTVRYETTNKTYKTVTISDH
GTVTYNGSICGDDQNGPKIAVQFGPGFSWIANFTKAASTYSIDSVSFSYNTGDNTTFPD
AEDKGILTVDELLAIRIPLNDLERCNSLSTLEKNDVVQHYWDVLVQAFVQNGTVSTNE
FLCDKDKTSTVAPTIHTTVPSPTTTPTPKEKPEAGTYSVNNGNDTCLLATMGLQLNITQ
DKVASVININPNTTHSTGSCRSHTALLRLNSSTIKYLDFVFAVKNENRFYLKEVNISMY
LVNGSVFSIANNNLSYWDAPLGSSYMCNKEQTVSVSGAFQINTFDLRVQPFNVTQGK
YSTAQECSLDDDTILIPIIVGAGLSGLIIVIVIAVMQRLFPRIPHMKDPIGDSFQNDKLVV
WEAGKAGLEECLVTEVQVVQKT; Peptide sequence of Isopeptide(3) + Isopeptide(1)
Lamp2-IL3RA VLM (chimeric)
211:
GGTTCCGATTACAAGGATGACGATGACAAGGGTTCCTCTTCTGGGTTGGTCCCCA
GAGGCAGCCACATGGCTTCTATGACCGGGGGACAACAAATGGGCAGAGGCTCAA
GCGGCCTTAGCGGTGAAACTGGACAGAGCGGTAATACCACGATTGAGGAGGACT
CCACTACCCATGTCAAATTTTCTAAAAGAGACGCGAACGGAAAGGAATTGGCTGG
CGCGATGATTGAACTGAGGAACCTCTCTGGACAGACCATACAAAGCTGGATTTCA
GACGGGACCGTTAAGGTTTTCTATCTGATGCCTGGCACCTACCAGTTTGTTGAAAC
TGCGGCGCCAGAAGGATATGAGTTGGCGGCTCCCATCACATTCACCATTGATGAA
AAGGGTCAAATTTGGGTCGACTCAGGCGGTGGCGGCTCTGGCGGAGGAGGCTCAG
ACAGCGCAACACACATCAAGTTCTCAAAACGGGATGAGGATGGAAAAGAACTGG
CCGGAGCGACAATGGAACTGAGAGATTCTTCCGGCAAGACTATCTCCACATGGAT
TAGTGACGGGCAAGTCAAAGACTTCTACTTGTACCCCGGTAAGTACACCTTCGTTG
AGACTGCCGCTCCTGACGGGTATGAAGTCGCCACGGCGATCACATTCACTGTGAA
TGAACAGGGACAGGTGACGGTCAATGGAGGCGGTGGCGGTTCAGGTGGGGGGGG
TAGTGGCGGAGGCGGAAGCTTGGAACTTAATTTGACAGATTCAGAAAATGCCACT
TGCCTTTATGCAAAATGGCAGATGAATTTCACAGTACGCTATGAAACTACAAATA
AAACTTATAAAACTGTAACCATTTCAGACCATGGCACTGTGACATATAATGGAAG
CATTTGTGGGGATGATCAGAATGGTCCCAAAATAGCAGTGCAGTTCGGACCTGGC
TTTTCCTGGATTGCGAATTTTACCAAGGCAGCATCTACTTATTCAATTGACAGCGT
CTCATTTTCCTACAACACTGGTGATAACACAACATTTCCTGATGCTGAAGATAAAG
GAATTCTTACTGTTGATGAACTTTTGGCCATCAGAATTCCATTGAATGACCTTTTT
AGATGCAATAGTTTATCAACTTTGGAAAAGAATGATGTTGTCCAACACTACTGGG
ATGTTCTTGTACAAGCTTTTGTCCAAAATGGCACAGTGAGCACAAATGAGTTCCTG
TGTGATAAAGACAAAACTTCAACAGTGGCACCCACCATACACACCACTGTGCCAT
CTCCTACTACAACACCTACTCCAAAGGAAAAACCAGAAGCTGGAACCTATTCAGT
TAATAATGGCAATGATACTTGTCTGCTGGCTACCATGGGGCTGCAGCTGAACATC
ACTCAGGATAAGGTTGCTTCAGTTATTAACATCAACCCCAATACAACTCACTCCAC
AGGCAGCTGCCGTTCTCACACTGCTCTACTTAGACTCAATAGCAGCACCATTAAGT
ATCTAGACTTTGTCTTTGCTGTGAAAAATGAAAACCGATTTTATCTGAAGGAAGTG
AACATCAGCATGTATTTGGTTAATGGCTCCGTTTTCAGCATTGCAAATAACAATCT
CAGCTACTGGGATGCCCCCCTGGGAAGTTCTTATATGTGCAACAAAGAGCAGACT
GTTTCAGTGTCTGGAGCATTTCAGATAAATACCTTTGATCTAAGGGTTCAGCCTTT
CAATGTGACACAAGGAAAGTATTCTACAGCCCAAGAGTGTTCGCTGGATGATGAC
ACCATTCTAATCCCAATTATAGTTGGTGCTGGTCTTTCAGGCTTGATTATCGTTATA
GTGATTGCTCGCCTCTCCCGCAAGGGCCACATGTACCCCGTGCGTAATTACTCCCC
CACCGAGATGGTCTGCATCTCATCCCTGTTGCCTGATGGGGGTGAGGGGCCCTCTG
CCACAGCCAATGGGGGCCTGTCCAAGGCCAAGAGCCCGGGCCTGACGCCAGAGC
CCAGGGAGGACCGTGAGGGGGATGACCTCACCCTGCACAGCTTCCTCCCTTAG;
Coding sequence of Isopeptide(3) + Isopeptide(1) Lamp2-SELPL VLM (chimeric)
212:
GSDYKDDDDKGSSSGLVPRGSHMASMTGGQQMGRGSSGLSGETGQSGNTTIEEDSTT
HVKFSKRDANGKELAGAMIELRNLSGQTIQSWISDGTVKVFYLMPGTYQFVETAAPE
GYELAAPITFTIDEKGQIWVDSGGGGSGGGGSDSATHIKFSKRDEDGKELAGATMELR
DSSGKTISTWISDGQVKDFYLYPGKYTFVETAAPDGYEVATAITFTVNEQGQVTVNG
GGGGSGGGGSGGGGSLELNLTDSENATCLYAKWQMNFTVRYETTNKTYKTVTISDH
GTVTYNGSICGDDQNGPKIAVQFGPGFSWIANFTKAASTYSIDSVSFSYNTGDNTTFPD
AEDKGILTVDELLAIRIPLNDLERCNSLSTLEKNDVVQHYWDVLVQAFVQNGTVSTNE
FLCDKDKTSTVAPTIHTTVPSPTTTPTPKEKPEAGTYSVNNGNDTCLLATMGLQLNITQ
DKVASVININPNTTHSTGSCRSHTALLRLNSSTIKYLDFVFAVKNENRFYLKEVNISMY
LVNGSVFSIANNNLSYWDAPLGSSYMCNKEQTVSVSGAFQINTFDLRVQPFNVTQGK
YSTAQECSLDDDTILIPIIVGAGLSGLIIVIVIARLSRKGHMYPVRNYSPTEMVCISSLLP
DGGEGPSATANGGLSKAKSPGLTPEPREDREGDDLTLHSFLP; Peptide sequence of
Isopeptide(3) + Isopeptide(1) Lamp2-SELPL VLM (chimeric)
213:
GGTTCCGATTACAAGGATGACGATGACAAGGGTTCCTCTTCTGGGTTGGTCCCCA
GAGGCAGCCACATGGCTTCTATGACCGGGGGACAACAAATGGGCAGAGGCTCAA
GCGGCCTTAGCGGTGAAACTGGACAGAGCGGTAATACCACGATTGAGGAGGACT
CCACTACCCATGTCAAATTTTCTAAAAGAGACGCGAACGGAAAGGAATTGGCTGG
CGCGATGATTGAACTGAGGAACCTCTCTGGACAGACCATACAAAGCTGGATTTCA
GACGGGACCGTTAAGGTTTTCTATCTGATGCCTGGCACCTACCAGTTTGTTGAAAC
TGCGGCGCCAGAAGGATATGAGTTGGCGGCTCCCATCACATTCACCATTGATGAA
AAGGGTCAAATTTGGGTCGACTCAGGCGGTGGCGGCTCTGGCGGAGGAGGCTCAG
ACAGCGCAACACACATCAAGTTCTCAAAACGGGATGAGGATGGAAAAGAACTGG
CCGGAGCGACAATGGAACTGAGAGATTCTTCCGGCAAGACTATCTCCACATGGAT
TAGTGACGGGCAAGTCAAAGACTTCTACTTGTACCCCGGTAAGTACACCTTCGTTG
AGACTGCCGCTCCTGACGGGTATGAAGTCGCCACGGCGATCACATTCACTGTGAA
TGAACAGGGACAGGTGACGGTCAATGGAGGCGGTGGCGGTTCAGGTGGGGGGGG
TAGTGGCGGAGGCGGAAGCTTGGAACTTAATTTGACAGATTCAGAAAATGCCACT
TGCCTTTATGCAAAATGGCAGATGAATTTCACAGTACGCTATGAAACTACAAATA
AAACTTATAAAACTGTAACCATTICAGACCATGGCACTGTGACATATAATGGAAG
CATTTGTGGGGATGATCAGAATGGTCCCAAAATAGCAGTGCAGTTCGGACCTGGC
TTTTCCTGGATTGCGAATTTTACCAAGGCAGCATCTACTTATTCAATTGACAGCGT
CTCATTTTCCTACAACACTGGTGATAACACAACATTTCCTGATGCTGAAGATAAAG
GAATTCTTACTGTTGATGAACTTTTGGCCATCAGAATTCCATTGAATGACCTTTTT
AGATGCAATAGTTTATCAACTTTGGAAAAGAATGATGTTGTCCAACACTACTGGG
ATGTTCTTGTACAAGCTTTTGTCCAAAATGGCACAGTGAGCACAAATGAGTTCCTG
TGTGATAAAGACAAAACTTCAACAGTGGCACCCACCATACACACCACTGTGCCAT
CTCCTACTACAACACCTACTCCAAAGGAAAAACCAGAAGCTGGAACCTATTCAGT
TAATAATGGCAATGATACTTGTCTGCTGGCTACCATGGGGCTGCAGCTGAACATC
ACTCAGGATAAGGTTGCTTCAGTTATTAACATCAACCCCAATACAACTCACTCCAC
AGGCAGCTGCCGTTCTCACACTGCTCTACTTAGACTCAATAGCAGCACCATTAAGT
ATCTAGACTTTGTCTTTGCTGTGAAAAATGAAAACCGATTTTATCTGAAGGAAGTG
AACATCAGCATGTATTTGGTTAATGGCTCCGTTTTCAGCATTGCAAATAACAATCT
CAGCTACTGGGATGCCCCCCTGGGAAGTTCTTATATGTGCAACAAAGAGCAGACT
GTTTCAGTGTCTGGAGCATTTCAGATAAATACCTTTGATCTAAGGGTTCAGCCTTT
CAATGTGACACAAGGAAAGTATTCTACAGCCCAAGAGTGTTCGCTGGATGATGAC
ACCATTCTAATCCCAATTATAGTTGGTGCTGGTCTTTCAGGCTTGATTATCGTTATA
GTGATTGCTAGCTCCCACTGGTGTTGTAAGAAGGAGGTTCAGGAGACACGGCGCG
AGCGCCGCAGGCTCATGTCGATGGAGATGGACTAG; Coding sequence of
Isopeptide(3) + Isopeptide(1) Lamp2-PTGFRN VLM (chimeric)
214:
GSDYKDDDDKGSSSGLVPRGSHMASMTGGQQMGRGSSGLSGETGQSGNTTIEEDSTT
HVKFSKRDANGKELAGAMIELRNLSGQTIQSWISDGTVKVFYLMPGTYQFVETAAPE
GYELAAPITFTIDEKGQIWVDSGGGGSGGGGSDSATHIKFSKRDEDGKELAGATMELR
DSSGKTISTWISDGQVKDFYLYPGKYTFVETAAPDGYEVATAITFTVNEQGQVTVNG
GGGGSGGGGSGGGGSLELNLTDSENATCLYAKWQMNFTVRYETTNKTYKTVTISDH
GTVTYNGSICGDDQNGPKIAVQFGPGFSWIANFTKAASTYSIDSVSFSYNTGDNTTFPD
AEDKGILTVDELLAIRIPLNDLFRCNSLSTLEKNDVVQHYWDVLVQAFVQNGTVSTNE
FLCDKDKTSTVAPTIHTTVPSPTTTPTPKEKPEAGTYSVNNGNDTCLLATMGLQLNITQ
DKVASVININPNTTHSTGSCRSHTALLRLNSSTIKYLDFVFAVKNENRFYLKEVNISMY
LVNGSVFSIANNNLSYWDAPLGSSYMCNKEQTVSVSGAFQINTFDLRVQPFNVTQGK
YSTAQECSLDDDTILIPIIVGAGLSGLIIVIVIASSHWCCKKEVQETRRERRRLMSMEMD;
Peptide sequence of Isopeptide(3) + Isopeptide(1) Lamp2-PTGFRN VLM (chimerie)

TABLE 8
Fusion Protein Comprising Isopeptide Tag and VLM

SEQ ID NO: Sequence; Source
215:
GACTACAAAGACCACGACGGGGATTATAAAGATCATGACATCGATTACAAGGAT
GACGATGATAAGGGCTCTGGCGCACACATCGTTATGGTCGACGCATACAAACCAA
CAAAGGGATCCCCCGCCAACCTGAAGGCCCTGGAGGCCCAGAAGCAGAAGGAGC
AGAGACAGGCCGCCGAGGAGCTGGCCAACGCCAAGAAGCTGAAGGAGCAGCTGG
AGAAGCGGGAGGTGCTGGTCCCCGAGGGGCCCTTGTACCGCGTGGCTGGCACAGC
TGTCTCCATCTCCTGCAATGTGACCGGCTATGAGGGCCCTGCCCAGCAGAACTTCG
AGTGGTTCCTGTATAGGCCCGAGGCCCCAGATACTGCACTGGGCATTGTCAGTAC
CAAGGATACCCAGTTCTCCTATGCTGTCTTCAAGTCCCGAGTGGTGGCGGGTGAG
GTGCAGGTGCAGCGCCTACAAGGTGATGCCGTGGTGCTCAAGATTGCCCGCCTGC
AGGCCCAGGATGCCGGCATTTATGAGTGCCACACCCCCTCCACTGATACCCGCTA
CCTGGGCAGCTACAGCGGCAAGGTGGAGCTGAGAGTTCTTCCAGATGTCCTCCAG
GTGTCTGCTGCCCCCCCAGGGCCCCGAGGCCGCCAGGCCCCAACCTCACCCCCAC
GCATGACGGTGCATGAGGGGCAGGAGCTGGCACTGGGCTGCCTGGCGAGGACAA
GCACACAGAAGCACACACACCTGGCAGTGTCCTTTGGGCGATCTGTGCCCGAGGC
ACCAGTTGGGCGGTCAACTCTGCAGGAAGTGGTGGGAATCCGGTCAGACTTGGCC
GTGGAGGCTGGAGCTCCCTATGCTGAGCGATTGGCTGCAGGGGAGCTTCGTCTGG
GCAAGGAAGGGACCGATCGGTACCGCATGGTAGTAGGGGGTGCCCAGGCAGGGG
ACGCAGGCACCTACCACTGCACTGCCGCTGAGTGGATTCAGGATCCTGATGGCAG
CTGGGCCCAGATTGCAGAGAAAAGGGCCGTCCTGGCCCACGTGGATGTGCAGACG
CTGTCCAGCCAGCTGGCAGTGACAGTGGGGCCTGGTGAACGTCGGATCGGCCCAG
GGGAGCCCTTGGAACTGCTGTGCAATGTGTCAGGGGCACTTCCCCCAGCAGGCCG
TCATGCTGCATACTCTGTAGGTTGGGAGATGGCACCTGCGGGGGCACCTGGGCCC
GGCCGCCTGGTAGCCCAGCTGGACACAGAGGGTGTGGGCAGCCTGGGCCCTGGCT
ATGAGGGCCGACACATTGCCATGGAGAAGGTGGCATCCAGAACATACCGGCTAC
GGCTAGAGGCTGCCAGGCCTGGTGATGCGGGCACCTACCGCTGCCTCGCCAAAGC
CTATGTTCGAGGGTCTGGGACCCGGCTTCGTGAAGCAGCCAGTGCCCGTTCCCGG
CCTCTCCCTGTACATGTGCGGGAGGAAGGTGTGGTGCTGGAGGCTGTGGCATGGC
TAGCAGGAGGCACAGTGTACCGCGGGGAGACTGCCTCCCTGCTGTGCAACATCTC
TGTGCGGGGGGCCCCCCAGGACTGCGGCTGGCCGCCAGCTGGTGGGTGGAGCGA
CCAGAGGACGGAGAGCTCAGCTCTGTCCCTGCCCAGCTGGTGGGTGGCGTAGGCC
AGGATGGTGTGGCAGAGCTGGGAGTCCGGCCTGGAGGAGGCCCTGTCAGCGTAG
AGCTGGTGGGGCCCCGAAGCCATCGGCTGAGACTACACAGCTTGGGGCCCGAGG
ATGAAGGCGTGTACCACTGTGCCCCCAGCGCCTGGGTGCAGCATGCCGACTACAG
CTGGTACCAGGCGGGCAGTGCCCGCTCAGGGCCTGTTACAGTCTACCCCTACATG
CATGCCCTGGACACCCTATTTGTGCCTCTGCTGGTGGGTACAGGGGTGGCCCTAGT
CACTGGTGCCACTGTCCTTGGTACCATCACTTGCTGCTTCATGAAGAGGCTTCGAA
AACGG; Coding sequence of Isopeptide(1) tag-IGSF8 VLM.
216;
DYKDHDGDYKDHDIDYKDDDDKGSGAHIVMVDAYKPTKGSPANLKALEAQKQKEQ
RQAAEELANAKKLKEQLEKREVLVPEGPLYRVAGTAVSISCNVTGYEGPAQQNFEWF
LYRPEAPDTALGIVSTKDTQFSYAVFKSRVVAGEVQVQRLQGDAVVLKIARLQAQDA
GIYECHTPSTDTRYLGSYSGKVELRVLPDVLQVSAAPPGPRGRQAPTSPPRMTVHEGQ
ELALGCLARTSTQKHTHLAVSFGRSVPEAPVGRSTLQEVVGIRSDLAVEAGAPYAERL
AAGELRLGKEGTDRYRMVVGGAQAGDAGTYHCTAAEWIQDPDGSWAQIAEKRAVL
AHVDVQTLSSQLAVTVGPGERRIGPGEPLELLCNVSGALPPAGRHAAYSVGWEMAPA
GAPGPGRLVAQLDTEGVGSLGPGYEGRHIAMEKVASRTYRLRLEAARPGDAGTYRC
LAKAYVRGSGTRLREAASARSRPLPVHVREEGVVLEAVAWLAGGTVYRGETASLLC
NISVRGGPPGLRLAASWWVERPEDGELSSVPAQLVGGVGQDGVAELGVRPGGGPVS
VELVGPRSHRLRLHSLGPEDEGVYHCAPSAWVQHADYSWYQAGSARSGPVTVYPY
MHALDTLFVPLLVGTGVALVTGATVLGTITCCFMKRLRKR; Peptide sequence of
Isopeptide(1) tag-IGSF8 VLM
217:
GACTACAAAGACCACGACGGGGATTATAAAGATCATGACATCGATTACAAGGAT
GACGATGATAAGGGCTCTGGCAAGTTAGGCGATATCGAATTCATTAAAGTCAATA
AGGGATCCCCCGCCAACCTGAAGGCCCTGGAGGCCCAGAAGCAGAAGGAGCAGA
GACAGGCCGCCGAGGAGCTGGCCAACGCCAAGAAGCTGAAGGAGCAGCTGGAGA
AGCGGGAGGTGCTGGTCCCCGAGGGGCCCTTGTACCGCGTGGCTGGCACAGCTGT
CTCCATCTCCTGCAATGTGACCGGCTATGAGGGCCCTGCCCAGCAGAACTTCGAG
TGGTTCCTGTATAGGCCCGAGGCCCCAGATACTGCACTGGGCATTGTCAGTACCA
AGGATACCCAGTTCTCCTATGCTGTCTTCAAGTCCCGAGTGGTGGCGGGTGAGGT
GCAGGTGCAGCGCCTACAAGGTGATGCCGTGGTGCTCAAGATTGCCCGCCTGCAG
GCCCAGGATGCCGGCATTTATGAGTGCCACACCCCCTCCACTGATACCCGCTACCT
GGGCAGCTACAGCGGCAAGGTGGAGCTGAGAGTTCTTCCAGATGTCCTCCAGGTG
TCTGCTGCCCCCCCAGGGCCCCGAGGCCGCCAGGCCCCAACCTCACCCCCACGCA
TGACGGTGCATGAGGGGCAGGAGCTGGCACTGGGCTGCCTGGCGAGGACAAGCA
CACAGAAGCACACACACCTGGCAGTGTCCTTTGGGCGATCTGTGCCCGAGGCACC
AGTTGGGCGGTCAACTCTGCAGGAAGTGGTGGGAATCCGGTCAGACTTGGCCGTG
GAGGCTGGAGCTCCCTATGCTGAGCGATTGGCTGCAGGGGAGCTTCGTCTGGGCA
AGGAAGGGACCGATCGGTACCGCATGGTAGTAGGGGGTGCCCAGGCAGGGGACG
CAGGCACCTACCACTGCACTGCCGCTGAGTGGATTCAGGATCCTGATGGCAGCTG
GGCCCAGATTGCAGAGAAAAGGGCCGTCCTGGCCCACGTGGATGTGCAGACGCTG
TCCAGCCAGCTGGCAGTGACAGTGGGGCCTGGTGAACGTCGGATCGGCCCAGGGG
AGCCCTTGGAACTGCTGTGCAATGTGTCAGGGGCACTTCCCCCAGCAGGCCGTCA
TGCTGCATACTCTGTAGGTTGGGAGATGGCACCTGCGGGGGCACCTGGGCCCGGC
CGCCTGGTAGCCCAGCTGGACACAGAGGGTGTGGGCAGCCTGGGCCCTGGCTATG
AGGGCCGACACATTGCCATGGAGAAGGTGGCATCCAGAACATACCGGCTACGGCT
AGAGGCTGCCAGGCCTGGTGATGCGGGCACCTACCGCTGCCTCGCCAAAGCCTAT
GTTCGAGGGTCTGGGACCCGGCTTCGTGAAGCAGCCAGTGCCCGTTCCCGGCCTC
TCCCTGTACATGTGCGGGAGGAAGGTGTGGTGCTGGAGGCTGTGGCATGGCTAGC
AGGAGGCACAGTGTACCGCGGGGAGACTGCCTCCCTGCTGTGCAACATCTCTGTG
CGGGGTGGCCCCCCAGGACTGCGGCTGGCCGCCAGCTGGTGGGTGGAGCGACCA
GAGGACGGAGAGCTCAGCTCTGTCCCTGCCCAGCTGGTGGGTGGCGTAGGCCAGG
ATGGTGTGGCAGAGCTGGGAGTCCGGCCTGGAGGAGGCCCTGTCAGCGTAGAGCT
GGTGGGGCCCCGAAGCCATCGGCTGAGACTACACAGCTTGGGGCCCGAGGATGA
AGGCGTGTACCACTGTGCCCCCAGCGCCTGGGTGCAGCATGCCGACTACAGCTGG
TACCAGGCGGGCAGTGCCCGCTCAGGGCCTGTTACAGTCTACCCCTACATGCATG
CCCTGGACACCCTATTTGTGCCTCTGCTGGTGGGTACAGGGGTGGCCCTAGTCACT
GGTGCCACTGTCCTTGGTACCATCACTTGCTGCTTCATGAAGAGGCTTCGAAAACG
G; Coding sequence of Isopeptide(2) tag-IGSF8 VLM
218:
DYKDHDGDYKDHDIDYKDDDDKGSGKIGDIEFIKVNKGSPANLKALEAQKQKEQRQ
AAEELANAKKLKEQLEKREVLVPEGPLYRVAGTAVSISCNVTGYEGPAQQNFEWFLY
RPEAPDTALGIVSTKDTQFSYAVFKSRVVAGEVQVQRLQGDAVVLKIARLQAQDAGI
YECHTPSTDTRYLGSYSGKVELRVLPDVLQVSAAPPGPRGRQAPTSPPRMTVHEGQEL
ALGCLARTSTQKHTHLAVSFGRSVPEAPVGRSTLQEVVGIRSDLAVEAGAPYAERLA
AGELRLGKEGTDRYRMVVGGAQAGDAGTYHCTAAEWIQDPDGSWAQIAEKRAVLA
HVDVQTLSSQLAVTVGPGERRIGPGEPLELLCNVSGALPPAGRHAAYSVGWEMAPAG
APGPGRLVAQLDTEGVGSLGPGYEGRHIAMEKVASRTYRLRLEAARPGDAGTYRCL
AKAYVRGSGTRLREAASARSRPLPVHVREEGVVLEAVAWLAGGTVYRGETASLLCNI
SVRGGPPGLRLAASWWVERPEDGELSSVPAQLVGGVGQDGVAELGVRPGGGPVSVE
LVGPRSHRLRLHSLGPEDEGVYHCAPSAWVQHADYSWYQAGSARSGPVTVYPYMH
ALDTLFVPLLVGTGVALVTGATVLGTITCCFMKRLRKR; Peptide sequence of
Isopeptide(2) tag-IGSF8 VLM
219:
GACTACAAAGACCACGACGGGGATTATAAAGATCATGACATCGATTACAAGGAT
GACGATGATAAGGGCTCTGGCGCACACATCGTTATGGTCGACGCATACAAACCAA
CAAAGGGCGGTGGCGGCTCTGGCGGAGGAGGCTCAAAGTTAGGCGATATCGAAT
TCATTAAAGTCAATAAGGGATCCCCCGCCAACCTGAAGGCCCTGGAGGCCCAGAA
GCAGAAGGAGCAGAGACAGGCCGCCGAGGAGCTGGCCAACGCCAAGAAGCTGAA
GGAGCAGCTGGAGAAGCGGGAGGTGCTGGTCCCCGAGGGGCCCTTGTACCGCGT
GGCTGGCACAGCTGTCTCCATCTCCTGCAATGTGACCGGCTATGAGGGCCCTGCCC
AGCAGAACTTCGAGTGGTTCCTGTATAGGCCCGAGGCCCCAGATACTGCACTGGG
CATTGTCAGTACCAAGGATACCCAGTTCTCCTATGCTGTCTTCAAGTCCCGAGTGG
TGGCGGGTGAGGTGCAGGTGCAGCGCCTACAAGGTGATGCCGTGGTGCTCAAGAT
TGCCCGCCTGCAGGCCCAGGATGCCGGCATTTATGAGTGCCACACCCCCTCCACT
GATACCCGCTACCTGGGCAGCTACAGCGGCAAGGTGGAGCTGAGAGTTCTTCCAG
ATGTCCTCCAGGTGTCTGCTGCCCCCCCAGGGCCCCGAGGCCGCCAGGCCCCAAC
CTCACCCCCACGCATGACGGTGCATGAGGGGCAGGAGCTGGCACTGGGCTGCCTG
GCGAGGACAAGCACACAGAAGCACACACACCTGGCAGTGTCCTTTGGGCGATCTG
TGCCCGAGGCACCAGTTGGGCGGTCAACTCTGCAGGAAGTGGTGGGAATCCGGTC
AGACTTGGCCGTGGAGGCTGGAGCTCCCTATGCTGAGCGATTGGCTGCAGGGGAG
CTTCGTCTGGGCAAGGAAGGGACCGATCGGTACCGCATGGTAGTAGGGGGTGCCC
AGGCAGGGGACGCAGGCACCTACCACTGCACTGCCGCTGAGTGGATTCAGGATCC
TGATGGCAGCTGGGCCCAGATTGCAGAGAAAAGGGCCGTCCTGGCCCACGTGGAT
GTGCAGACGCTGTCCAGCCAGCTGGCAGTGACAGTGGGGCCTGGTGAACGTCGGA
TCGGCCCAGGGGAGCCCTTGGAACTGCTGTGCAATGTGTCAGGGGCACTTCCCCC
AGCAGGCCGTCATGCTGCATACTCTGTAGGTTGGGAGATGGCACCTGCGGGGGCA
CCTGGGCCCGGCCGCCTGGTAGCCCAGCTGGACACAGAGGGTGTGGGCAGCCTGG
GCCCTGGCTATGAGGGCCGACACATTGCCATGGAGAAGGTGGCATCCAGAACATA
CCGGCTACGGCTAGAGGCTGCCAGGCCTGGTGATGCGGGCACCTACCGCTGCCTC
GCCAAAGCCTATGTTCGAGGGTCTGGGACCCGGCTTCGTGAAGCAGCCAGTGCCC
GTTCCCGGCCTCTCCCTGTACATGTGCGGGAGGAAGGTGTGGTGCTGGAGGCTGT
GGCATGGCTAGCAGGAGGCACAGTGTACCGCGGGGAGACTGCCTCCCTGCTGTGC
AACATCTCTGTGCGGGGTGGCCCCCCAGGACTGCGGCTGGCCGCCAGCTGGTGGG
TGGAGCGACCAGAGGACGGAGAGCTCAGCTCTGTCCCTGCCCAGCTGGTGGGTGG
CGTAGGCCAGGATGGTGTGGCAGAGCTGGGAGTCCGGCCTGGAGGAGGCCCTGTC
AGCGTAGAGCTGGTGGGGCCCCGAAGCCATCGGCTGAGACTACACAGCTTGGGGC
CCGAGGATGAAGGCGTGTACCACTGTGCCCCCAGCGCCTGGGTGCAGCATGCCGA
CTACAGCTGGTACCAGGCGGGCAGTGCCCGCTCAGGGCCTGTTACAGTCTACCCC
TACATGCATGCCCTGGACACCCTATTTGTGCCTCTGCTGGTGGGTACAGGGGTGGC
CCTAGTCACTGGTGCCACTGTCCTTGGTACCATCACTTGCTGCTTCATGAAGAGGC
TTCGAAAACGG; Coding sequence of Isopeptide(1) tag + Isopeptide(2)
tag-IGSF8 VLM
220:
DYKDHDGDYKDHDIDYKDDDDKGSGAHIVMVDAYKPTKGGGGSGGGGSKLGDIEFI
KVNKGSPANLKALEAQKQKEQRQAAEELANAKKLKEQLEKREVLVPEGPLYRVAGT
AVSISCNVTGYEGPAQQNFEWFLYRPEAPDTALGIVSTKDTQFSYAVFKSRVVAGEV
QVQRLQGDAVVLKIARLQAQDAGIYECHTPSTDTRYLGSYSGKVELRVLPDVLQVSA
APPGPRGRQAPTSPPRMTVHEGQELALGCLARTSTQKHTHLAVSFGRSVPEAPVGRST
LQEVVGIRSDLAVEAGAPYAERLAAGELRLGKEGTDRYRMVVGGAQAGDAGTYHC
TAAEWIQDPDGSWAQIAEKRAVLAHVDVQTLSSQLAVTVGPGERRIGPGEPLELLCN
VSGALPPAGRHAAYSVGWEMAPAGAPGPGRLVAQLDTEGVGSLGPGYEGRHIAMEK
VASRTYRLRLEAARPGDAGTYRCLAKAYVRGSGTRLREAASARSRPLPVHVREEGVV
LEAVAWLAGGTVYRGETASLLCNISVRGGPPGLRLAASWWVERPEDGELSSVPAQLV
GGVGQDGVAELGVRPGGGPVSVELVGPRSHRLRLHSLGPEDEGVYHCAPSAWVQHA
DYSWYQAGSARSGPVTVYPYMHALDTLFVPLLVGTGVALVTGATVLGTITCCFMKR
LRKR; Peptide sequence of Isopeptide(1) tag+Isopeptide(2) tag-IGSF8 VLM
221:
GACTACAAAGACCACGACGGGGATTATAAAGATCATGACATCGATTACAAGGAT
GACGATGATAAGGATCCGATCGTAATGATAGACAACGACAAACCAATCACCGCC
ATGGTTGATACCTTATCAGGTTTATCAAGTGAGCAAGGTCAGTCCGGTGATGCAC
ACATCGTTATGGTCGACGCATACAAACCAACAAAGGGCGGTGGCGGCTCTGGCGG
AGGAGGCTCAAAGTTAGGCGATATCGAATTCATTAAAGTCAATAAGGGATCCCCC
GCCAACCTGAAGGCCCTGGAGGCCCAGAAGCAGAAGGAGCAGAGACAGGCCGCC
GAGGAGCTGGCCAACGCCAAGAAGCTGAAGGAGCAGCTGGAGAAGCGGGAGGTG
CTGGTCCCCGAGGGGCCCTTGTACCGCGTGGCTGGCACAGCTGTCTCCATCTCCTG
CAATGTGACCGGCTATGAGGGCCCTGCCCAGCAGAACTTCGAGTGGTTCCTGTAT
AGGCCCGAGGCCCCAGATACTGCACTGGGCATTGTCAGTACCAAGGATACCCAGT
TCTCCTATGCTGTCTTCAAGTCCCGAGTGGTGGCGGGTGAGGTGCAGGTGCAGCG
CCTACAAGGTGATGCCGTGGTGCTCAAGATTGCCCGCCTGCAGGCCCAGGATGCC
GGCATTTATGAGTGCCACACCCCCTCCACTGATACCCGCTACCTGGGCAGCTACA
GCGGCAAGGTGGAGCTGAGAGTTCTTCCAGATGTCCTCCAGGTGTCTGCTGCCCC
CCCAGGGCCCCGAGGCCGCCAGGCCCCAACCTCACCCCCACGCATGACGGTGCAT
GAGGGGCAGGAGCTGGCACTGGGCTGCCTGGCGAGGACAAGCACACAGAAGCAC
ACACACCTGGCAGTGTCCTTTGGGCGATCTGTGCCCGAGGCACCAGTTGGGCGGT
CAACTCTGCAGGAAGTGGTGGGAATCCGGTCAGACTTGGCCGTGGAGGCTGGAGC
TCCCTATGCTGAGCGATTGGCTGCAGGGGAGCTTCGTCTGGGCAAGGAAGGGACC
GATCGGTACCGCATGGTAGTAGGGGGTGCCCAGGCAGGGGACGCAGGCACCTAC
CACTGCACTGCCGCTGAGTGGATTCAGGATCCTGATGGCAGCTGGGCCCAGATTG
CAGAGAAAAGGGCCGTCCTGGCCCACGTGGATGTGCAGACGCTGTCCAGCCAGCT
GGCAGTGACAGTGGGGCCTGGTGAACGTCGGATCGGCCCAGGGGAGCCCTTGGA
ACTGCTGTGCAATGTGTCAGGGGCACTTCCCCCAGCAGGCCGTCATGCTGCATACT
CTGTAGGTTGGGAGATGGCACCTGCGGGGGCACCTGGGCCCGGCCGCCTGGTAGC
CCAGCTGGACACAGAGGGTGTGGGCAGCCTGGGCCCTGGCTATGAGGGCCGACA
CATTGCCATGGAGAAGGTGGCATCCAGAACATACCGGCTACGGCTAGAGGCTGCC
AGGCCTGGTGATGCGGGCACCTACCGCTGCCTCGCCAAAGCCTATGTTCGAGGGT
CTGGGACCCGGCTTCGTGAAGCAGCCAGTGCCCGTTCCCGGCCTCTCCCTGTACAT
GTGCGGGAGGAAGGTGTGGTGCTGGAGGCTGTGGCATGGCTAGCAGGAGGCACA
GTGTACCGCGGGGAGACTGCCTCCCTGCTGTGCAACATCTCTGTGCGGGGTGGCC
CCCCAGGACTGCGGCTGGCCGCCAGCTGGTGGGTGGAGCGACCAGAGGACGGAG
AGCTCAGCTCTGTCCCTGCCCAGCTGGTGGGTGGCGTAGGCCAGGATGGTGTGGC
AGAGCTGGGAGTCCGGCCTGGAGGAGGCCCTGTCAGCGTAGAGCTGGTGGGGCC
CCGAAGCCATCGGCTGAGACTACACAGCTTGGGGCCCGAGGATGAAGGCGTGTAC
CACTGTGCCCCCAGCGCCTGGGTGCAGCATGCCGACTACAGCTGGTACCAGGCGG
GCAGTGCCCGCTCAGGGCCTGTTACAGTCTACCCCTACATGCATGCCCTGGACACC
CTATTTGTGCCTCTGCTGGTGGGTACAGGGGTGGCCCTAGTCACTGGTGCCACTGT
CCTTGGTACCATCACTTGCTGCTTCATGAAGAGGCTTCGAAAACGG; Coding
sequence of Isopeptide(1) tag + Isopeptide(2) tag + Isopeptide(3) tag-
IGSF8 VLM
222:
DYKDHDGDYKDHDIDYKDDDDKDPIVMIDNDKPITAMVDTLSGLSSEQGQSGDAHIV
MVDAYKPTKGGGGSGGGGSKLGDIEFIKVNKGSPANLKALEAQKQKEQRQAAEELA
NAKKLKEQLEKREVLVPEGPLYRVAGTAVSISCNVTGYEGPAQQNFEWFLYRPEAPD
TALGIVSTKDTQFSYAVFKSRVVAGEVQVQRLQGDAVVLKIARLQAQDAGIYECHTP
STDTRYLGSYSGKVELRVLPDVLQVSAAPPGPRGRQAPTSPPRMTVHEGQELALGCL
ARTSTQKHTHLAVSFGRSVPEAPVGRSTLQEVVGIRSDLAVEAGAPYAERLAAGELRL
GKEGTDRYRMVVGGAQAGDAGTYHCTAAEWIQDPDGSWAQIAEKRAVLAHVDVQ
TLSSQLAVTVGPGERRIGPGEPLELLCNVSGALPPAGRHAAYSVGWEMAPAGAPGPG
RLVAQLDTEGVGSLGPGYEGRHIAMEKVASRTYRLRLEAARPGDAGTYRCLAKAYV
RGSGTRLREAASARSRPLPVHVREEGVVLEAVAWLAGGTVYRGETASLLCNISVRGG
PPGLRLAASWWVERPEDGELSSVPAQLVGGVGQDGVAELGVRPGGGPVSVELVGPR
SHRLRLHSLGPEDEGVYHCAPSAWVQHADYSWYQAGSARSGPVTVYPYMHALDTLF
VPLLVGTGVALVTGATVLGTITCCFMKRLRKR; Peptide sequence of Isopeptide(1)
tag + Isopeptide(2) tag + Isopeptide(3) tag-IGSF8 VLM

TABLE 9
Fusion Protein or Peptide Comprising Isopeptide Tag and Targeting Moiety

SEQ ID NO: Sequence; Source
223:
GCTTCCGATTACAAGGATGACGATGACAAGGGTTCCCAAATGCAGCTGGTTCAAA
GTGGTGCTGAGGTCAAAAAACCAGGCGCGAGCGTAAAACTGTCCTGTAAAGCCA
GCGGATACACCTTCTCCAGCTACTGGATGCACTGGGTCCGACAGGCCCCAGGGCA
GAGGCTCGAATGGATGGGCGAGATCAACCCCGGCAATGGTCACACCAATTACAAT
GAAAAGTTCAAGAGCCGCGTGACCATTACTGTCGATAAATCTGCATCTACAGCAT
ACATGGAACTTTCCAGCCTTAGATCAGAGGACACAGCCGTATATTATTGTGCCAA
GATCTGGGGACCGTCCCTTACAAGTCCTTTCGATTACTGGGGTCAGGGGACGCTTG
TAACGGTATCCGGGGGGGGAGGTTCCGGCGGAGGCGGTTCAGGAGGGGGGGGTT
CCGGGGGCGGTGGATCTAATTTTATGCTTACGCAACCCCCGTCTGTAAGTGTTTCC
CCAGGGAAAACCGCTCGAATAACCTGTCGAGGAGACAACCTCGGCGATGTTAATG
TCCATTGGTATCAACAGCGACCTGGGCAAGCCCCGGTTTTGGTCATGTACTATGAT
GCGGACCGCCCCAGTGGCATACCGGAACGGTTCAGTGGAAGTAACTCTGGCAATA
CTGCAACGCTGACCATCAGTGGCGTTGAGGCGGGTGACGAGGCAGATTATTACTG
TCAGGTCTGGGACCGGACCAGTGAGTATGTTTTCGGAACCGGCACAAAAGTAACT
GTACTCGGGGGCGGTGGCGGTTCAGGTGGGGGGGGTAGTGGCGGAGGCGGAAGC
GGTTCTCATCACCATCACCACCATGGAGGAGGGGGCTCTGGCGGTGGGGGTTCCG
CCCATATCGTCATGGTCGATGCGATTAAGCCTACCAAG; Coding sequence of FV6A6-
Isopeptide(1) tag
224:
GSDYKDDDDKGSQMQLVQSGAEVKKPGASVKLSCKASGYTFSSYWMHWVRQAPG
QRLEWMGEINPGNGHTNYNEKFKSRVTITVDKSASTAYMELSSLRSEDTAVYYCAKI
WGPSLTSPFDYWGQGTLVTVSGGGGSGGGGSGGGGSGGGGSNFMLTQPPSVSVSPG
KTARITCRGDNLGDVNVHWYQQRPGQAPVLVMYYDADRPSGIPERFSGSNSGNTAT
LTISGVEAGDEADYYCQVWDRTSEYVFGTGTKVTVLGGGGGSGGGGSGGGGSGSHH
HHHHGGGGSGGGGSAHIVMVDAIKPTK; Peptide sequence of FV6A6-Isopeptide(1) tag
225:
GGTTCCGATTACAAGGATGACGATGACAAGGGTTCCGAAGTGCAGCTTGTAGAAA
GTGGGGGCGGACTGGTACAGCCGGGCGGGAGCCTCAGATTGTCATGCGCCGCTTC
TGGTTTCACTTTTTCTTCCTACGGTATGTCCTGGGTTAGACAAGCTCCTGGGAAGG
GTCTTGAGTGGGTGGCTACAATTACTAGTGGTGGTTCATACACGTACTATGTTGAC
AGTGTTAAGGGGCGATTTACTATAAGTAGAGATAATGCCAAGAACACACTCTACC
TTCAGATGAATAGCTTGGGGGGGGAAGATACAGCAGTTTATTATTGCGTTCGGATT
GGCGAGGACGCACTCGACTATTGGGGACAAGGGACTCTTGTTACGGTGTCTAGTG
GGGGGGGAGGTTCCGGCGGAGGCGGTTCAGGAGGGGGGGGTTCCGGGGGCGGTG
GATCTGACATCCAGATGACGCAATCCCCAAGTTCACTTAGCGCTTCAGTCGGCGA
CCGCGTTACCATAACATGCAGAGCAAGTCAAGACATTGCAGGGAGTCTTAATTGG
TTGCAGAAGCCAGGTAAAGCTATAAAGCGCCTTATATATGCCACCAGCAGTCTGG
ATTCTGGTGTACCGAAGAGATTCAGCGGTTCCAGAAGTGGCAGTGACTATACTCT
GACCATTTCTTCTCTCCAGCCTGAAGATTTCGCCACTTACTATTGTCTGCAATATG
GTTCTTTCCCACCAACATTCGGACAAGGTACTAAGGTCGAGATTAAGGGCGGTGG
CGGTTCAGGTGGGGGGGGTAGTGGCGGAGGCGGAAGCGGTTCTCATCACCATCAC
CACCATGGAGGAGGGGGCTCTGGCGGTGGGGGTTCCGCCCATATCGTCATGGTCG
ATGCGATTAAGCCTACCAAG; Coding sequence of FVALAC-Isopeptide(1) tag
226:
GSDYKDDDDKGSEVQLVESGGGLVQPGGSLRLSCAASGFTFSSYGMSWVRQAPGKG
LEWVATITSGGSYTYYVDSVKGRFTISRDNAKNTLYLQMNSLRAEDTAVYYCVRIGE
DALDYWGQGTLVTVSSGGGGSGGGGSGGGGSGGGGSDIQMTQSPSSLSASVGDRVTI
TCRASQDIAGSLNWLQKPGKAIKRLIYATSSLDSGVPKRFSGSRSGSDYTLTISSLQPED
FATYYCLQYGSFPPTFGQGTKVEIKGGGGSGGGGSGGGGSGSHHHHHHGGGGSGGG
GSAHIVMVDAIKPTK; Peptide sequence of FVALAC-Isopeptide(1) tag
227:
GCCCATATCGTCATGGTCGATGCGATTAAGCCTACCAAGGGAGGAGGGGGCTCTG
GCGGTGGGGGTTCCGGTTCCGATTACAAGGATGACGATGACAAGGGTTCCCAAAT
GCAGCTGGTTCAAAGTGGTGCTGAGGTCAAAAAACCAGGCGCGAGCGTAAAACT
GTCCTGTAAAGCCAGCGGATACACCTTCTCCAGCTACTGGATGCACTGGGTCCGA
CAGGCCCCAGGGCAGAGGCTCGAATGGATGGGCGAGATCAACCCCGGCAATGGT
CACACCAATTACAATGAAAAGTTCAAGAGCCGCGTGACCATTACTGTCGATAAAT
CTGCATCTACAGCATACATGGAACTTTCCAGCCTTAGATCAGAGGACACAGCCGT
ATATTATTGTGCCAAGATCTGGGGACCGTCCCTTACAAGTCCTTTCGATTACTGGG
GTCAGGGGACGCTTGTAACGGTATCCGGGGGGGGAGGTTCCGGCGGAGGGGGTTC
AGGAGGGGGGGGTTCCGGGGGCGGTGGATCTAATTTTATGCTTACGCAACCCCCG
TCTGTAAGTGTTTCCCCAGGGAAAACCGCTCGAATAACCTGTCGAGGAGACAACC
TCGGCGATGTTAATGTCCATTGGTATCAACAGCGACCTGGGCAAGCCCCGGTTTTG
GTCATGTACTATGATGCGGACCGCCCCAGTGGCATACCGGAACGGTTCAGTGGAA
GTAACTCTGGCAATACTGCAACGCTGACCATCAGTGGCGTTGAGGCGGGTGACGA
GGCAGATTATTACTGTCAGGTCTGGGACCGGACCAGTGAGTATGTTTTCGGAACC
GGCACAAAAGTAACTGTACTCGGGGGCGGTGGCGGTTCAGGTGGGGGGGGTAGT
GGCGGAGGCGGAAGCGGTTCTCATCACCATCACCACCAT; Coding sequence of
Isopeptide(1) tag-FV6A6
228:
AHIVMVDAIKPTKGGGGSGGGGSGSDYKDDDDKGSQMQLVQSGAEVKKPGASVKL
SCKASGYTFSSYWMHWVRQAPGQRLEWMGEINPGNGHTNYNEKFKSRVTITVDKSA
STAYMELSSLRSEDTAVYYCAKIWGPSLTSPFDYWGQGTLVTVSGGGGSGGGGSGG
GGSGGGGSNFMLTQPPSVSVSPGKTARITCRGDNLGDVNVHWYQQRPGQAPVLVMY
YDADRPSGIPERFSGSNSGNTATLTISGVEAGDEADYYCQVWDRTSEYVFGTGTKVT
VLGGGGGSGGGGSGGGGSGSHHHHHH; Peptide sequence of Isopeptide(1) tag-FV6A6
229:
CAGGTGCAGCTGGTGCAGAGCGGCGCCGAGGTGAAGAAGCCCGGCGCCAGCGTG
AAGGTGAGCTGCAAGGCCAGCGGCTACACCTTCACCGACTACGAGATGCACTGGG
TGAGGCAGGCCCCCGGCCAGGGCCTGGAGTGGATGGGCGCCCTGGACCCCAAGA
CCGGCGACACCGCCTACAGCCAGAAGTTCAAGGGCAGGGTGACCCTGACCGCCG
ACAAGAGCACCAGCACCGCCTACATGGAGCTGAGCAGCCTGACCAGCGAGGACA
CCGCCGTGTACTACTGCACCAGGTTCTACAGCTACACCTACTGGGGCCAGGGCAC
CCTGGTGACCGTGAGCAGCAGCAGCGGCGGCAGCAGCAGGAGCAGCAGCAGCGG
CGGCGGCGGCAGCGGCGGCGGCGGCGACGTGGTGATGACCCAGAGCCCCCTGAG
CCTGCCCGTGACCCCCGGCGAGCCCGCCAGCATCAGCTGCAGGAGCAGCCAGAGC
CTGGTGCACAGCAACGGCAACACCTACCTGCACTGGTACCTGCAGAAGCCCGGCC
AGAGCCCCCAGCTGCTGATCTACAAGGTGAGCAACAGGTTCAGCGGCGTGCCCGA
CAGGTTCAGCGGCAGCGGCAGCGGCACCGACTTCACCCTGAAGATCAGCAGGGTG
GAGGCCGAGGACGTGGGCGTGTACTACTGCAGCCAGAACACCCACGTGCCCCCCA
CCTTCGGCCAGGGCACCAAGCTGGAGATCAAGAGCGGCGGCGGCGGCAGCGGCG
GCGGCGGCAAGGAGACCGCCGCCGCCAAGTTCGAGAGGCAGCACATGGACAGCG
CCCACATCGTGATGGTGGACGCCTACAAGCCCACCAAG; Coding sequence of GC33 +
Isopeptide(1) tag
230:
QVQLVQSGAEVKKPGASVKVSCKASGYTFTDYEMHWVRQAPGQGLEWMGALDPK
TGDTAYSQKFKGRVTLTADKSTSTAYMELSSLTSEDTAVYYCTRFYSYTYWGQGTLV
TVSSSSGGSSRSSSSGGGGSGGGGDVVMTQSPLSLPVTPGEPASISCRSSQSLVHSNGN
TYLHWYLQKPGQSPQLLIYKVSNRFSGVPDRFSGSGSGTDFTLKISRVEAEDVGVYYC
SQNTHVPPTFGQGTKLEIKSGGGGSGGGGKETAAAKFERQHMDSAHIVMVDAYKPT
K; Peptide sequence of GC33 + Isopeptide(1) tag
231:
CAGGTGCAGCTGGTGCAGAGCGGCGCCGAGGTGAAGAAGCCCGGCGCCAGCGTG
AAGGTGAGCTGCAAGGCCAGCGGCTACACCTTCACCGACTACGAGATGCACTGGG
TGAGGCAGGCCCCCGGCCAGGGCCTGGAGTGGATGGGCGCCCTGGACCCCAAGA
CCGGCGACACCGCCTACAGCCAGAAGTTCAAGGGCAGGGTGACCCTGACCGCCG
ACAAGAGCACCAGCACCGCCTACATGGAGCTGAGCAGCCTGACCAGCGAGGACA
CCGCCGTGTACTACTGCACCAGGTTCTACAGCTACACCTACTGGGGCCAGGGCAC
CCTGGTGACCGTGAGCAGCAGCAGCGGCGGCAGCAGCAGGAGCAGCAGCAGCGG
CGGCGGCGGCAGCGGCGGCGGCGGCGACGTGGTGATGACCCAGAGCCCCCTGAG
CCTGCCCGTGACCCCCGGCGAGCCCGCCAGCATCAGCTGCAGGAGCAGCCAGAGC
CTGGTGCACAGCAACGGCAACACCTACCTGCACTGGTACCTGCAGAAGCCCGGCC
AGAGCCCCCAGCTGCTGATCTACAAGGTGAGCAACAGGTTCAGCGGCGTGCCCGA
CAGGTTCAGCGGCAGCGGCAGCGGCACCGACTTCACCCTGAAGATCAGCAGGGTG
GAGGCCGAGGACGTGGGCGTGTACTACTGCAGCCAGAACACCCACGTGCCCCCCA
CCTTCGGCCAGGGCACCAAGCTGGAGATCAAGAGCGGCGGCGGCGGCAGCGGCG
GCGGCGGCGAGCAGAAGCTGATCAGCGAGGAGGACCTGAAGCTGGGCGACATCG
AGTTCATCAAGGTGAACAAG; Coding sequence of GC33 + Isopeptide(2) tag
232:
QVQLVQSGAEVKKPGASVKVSCKASGYTFTDYEMHWVRQAPGQGLEWMGALDPK
TGDTAYSQKFKGRVTLTADKSTSTAYMELSSLTSEDTAVYYCTRFYSYTYWGQGTLV
TVSSSSGGSSRSSSSGGGGSGGGGDVVMTQSPLSLPVTPGEPASISCRSSQSLVHSNGN
TYLHWYLQKPGQSPQLLIYKVSNRFSGVPDRFSGSGSGTDFTLKISRVEAEDVGVYYC
SQNTHVPPTFGQGTKLEIKSGGGGSGGGGEQKLISEEDLKLGDIEFIKVNK; Peptide
sequence of GC33 + Isopeptide(2) tag
233:
CAGGTGCAGCTGGTGCAGAGCGGCGCCGAGGTGAAGAAGCCCGGCGCCAGCGTG
AAGGTGAGCTGCAAGGCCAGCGGCTACACCTTCACCGACTACGAGATGCACTGGG
TGAGGCAGGCCCCCGGCCAGGGCCTGGAGTGGATGGGCGCCCTGGACCCCAAGA
CCGGCGACACCGCCTACAGCCAGAAGTTCAAGGGCAGGGTGACCCTGACCGCCG
ACAAGAGCACCAGCACCGCCTACATGGAGCTGAGCAGCCTGACCAGCGAGGACA
CCGCCGTGTACTACTGCACCAGGTTCTACAGCTACACCTACTGGGGCCAGGGCAC
CCTGGTGACCGTGAGCAGCAGCAGCGGCGGCAGCAGCAGGAGCAGCAGCAGCGG
CGGCGGCGGCAGCGGCGGCGGCGGCGACGTGGTGATGACCCAGAGCCCCCTGAG
CCTGCCCGTGACCCCCGGCGAGCCCGCCAGCATCAGCTGCAGGAGCAGCCAGAGC
CTGGTGCACAGCAACGGCAACACCTACCTGCACTGGTACCTGCAGAAGCCCGGCC
AGAGCCCCCAGCTGCTGATCTACAAGGTGAGCAACAGGTTCAGCGGCGTGCCCGA
CAGGTTCAGCGGCAGCGGCAGCGGCACCGACTTCACCCTGAAGATCAGCAGGGTG
GAGGCCGAGGACGTGGGCGTGTACTACTGCAGCCAGAACACCCACGTGCCCCCCA
CCTTCGGCCAGGGCACCAAGCTGGAGATCAAGAGCGGCGGCGGCGGCAGCGGCG
GCGGCGGCGGCAAGCCCATCCCCAACCCCCTGCTGGGCCTGGACAGCACCGACCC
CATCGTGATGATCGACAACGACAAGCCCATCACC; Coding sequence of GC33 +
Isopeptide(3) tag
234
QVQLVQSGAEVKKPGASVKVSCKASGYTFTDYEMHWVRQAPGQGLEWMGALDPK
TGDTAYSQKFKGRVTLTADKSTSTAYMELSSLTSEDTAVYYCTRFYSYTYWGQGTLV
TVSSSSGGSSRSSSSGGGGSGGGGDVVMTQSPLSLPVTPGEPASISCRSSQSLVHSNGN
TYLHWYLQKPGQSPQLLIYKVSNRFSGVPDRFSGSGSGTDFTLKISRVEAEDVGVYYC
SQNTHVPPTFGQGTKLEIKSGGGGSGGGGGKPIPNPLLGLDSTDPIVMIDNDKPIT;
Peptide sequence of GC33 + Isopeptide(3) tag
235:
ACCCACGTGAGCCCCAACCAGGGCGGCCTGCCCAGCAGCGGCGGCGGCGGCAGC
GGCGGCGGCGGCAAGGAGACCGCCGCCGCCAAGTTCGAGAGACAGCACATGGAC
AGCGCCCACATCGTGATGGTGGACGCCTACAAGCCCACCAAG; Coding sequence of
N-terminal PEPN + Isopeptide(1) tag
236: THVSPNQGGLPSSGGGGSGGGGKETAAAKFERQHMDSAHIVMVDAYKPTK;
Peptide sequence of N-terminal PEPN + Isopeptide(1) tag
237:
GCCCACATCGTGATGGTGGACGCCTACAAGCCCACCAAGAGCGGCGGCGGCGGC
AGCGGCGGCGGCGGCAAGGAGACCGCCGCCGCCAAGTTCGAGAGACAGCACATG
GACAGCACCCACGTGAGCCCCAACCAGGGCGGCCTGCCCAGC; Coding sequence of
C-terminal PEPN + Isopeptide(1) tag
238: AHIVMVDAYKPTKSGGGGSGGGGKETAAAKFERQHMDSTHVSPNQGGLPS;
Peptide sequence of C-terminal PEPN + Isopeptide(1) tag
239:
ACCCACGTGAGCCCCAACCAGGGCGGCCTGCCCAGCAGCGGCGGCGGCGGCAGC
GGCGGCGGCGGCGAGCAGAAGCTGATCAGCGAGGAGGACCTGAAGCTGGGCGAC
ATCGAGTTCATCAAGGTGAACAAG; Coding sequence of N-terminal PEPN +
Isopeptide(2) tag
240: THVSPNQGGLPSSGGGGSGGGGEQKLISEEDLKLGDIEFIKVNK; Peptide sequence
of N-terminal PEPN + Isopeptide(2) tag
241:
AAGCTGGGCGACATCGAGTTCATCAAGGTGAACAAGAGCGGCGGCGGCGGCAGC
GGCGGCGGCGGCGAGCAGAAGCTGATCAGCGAGGAGGACCTGACCCACGTGAGC
CCCAACCAGGGCGGCCTGCCCAGC; Coding sequence of C-terminal PEPN +
Isopeptide(2) tag
242: KLGDIEFIKVNKSGGGGSGGGGEQKLISEEDLTHVSPNQGGLPS; Peptide sequence
of C-terminal PEPN + Isopeptide(2) tag
243:
ACCCACGTGAGCCCCAACCAGGGCGGCCTGCCCAGCAGCGGCGGCGGCGGCAGC
GGCGGCGGCGGCGGCAAGCCCATCCCCAACCCCCTGCTGGGCCTGGACAGCACCG
ACCCCATCGTGATGATCGACAACGACAAGCCCATCACC; Coding sequence of N~
terminal PEPN + Isopeptide(3) tag
244: THVSPNQGGLPSSGGGGSGGGGGKPIPNPLLGLDSTDPIVMIDNDKPIT; Peptide
sequence of N-terminal PEPN + Isopeptide(3) tag
245:
GACCCCATCGTGATGATCGACAACGACAAGCCCATCACCAGCGGCGGCGGCGGC
AGCGGCGGCGGCGGCGGCAAGCCCATCCCCAACCCCCTGCTGGGCCTGGACAGCA
CCACCCACGTGAGCCCCAACCAGGGCGGCCTGCCCAGC, Coding sequence of C-
terminal PEPN + Isopeptide(3) tag
246: DPIVMIDNDKPITSGGGGSGGGGGKPIPNPLLGLDSTTHVSPNQGGLPS; Peptide
sequence of C-terminal PEPN + Isopeptide(3) tag

TABLE 10
Fusion Protein Comprising Isopeptide Domain and Targeting Moiety

SEQ ID NO: Sequence; Source
247:
CAGGTGCAGCTGGTGCAGAGCGGCGCCGAGGTGAAGAAGCCCGGCGCCAGCGTG
AAGGTGAGCTGCAAGGCCAGCGGCTACACCTTCACCGACTACGAGATGCACTGGG
TGAGGCAGGCCCCCGGCCAGGGCCTGGAGTGGATGGGCGCCCTGGACCCCAAGA
CCGGCGACACCGCCTACAGCCAGAAGTTCAAGGGCAGGGTGACCCTGACCGCCG
ACAAGAGCACCAGCACCGCCTACATGGAGCTGAGCAGCCTGACCAGCGAGGACA
CCGCCGTGTACTACTGCACCAGGTTCTACAGCTACACCTACTGGGGCCAGGGCAC
CCTGGTGACCGTGAGCAGCAGCAGCGGCGGCAGCAGCAGGAGCAGCAGCAGCGG
CGGCGGCGGCAGCGGCGGCGGCGGCGACGTGGTGATGACCCAGAGCCCCCTGAG
CCTGCCCGTGACCCCCGGCGAGCCCGCCAGCATCAGCTGCAGGAGCAGCCAGAGC
CTGGTGCACAGCAACGGCAACACCTACCTGCACTGGTACCTGCAGAAGCCCGGCC
AGAGCCCCCAGCTGCTGATCTACAAGGTGAGCAACAGGTTCAGCGGCGTGCCCGA
CAGGTTCAGCGGCAGCGGCAGCGGCACCGACTTCACCCTGAAGATCAGCAGGGTG
GAGGCCGAGGACGTGGGCGTGTACTACTGCAGCCAGAACACCCACGTGCCCCCCA
CCTTCGGCCAGGGCACCAAGCTGGAGATCAAGAGCGGCGGCGGCGGCAGCGGCG
GCGGCGGCAAGGAGACCGCCGCCGCCAAGTTCGAGAGGCAGCACATGGACAGCG
ACAGCGCCACCCACATCAAGTTCAGCAAGAGGGACGAGGACGGCAAGGAGCTGG
CCGGCGCCACCATGGAGCTGAGGGACAGCAGCGGCAAGACCATCAGCACCTGGA
TCAGCGACGGCCAGGTGAAGGACTTCTACCTGTACCCCGGCAAGTACACCTTCGT
GGAGACCGCCGCCCCCGACGGCTACGAGGTGGCCACCGCCATCACCTTCACCGTG
AACGAGCAGGGCCAGGTGACCGTGAACGGC; Coding sequence of GC33 +
Isopeptide(1) domain
248:
QVQLVQSGAEVKKPGASVKVSCKASGYTFTDYEMHWVRQAPGQGLEWMGALDPK
TGDTAYSQKFKGRVTLTADKSTSTAYMELSSLTSEDTAVYYCTRFYSYTYWGQGTLV
TVSSSSGGSSRSSSSGGGGSGGGGDVVMTQSPLSLPVTPGEPASISCRSSQSLVHSNGN
TYLHWYLQKPGQSPQLLIYKVSNRFSGVPDRFSGSGSGTDFTLKISRVEAEDVGVYYC
SQNTHVPPTFGQGTKLEIKSGGGGSGGGGKETAAAKFERQHMDSDSATHIKFSKRDE
DGKELAGATMELRDSSGKTISTWISDGQVKDFYLYPGKYTFVETAAPDGYEVATAITF
TVNEQGQVTVNG; Peptide sequence of GC33 + Isopeptide(1) domain
249:
CAGGTGCAGCTGGTGCAGAGCGGCGCCGAGGTGAAGAAGCCCGGCGCCAGCGTG
AAGGTGAGCTGCAAGGCCAGCGGCTACACCTTCACCGACTACGAGATGCACTGGG
TGAGGCAGGCCCCCGGCCAGGGCCTGGAGTGGATGGGCGCCCTGGACCCCAAGA
CCGGCGACACCGCCTACAGCCAGAAGTTCAAGGGCAGGGTGACCCTGACCGCCG
ACAAGAGCACCAGCACCGCCTACATGGAGCTGAGCAGCCTGACCAGCGAGGACA
CCGCCGTGTACTACTGCACCAGGTTCTACAGCTACACCTACTGGGGCCAGGGCAC
CCTGGTGACCGTGAGCAGCAGCAGCGGCGGCAGCAGCAGGAGCAGCAGCAGCGG
CGGCGGCGGCAGCGGCGGCGGCGGCGACGTGGTGATGACCCAGAGCCCCCTGAG
CCTGCCCGTGACCCCCGGCGAGCCCGCCAGCATCAGCTGCAGGAGCAGCCAGAGC
CTGGTGCACAGCAACGGCAACACCTACCTGCACTGGTACCTGCAGAAGCCCGGCC
AGAGCCCCCAGCTGCTGATCTACAAGGTGAGCAACAGGTTCAGCGGCGTGCCCGA
CAGGTTCAGCGGCAGCGGCAGCGGCACCGACTTCACCCTGAAGATCAGCAGGGTG
GAGGCCGAGGACGTGGGCGTGTACTACTGCAGCCAGAACACCCACGTGCCCCCCA
CCTTCGGCCAGGGCACCAAGCTGGAGATCAAGAGCGGCGGCGGCGGCAGCGGCG
GCGGCGGCGAGCAGAAGCTGATCAGCGAGGAGGACCTGGGCAGCCACATGAAGC
CCCTGAGGGGCGCCGTGTTCAGCCTGCAGAAGCAGCACCCCGACTACCCCGACAT
CTACGGCGCCATCGACCAGAACGGCACCTACCAGAACGTGAGGACCGGCGAGGA
CGGCAAGCTGACCTTCAAGAACCTGAGCGACGGCAAGTACAGGCTGTTCGAGAAC
AGCGAGCCCGCCGGCTACAAGCCCGTGCAGAACAAGCCCATCGTGGCCTTCCAGA
TCGTGAACGGCGAGGTGAGGGACGTGACCAGCATCGTGCCCCAGGACATCCCCGC
CACCTACGAGTTCACCAACGGCAAGCACTACATCACCAACGAGCCCATCCCCCCC
AAG; Coding sequence of GC33 + Isopeptide(2) domain
250:
QVQLVQSGAEVKKPGASVKVSCKASGYTFTDYEMHWVRQAPGQGLEWMGALDPK
TGDTAYSQKFKGRVTLTADKSTSTAYMELSSLTSEDTAVYYCTRFYSYTYWGQGTLV
TVSSSSGGSSRSSSSGGGGSGGGGDVVMTQSPLSLPVTPGEPASISCRSSQSLVHSNGN
TYLHWYLQKPGQSPQLLIYKVSNRFSGVPDRFSGSGSGTDFTLKISRVEAEDVGVYYC
SQNTHVPPTFGQGTKLEIKSGGGGSGGGGEQKLISEEDLGSHMKPLRGAVFSLQKQHP
DYPDIYGAIDQNGTYQNVRTGEDGKLTFKNLSDGKYRLFENSEPAGYKPVQNKPIVA
FQIVNGEVRDVTSIVPQDIPATYEFTNGKHYITNEPIPPK; Peptide sequence of GC33 +
Isopeptide(2) domain
251:
CAGGTGCAGCTGGTGCAGAGCGGCGCCGAGGTGAAGAAGCCCGGCGCCAGCGTG
AAGGTGAGCTGCAAGGCCAGCGGCTACACCTTCACCGACTACGAGATGCACTGGG
TGAGGCAGGCCCCCGGCCAGGGCCTGGAGTGGATGGGCGCCCTGGACCCCAAGA
CCGGCGACACCGCCTACAGCCAGAAGTTCAAGGGCAGGGTGACCCTGACCGCCG
ACAAGAGCACCAGCACCGCCTACATGGAGCTGAGCAGCCTGACCAGCGAGGACA
CCGCCGTGTACTACTGCACCAGGTTCTACAGCTACACCTACTGGGGCCAGGGCAC
CCTGGTGACCGTGAGCAGCAGCAGCGGCGGCAGCAGCAGGAGCAGCAGCAGCGG
CGGCGGCGGCAGCGGCGGCGGCGGCGACGTGGTGATGACCCAGAGCCCCCTGAG
CCTGCCCGTGACCCCCGGCGAGCCCGCCAGCATCAGCTGCAGGAGCAGCCAGAGC
CTGGTGCACAGCAACGGCAACACCTACCTGCACTGGTACCTGCAGAAGCCCGGCC
AGAGCCCCCAGCTGCTGATCTACAAGGTGAGCAACAGGTTCAGCGGCGTGCCCGA
CAGGTTCAGCGGCAGCGGCAGCGGCACCGACTTCACCCTGAAGATCAGCAGGGTG
GAGGCCGAGGACGTGGGCGTGTACTACTGCAGCCAGAACACCCACGTGCCCCCCA
CCTTCGGCCAGGGCACCAAGCTGGAGATCAAGAGCGGCGGCGGCGGCAGCGGCG
GCGGCGGCGGCAAGCCCATCCCCAACCCCCTGCTGGGCCTGGACAGCACCAGCAG
CGGCCTGGTGCCCAGGGGCAGCCACATGGCCAGCATGACCGGCGGCCAGCAGAT
GGGCAGGGGCAGCAGCGGCCTGAGCGGCGAGACCGGCCAGAGCGGCAACACCAC
CATCGAGGAGGACAGCACCACCCACGTGAAGTTCAGCAAGAGGGACGCCAACGG
CAAGGAGCTGGCCGGCGCCATGATCGAGCTGAGGAACCTGAGCGGCCAGACCAT
CCAGAGCTGGATCAGCGACGGCACCGTGAAGGTGTTCTACCTGATGCCCGGCACC
TACCAGTTCGTGGAGACCGCCGCCCCCGAGGGCTACGAGCTGGCCGCCCCCATCA
CCTTCACCATCGACGAGAAGGGCCAGATCTGGGTGGACAGC; Coding sequence of
GC33 + Isopeptide(3) domain
252:
QVQLVQSGAEVKKPGASVKVSCKASGYTFTDYEMHWVRQAPGQGLEWMGALDPK
TGDTAYSQKFKGRVTLTADKSTSTAYMELSSLTSEDTAVYYCTRFYSYTYWGQGTLV
TVSSSSGGSSRSSSSGGGGSGGGGDVVMTQSPLSLPVTPGEPASISCRSSQSLVHSNGN
TYLHWYLQKPGQSPQLLIYKVSNRFSGVPDRFSGSGSGTDFTLKISRVEAEDVGVYYC
SQNTHVPPTFGQGTKLEIKSGGGGSGGGGGKPIPNPLLGLDSTSSGLVPRGSHMASMT
GGQQMGRGSSGLSGETGQSGNTTIEEDSTTHVKFSKRDANGKELAGAMIELRNLSGQ
TIQSWISDGTVKVFYLMPGTYQFVETAAPEGYELAAPITFTIDEKGQIWVDS; Peptide
sequence of GC33 + Isopeptide(3) domain
253:
ACCCACGTGAGCCCCAACCAGGGCGGCCTGCCCAGCAGCGGCGGCGGCGGCAGC
GGCGGCGGCGGCAAGGAGACCGCCGCCGCCAAGTTCGAGAGGCAGCACATGGAC
AGCGACAGCGCCACCCACATCAAGTTCAGCAAGAGGGACGAGGACGGCAAGGAG
CTGGCCGGCGCCACCATGGAGCTGAGGGACAGCAGCGGCAAGACCATCAGCACC
TGGATCAGCGACGGCCAGGTGAAGGACTTCTACCTGTACCCCGGCAAGTACACCT
TCGTGGAGACCGCCGCCCCCGACGGCTACGAGGTGGCCACCGCCATCACCTTCAC
CGTGAACGAGCAGGGCCAGGTGACCGTGAACGGC; Coding sequence of N-terminal
PEPN + Isopeptide(1) domain
254:
THVSPNQGGLPSSGGGGSGGGGKETAAAKFERQHMDSDSATHIKFSKRDEDGKELAG
ATMELRDSSGKTISTWISDGQVKDFYLYPGKYTFVETAAPDGYEVATAITFTVNEQGQ
VTVNG; Peptide sequence of N-terminal PEPN + Isopeptide(1) domain
255:
GACAGCGCCACCCACATCAAGTTCAGCAAGAGGGACGAGGACGGCAAGGAGCTG
GCCGGCGCCACCATGGAGCTGAGGGACAGCAGCGGCAAGACCATCAGCACCTGG
ATCAGCGACGGCCAGGTGAAGGACTTCTACCTGTACCCCGGCAAGTACACCTTCG
TGGAGACCGCCGCCCCCGACGGCTACGAGGTGGCCACCGCCATCACCTTCACCGT
GAACGAGCAGGGCCAGGTGACCGTGAACGGCAGCGGCGGCGGCGGCAGCGGCGG
CGGCGGCAAGGAGACCGCCGCCGCCAAGTTCGAGAGGCAGCACATGGACAGCAC
CCACGTGAGCCCCAACCAGGGCGGCCTGCCCAGC; Coding sequence of C-terminal
PEPN + Isopeptide(1) domain
256:
DSATHIKFSKRDEDGKELAGATMELRDSSGKTISTWISDGQVKDFYLYPGKYTFVETA
APDGYEVATAITFTVNEQGQVTVNGSGGGGSGGGGKETAAAKFERQHMDSTHVSPN
QGGLPS; Peptide sequence of C-terminal PEPN + Isopeptide(1) domain
257:
ACCCACGTGAGCCCCAACCAGGGCGGCCTGCCCAGCAGCGGCGGCGGCGGCAGC
GGCGGCGGCGGCGAGCAGAAGCTGATCAGCGAGGAGGACCTGGGCAGCCACATG
AAGCCCCTGAGGGGCGCCGTGTTCAGCCTGCAGAAGCAGCACCCCGACTACCCCG
ACATCTACGGCGCCATCGACCAGAACGGCACCTACCAGAACGTGAGGACCGGCG
AGGACGGCAAGCTGACCTTCAAGAACCTGAGCGACGGCAAGTACAGGCTGTTCG
AGAACAGCGAGCCCGCCGGCTACAAGCCCGTGCAGAACAAGCCCATCGTGGCCTT
CCAGATCGTGAACGGCGAGGTGAGGGACGTGACCAGCATCGTGCCCCAGGACAT
CCCCGCCACCTACGAGTTCACCAACGGCAAGCACTACATCACCAACGAGCCCATC
CCCCCCAAG; Coding sequence of N-terminal PEPN + Isopeptide(2) domain
258:
THVSPNQGGLPSSGGGGSGGGGEQKLISEEDLGSHMKPLRGAVFSLQKQHPDYPDIY
GAIDQNGTYQNVRTGEDGKLTFKNLSDGKYRLFENSEPAGYKPVQNKPIVAFQIVNG
EVRDVTSIVPQDIPATYEFTNGKHYITNEPIPPK; Peptide sequence of N-terminal PEPN +
Isopeptide(2) domain
259:
GGCAGCCACATGAAGCCCCTGAGGGGCGCCGTGTTCAGCCTGCAGAAGCAGCACC
CCGACTACCCCGACATCTACGGCGCCATCGACCAGAACGGCACCTACCAGAACGT
GAGGACCGGCGAGGACGGCAAGCTGACCTTCAAGAACCTGAGCGACGGCAAGTA
CAGGCTGTTCGAGAACAGCGAGCCCGCCGGCTACAAGCCCGTGCAGAACAAGCC
CATCGTGGCCTTCCAGATCGTGAACGGCGAGGTGAGGGACGTGACCAGCATCGTG
CCCCAGGACATCCCCGCCACCTACGAGTTCACCAACGGCAAGCACTACATCACCA
ACGAGCCCATCCCCCCCAAGAGCGGCGGCGGCGGCAGCGGCGGCGGCGGCGAGC
AGAAGCTGATCAGCGAGGAGGACCTGACCCACGTGAGCCCCAACCAGGGGGGCC
TGCCCAGC, Coding sequence of C-terminal PEPN + Isopeptide(2) domain
260:
GSHMKPLRGAVFSLQKQHPDYPDIYGAIDQNGTYQNVRTGEDGKLTFKNLSDGKYR
LFENSEPAGYKPVQNKPIVAFQIVNGEVRDVTSIVPQDIPATYEFTNGKHYITNEPIPPK
SGGGGSGGGGEQKLISEEDLTHVSPNQGGLPS; Peptide sequence of C-terminal PEPN +
Isopeptide(2) domain
261:
ACCCACGTGAGCCCCAACCAGGGCGGCCTGCCCAGCAGCGGCGGCGGCGGCAGC
GGCGGCGGCGGCGGCAAGCCCATCCCCAACCCCCTGCTGGGCCTGGACAGCACCA
GCAGCGGCCTGGTGCCCAGGGGCAGCCACATGGCCAGCATGACCGGCGGCCAGC
AGATGGGCAGGGGCAGCAGCGGCCTGAGCGGCGAGACCGGCCAGAGCGGCAACA
CCACCATCGAGGAGGACAGCACCACCCACGTGAAGTTCAGCAAGAGGGACGCCA
ACGGCAAGGAGCTGGCCGGCGCCATGATCGAGCTGAGGAACCTGAGCGGCCAGA
CCATCCAGAGCTGGATCAGCGACGGCACCGTGAAGGTGTTCTACCTGATGCCCGG
CACCTACCAGTTCGTGGAGACCGCCGCCCCCGAGGGCTACGAGCTGGCCGCCCCC
ATCACCTTCACCATCGACGAGAAGGGCCAGATCTGGGTGGACAGC; Coding
sequence of N-terminal PEPN + Isopeptide(3) domain
262:
THVSPNQGGLPSSGGGGSGGGGGKPIPNPLLGLDSTSSGLVPRGSHMASMTGGQQMG
RGSSGLSGETGQSGNTTIEEDSTTHVKFSKRDANGKELAGAMIELRNLSGQTIQSWISD
GTVKVFYLMPGTYQFVETAAPEGYELAAPITFTIDEKGQIWVDS; Peptide sequence of
N-terminal PEPN + Isopeptide(3) domain
263:
AGCAGCGGCCTGGTGCCCAGGGGCAGCCACATGGCCAGCATGACCGGCGGCCAG
CAGATGGGCAGGGGCAGCAGCGGCCTGAGCGGCGAGACCGGCCAGAGCGGCAAC
ACCACCATCGAGGAGGACAGCACCACCCACGTGAAGTTCAGCAAGAGGGACGCC
AACGGCAAGGAGCTGGCCGGCGCCATGATCGAGCTGAGGAACCTGAGCGGCCAG
ACCATCCAGAGCTGGATCAGCGACGGCACCGTGAAGGTGTTCTACCTGATGCCCG
GCACCTACCAGTTCGTGGAGACCGCCGCCCCCGAGGGCTACGAGCTGGCCGCCCC
CATCACCTTCACCATCGACGAGAAGGGCCAGATCTGGGTGGACAGCAGCGGCGGC
GGCGGCAGCGGCGGCGGCGGCGGCAAGCCCATCCCCAACCCCCTGCTGGGCCTGG
ACAGCACCACCCACGTGAGCCCCAACCAGGGCGGCCTGCCCAGC; Coding sequence
of C-terminal PEPN + Isopeptide(3) domain
264:
SSGLVPRGSHMASMTGGQQMGRGSSGLSGETGQSGNTTIEEDSTTHVKFSKRDANGK
ELAGAMIELRNLSGQTIQSWISDGTVKVFYLMPGTYQFVETAAPEGYELAAPITFTIDE
KGQIWVDSSGGGGSGGGGGKPIPNPLLGLDSTTHVSPNQGGLPS; Peptide sequence of
C-terminal PEPN + Isopeptide(3) domain

TABLE 11
Isopeptide Protein*

SEQ ID NO: Sequence; Source
265:
MTKSVKFLVLLLVMILPIAGALLIGPISFGAELSKSSIVDKVELDHTTLYQGEMTSIKVS
FSDKENQKIKPGDTITLTLPNALVGMTENDGSPRKINLNGLGEVFIYKDHVVATFNEK
VESLHNVNGHFSFGIKTLITNSSQPNVIETDFGTATATQRLTIEGVTNTETGQIERDYPF
FYKVGDLAGESNQVRWFLNVNLNKSDVTEDISIADRQGSGQQLNKESFTFDIVNDKE
TKYISLAEFEQQGYGKIDFVTDNDFNLRFYRDKARFTSFIVRYTSTITEAGQHQATFEN
SYDINYQLNNQDATNEKNTSQVKNVFVEGEASGNQNVEMPTEESLDIPLETIDEWEPK
TPTSEQATETSEKTGATETAESSQPEVHVSPTEEENPDESETLGTIEPIIPEKPSVTTKEN
GVTETAESSQPEVHVSPTEEENPDESETLGTIAPILPEKPSVTTEENGTTETAESSQPKV
HVSPTEEENPDESETLGTIAPILPEKPSVTTEENGTTETAESSQPEVHVSSAEEENPDESE
TLGTIAPILPEKPSVTTEENGATETAESSQSEVHVSPTKEITITEKKQPSTETTVETNKNV
TSKNQPQILNAPLNTVKNEGSPQLAPQLLSEPIQKLNEANGQRELPKTGTTKTPFMLIA
GILASTFAVLGVSYLQIRKN; ACE19_Q9F865
266:
MKKIFSVLLVLFLTFSTWSSVLVKADSPSKGTLTIHKYEQEKDGAQGLEGDGSANQEV
PKDVKPLKGVTFEVKRVASFEKISNDGKIVKEDVKPVMGATPNQVVTDDNGQAVLK
DLPLGRYEVKEVAGPPHVNLNPNTYTVDIPLINKEGKVLNYDVHMYPKNEIKRGAV
DLIKTGVNEKALAGAVFSLFKKDGTEVKKELATDANGHIRVQGLEYGEYYFQETKAP
KGYVIDPTKREFFVKNSGTINEDGTITSGTVVKIEVKNNEEPTIDKKINGKLEALPINPL
TNYNYDIKTLIPEDIKEYKKYVVTDTLDNRLVIQGKPIVKIDGAEVNANVVEVAIEGQ
KVTATVKDFTKLDGKKEFHLQIKSQVKEGVPSGSEILNTAKIHFTNKNDVIGEKESKP
VVVIPTTGHIELTKIDSANKNKLKGAEFVLKDNNGKIVVVAGKEVTGVSDENGVIKWS
NIPYGDYQIFETKAPTYTKEDGTKTSYQLLKDPIDVKISENNQTVKLTIENNKSGWILP
VTGGIGTTLFTVIGLTLMLTAAFVFFRKKFARN; BCPA_Q81D71
267:
MNKNVLKFMVFIMLLNIITPLFNKNEAFAARDISSTNVTDLTVSPSKIEDGGKTTVKM
TFDDKNGKIQNGDMIKVAWPTSGTVKIEGYSKTVPLTVKGEQVGQAVITPDGATITEN
DKVEKLSDVSGFAEFEVQGRNLTQTNTSDDKVATITSGNKSTNVTVHKSEAGTSSVF
YYKTGDMLPEDTTHVRWFLNINNEKSYVSKDITIKDQIQGGQQLDLSTLNINVTGTHS
NYYSGQSAITDFEKAFPGSKITVDNTKNTIDVTIPQGYGSYNSFSINYKTKITNEQQKEF
VNNSQAWYQEHGKEEVNGKSFNHTVHNINANAGIEGTVKGELKVLKQDKDTKAPIA
NVKFKLSKKDGSVVKDNQKEIEIITDANGIANIKALPSGDYILKEIEAPRPYTFDKDKE
YPFTMKDTDNQGYFTTIENAKAIEKTKDVSAQKVWEGTQKVKPTIYFKLYKQDDNQ
NTTPVDKAEIKKLEDGTTKVTWSNLPENDKNGKAIKYLVKEVNAQGEDTTPEGYTK
KENGLVVTNTEKPIETTSISGEKVWDDKDNQDGKRPEKVSVNLLANGEKVKTLDVTS
ETNWKYEFKDLPKYDEGKKIEYTVTEDHVKDYTTDINGTTITNKYTPGETSATVTKN
WDDNNNQDGKRPTEIKVELYQDGKATGKTAILNESNNWTHTWTGLDEKAKGQQVK
YTVEELTKVKGYTTHVDNNDMGNLIVTNKYTPETTSISGEKVWDDKDNQDGKRPEK
VSVNLLADGEKVKTLDVTSETNWKYEFKDLPKYDEGKKIEYTVTEDHVKDYTTDIN
GTTITNKYTPGETSATVTKNWDDNNNQDGKRPTEIKVELYQDGKATGKTAILNESNN
WTHTWTGLDEKAKGQQVKYTVEELTKVKGYTTHVDNNDMGNLIVINKYTPETTSIS
GEKVWDDKDNQDGKRPEKVSVNLLANGEKVKTLDVTSETNWKYEFKDLPKYDEGK
KIEYTVTEDHVKDYTTDINGTTITNKYTPGETSATVTKNWDDNNNQDGKRPTEIKVEL
YQDGKATGKTAILNESNNWTHTWTGLDEKAKGQQVKYTVDELTKVNGYTTHVDNN
DMGNLIVTNKYTPKKPNKPIYPEKPKDKTPPTKPDHSNKVKPTPPDKPSKVDKDDQPK
DNKTKPENPLKELPKTGMKIITSWITWVFIGILGLYLILRKRFNS; Cna_Q53654
268:
MTQKNSYKLSFLLSLTGFILGLLLVFIGLSGVSVGHAETRNGANKQGAFEIKKNKSQE
EYNYEVYDNRNILQDGEHKLEIKRVDGTGKTYQGFCFQLTKNFPTAQGVSKKLYKKL
SSSDEETLKQYASKYTSNRRGDTSGNLKKQIAKVLTEGYPTNKSDWLNGLTENEKIEV
TQDAIWYFTETTVPADRSYTNRNVNSQKMKEVYQKLIDTTDIDKYEDVQFDLFVPQD
TNLQAVISVEPVIESLPWTSLKPIAQKDITAKKIWVDAPKEKPIIYFKLYRQLPGEKEVA
VDDAELKQINSEGQQEISVTWTNQLVTDEKGMAYIYSVKEVDKNGELLEPKDYIKKE
DGLTVTNTYVKPTSGHYDIEVTFGNGHIDITEDTTPDIVSGENQMKQIEGEDSKPIDEV
TENNLIEFGKNTMPGEEDGTNSNKYEEVEDSRPVDTLSGLSSEQGQSGDMTIEEDSAT
HIKFSKRDIDGKELAGATMELRDSSGKTISTWISDGQVKDFYLMPGKYTFVETAAPDG
YEIATAITFTVNEQGQVTVNGKATKGDAHIVMVDAYKPTKGSGQVIDIEEKLPDEQG
HSGSTTEIEDSKSSDLIIGGQGEVVDTTEDTQSGMTGHSASTTEIEDSKSSDVIVGGQG
QIVETTEDTQTGMHGDSGRKTEVEDTKLVQSFHFDNKESESNSEIPKKDKPKSNTSLP
ATGEKQHNKFFWMVTSCSLISSVFVISLKTKKCLSSC; FbabB_Q6A1F3
269:
MRGEKMKKTRFPNKLNTLNTQRVLSKNSKRFTVTLVGVFLMIFALVTSMVGAKTVF
GLVESSTPNAINPDSSSEYRWYGYESYVRGHPYYKQFRVAHDLRVNLEGSRSYQVYC
FNLKKAFPLGSDSSVKKWYKKHDGISTKFEDYAISPRITGDELNQKLRAVMYNGHPQ
NANGIMEGLEPLNAIRVTQEAVWYYSDNAPISNPDESFKRESESNLVSTSQLSLMRQA
LKQLIDPNLATKMPKQVPDDFQLSIFESEDKGDKYNKGYQNLLSGGLVPTKPPTPGDP
PMPPNQPQTTSVLIRKYAIGDYSKLLEGATLQLTGDNVNSFQARVFSSNDIGERIELSD
GTYTLTELNSPAGYSIAEPITFKVEAGKVYTIIDGKQIENPNKEIVEPYSVEAYNDFEEF
SVLTTQNYAKFYYAKNKNGSSQVVYCFNADLKSPPDSEDGGKTMTPDFTTGEVKYT
HIAGRDLFKYTVKPRDTDPDTFLKHIKKVIEKGYREKGQAIEYSGLTETQLRAATQLAI
YYFTDSAELDKDKLKDYHGFGDMNDSTLAVAKILVEYAQDSNPPQLTDLDFFIPNNN
KYQSLIGTQWHPEDLVDIIRMEDKKEVIPVTHNLTLRKTVTGLAGDRTKDFHFEIELK
NNKQELLSQTVKTDKTNLEFKDGKATINLKHGESLTLQGLPEGYSYLVKETDSEGYK
VKVNSQEVANATVSKTGITSDETLAFENNKEPVVPTGVDQKINGYLALIVIAGISLGIW
GIHTIRIRKHD; FCT-2_full
270:
MKQTLKLMFSFLLMLGTMFGISQTVLAQETHQLTIVHLEARDIDRPNPQLEIAPKEGTP
IEGVLYQLYQLKSTEDGDLLAHWNSLTITELKKQAQQVFEATTNQQGKATFNQLPDG
IYYGLAVKAGEKNRNVSAFLVDLSEDKVIYPKIIWSTGELDLLKVGVDGDTKKPLAG
VVFELYEKNGRTPIRVKNGVHSQDIDAAKHLETDSSGHIRISGLIHGDYVLKEIETQSG
YQIGQAETAVTIEKSKTVTVTIENKKVPTPKVPSRGGLIPKTGEQQAMALVIIGGILIAL
ALRLLSKHRKHQNKD; GBS52_Q8E0S8
271:
MLNRETHMKKVRKIFQKAVAGLCCISQLTAFSSIVALAETPETSPAIGKVVIKETGEGG
ALLGDAVFELKNNTDGTTVSQRTEAQTGEAIFSNIKPGTYTLTEAQPPVGYKPSTKQW
TVEVEKNGRTTVQGEQVENREEALSDQYPQTGTYPDVQTPYQIIKVDGSEKNGQHKA
LNPNPYERVIPEGTLSKRIYQVNNLDDNQYGIELTVSGKTVYEQKDKSVPLDVVILLD
NSNSMSNIRNKNARRAERAGEATRSLIDKITSDSENRVALVTYASTIFDGTEFTVEKGV
ADKNGKRENDSLFWNYDQTSFTTNTKDYSYLKLINDKNDIVELKNKVPTEAEDHDG
NRLMYQFGATFTQKALMKADEILTQQARQNSQKVIFHITDGVPTMSYPINFNHATFAP
SYQNQLNAFFSKSPNKDGILLSDFITQATSGEHTIVRGDGQSYQMFTDKTVYEKGAPA
AFPVKPEKYSEMKAAGYAVIGDPINGGYIWLNWRESILAYPENSNTAKITNHGDPTR
WYYNGNIAPDGYDVFTVGIGINGDPGTDEATATSFMQSISSKPENYTNVTDTTKILEQ
LNRYFHTIVTEKKSIENGTITDPMGELIDLQLGTDGRFDPADYTLTANDGSRLENGQA
VGGPQNDGGLLKNAKVLYDTTEKRIRVTGLYLGTDEKVTLTYNVRLNDEFVSNKFY
DTNGRTTLHPKEVEQNTVRDFPIPKIRDVRKYPEITISKEKKLGDIEFIKVNKNDKKPLR
GAVFSLQKQHPDYPDIYGAIDQNGTYQNVRTGEDGKLTFKNLSDGKYRLFENSEPAG
YKPVQNKPIVAFQIVNGEVRDVTSIVPQDIPAGYEFTNDKHYITNEPIPPKREYPRTGGI
GMLPFYLIGCMMMGGVLLYTRKHP; Rrga_AAK74622.1
272:
MKSINKFLTMLAALLLTASSLFSAATVFAAGTTTTSVTVHKLLATDGDMDKIANELET
GNYAGNKVGVLPANAKEIAGVMFVWTNTNNEIIDENGQTLGVNIDPQTFKLSGAMP
ATAMKKLTEAEGAKFNTANLPAAKYKIYEIHSLSTYVGEDGATLTGSKAVPIEIELPL
NDVVDAHVYPKNTEAKPKIDKDFKGKANPDTPRVDKDTPVNHQVGDVVEYEIVTKIP
ALANYATANWSDRMTEGLAFNKGTVKVTVDDVALEAGDYALTEVATGFDLKLTDA
GLAKVNDQNAEKTVKITYSATLNDKAIVEVPESNDVTFNYGNNPDHGNTPKPNKPNE
NGDLTLTKTWVDATGAPIPAGAEATFDLVNAQTGKVVQTVTLTTDKNTVTVNGLDK
NTEYKFVERSIKGYSADYQEITTAGEIAVKNWKDENPKPLDPTEPKVVTYGKKFVKV
NDKDNRLAGAEFVIANADNAGQYLARKADKVSQEEKQLVVTTKDALDRAVAAYNA
LTAQQQTQQEKEKVDKAQAAYNAAVIAANNAFEWVADKDNENVVKLVSDAQGRFE
ITGLLAGTYYLEETKQPAGYALLTSRQKFEVTATSYSATGQGIEYTAGSGKDDATKV
VNKKITIPQTGGIGTIIFAVAGAAIMGIAVYAYVKNNKDEDQLA;
Rrgb_WP_000836217.1
273:
MTMQKMQKMISRIFFVMALCFSLVWGAHAVQAQEDHTLVLQLENYQEVVSQLPSRD
GHRLQVWKLDDSYSYDDRVQIVRDLHSWDENKLSSFKKTSFEMTFLENQIEVSHIPN
GLYYVRSIIQTDAVSYPAEFLFEMTDQTVEPLVIVAKKTDTMTTKVKLIKVDQDHNRL
EGVGFKLVSVARDVSEKEVPLIGEYRYSSSGQVGRTLYTDKNGEIFVTNLPLGNYRFK
EVEPLAGYAVTTLDTDVQLVDHQLVTITVVNQKLPRGNVDFMKVDGRTNTSLQGAM
FKVMKEESGHYTPVLQNGKEVVVTSGKDGRFRVEGLEYGTYYLWELQAPTGYVQLT
SPVSFTIGKDTRKELVTVVKNNKRPRIDVPDTGEETLYILMLVAILLFGSGYYLTKKPN
N; Rrgc_WP_000178714.1
274:
MNFGFTRHRSQLSHAALPAVLFLAITSTAITTPVDATPITSTNIDTVVEDAAENPPLDQE
SAALPTEVTDDNLQHVKLIITNNMISAGFATIERKNGESEFYGHDQVLIDGTEVPDSSV
YSAPATENQGDLITINFAGLSIQSGQTISFSYRSSATSQNDTWLPSEANAFKYLAAPAES
AIANTDEREAQSFLQGNFELSMSVSPALVQVGQPVTYTYTFKNTSSRYRLFWSNYAD
GAKGVIEDRLNLNDDVKCKWDEGNGWLKSKTGQRYIDINSEATFSCVRTFEHVGNY
TNSVNIKEAKQRTGPLNGQIGLTLAPTTIDDALSVKVVNVVNSQTDPQWSLTISSSKFY
VDPSGEDVTYTYTVTNLSNDKIYYEALKHDVCSPIKIENSLQFDPENRKYYIPQNGTAT
WECATRINHETTGLVSGTFSDNKGNRSTVKASTQTKVKTPTLSNGTSYGIPRCDVIDF
TTVNKSTGIGTLGSIEQQNGQFKKIEQSNIFPEKGSPAHHDRRIKKKGRMTTASATSAQ
HPEYVYYAALALGDSISISSDANMGIYRIHKISGTVEKITAPHFSQSALQNNQRFGATL
TNRLAFDATGKLWSFAQDGHLYSLPMDGDGKAAGEWFDHGAVAGEAINGEGNAVG
FESLVFGDIAFDGNGAMWILGSIRGLTKEDTNGRVIKDEVDPTTYLFTLKPPRDNTPIT
EKVQIVQKITGVGTSIDQKGFFGLAFGVDGTLYGSYDTSGDGISDSPGELYSFNLRDG
KVTKVFSSPLMARVQDLSSCAFPAPRISAEKTAAHEVDKDTITYTITVRNSGNLEATGT
KFTDNLPGSYVPNSAKLNGKPIPDLPVNSTNPTGNPFHDGLYIKSPDAAPGTIDPQSEA
VIEMTINKLNSTNDGRVCNQAEINAVGQQVKTDDPTLPGHEDPTCVSVPISLKMSLKK
AIYDPSAQSPKILDNLGGAKFAIYARTDTGDLGELRKEVSDEEPFEISPGTYLLVETQSP
AGLSLLPKPVEFTITKSSSGFDVKSNSPLTVSFTKTDGIIVATVSDVKNGTLPKTGSTGF
LPFVFVGLSIIVLTALWVQRRSQYKL; SpaA_A0A5E5PJF1
275:
MKVKKTYGFRKSKISKTLCGAVLGTVAAVSVAGQKVFADETTTTSDVDTKVVGTQT
GNPATNLPEAQGSASKEAEQSQNQAGETNGSIPVEVPKTDLDQAAKDAKSAGVNVV
QDADVNKGTVKTABEAVQKETEIKEDYTKQAEDIKKTTDQYKSDVAAHEAEVAKIK
AKNQATKEQYEKDMAAHKAEVERINAANAASKTAYEAKLAQYQADLAAVQKTNA
ANQAAYQKALAAYQAELKRVQEANAAAKAAYDTAVAANNAKNTEIAAANEEIRKR
NATAKAEYETKLAQYQAELKRVQEANVANEADYQAKLTAYQTELARVQKANADA
KAAYEAAVAANNAKNAALTAENTAIKQRNENAKATYEAALKQYEADLAAAKKAN
AANEADYQAKLTAYQTELARVQKANADAKAAYEAAVAANNAANAALTAENTAIK
KRNADAKADYEAKLAKYQADLAKYQKDLADYPVKLKAYEDEQASIKAALAELEKH
KNEDGNLTEPSAQNLVYDLEPNANLSLTTDGKFLKASAVDDAFSKSTSKAKYDQKIL
QLDDLDITNLEQSNDVASSMELYGNFGDKAGWSTTVSNNSQVKWGSVLLERGQSAT
ATYTNLQNSYYNGKKISKIVYKYTVDPKSKFQGQKVWLGIFTDPTLGVFASAYTGQV
EKNTSIFIKNEFTFYDEDGKPINFDNALLSVASLNREHNSIEMAKDYSGKFVKISGSSIG
EKNGMIYATDTLNFKQGEGGSRWTMYKNSQAGSGWDSSDAPNSWYGAGAIKMSGP
NNHVTVGATSATNVMPVSDMPVVPGKDNTDGKKPNIWYSLNGKIRAVNVPKVTKE
KPTPPVKPTAPTKPTYETEKPLKPAPVAPNYEKEPTPPTRTPDQAEPNKPTPPTYETEKP
LEPAPVEPSYEAEPTPPTRTPDQAEPNKPTPPTYETEKPLEPAPVEPSYEAEPTPPTPTPD
QPEPNKPVEPTYEVIPTPPTDPVYQDLPTPPSVPTVHFHYFKLAVQPQVNKEIRNNNDV
NIDRTLVAKQSVVKFQLKTADLPAGRDETTSFVLVDPLPSGYQFNPEATKAASPGFDV
AYDNATNTVTFKATAATLATFNADLTKSVATIYPTVVGQVLNDGATYKNNFTLTVN
DAYGIKSNVVRVTTPGKPNDPDNPNNNYIKPTKVNKNENGVVIDGKTVLAGSTNYYE
LTWDLDQYKNDRSSADTIQKGFYYVDDYPEEALELRQDLVKITDANGNEVTGVSVD
NYTSLEAAPQEIRDVLSKAGIRPKGAFQIFRADNPREFYDTYVKTGIDLKIVSPMVVKK
QMGQTGGSYENQAYQIDFGNGYASNIVINNVPKINPKKDVTLTLDPADTNNVDGQTI
PLNTVFNYRLIGGIIPANHSEELFEYNFYDDYDQTGDHYTGQYKVFAKVDITLKNGVII
KSGTELTQYTTAEVDTTKGAITIKFKEAFLRSVSIDSAFQAESYIQMKRIAVGTFENTYI
NTVNGVTYSSNTVKTTTPEDPTDPTDPQDPSSPRTSTVINYKPQSTAYQPSSVQETLLN
TGVTNNAYMPLLGIIGLVTSFSLLGLKAKKD; SpaP_BAF91892.1
276:
MKLRHLLLTGAALTSFAATTVHGETVVNGAKLTVTKNLDLVNSNALIPNTDFTFKIEP
DTTVNEDGNKFKGVALNTPMTKVTYTNSDKGGSNTKTAEFDFSEVTFEKPGVYYYK
VTEEKIDKVPGVSYDTTSYTVQVHVLWNEEQQKPVATYIVGYKEGSKVPIQFKNSLD
STTLTVKKKVSGTGGDRSKDFNFGLTLKANQYYKASEKVMIEKTTKGGQAPVQTEAS
IDQLYHFTLKDGESIKVTNLPVGVDYVVTEDDYKSEKYTINVEVSPQDGAVKNIAGN
STEQETSTDKDMTITFTNKKDFEVPTGVAMTVAPYIALGIVAVGGALYFVKKKNA;
Spy0128
277:
MKNKKEVYGFRKSKVAKTLCGAVLGTALIAFADKAVFADEVTETTSTSTVEVATTG
NPATNLAEAQGDMSQAAKESQAKAGSKDSALPVEVSSADLDKAVADAKTAGVKVV
QDETKDKGTATTATENAQKQDEIKSDYAKQAEEIKTSTEAYKKAAATHQAETDKINA
ENKAADDKYQKDLKSHQEEVEKINTANATAKAEYEAKLAQYQKDLATVKKANEDS
QQDYQNKLSAYQTELARVQKANAEAKEAYEKAVKENTEKNEALQAENEAIKQRNET
AKANYDAAMKQYEADLAAIKKAKEDNDADYQAKLAAYQTELARVQKANADAKAA
YEKAVEENTAKNNAIQAENEAIKQRNATAKSTYDAAMKKYEADLVAVKQANATNE
TDYQTKLAAYQTELARVQKANADAKAAYEKAVEDNKAKNAALKAENEEIKQRNAV
AKTDYEAKLAKYEADLAKYKKEFAAYTAALAEAESKKKQDGYLSEPRSQSLNFKSE
PNAIRTIDPSVHQYGQQELDALVKSWGISPTNPDRTKSTAYSYFNAINSNNTYAKLVL
EKDKPVDVTYTGLKNSSFNGKKISKVVYTYTLKETGENDGTKMTMFASSDPTVTAW
YNDYFTSTNINVKVKFYDEEGQLMNLTGGLVNFSSLNRGNGSGAIDKDAIESVRNFN
GRYIPISGSSIKIHENNSAYADSSNAEKSLGARWNTSEWDTTSSPNNWYGAIVGEITQS
EISENMASSKSGNIWFAFNSNINAIGVPTKPVAPTAPTQPMYETEKPLEPAPVAPTYEN
EPTPPVKTPDQPEPSKPEEPKYETEKPLEPAPVAPSYENEPTPPEL; Sspb_EUC80876.1
*capable of intramolecular isopeptide bond formation from which isopeptide domain and isopeptide tag can isolated for the purpose of making fusion protein, peptide or conjugate that can participate in intermolecular isopeptide bond formation

TABLE 12
Signal Sequences

SEQ ID NO: Sequence; Source
278:
ATGTGGTGGCGACTCTGGTGGCTCCTTCTTCTGCTCCTTCTCCTTTGGCCAATGGTG
TGGGCC; Signal sequence-Coding sequence from secreted alkaline phosphatase [synthetic
construct] Sequence ID: BBD75655.1; Artificial Sequence
279: MWWRLWWLLLLLLLLWPMVWA; Signal sequence-Peptide sequence from
secreted alkaline phosphatase [synthetic construct] Sequence ID: BBD75655.1; Artificial
Sequence
280:
ATGGAAACGGATACGTTGCTGCTCTGGGTCCTGCTTCTTTGGGTTCCCGGGTCAAC
TGGTGAT; Signal sequence-Coding sequence from Igk protein [Mus musculus] Sequence
ID: AAH80787.1
281: METDTLLLWVLLLWVPGSTGD; Signal sequence-Peptide sequence from Igk protein
[Mus musculus] Sequence ID: AAH80787.1
282:
ATGGCAGTGGGGGCCAGTGGTCTAGAAGGAGATAAGATGGCTGGTGCCATGCCTC
TGCAACTCCTCCTGTTGCTGATCCTACTGGGCCCTGGCAACAGC; Signal sequence-
Coding sequence from NCBI Reference Sequence: NP_001193538.1; CCDS55881.1
283: MAVGASGLEGDKMAGAMPLQLLLLLILLGPGNS; Signal sequence-Peptide
sequence from NCBI Reference Sequence: NP_001193538.1
284:
ATGGAATCCAAGGGGGCCAGTTCCTGCCGTCTGCTCTTCTGCCTCTTGATCTCCGC
CACCGTCTTCAGGCCAGGCCTTGGA; Signal sequence-Coding sequence from NCBI
Reference Sequence: NP_001618.2; CCDS33810.1
285: MESKGASSCRLLFCLLISATVFRPGLG; Signal sequence-Peptide sequence from
NCBI Reference Sequence: NP_001618.2
286:
ATGGTCCTCCTTTGGCTCACGCTGCTCCTGATCGCCCTGCCCTGTCTCCTGCAAAC
G; Signal sequence-Coding sequence from NCBI Reference Sequence: XP_005274837.1;
CCDS59158.1
287: MVLLWLTLLLIALPCLLQT; Signal sequence-Peptide sequence from NCBI
Reference Sequence: XP 005274837.1

TABLE 13
Nucleic Acid Payload

| Class of payload | Payload details | Target |
|---|---|---|
| anti-miRNA | antimiR-494 | Targets the “oncomiR”, miR-494 |
| | | miRNA |
| anti-miRNA | antimiR-221/222 | Targets the “oncomiR”, miR-221/222 |
| | | miRNA |
| anti-miRNA | antimiR-132 | Targets the “oncomiR”, miR-132 |
| | | miRNA |
| anti-miRNA | antimiR-155 | Targets the “oncomiR”, miR-155 |
| | | miRNA |
| Antisense | ASO, OGX-011 | Clusterin |
| Oligonucleotide (ASO) | | |
| Antisense | EGFR antisense DNA | EGFR |
| Oligonucleotide (ASO) | | |
| Antisense | ASO, OGX-427 | Hsp27 |
| Oligonucleotide (ASO) | | |
| Antisense | ASO, ISIS-STAT3Rx | STAT3 |
| Oligonucleotide (ASO) | | |
| Antisense | ASO, AP 12009 | TGFB2 |
| Oligonucleotide (ASO) | | |
| Antisense | ASO, EZN-2968 | HIF-1a |
| Oligonucleotide (ASO) | | |
| Antisense | ASO, LErafAON-ETU | c-raf |
| Oligonucleotide (ASO) | | |
| Antisense | ASO, K-Ras mutation | Mutated K-Ras |
| Oligonucleotide (ASO) | matched | |
| Antisense | ASO, Wnt/beta-catenin | WNT/beta-catenin signaling |
| Oligonucleotide (ASO) | | |
| Antisense | ASO, myc | Estrogen induced c-myc expression |
| Oligonucleotide (ASO) | | |
| Antisense | ASO, Raf1 | Raf-1 |
| Oligonucleotide (ASO) | | |
| Aptamer | DNA Aptamer, AS1411 | Nucleolin |
| Aptamer | RNA Apatamer, NOX- | CXCL12/SDF-1 (CXC chemokine |
| | A12 | ligand 12/stromal cell derived factor-1) |
| CRISPR/Cas9 | CRISPR/Cas9 | E6, E7 HPV oncogenes |
| CRISPR/Cas9 | CRISPR/Cas9 | EBV genome, EBNA1 |
| CRISPR/Cas9 | CRISPR/Cas9 under an | sgRNA to LacI gene, only in the |
| | AND logic gate | presence of the cancer-specific human |
| | | telomerase reverse transcriptase |
| | | promoter and urothelium-specific |
| | | human uroplakin II promoter (AND |
| | | logic gate, both promotors only present |
| | | in bladder cancer cells). |
| Cytotoxic trans-genes | Herpes Simplex Type 1 | Converts the prodrug ganciclovir (or |
| | thymidine kinase (TK) | valacyclovir) into the highly toxic |
| | | deoxyguanosine triphosphate causing |
| | | early chain termination of nascent DNA |
| | | strands |
| miRNA | miRNA-34a | Poorly understood tumor suppressor |
| | | gene. Targets include SIRT1, BCL2, |
| | | YY1, MYC, CDK6, CCND1, FOXP1, |
| | | HNF4a, CDKN2C, ACSL4, LEF1, |
| | | ACSL1, MTA2, AXL, LDHA, HDAC1, |
| | | CD44, BCL2, E2F3 |
| miRNA | miR-200 | Poorly understood tumor suppressor |
| | | gene. Targets include ZEB1, CTNNB1, |
| | | BAP1, GEMIN2, PTPRD, WDR37, |
| | | KLF11, SEPT9, HOXB5, ERBB2IP. |
| | | KLHL20, FOG2, RIN2, RASSF2, |
| | | ELMO2, TCF7L1, VAC14, SHC1, |
| | | SEPT7, FOG2 |
| miRNA | miR-15/16 | Poorly understood tumor suppressor |
| | | gene. Targets include BACE1, DMTF1, |
| | | C22orf5, BCL2, ARL2, CCNT2, |
| | | TPPP3, VEGFA, RARS, FGF2, |
| | | ZNF622, DNAJB4, PURA, SHOC2, |
| | | LUZP1, FNDC3B, ITGA2, ATG9A, |
| | | CA12, TMEM43, YIF1B, TMEM189, |
| | | VTI1B, RTN4, TOMM34, NAA15, |
| | | PNP, SRPR, IPO4, NAPg, PFAH1B2, |
| | | SLC12A2, SEC24A, NOTCH2, |
| | | PPP2RSC, KCNN4, UBE4A, KPNA3, |
| | | RAB30, ACP2, SRPRB, EIF4E, |
| | | ABCF2, TPM3, ARHGDIA, GALNT7, |
| | | LYPLA2, CHORDC1, TMEM109, |
| | | LAMC1, EGFR, GPAM, ADSS, PPIF, |
| | | RFT1, TNFSF9, IGF2R, TXN2, |
| | | GFPT1, SLC7A1, SQSTM1, PANX1, |
| | | UTP15, NPR3, SLC16A3, PTGS2, |
| | | HARS, LAMTOR3, HSPA1B |
| miRNA | let-7 | Poorly understood tumor suppressor |
| | | gene. Targets include NIRF, NF2, |
| | | CASP3, TRIM71 |
| miRNA | miR-26a | Induces cell-cycle arrest associated with |
| | | direct targeting of cyclins D2 and E2 |
| miRNA | miR-143 | MACC1 |
| miRNA | miR-145; miR-33a | ERK5, c-Myc |
| mRNA | mRNAs encoding | OX40L, IL-36γ, and IL-23 |
| | OX40L, IL-36γ, and | |
| | IL-23 | |
| siRNA | siRNA against targets | Knockdown c-Myc/MDM2/VEGF |
| siRNA | siRNA against targets | EphA2 oncoprotein |
| siRNA | siRNA against targets | Oncogenic KRAS(G12D) |
| siRNA | siRNA against targets | PLK1 (polo-like kinase-1) |
| siRNA | siRNA against targets | protein kinase N3 (PKN3) gene |
| | | expression in vascular endothelial cells |
| siRNA | siRNA against targets | VEGF gene, kinesin spindle (KSP) |
| | | protein gene |
| Splice-switching | SSO to Bcl-x | Apoptotic regulator Bcl-x is |
| oligonucleotides (SSOs) | | alternatively spliced to express anti- |
| | | apoptotic Bcl-xL and pro-apoptotic Bcl- |
| | | xS |
| Splice-switching | SSO, SSO111 | HER2 Exon 15, transmembrane |
| oligonucleotides (SSOs) | | domain. |
| Transgene encoding | Pseudomonas exotoxin | IL12 variant, IL13Rα2, common in |
| toxic proteins | encoded transgene | GBM |
| | connected to human IL- | |
| | 13. 50-80% of human | |
| | GBM cells overexpress | |
| | a variant of the IL-13 | |
| | receptor not found in | |
| | normal tissue. | |

1. An extracellular vesicle comprising: a vesicle localization moiety and one or more isopeptide domain(s), wherein the isopeptide domain is fused to the vesicle localization moiety so that the isopeptide domain is displayed by the vesicle localization moiety on the outside of the extracellular vesicle, whereby the isopeptide domain can form a covalent bond with an isopeptide tag.

2. The extracellular vesicle of claim 1, wherein the vesicle localization moiety is CLSTN1, IL3RA, ITGB1, SELPLG, LAMP2B or PTGFRN, or a variant thereof and/or a fragment thereof.

3. The extracellular vesicle of claim 1, wherein the vesicle localization moiety is a chimeric vesicle localization moiety comprising a surface-and-transmembrane domain of a first vesicle localization moiety and a cytosolic domain of a second vesicle localization moiety.

4. The extracellular vesicle of claim 1, wherein the isopeptide domain is selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, and 60.

5. The extracellular vesicle of claim 3, wherein the first and second vesicle localization moieties are from different or unrelated proteins and wherein the first or second vesicle localization moiety is selected from the group consisting of CLSTN1, IL3RA, ITGB1, LAMP2B, PTGFRN, and SELPLG, or a variant thereof and/or a fragment thereof.

6. The extracellular vesicle of claim 1, further comprising a spacer polypeptide wherein the spacer polypeptide is located between the isopeptide domain and the vesicle localization moiety.

7. The extracellular vesicle of claim 1, wherein the vesicle localization moiety comprises two isopeptide domains.

8. The extracellular vesicle of claim 7, further comprising a spacer polypeptide located between the two isopeptide domains.

9. The extracellular vesicle of claim 1, wherein the isopeptide domain is located at a N-terminus of the vesicle localization moiety.

10. The extracellular vesicle of claim 1, wherein isopeptide domain is located at N-terminal to a transmembrane domain of the vesicle localization moiety.

11. The extracellular vesicle of claim 1, further comprising a second isopeptide domain.

12. The extracellular vesicle of claim 11, wherein the first isopeptide domain is a SEQ ID NO: 32, and the second isopeptide domain is a SEQ ID NO: 58.

13. The extracellular vesicle of claim 1, further comprising a second and third isopeptide domains.

14. The extracellular vesicle of claim 13, wherein the first isopeptide domain is a SEQ ID NO: 32, the second domain is a SEQ ID NO: 58, and the third domain is a SEQ ID NO: 60.

15.-33. (canceled)

34. A nucleic acid encoding a vesicle localization moiety and an isopeptide domain, wherein the isopeptide domain is fused to the vesicle localization moiety so that the isopeptide domain is displayed by the vesicle localization moiety on the outside of an extracellular vesicle.

35.-42. (canceled)

43. A cell comprising the nucleic acid of claim 34.

44.-52. (canceled)

53. A method for making the vesicle of claim 1 comprising: expressing a nucleic acid encoding a vesicle localization moiety and an isopeptide domain, wherein the isopeptide domain is fused to the vesicle localization moiety so that the isopeptide domain is displayed by the vesicle localization moiety on the outside of an extracellular vesicle in a producer cell; and isolating a vesicle secreted into a culture medium by the producer cell.

54. A pharmaceutical composition comprising the vesicle of claim 1, and one or more pharmaceutically acceptable excipients.

55.-57. (canceled)

58. An extracellular vesicle comprising: a vesicle localization moiety and one or more isopeptide tag, wherein the isopeptide tag is fused to the vesicle localization moiety so that the isopeptide tag is displayed by the vesicle localization moiety on the outside of the extracellular vesicle, whereby the isopeptide tag can form a covalent bond with an isopeptide domain.

59.-122. (canceled)

123. The extracellular vesicle of claim 5, wherein the surface-and-transmembrane domain of a first vesicle localization moiety is the surface-and-transmembrane domain selected from the group consisting of LAM P2 and CLSTN1, or a homologue thereof.

124. The extracellular vesicle of claim 5, wherein the cytosolic domain of the second vesicle localization moiety is the cytosolic domain selected from the group consisting of PTGFRN, ITGA3, IL3RA, SELPLG, ITGB1 and CLSTN1, or a homologue thereof.
